(19) United States
(12) Patent Application Publication
(10) Pub. No.: US 2023/0422617 A1
(43) Pub. Date: Dec. 28, 2023

(54) ORGANIC ELECTROLUMINESCENT DEVICE AND DISPLAY APPARATUS

(71) Applicants: KunShan Go-Visionox Opto-Electronics Co., Ltd, Kunshan (CN); Tsinghua University, Beijing (CN)

(72) Inventors: Guomeng LI, Kunshan (CN); Lian DUAN, Kunshan (CN); Yuewei ZHANG, Kunshan (CN); Dongdong ZHANG, Kunshan (CN); Minghan CAI, Kunshan (CN); Baoyu LI, Kunshan (CN); Mengzhen LI, Kunshan (CN)

(73) Assignees: KunShan Go-Visionox Opto-Electronics Co., Ltd, Kunshan (CN); Tsinghua University, Beijing (CN)

(21) Appl. No.: 18/466,253

(22) Filed: Sep. 13, 2023

Related U.S. Application Data
(63) Continuation of application No. PCT/CN2022/107504, filed on Jul. 22, 2022.

(30) Foreign Application Priority Data
Nov. 26, 2021 (CN) 202111423658.X

(51) Int. Cl. H10K 85/60; H10K 85/40; H10K 85/20; C09K 11/06; C09K 11/02

(52) U.S. Cl. CPC H10K 85/658; H10K 85/6572; H10K 85/633; H10K 85/40; H10K 85/654; H10K 85/615; H10K 85/20; H10K 85/6574; H10K 85/6576; H10K 85/622; C09K 11/06; C09K 11/02; C07B 2200/05

(57) ABSTRACT

An organic electroluminescent device and a display apparatus. The organic electroluminescent device includes a light-emitting layer, including a triplet-triplet annihilation type host and a fluorescent dye, where the fluorescent dye has a structure represented by the following Formula (1) or Formula (2). When the fluorescent dye in the light-emitting layer of the organic electroluminescent device combines with the triplet-triplet annihilation type host, the voltage of the device can be reduced, and the luminescence efficiency of the device can be improved.

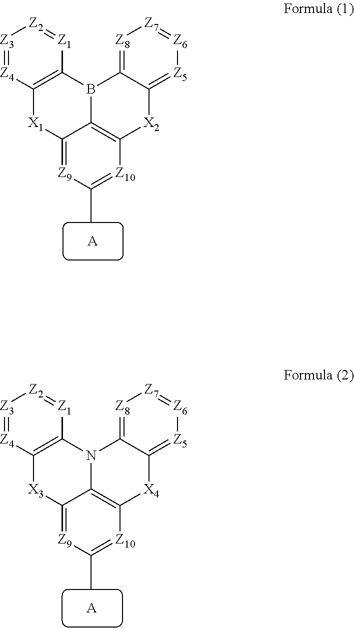

### CROSS-REFERENCE TO RELATED APPLICATIONS

The present application is a continuation of International Application No. PCT/CN2022/107504, filed on Jul. 22, 2022, which claims priority to Chinese Patent Application No. 202111423658.X, filed with the China National Intellectual Property Administration (CNIPA) on Nov. 26, 2021, entitled with “ORGANIC ELECTROLUMINESCENT DEVICE AND DISPLAY APPARATUS”. Both of the above applications are hereby incorporated by reference in their entireties.

### TECHNICAL FIELD

The present application relates to an organic electroluminescent device and a display apparatus, belonging to the field of organic electroluminescent technologies.

### BACKGROUND

Organic Light Emitting Diode (OLED) is a device that achieves a purpose of emitting light by current driving. Its main characteristics are derived from an organic light-emitting layer. When applying an appropriate voltage, electrons and holes are combined in the organic light-emitting layer to produce excitons and emit light with different wavelengths based on the characteristics of the organic light-emitting layer.

Currently, the light-emitting layer is composed of a host material and a dye, and the dye is mostly selected from a traditional fluorescent material and a traditional phosphorescent material. Among them, although the traditional phosphorescent material has high efficiency, it is expensive and has poor stability; while the traditional fluorescent material has extremely low efficiency although it is cheap. The existing display apparatuses still have problems of low efficiency, high driving voltage, etc.

In recent years, the Multi-Resonance (MR) material with the high efficiency and narrow-band emission has attracted widespread attention from the scientific and industrial communities. Although this type of material has a certain degree of promoting effect on the device performance relative to the traditional fluorescent material and the traditional phosphorescent material. However, this type of material is difficult to fully utilize the energy between the host and guest under low concentration conditions, and decreased efficiency and other issues will be caused by further increasing the concentration. The evaporation window is relatively narrow, and the process requirements are complex.

### SUMMARY

The present application provides an organic electroluminescent device and a display apparatus with high luminescence efficiency and high stability of driving voltage.

The present application provides an organic electroluminescent device including a light-emitting layer, the light-emitting layer includes a triplet-triplet annihilation type host and a fluorescent dye, where the fluorescent dye has a structure represented by the following Formula (1) or Formula (2):

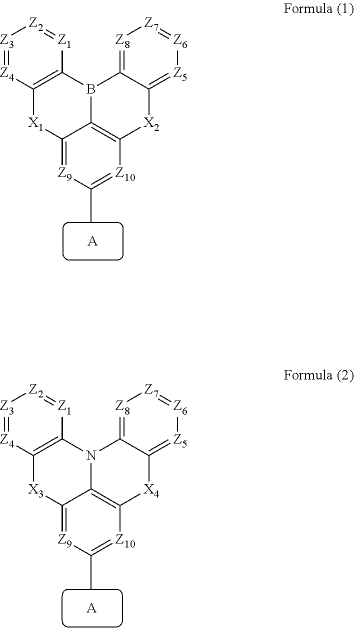

in Formula (1), X₁ and X₂ are each independently represented as O, S, or N(R₁).
in Formula (2), X₃ and X₄ are each independently represented as B(R₂) or C(═O);
in Formula (1) or (2), A represents one of a substituted or unsubstituted C₆-C₆₀ carbocyclic group, and a substituted or unsubstituted C₃-C₆₀ heterocyclyl; a substituted group in A is one or a combination of at least two selected from deuterium, tritium, cyano, halogen, C₁-C₁₀ alkyl, C₃-C₁₀ cycloalkyl, silyl, C₆-C₃₀ arylamino, C₆-C₃₀ aryl, and C₂-C₃₀ heteroaryl, and the substituted group is independently connected to a connected aromatic ring or heteroaromatic ring to form a ring or not to form a ring;
in Formula (1) or (2), Z₁-Z₁₀ are each independently represented as N or CR, with R being the same or different each time, two adjacent R can bond to each other to form a ring;
R₁ is connected to adjacent R through a single bond, while R₂ is connected to adjacent R through a single bond;
R₁ represents one of a substituted or unsubstituted C₁-C₁₀ alkyl, a substituted or unsubstituted C₃-C₁₀ cycloalkyl, a substituted or unsubstituted C₆-C₃₀ aryl, and a substituted or unsubstituted C₂-C₃₀ heteroaryl;
R₂ represents one of a substituted or unsubstituted C₆-C₃₀ aryl, and a substituted or unsubstituted C₂-C₃₀ heteroaryl;
R represents one of hydrogen, deuterium, tritium, cyano, halogen, a substituted or unsubstituted C₁-C₁₀ alkyl, a substituted or unsubstituted C₃-C₁₀ cycloalkyl, a substituted or unsubstituted C₁-C₁₀ alkoxy, a substituted or unsubstituted C₆-C₃₀ aryloxy, a substituted or unsubstituted C₆-C₃₀ arylamino, a substituted or unsubstituted C₆-C₃₀ aryl, and a substituted or unsubstituted C₂-C₃₀ heteroaryl;
a substituted group in the each above substituted R₁, R₂, and R is one or a combination of at least two selected from deuterium, tritium, cyano, halogen, C₁-C₁₀ alkyl, C₃-C₁₀ cycloalkyl, silyl, C₆-C₃₀ arylamino, C₆-C₃₀ aryl, and C₂-C₃₀ heteroaryl; and the substituted group is independently connected to the connected aromatic ring or heteroaromatic ring to form a ring or not to form a ring.

The present application further provides a display apparatus, including the above organic electroluminescent device.

In the organic electroluminescent device of the present application, the light-emitting layer includes a triplet-triplet annihilation material and a fluorescent dye with a structure represented by Formula (1) or Formula (2). Among them, the triplet-triplet annihilation material with a triplet state annihilation effect and a lower triplet state energy level combined with the fluorescent dye with a reverse intersystem crossing property, can realize a highly efficient utilization of excitons in the system and reduce the concentration of triplet excitons in the system, achieve the improvement of luminescence efficiency and driving voltage of the device, and inhibit the roll-off of efficiency. In addition, the fluorescent dye of the present application can effectively suppress an intermolecular interaction of a planar multi-resonance compound, and inhibit the Dexter energy transfer between host and guest materials in the light-emitting layer and the influence of intermolecular interactions, further achieving the improvement of luminescence efficiency and driving voltage of the device.

### DETAILED DESCRIPTION OF EMBODIMENTS

In order to make the purpose, technical solution and advantages of the present application clearer, the following will provide a clear and complete description of the technical solution in the embodiments of the present application, with reference to the embodiments of the present application. Obviously, the described embodiments are a part of the embodiments of the present application, not all of them. Based on the embodiments in the present application, all other embodiments obtained by ordinary technical personnel in the art without creative work fall within the protection scope of the present application.

The present application provides an organic electroluminescent device including a light-emitting layer, the light-emitting layer includes a triplet-triplet annihilation type host and a fluorescent dye, where the fluorescent dye has a structure represented by the following Formula (1) or Formula (2):

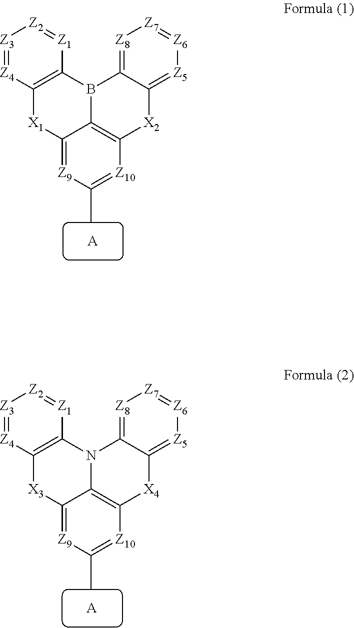

in Formula (1), X₁ and X₂ are each independently represented as O, S, or N(R₁);
in Formula (2), X₃ and X₄ are each independently represented as B(R₂) or C(═O);
in Formula (1) or (2), A represents one of a substituted or unsubstituted C₆-C₆₀ carbocyclic group, and a substituted or unsubstituted C₃-C₆₀ heterocyclyl; a substituted group in A is one or a combination of at least two selected from deuterium, tritium, cyano, halogen, C₁-C₁₀ alkyl, C₃-C₁₀ cycloalkyl, silyl, C₆-C₃₀ arylamino, C₆-C₃₀ aryl, and C₂-C₃₀ heteroaryl, and the substituted group is independently connected to a connected aromatic ring or heteroaromatic ring to form a ring or not to form a ring;
in Formula (1) or (2), Z₁-Z₁₀ are each independently represented as N or CR, with R being the same or different each time, two adjacent R can bond to each other to form a ring;
R₁ is connected to adjacent R through a single bond, while R₂ is connected to adjacent R through a single bond;
R₁ represents one of a substituted or unsubstituted C₁-C₁₀ alkyl, a substituted or unsubstituted C₃-C₁₀ cycloalkyl, a substituted or unsubstituted C₆-C₃₀ aryl, and a substituted or unsubstituted C₂-C₃₀ heteroaryl;
R₂ represents one of a substituted or unsubstituted C₆-C₃₀ aryl, and a substituted or unsubstituted C₂-C₃₀ heteroaryl;

The expression “R being the same or different each time” means that when at least two of Z₁-Z₁₀ are selected from CR, R in any two CRs are the same or different. Exemplarily, R represents one of hydrogen, deuterium, tritium, cyano, halogen, a substituted or unsubstituted C₁-C₁₀ alkyl, a substituted or unsubstituted C₃-C₁₀ cycloalkyl, a substituted or unsubstituted C₁-C₁₀ alkoxy, a substituted or unsubstituted C₆-C₃₀ aryloxy, a substituted or unsubstituted C₆-C₃₀ arylamino, a substituted or unsubstituted C₆-C₃₀ aryl, and a substituted or unsubstituted C₂-C₃₀ heteroaryl;
a substituted group in the each above substituted R₁, R₂, and R is one or a combination of at least two selected from deuterium, tritium, cyano, halogen, C₁-C₁₀ alkyl, C₃-C₁₀ cycloalkyl, silyl, C₆-C₃₀ arylamino, C₆-C₃₀ aryl, and C₂-C₃₀ heteroaryl; and the substituted group is independently connected to the connected aromatic ring or heteroaromatic ring to form a ring or not to form a ring.

It should be noted that in the present application, the expression of Ca-Cb represents that the carbon atom number of the group is a-b. Unless otherwise specified, the carbon atom number generally does not include the carbon atom number of the substituted group. In the present application, the expression of chemical elements, unless otherwise specified, generally includes the concept of isotopes with same chemical properties. For example, the expression of “hydrogen” also includes the concepts of “deuterium” and “tritium” with same chemical properties; and carbon (C) includes ¹²C, ¹³C, etc., which will not be repeated.

In the disclosed Formulas of the present application, the expression of a ring structure streaked by “-” represents any position on the ring structure where a connectable site can form a bond.

The term “heteroaryl” in the present application refers to an aromatic cyclic group containing a heteroatom. The so-called heteroatom usually refers to N, O, S, P, Si, and Se, in an implementation, it refers to N, O, and S.

The above C₆-C₆₀ aryl and C₃-C₆₀ heteroaryl in the present application, unless otherwise specified, are the aromatic group that meets R conjugated system, and the C₆-C₆₀ aryl and C₃-C₆₀ heteroaryl both include cases of a single ring and a fused ring. The so-called single ring means that there is at least one phenyl in the molecule; when there are at least two phenyls in the molecule, the phenyls are independent from each other and connected through a single bond, for example, phenyl, diphenyl, terphenyl, etc. A fused ring refers to a molecule that contains at least two benzene rings, which are not independent from each other, but rather share a common ring edge and are fused with each other, for example, naphthyl, anthryl, and phenanthryl, etc. A monocyclic heteroaryl refers to a molecule containing at least one heteroaryl, and when the molecule contains one heteroaryl and other groups (such as aryl, heteroaryl, alkyl, etc.), the heteroaryl and other groups are independent from each other and connected through a single bond, exemplarily, such as pyridine, furan, thiophene, etc. A fused ring heteroaryl means that it is formed by fusing at least one phenyl and at least one heteroaryl, or it is formed by fusing at least two heteroaryl, exemplarily, such as quinoline, isoquinoline, benzofuran, dibenzofuran, benzothiophene, dibenzothiophene, etc.

In the present application, in an implementation, the substituted or unsubstituted C₆-C₆₀ aryl is C6-C30 aryl, and the carbon number of the aryl includes but is not limited to C6, C8, C10, C12, C14, C16, C18, C20, C22, C24, C26, C28, etc. Exemplarily, the aryl is selected from a group consisting of phenyl, naphthyl, anthryl, benzoanthryl, phenanthryl, benzophenanthryl, pyrenyl, chrysenyl, perylenyl, fluoranthenyl, tetraphenyl, pentaphenyl, benzopyrenyl, biphenylyl, diphenyl, terphenyl, trimeriephenyl, tetraphenyl, fluorenyl, spirodifluorenyl, dihydrophenanthryl, dihydropyrenyl, tetrahydropyrenyl, cis or trans indenofluorenyl, trimericindenyl, isotrimericindenyl, spiro-trimericindenyl, and spiro-isotrimericindenyl. Specifically, the biphenylyl is selected from 2-biphenylyl, 3-biphenylyl, and 4-biphenylyl; the terphenyl includes p-terphenyl-4-yl, p-terphenyl-3-yl, p-terphenyl-2-yl, m-terphenyl-4-yl, m-terphenyl-3-yl, and m-terphenyl-2-yl; the naphthyl includes 1-naphthyl or 2-naphthyl; the anthryl is selected from 1-anthryl, 2-anthryl, and 9-anthryl; the fluorenyl is selected from 1-fluorenyl, 2-fluorenyl, 3-fluorenyl, 4-fluorenyl, and 9-fluorenyl; the pyrenyl is selected from 1-pyrenyl, 2-pyrenyl, and 4-pyrenyl; the tetraphenyl is selected from 1-tetraphenyl, 2-tetraphenyl, and 9-tetraphenyl. As an example of the aromatic ring in the present application, it is a group selected from a group consisting of phenyl, biphenylyl, terphenyl, naphthyl, anthryl, phenanthryl, indenyl, fluorenyl and derivatives thereof, fluoranthenyl, terphenylene, pyrenyl, pyrrolo, chrysenyl, and tetraphenyl. The biphenylyl is selected from 2-biphenylyl, 3-biphenylyl, and 4-biphenylyl; the terphenyl includes p-terphenyl-4-yl, p-terphenyl-3-yl, p-terphenyl-2-yl, m-terphenyl-4-yl, m-terphenyl-3-yl, and m-terphenyl-2-yl; the naphthyl includes 1-naphthyl and 2-naphthyl; the anthryl is selected from a group consisting of 1-anthryl, 2-anthryl, and 9-anthryl; the fluorenyl is selected from a group consisting of 1-fluorenyl, 2-fluorenyl, 3-fluorenyl, 4-fluorenyl, and 9-fluorenyl; the fluorenyl derivative is selected from a group consisting of 9,9-dimethylfluorene, 9,9-spirodifluorene, and benzofluorene; the pyrenyl is selected from a group consisting of 1-pyrenyl, 2-pyrenyl, and 4-pyrenyl; the tetraphenyl is selected from a group consisting of 1-tetraphenyl, 2-tetraphenyl, and 9-tetraphenyl. The C6-C60 aryl in the present application may also be a group formed from the aforementioned groups via a single bond connection or/and by fusing.

In the presents application, in an implementation, the substituted or unsubstituted C3-C60 heteroaryl is C3-C30 heteroaryl. In the present application, the carbon number of the heteroaryl includes but is not limited to C4, C5, C6, C8, C10, C12, C14, C16, C18, C20, C22, C24, C26, C28, etc., and the heteroaryl is a N-containing heteroaryl, a 0-containing heteroaryl, a S-containing heteroaryl, etc. Specific examples are: furyl, thienyl, pyrryl, pyridinyl benzofuryl, benzothienyl, isobenzofuryl, isobenzothienyl, indolyl, isoindolyl, dibenzofuryl, dibenzothienyl, carbazolyl and derivatives thereof, quinolyl, isoquinolyl, acridinyl, phenanthridinyl, benzo-5,6-quinolyl, benzo-6,7-quinolyl, benzo-7,8-quinolyl, phenothiazinyl, phenazinyl, pyrazolyl, indazolyl, imidazolyl, benzimidazolyl, naphthoimidazolyl, phenanthroimidazolyl, pyridino imidazolyl, pyrazino imidazolyl, quinoxalino imidazolyl, oxazolyl, benzo-oxazolyl, naphtho-oxazolyl, anthro-oxazolyl, phenanthro-oxazolyl, 1,2-thiazolyl, 1,3-thiazolyl, benzothiazolyl, pyridazinyl, benzopyridazinyl, pyrimidinyl, benzopyrimidinyl, quinoxalinyl, 1,5-diaza anthryl, 2,7-diaza pyrenyl, 2,3-diaza pyrenyl, 1,6-diaza pyrenyl, 1,8-diaza pyrenyl, 4,5-diaza pyrenyl, 4,5,9,10-tetraaza perylenyl, pyrazinyl, phenazinyl, phenothiazinyl, naphthyridyl, azacarbazolyl, benzocarbolinyl, phenanthrolinyl, 1,2,3-triazolyl, 1,2,4-triazolyl, benzotriazolyl, 1,2,3-oxadiazolyl, 1,2,4-oxadiazolyl, 1,2,5-oxadiazolyl, 1,2,3-thiadiazolyl, 1,2,4-thiadiazolyl, 1,2,5-thiadiazolyl, 1,3,4-thiadiazolyl, 1,3,5-triazinyl, 1,2,4-triazinyl, 1,2,3-triazinyl, tetrazolyl, 1,2,4,5-tetrazinyl, 1,2,3,4-tetrazinyl, 1,2,3,5-tetrazinyl, purinyl, pteridyl, indozinyl, benzothiadiazolyl, etc. As an example of the heterocyclyl in the present application, it is, for example, furyl, thienyl, pyrryl, benzofuryl, benzothienyl, isobenzofuryl, indolyl, dibenzofuryl, dibenzothienyl, carbazolyl and derivatives thereof, where in an implementation, the carbazolyl derivatives are 9-phenyl carbazole, 9-naphthyl benzocarbazolyl, benzocarbazolyl, dibenzocarbazolyl or indolo carbazolyl. The C3-C60 heteroaryl in the present application may also be a group formed from the aforementioned groups via a single bond connection or/and by fusing.

In the present application, alkyl is not specified, and includes the concepts of linear alkyl and branched alkyl, as well as cycloalkyl. The carbon number of alkyl includes but is not limited to C1, C2, C3, C4, C5, C6, C7, C8, C9, C10, C11, C12, C13, C14, C15, C16, C17, C18, C19, C20, C22, C24, C26, C28, etc. As a C1-C30 alkyl, in an implementation, it is C1-C20 alkyl, for example, methyl, ethyl, n-propyl, isopropyl, n-butyl, isobutyl, sec-butyl, tert-butyl, 2-methyl butyl, n-pentyl, sec-pentyl, cyclopentyl, neopentyl, n-hexyl, cyclohexyl, adamantyl, neo-hexyl, n-heptyl, cycloheptyl, n-octyl, cyclooctyl, 2-ethyl hexyl, trifluoromethyl, pentafluoroethyl, 2,2,2-trifluoroethyl, etc., in an implementation, it is C1-C10 alkyl.

In the present application, the cycloalkyl includes monocyclic alkyl and polycyclic alkyl, and the carbon number of cycloalkyl includes but is not limited to C4, C5, C6, C7, C8, C9, etc. For example, it is cyclopropyl, cyclobutyl, cyclopentyl, cyclohexyl, cycloheptyl, etc.

In the present application, as an example of C1-C20 alkoxy, it is, methoxy, ethoxy, n-propoxy, isopropoxy, n-butoxy, sec-butoxy, isobutoxy, tert-butoxy, pentyloxy, isopentyloxy, hexyloxy, heptyloxy, octyloxy, nonyloxy, decyloxy, undecyloxy, dodecyloxy, and etc., in an implementation, it is, methoxy, ethoxy, n-propoxy, isopropoxy, tert-butoxy, sec-butoxy, isobutoxy, isopentyloxy, in other implementations, it is, methoxy.

In the present application, for an example of C1-C20 silyl group, it is the silyl that is substituted by the group exemplified in the above C1-C20 alkyl. Specifically, for example, methyl silyl, dimethyl silyl, trimethyl silyl, ethyl silyl, diethyl silyl, triethyl silyl, tert-butyl dimethyl silyl, tert-butyl diphenyl silyl and other groups are enumerated.

In the present application, for a C6-C60 aryloxy, the groups that are formed by connecting the group listed in the substituted or unsubstituted C6-C60 aryl with oxygen can be enumerated. Specific examples may refer to the above examples and will not be repeated here.

In the present application, as an example of halogens, fluorine, chlorine, bromine, iodine, etc. can be enumerated.

In the present application, a C6-C60 arylamino or a C3-C60 heteroarylamino refers to a group obtained by substituting one or two H in amino group (—NH₂) with the above illustrative C6-C60 aryl or C3-C60 heteroaryl.

The light-emitting layer of the organic electroluminescent device of the present application includes a host material and a fluorescent dye, where the host material is a triplet-triplet annihilation (TTA) material, and the fluorescent dye is a planar multi-resonance type compound represented by Formula (1) or Formula (2) and with an inverse intersystem crossing property. Specifically, the energy level of the first excited singlet state of the host material is greater than that of the first excited singlet state of the fluorescent dye, and the energy level of the first excited triplet state of the host material is less than that of the first excited triplet state of the fluorescent dye. Because the host material and the fluorescent dye have such relationship of energy level, after the organic electroluminescent device is electrically excited, the first excited singlet exciton of the host material will undergo Föster transition to the first excited singlet state of the low-energy level fluorescent dye. Although the fluorescent dye has the first excited triplet state with a higher energy level, the fluorescent dye will generate an up-conversion process due to its inverse intersystem crossing property. Therefore, the first excited triplet excitons and the first excited singlet excitons from the fluorescent dye and the first excited singlet excitons from the host material will jump to the ground state for emitting fluorescence. In addition, the excitons in the energy level of the first excited triplet state of the fluorescent dye that are too late for upconversion will also jump to the first excited triplet state of the low-energy host material, and then a phenomena that the triplet excitons are annihilated in pairs to generate singlet excitons occurs. Based on the energy transfer process, the organic electroluminescent device of the present application can not only effectively utilize the triplet excitons, but also have a low concentration of the triplet excitons in the system. Therefore, the organic electroluminescent device of the present application has excellent luminescence efficiency, roll-off of efficiency and low driving voltage.

In addition to the above reasons, the inventor believes that the improvement of device performance may also be related to the fluorescent dye used in the present application. On the one hand, the molecular structure of Formula (1) and Formula (2) introduces a carbon ring or heterocyclic that is represented by A and coated by a group with large steric effect. The group with large steric effect not only does not have a significant impact on the emitting color and half-peak width of the parent nucleus, but also can effectively inhibit the interaction between planar type multi-resonance compounds, so as to effectively inhibit the reduction of the luminescence efficiency and spectral broadening of compounds at a high concentration. On the other hand, the molecular structures of Formula (1) and Formula (2) can effectively inhibit the influence between host and guest materials in the light-emitting layer, including Dexter energy transfer and intermolecular interaction, thereby greatly improving the luminescence efficiency of the device and reducing the driving voltage, and realizing the optimization of the efficiency process window qualification, enhancing the stability of the luminescence efficiency and driving voltage. Moreover, the feasibility of chemical synthesis of the fluorescent dye represented by Formulas (1) and (2) is higher, and it is easy to make various functional modifications, allowing for further structural adjustments according to different application requirements.

In one embodiment, the fluorescent dye has a structure represented by any one of the following (1-1), (1-2), (1-3), (1-4), (1-5), (1-6), (2-1), (2-2), or (2-3):

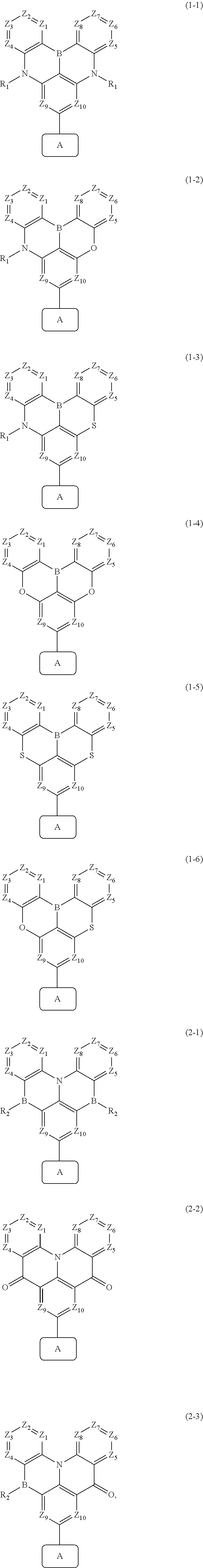

where, Z₁-Z₁₀ are each independently represented as CR, and the definitions of A, R, R₁, and R₂ are same as those in Formula (1) or (2); in an implementation, R₁ is connected to adjacent R through a single bond, and R₂ is connected to adjacent R through a single bond.

Further, in the aforementioned structure of the fluorescent dye, A represents a substituted group represented by any one of the following (3-1), (3-2), (3-3), (3-4), (3-5), (3-6), (3-7), (3-8) or (3-9):

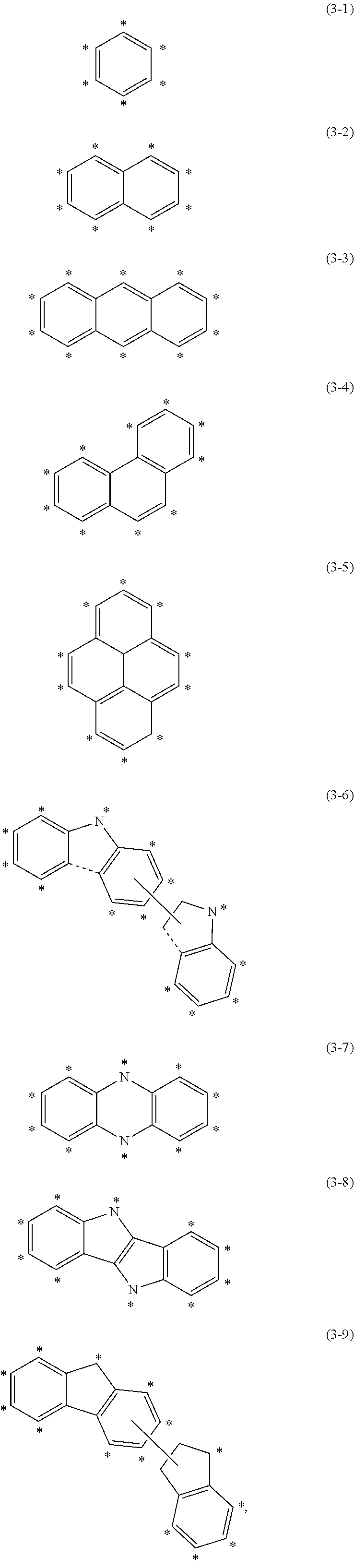

an asterisks in the above Formulas denotes a connectable site, such “connection” denotes connection to a parent nucleus and/or connection with a substituted group; an expression of a ring structure streaked by “-” denotes any position on the ring structure where the connectable site can form a bond; a dashed line in the above Formulas represents being connected or being unconnected; a substituted group in A is one or a combination of at least two selected from deuterium, tritium, cyano, halogen, C₁-C₁₀ alkyl, C₃-C₁₀ cycloalkyl, C₆-C₃₀ arylamino, C₆-C₃₀ aryl, C₂-C₃₀ heteroaryl, and the substituted group is independently connected to a connected aromatic ring or heteroaromatic ring to form a ring or not to form a ring.

Furthermore, A is represented by any one of the following structural formulas:

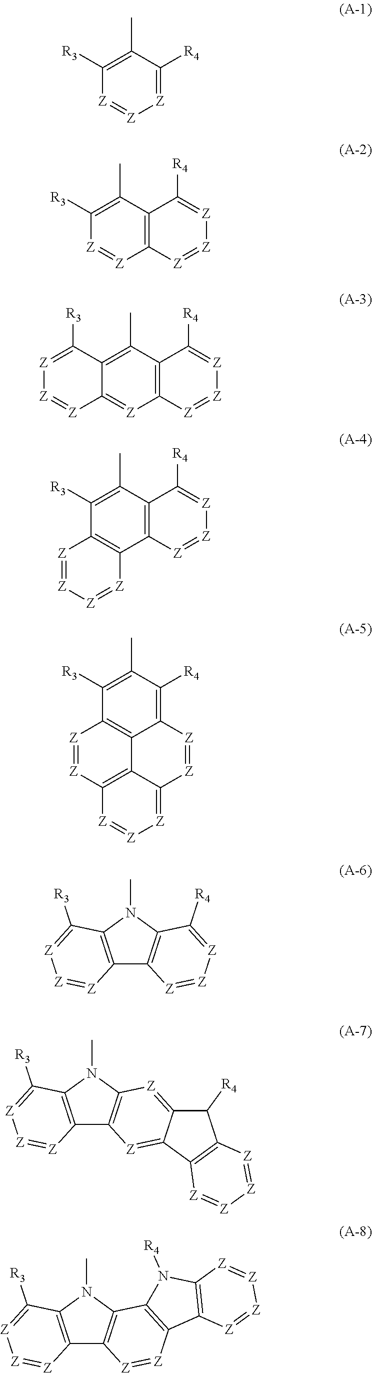

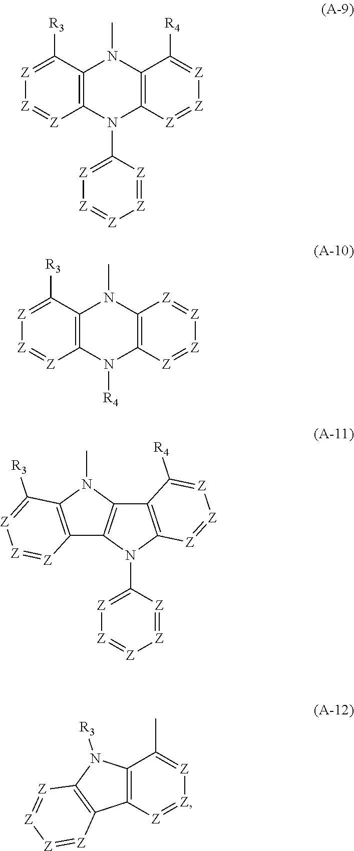

where, R₃ and R₄ are each independently represented as any one of hydrogen, deuterium, tritium, a substituted or unsubstituted C₁-C₃₀ alkyl, a substituted or unsubstituted C₃-C₃₀ cycloalkyl, a silyl, a substituted or unsubstituted C₁-C₃₀ alkoxy, a substituted or unsubstituted C₆-C₆₀ aryloxy, a substituted or unsubstituted C₆-C₆₀ arylamino, a substituted or unsubstituted C₆-C₆₀ aryl, and a substituted or unsubstituted C₂-C₆₀ heteroaryl; Z is independently represented as N or CR₅, with R₅ being the same or different each time, and two adjacent R₅ can bond with each other to form a ring; R₅ is represented as one of hydrogen, deuterium, tritium, cyano, halogen, a substituted or unsubstituted C₁-C₁₀ alkyl, a substituted or unsubstituted C₃-C₁₀ cycloalkyl, a substituted or unsubstituted C₁-C₁₀ alkoxy, a substituted or unsubstituted C₆-C₃₀ aryloxy, a substituted or unsubstituted C₆-C₃₀ arylamino, a substituted or unsubstituted C₆-C₃₀ aryl, or a substituted or unsubstituted C₂-C₃₀ heteroaryl; a substituted group in the each substituted R₃, R₄, and R₅ is one or a combination of at least two selected from deuterium, tritium, cyano, halogen, C₁-C₁₀ alkyl, C₃-C₁₀ cycloalkyl, silyl, C₆-C₃₀ arylamino, C₆-C₃₀ aryl, C₂-C₃₀ heteroaryl, and the substituted group is independently connected to the connected aromatic ring or heteroaromatic ring to form a ring or not to form a ring.

Further, above R₃ and R₄ are each independently represented as any one of a substituted or unsubstituted C₁-C₃₀ alkyl, a substituted or unsubstituted C₃-C₃₀ cycloalkyl, a substituted or unsubstituted C₆-C₆₀ aryl, and a substituted or unsubstituted C₂-C₆₀ heteroaryl; in an implementation, at least one of the above R₃ and R₄ is selected from one of the following groups with large steric effect: terphenyl, trimericphenyl, tetraphenyl, fluorenyl, spirodifluorenyl, dihydrophenanthryl, dihydropyrenyl, tetrahydropyrenyl, cis or trans indenofluorenyl, trimericindenyl, isotrimericindenyl, spiro-trimericindenyl, spiro-isotrimericindenyl, furyl, benzofuryl, isobenzofuryl, dibenzofuryl, benzothienyl, isobenzothienyl, dibenzothienyl, isoindolyl, carbazolyl, indenocarbazolyl, isoquinolyl, acridinyl, phenanthridinyl, benzo-5,6-quinolyl, benzo-6,7-quinolyl, benzo-7,8-quinolyl, benzimidazolyl, naphthoimidazolyl, phenanthroimidazolyl, pyridino imidazolyl, pyrazino imidazolyl, quinoxalino imidazolyl, oxazolyl, benzo-oxazolyl, naphtho-oxazolyl, anthro-oxazolyl, phenanthro-oxazolyl, 1,2-thiazolyl, 1,3-thiazolyl, benzothiazolyl, pyridazinyl, benzopyridazinyl, 1,5-diaza anthryl, 2,7-diaza pyrenyl, 2,3-diaza pyrenyl, 1,6-diaza pyrenyl, 1,8-diaza pyrenyl, 4,5-diaza pyrenyl, 4,5,9,10-tetraaza perylenyl, phenazinyl, phenothiazinyl, azacarbazolyl, benzocarbolinyl, phenanthrolinyl, 1,2,3-triazolyl, 1,2,4-triazolyl, benzotriazolyl, 1,2,3-oxadiazolyl, 1,2,4-oxadiazolyl, 1,2,5-oxadiazolyl, 1,2,3-thiadiazolyl, 1,2,4-thiadiazolyl, 1,2,5-thiadiazolyl, 1,3,4-thiadiazolyl, 1,3,5-triazinyl, 1,2,4-triazinyl, 1,2,3-triazinyl, tetrazolyl, 1,2,4,5-tetrazinyl, 1,2,3,4-tetrazinyl, 1,2,3,5-tetrazinyl, purinyl, pteridyl, indozinyl, benzothiadiazolyl, 9,9-dimethylacridinyl, diphenylamindo, adamantyl, fluorophenyl, methylphenyl, trimethylphenyl, cyanophenyl, silyl; or at least one of R₃ or R₄ is selected from a combination of two or more from the aforementioned groups with large steric effect.

In addition, in the fluorescent dye of the present application, R denotes one of hydrogen, deuterium, tritium, fluorine atom, cyano, methyl, deuterated methyl, tritiated methyl, ethyl, deuterated ethyl, tritiated ethyl, isopropyl, deuterated isopropyl, tritiated isopropyl, tert-butyl, deuterated tert-buty, tritiated tert-butyl, deuterated cyclopentyl, tritiated cyclopentyl, cyclohexyl, cyclopentyl, adamantyl, phenyl, deuterated phenyl, tritiated phenyl, biphenyl, deuterated biphenyl, tritiated biphenyl, deuterated terphenyl, tritiated terphenyl, terphenyl, naphthyl, anthryl, phenanthryl, pyridyl, quinolyl, furyl, thienyl, dibenzofuryl, dibenzothienyl, carbazolyl, N-phenylcarbazolyl, 9,9-dimethylfluorenyl, 9,9-diphenylfluorenyl, spirofluorenyl, methyl-substituted phenyl, ethyl-substituted phenyl, isopropyl-substituted phenyl, tert-butyl-substituted phenyl, methyl-substituted biphenyl, ethyl-substituted biphenyl, isopropyl-substituted biphenyl, tert-butyl-substituted biphenyl, deuterated methyl-substituted phenyl, deuterated ethyl-substituted phenyl, deuterated isopropyl-substituted phenyl, deuterated tert-butyl-substituted phenyl, deuterated methyl-substituted biphenyl, deuterated ethyl-substituted biphenyl, deuterated isopropyl-substituted biphenyl, deuterated tert-butyl-substituted diphenyl, tritiated methyl-substituted phenyl, tritiated ethyl-substituted phenyl, tritiated isopropyl-substituted phenyl, tritiated tert-butyl-substituted phenyl, tritiated methyl-substituted diphenyl, tritiated ethyl-substituted diphenyl, tritiated isopropyl-substituted diphenyl, tritiated tert-butyl-substituted diphenyl, diphenylamido, di-biphenylamido, and triphenylamino.

R₁ represents one of methyl, deuterated methyl, tritiated methyl, ethyl, deuterated ethyl, tritiated ethyl, isopropyl, deuterated isopropyl, tritiated isopropyl, tert-butyl, deuterated tert-butyl, tritiated tert-butyl, deuterated cyclopentyl, tritiated cyclopentyl, cyclopentyl, adamantyl, phenyl, deuterated phenyl, tritiated phenyl, biphenyl, deuterated biphenyl, tritiated biphenyl, deuterated terphenyl, tritiated terphenyl, terphenyl, naphthyl, anthryl, phenanthryl, pyridyl, quinolyl, furyl, thienyl, dibenzofuryl, dibenzothienyl, carbazolyl, N-phenylcarbazolyl, 9,9-dimethylfluorenyl, 9,9-diphenylfluorenyl, spirofluorenyl, methyl-substituted phenyl, ethyl-substituted phenyl, isopropyl-substituted phenyl, tert-butyl-substituted phenyl, methyl-substituted phenyl, ethyl-substituted diphenyl, isopropyl-substituted diphenyl, tert-butyl-substituted diphenyl, deuterated methyl-substituted phenyl, deuterated ethyl-substituted phenyl, deuterated isopropyl-substituted phenyl, deuterated tert-butyl-substituted phenyl, deuterated methyl-substituted diphenyl, deuterated ethyl-substituted diphenyl, deuterated isopropyl-substituted diphenyl, deuterated tert-butyl-substituted diphenyl, tritiated methyl-substituted phenyl, tritiated ethyl-substituted phenyl, tritiated isopropyl-substituted phenyl, tritiated tert-butyl-substituted phenyl, tritiated methyl-substituted diphenyl, tritiated ethyl-substituted diphenyl, tritiated isopropyl-substituted diphenyl, and tritiated tert-butyl-substituted diphenyl.

R₂ represents one of phenyl, deuterated phenyl, tritiated phenyl, biphenyl, deuterated biphenyl, tritiated biphenyl, deuterated terphenyl, tritiated terphenyl, terphenyl, naphthyl, anthryl, phenanthryl, pyridyl, quinolyl, dibenzofuryl, dibenzothienyl, N-phenylcarbazolyl, methyl-substituted phenyl, amidine, ethyl-substituted phenyl, isopropyl-substituted phenyl, tert-butyl-substituted phenyl, methyl-substituted biphenyl, ethyl-substituted biphenyl, isopropyl-substituted biphenyl, tert-butyl-substituted biphenyl, deuterated methyl-substituted phenyl, deuterated ethyl-substituted phenyl, deuterated isopropyl-substituted phenyl, deuterated tert-butyl-substituted phenyl, deuterated methyl-substituted biphenyl, deuterated ethyl-substituted biphenyl, deuterated isopropyl-substituted biphenyl, deuterated tert-butyl-substituted biphenyl, tritiated methyl-substituted phenyl, tritiated ethyl-substituted phenyl, tritiated isopropyl-substituted phenyl, tritiated tert-butyl-substituted phenyl, tritiated methyl-substituted diphenyl, tritiated ethyl-substituted diphenyl, tritiated isopropyl-substituted diphenyl, and tritiated tert-butyl-substituted diphenyl.

In a specific implementation, in Formula (1) or (2), Z₉ and Z₁₀ are each CR, and R is hydrogen, while Z₁-Z₈ are each CR, the definition of R is the same as that in Formula (1) or (2).

In another specific implementation, in Formula (1) or (2), Z₂ and Z₇ are each CR, and R is tert-butyl, while Z₁, Z₃-Z₆, and Z₈-Z₁₀ are each CR, and R is hydrogen.

More specifically, the fluorescent dye in the present application is selected from a compound represented by the following structural formulas:

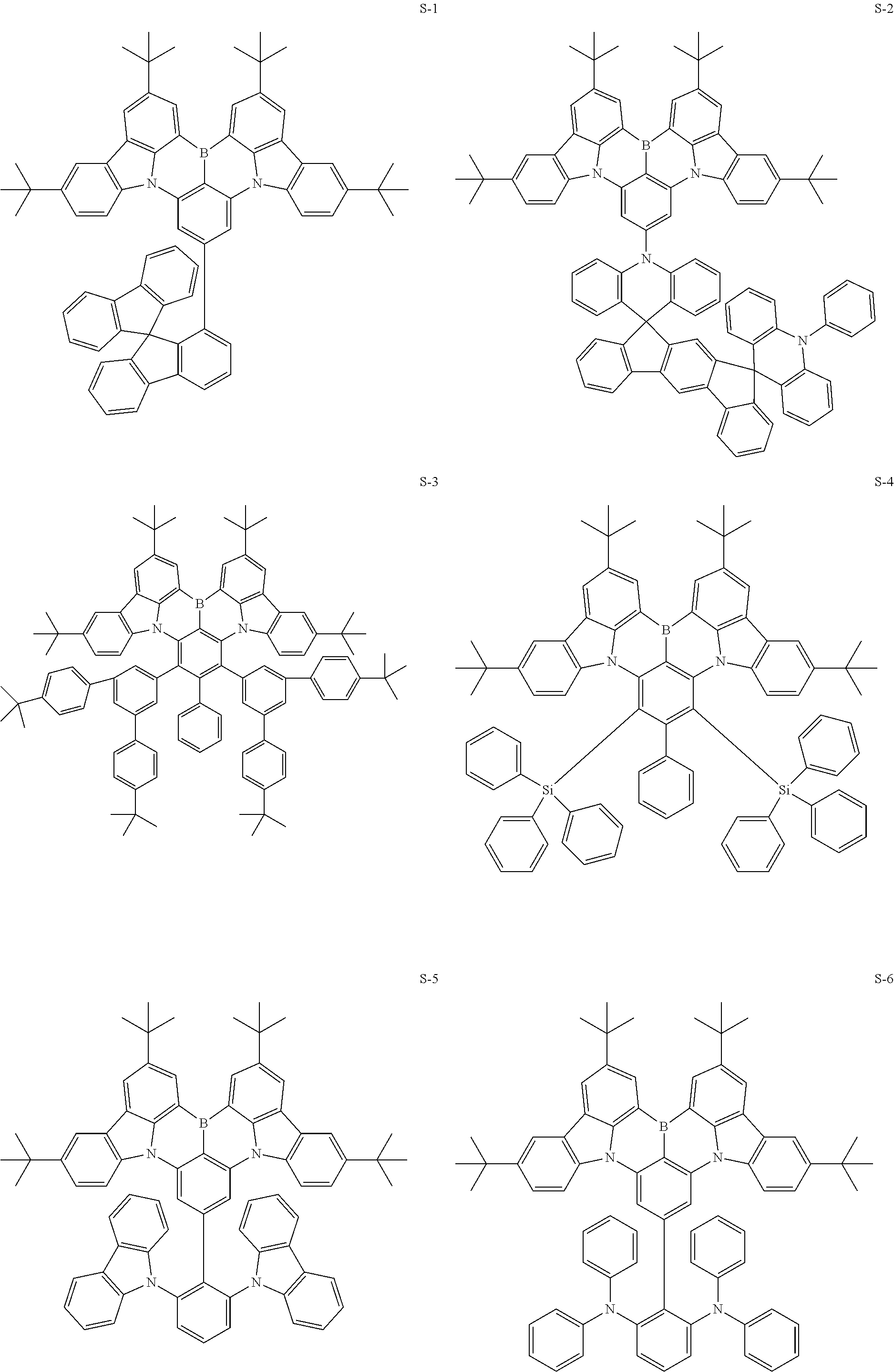

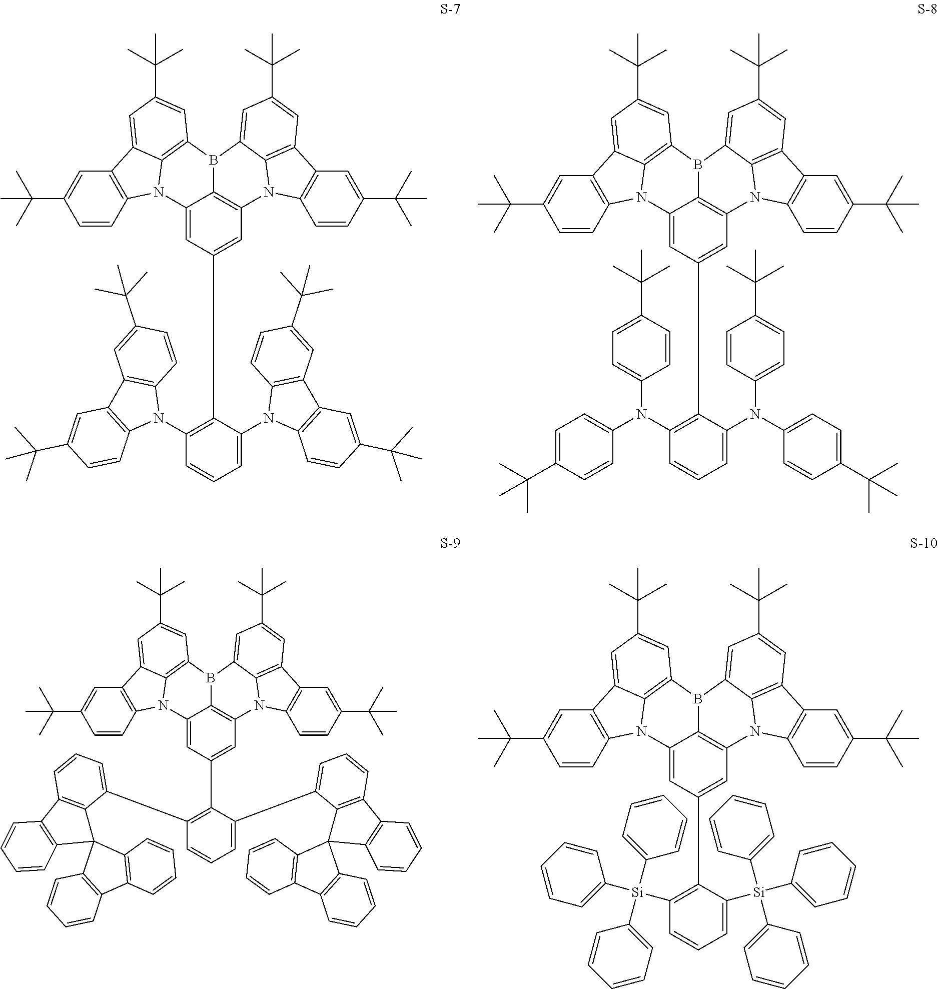

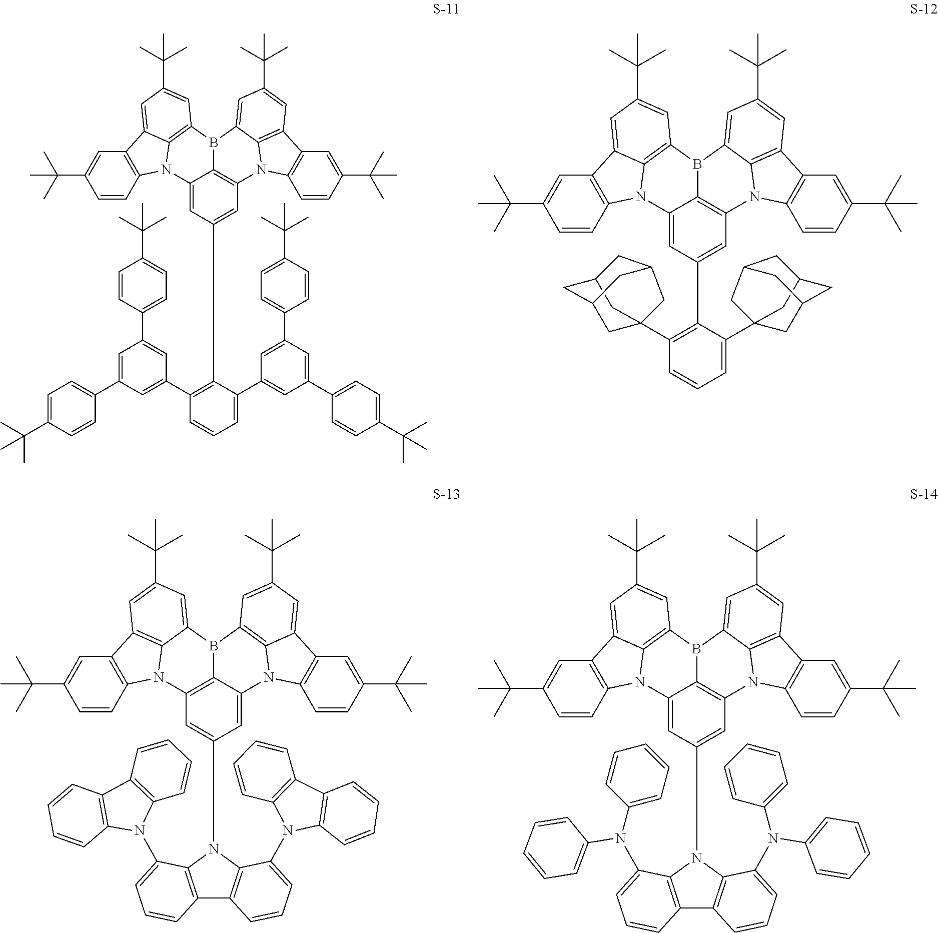

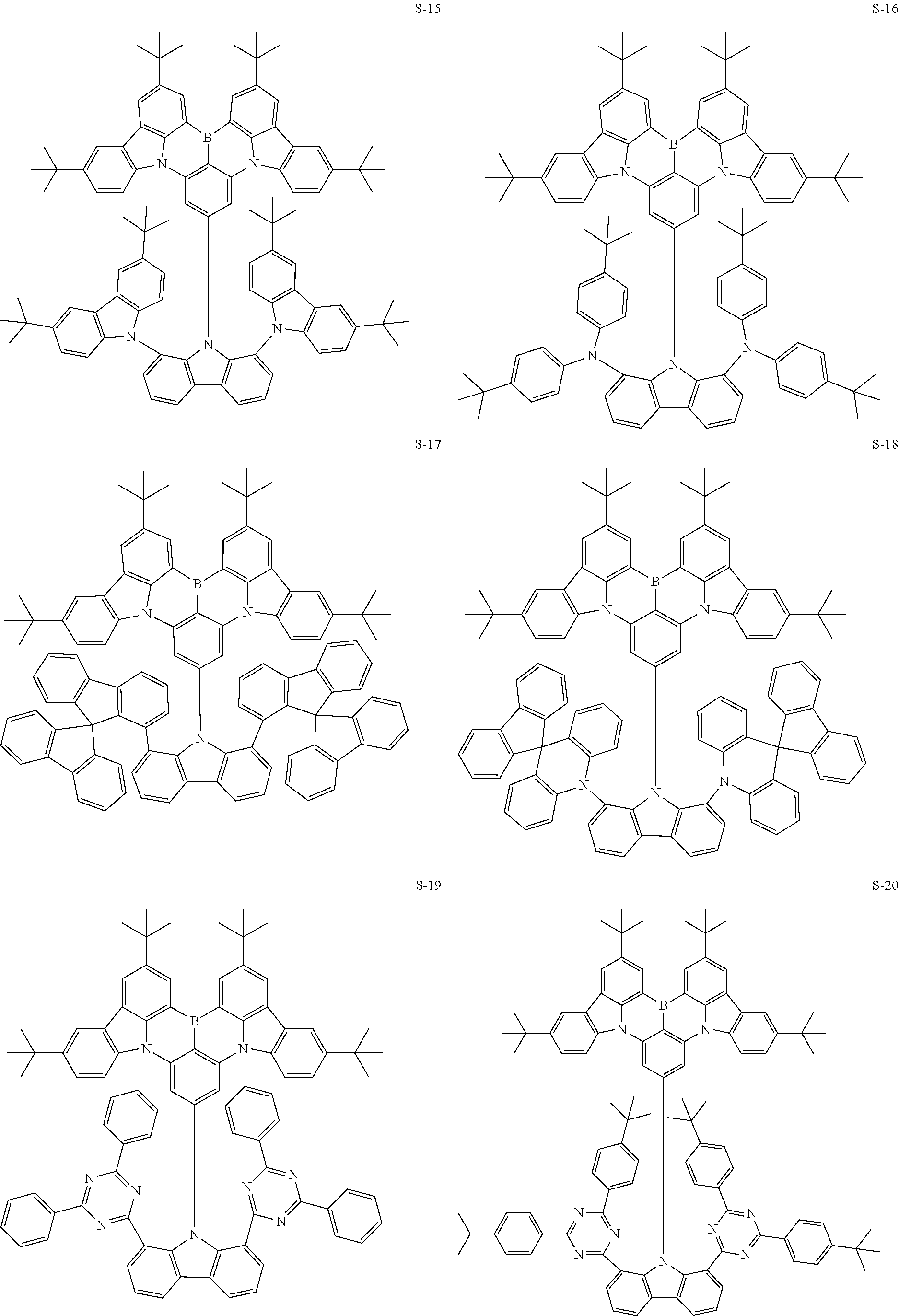

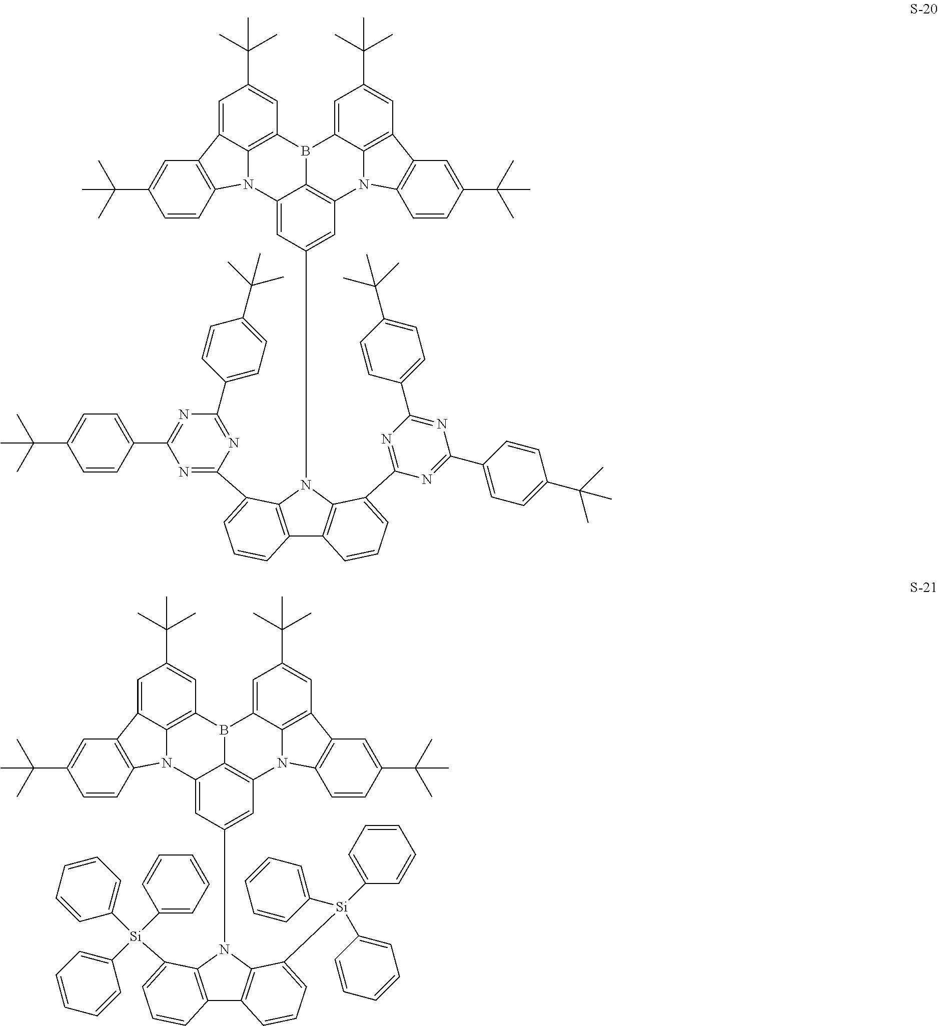

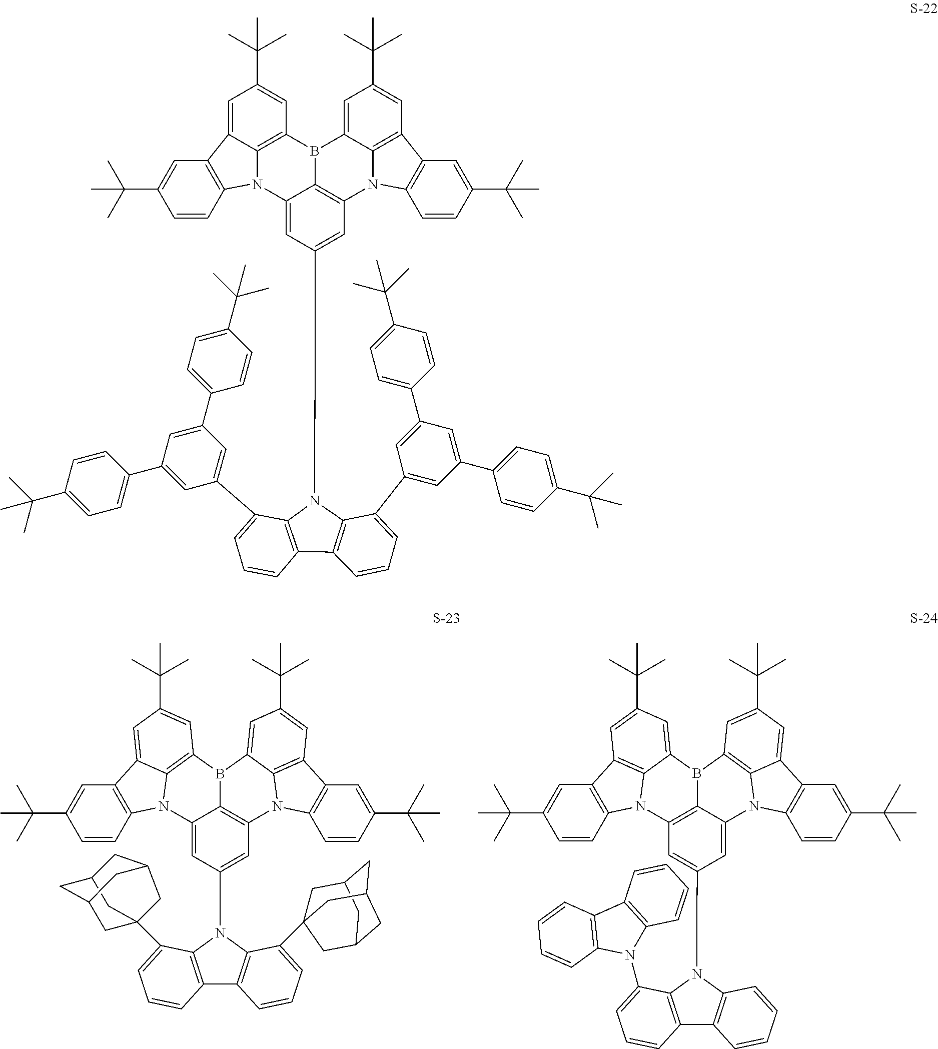

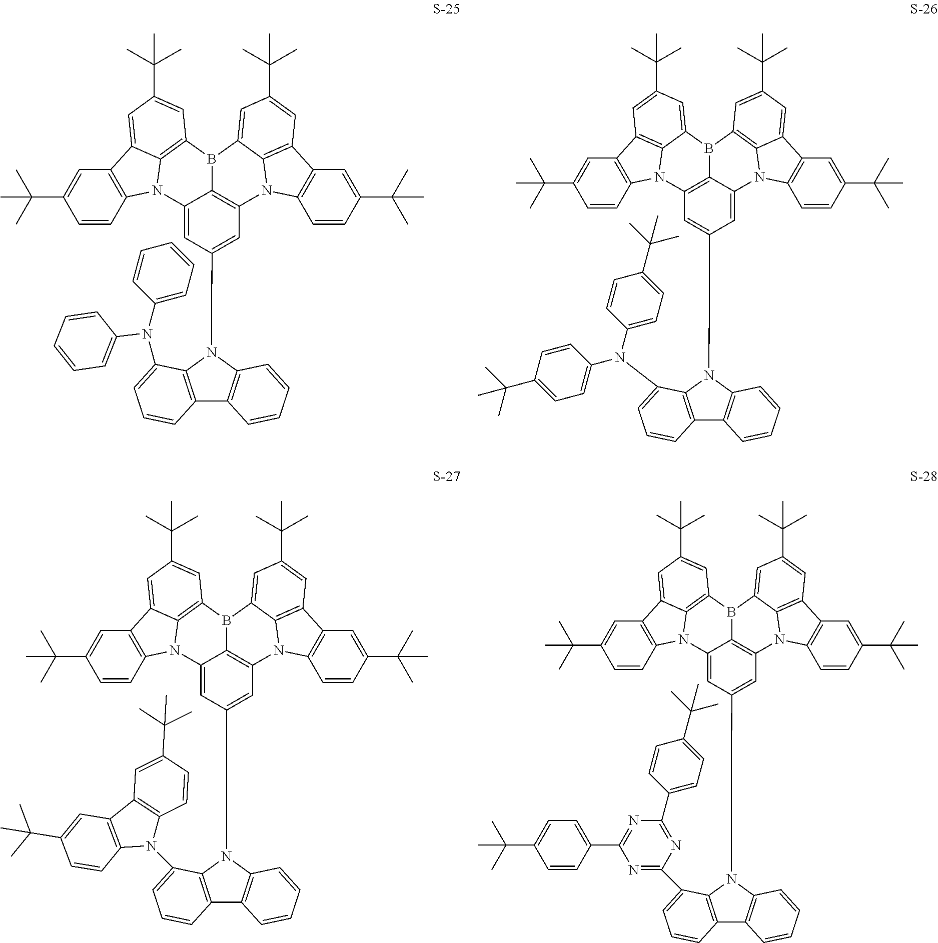

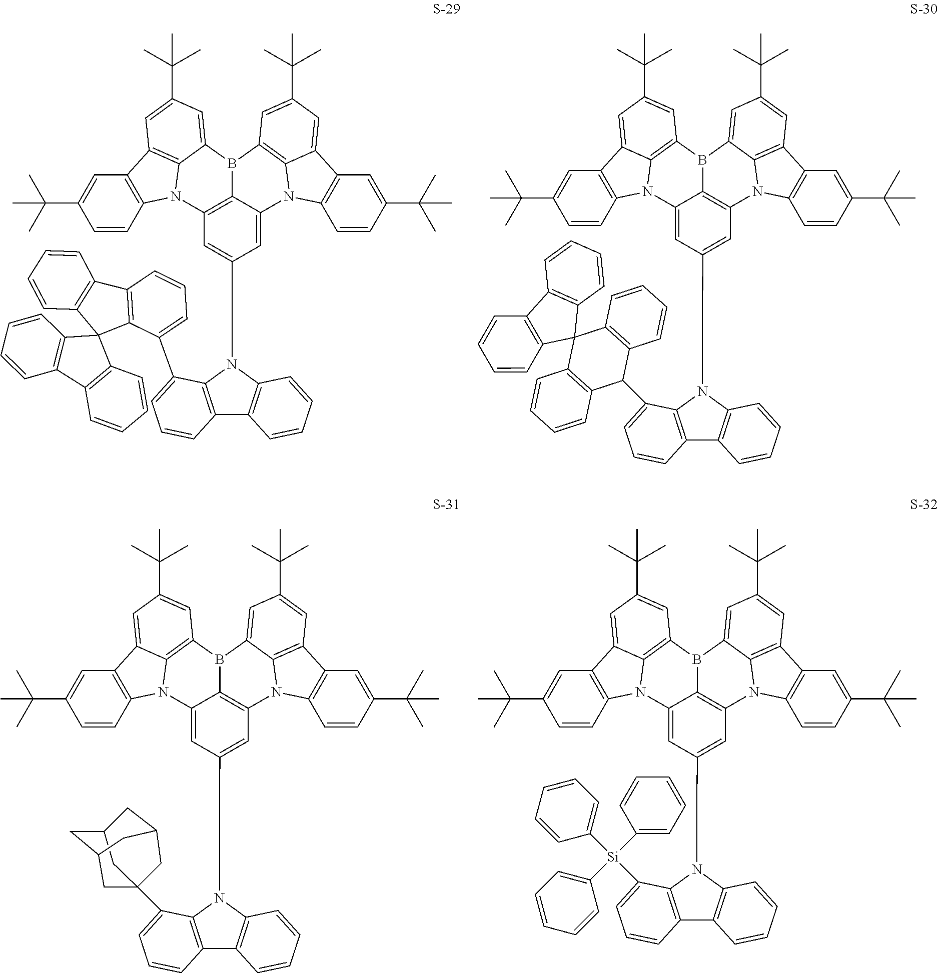

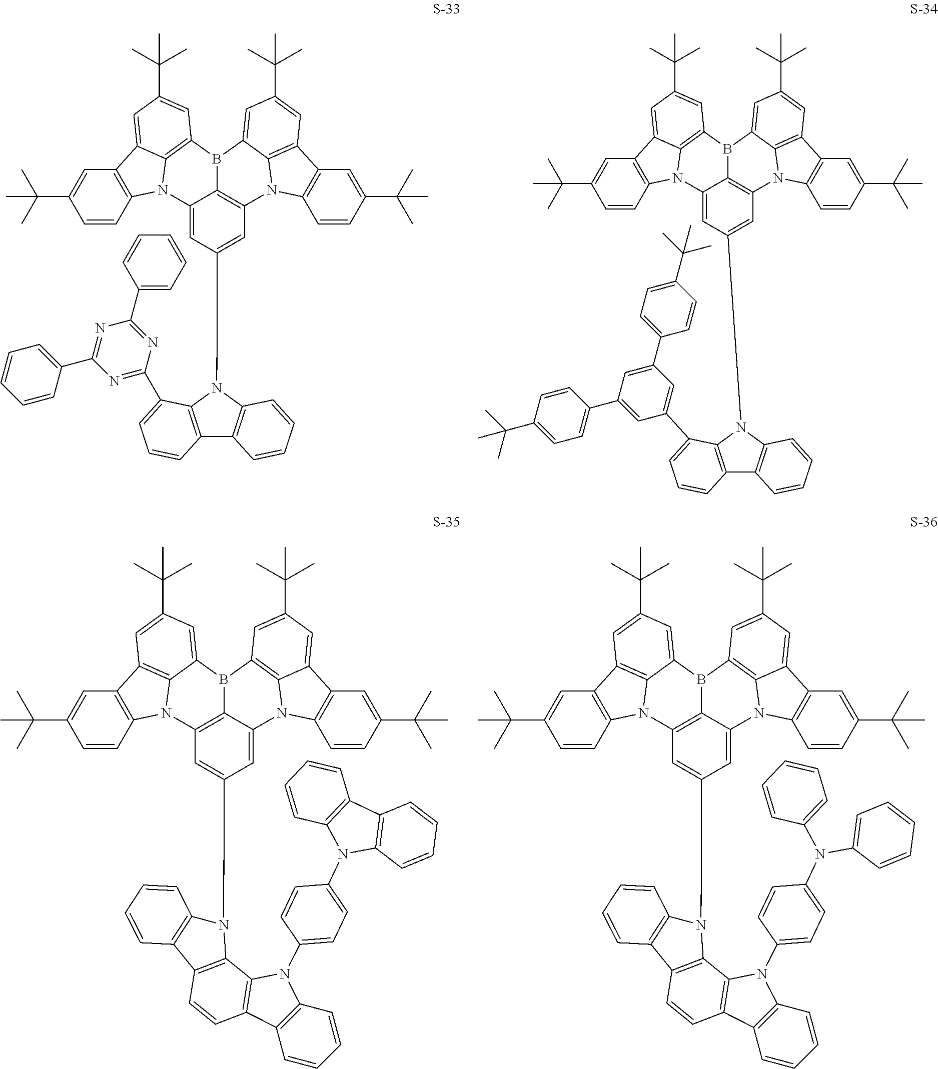

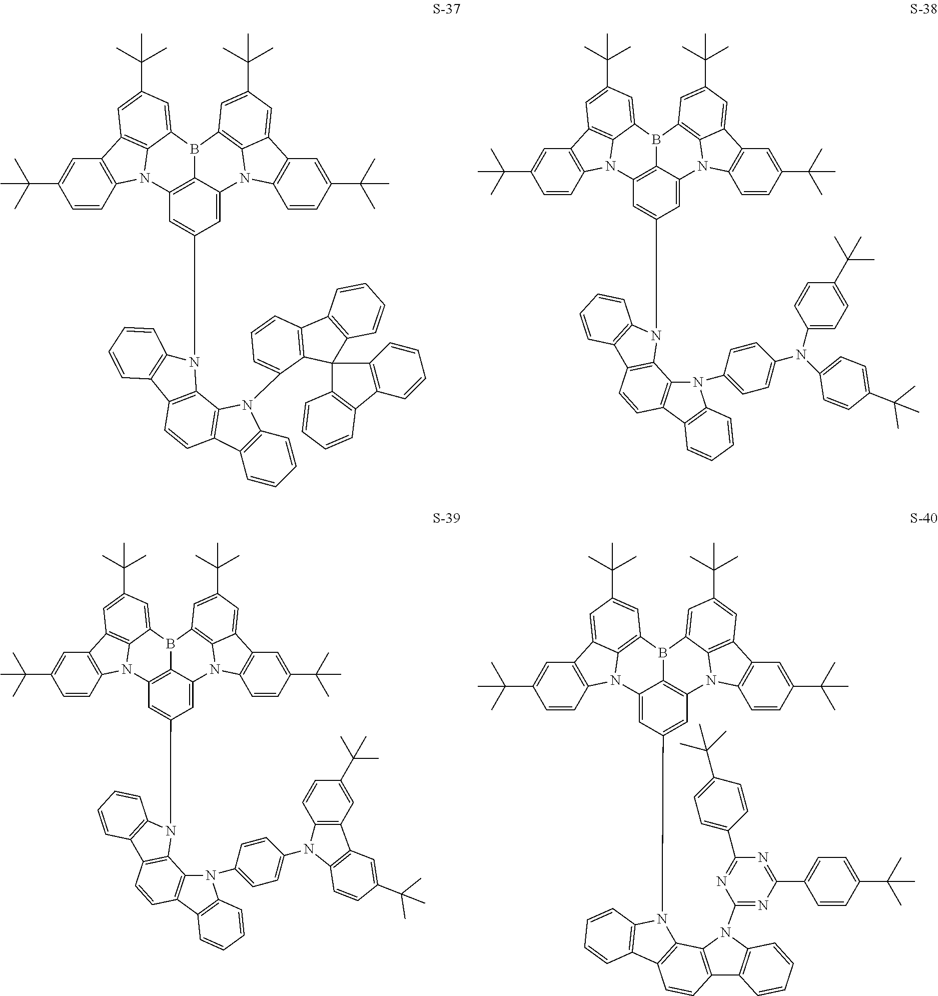

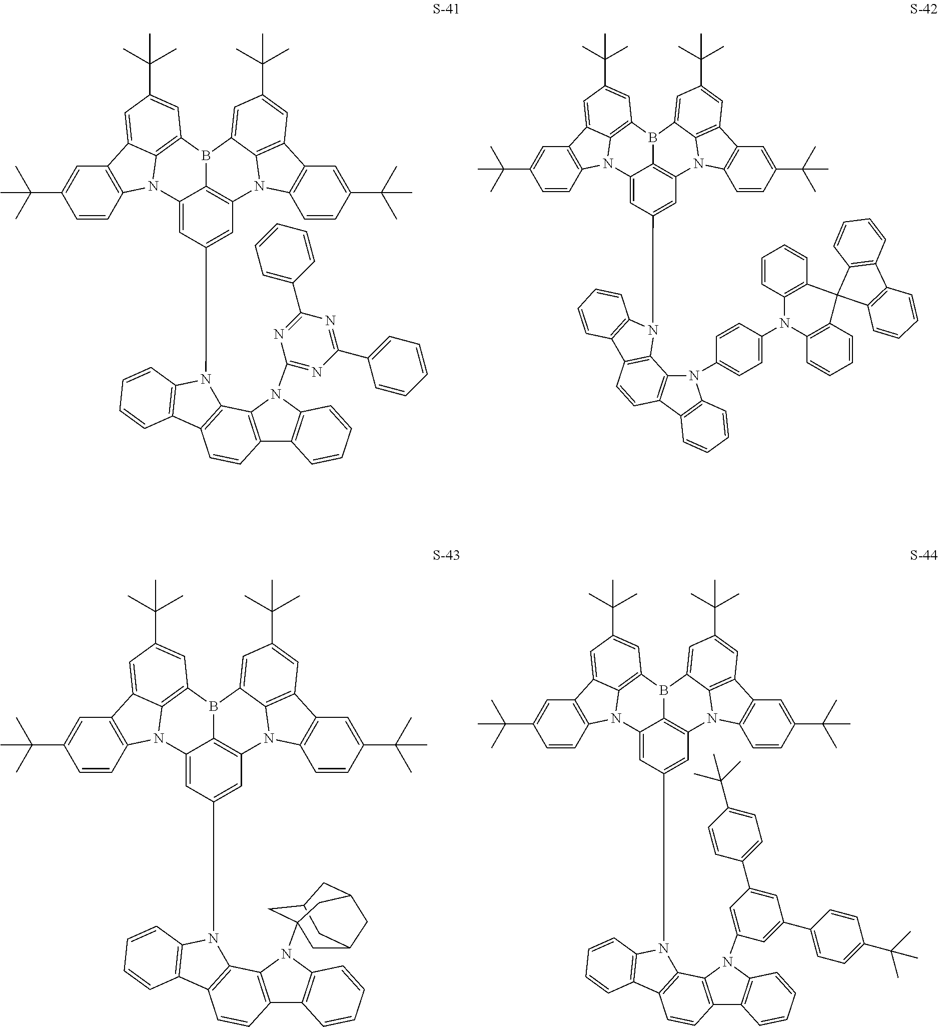

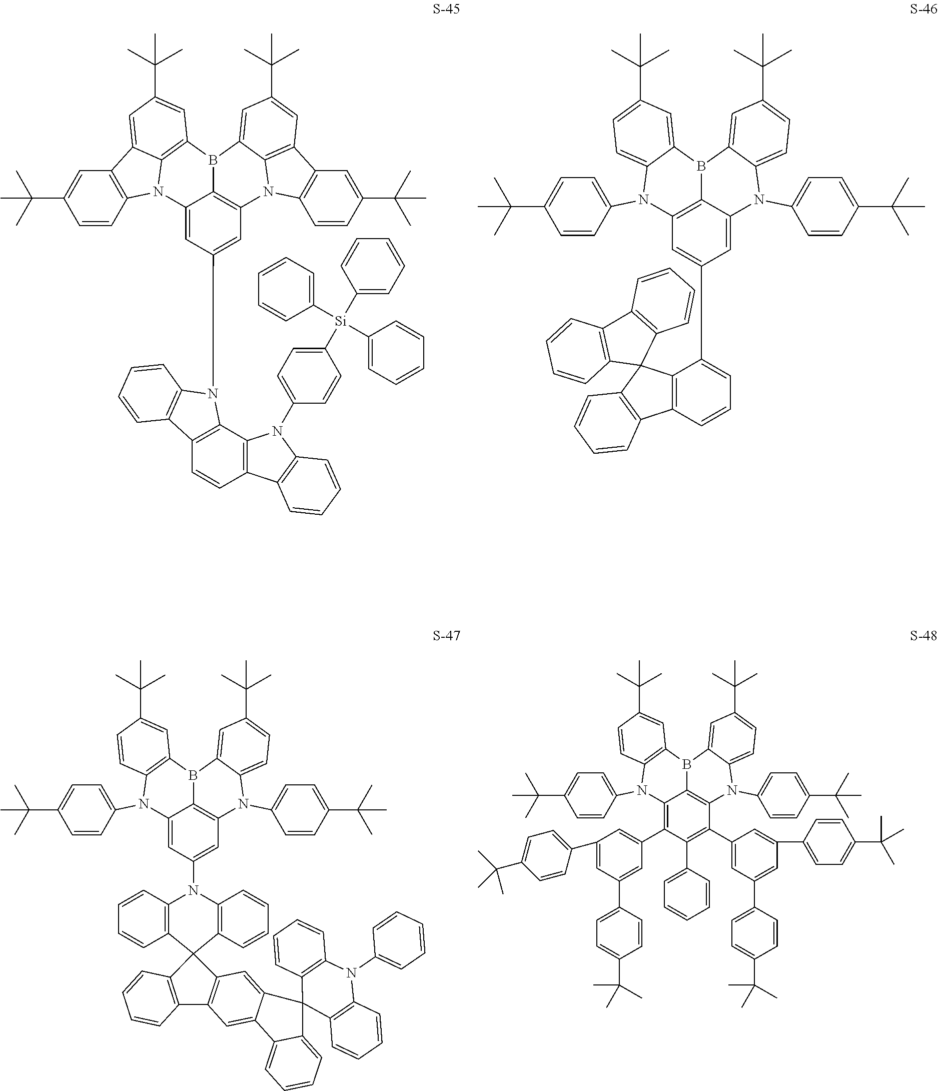

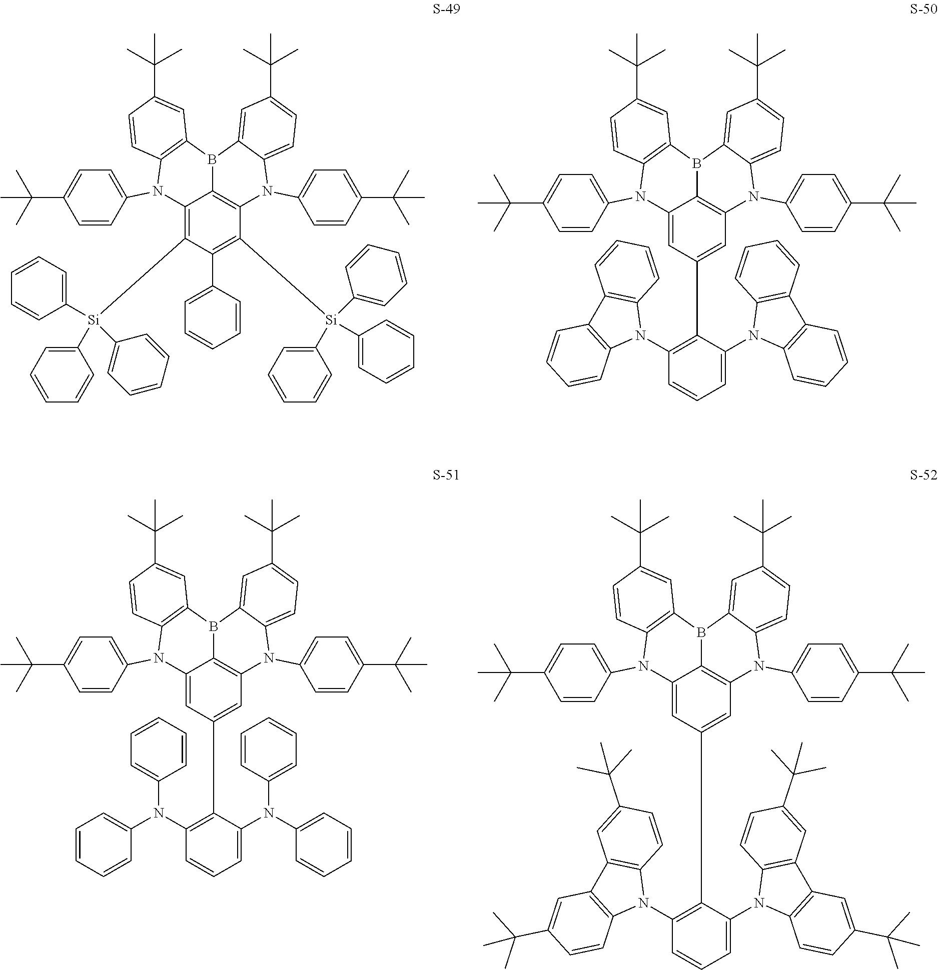

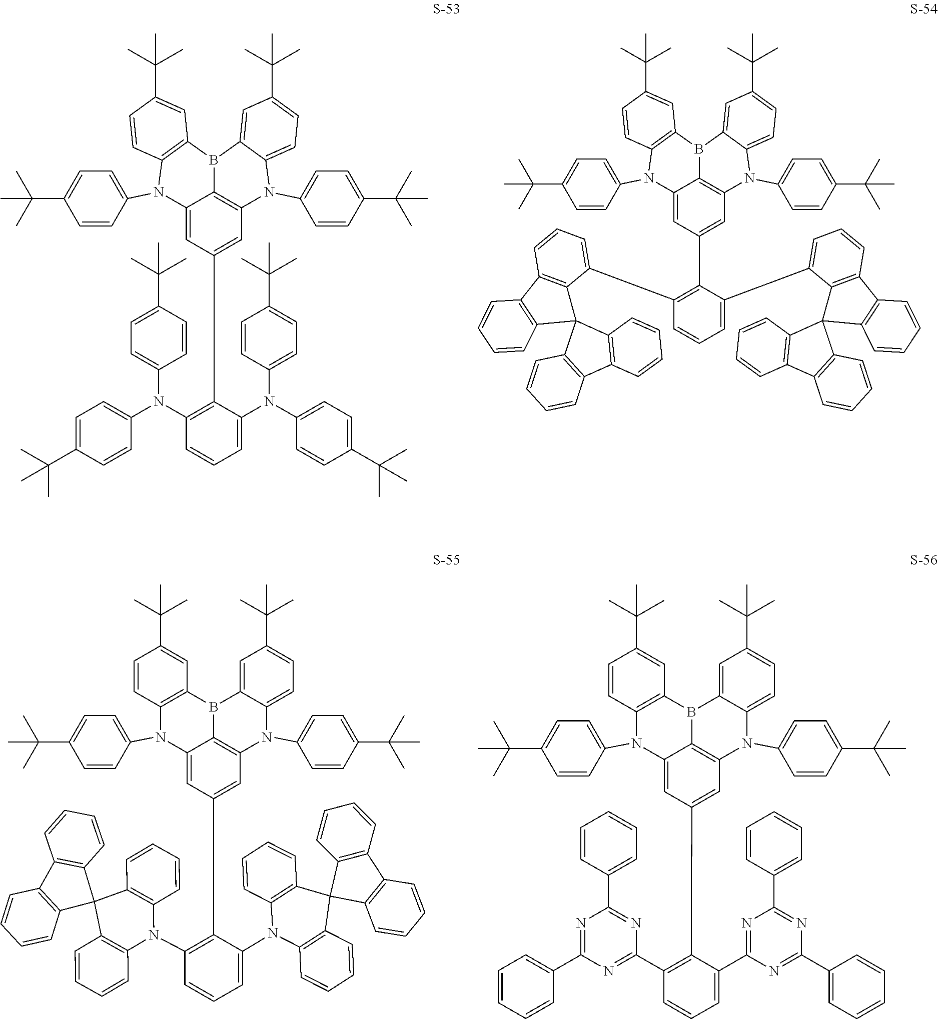

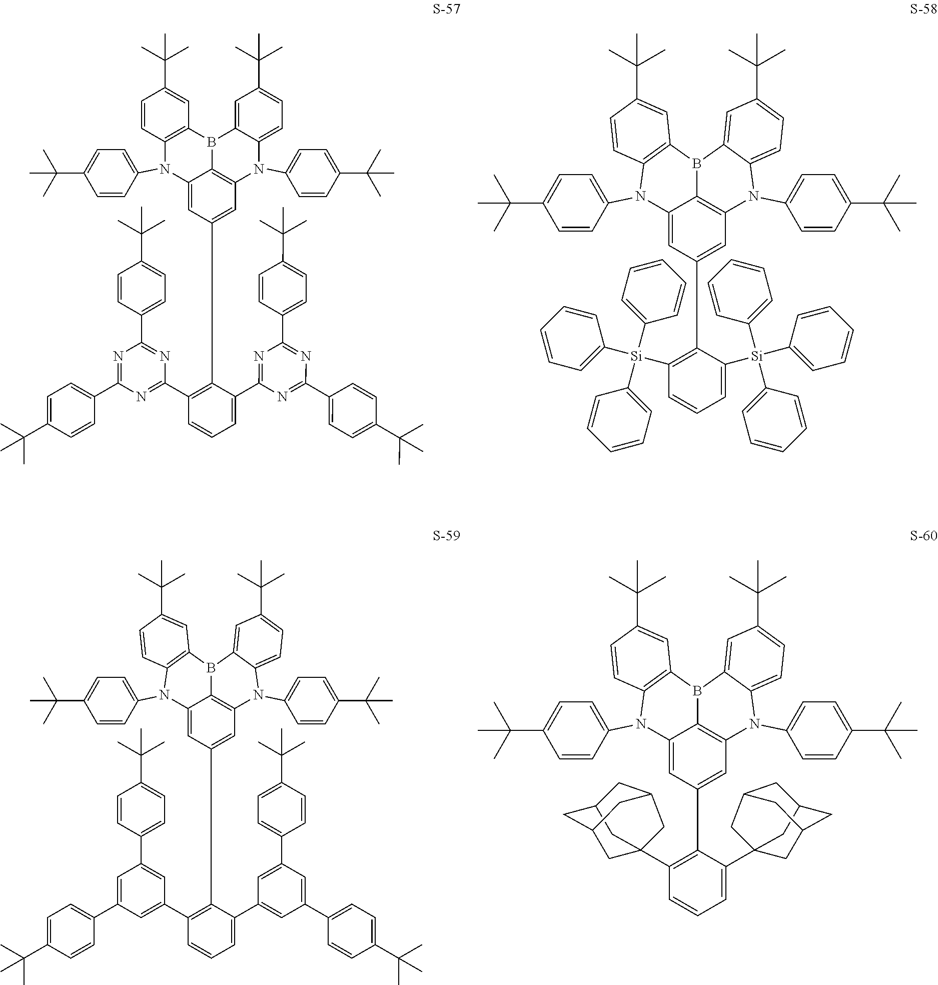

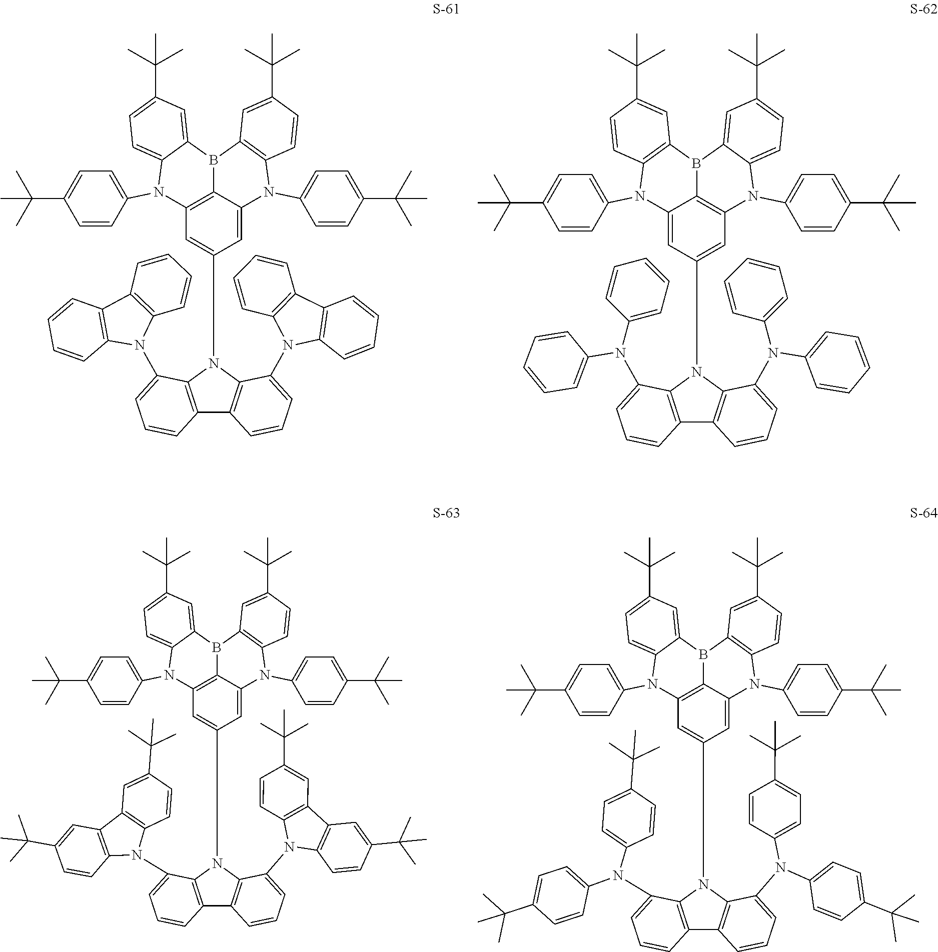

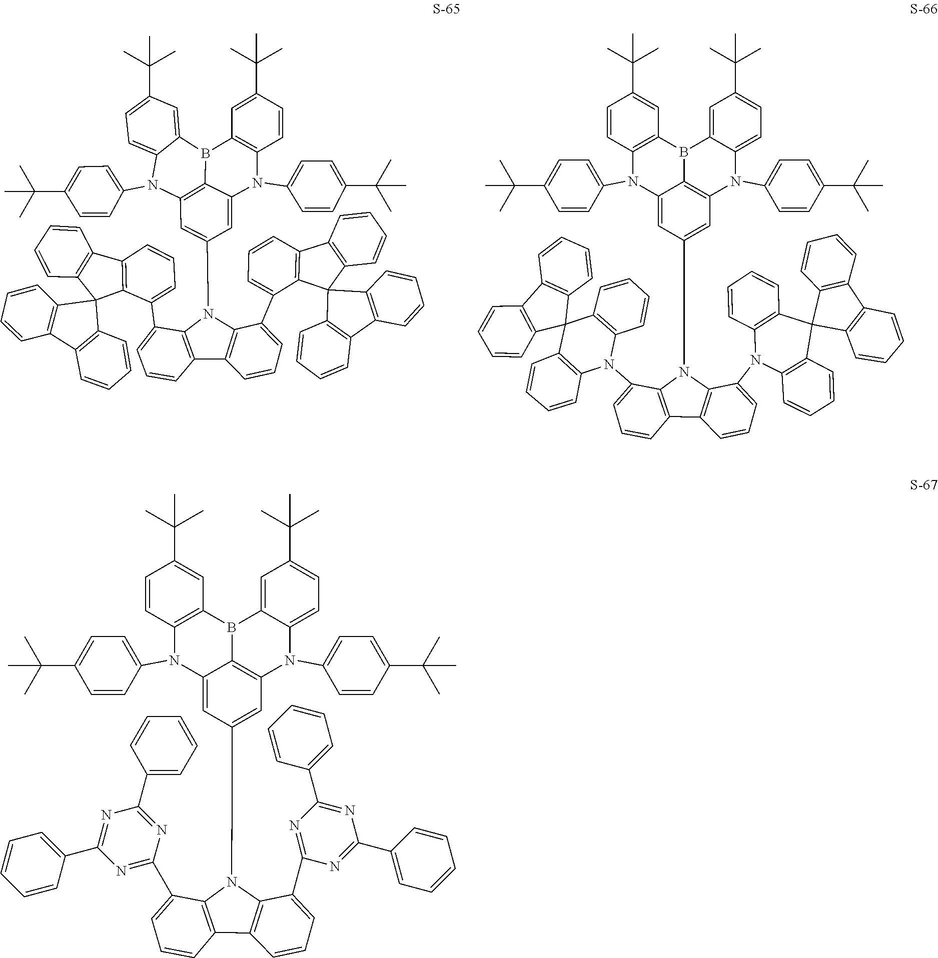

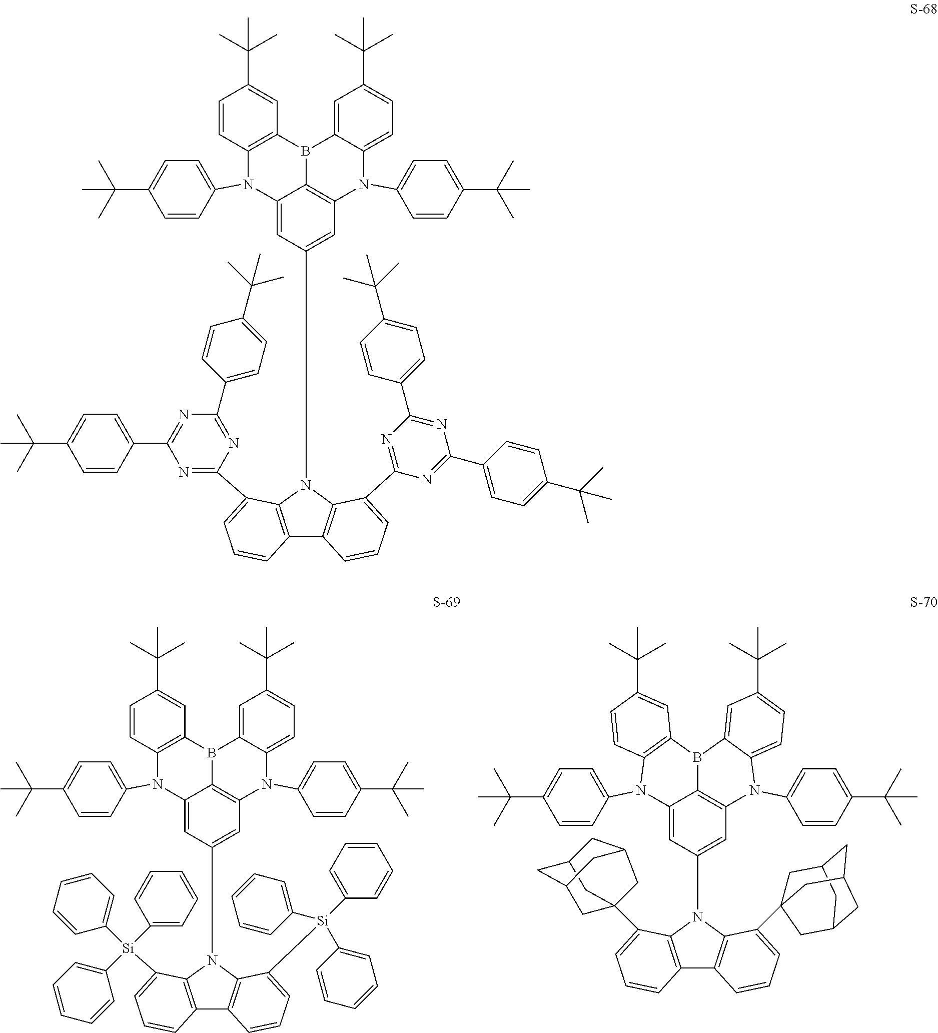

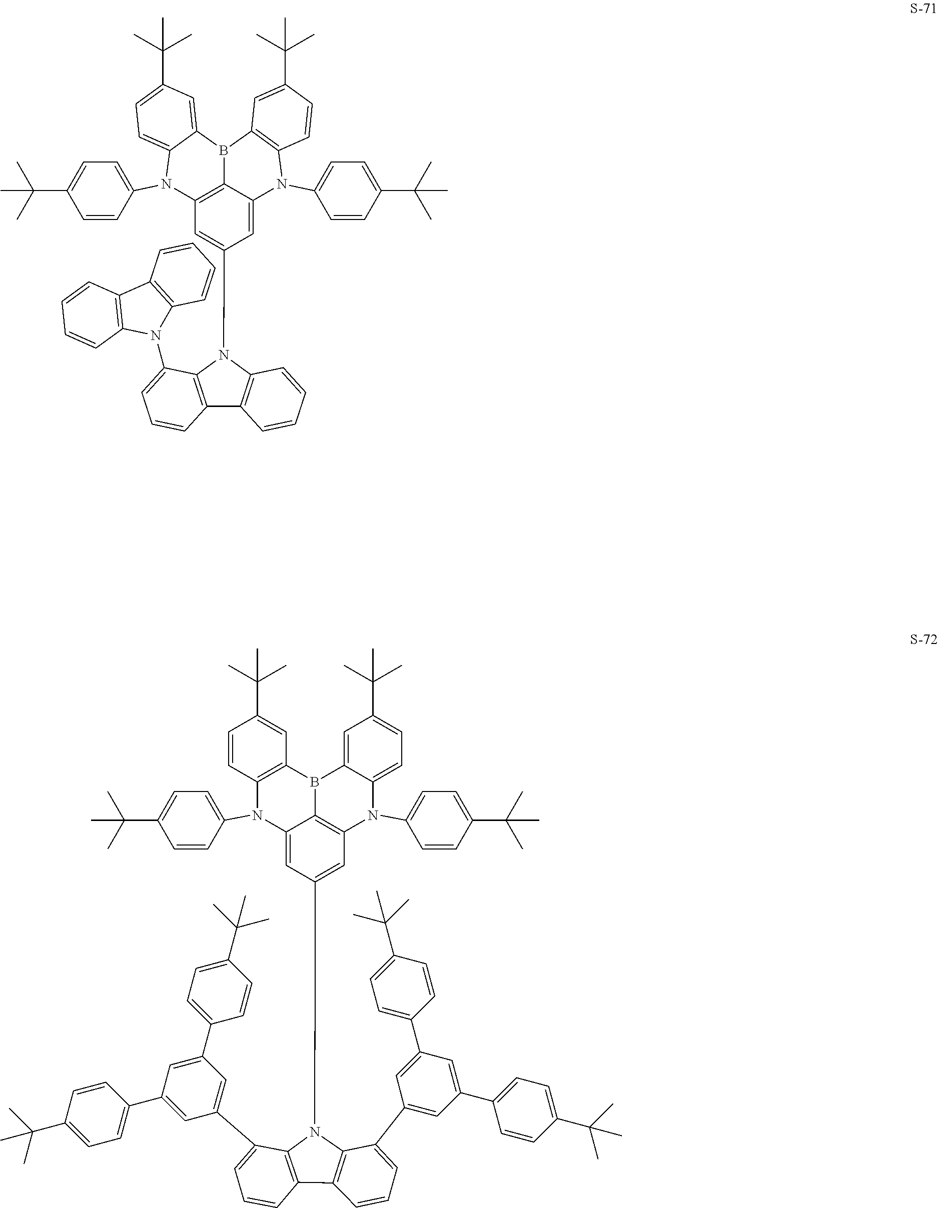

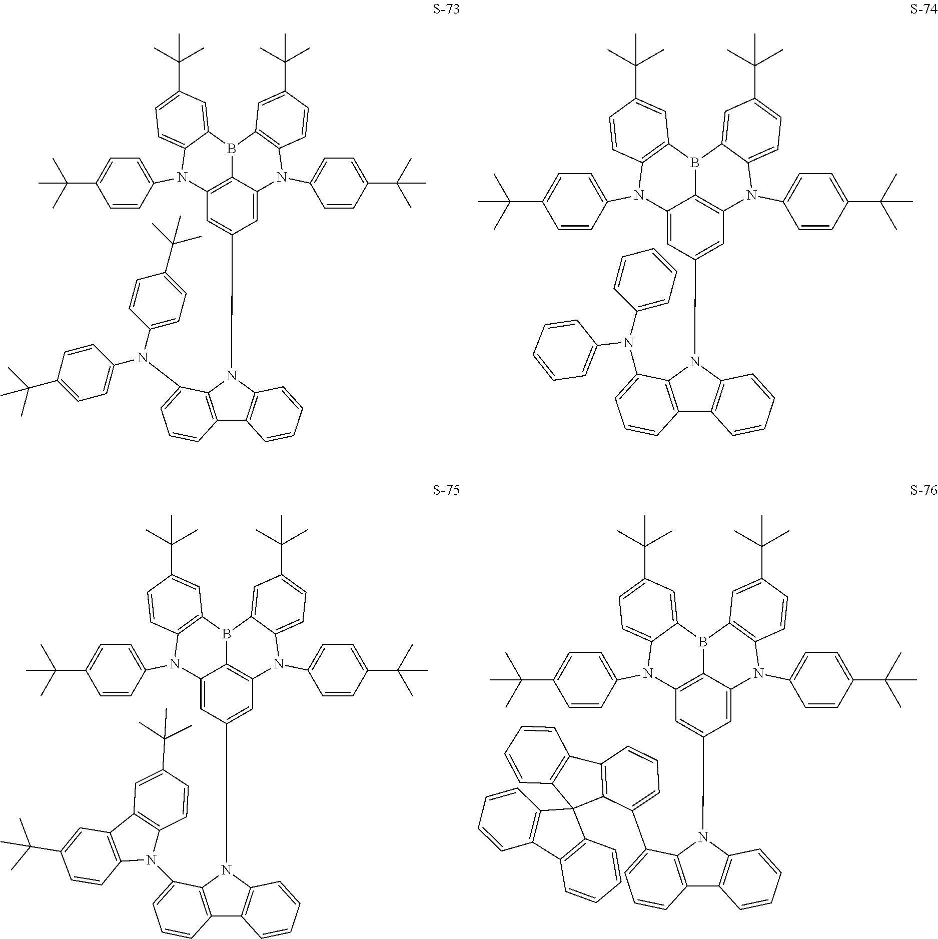

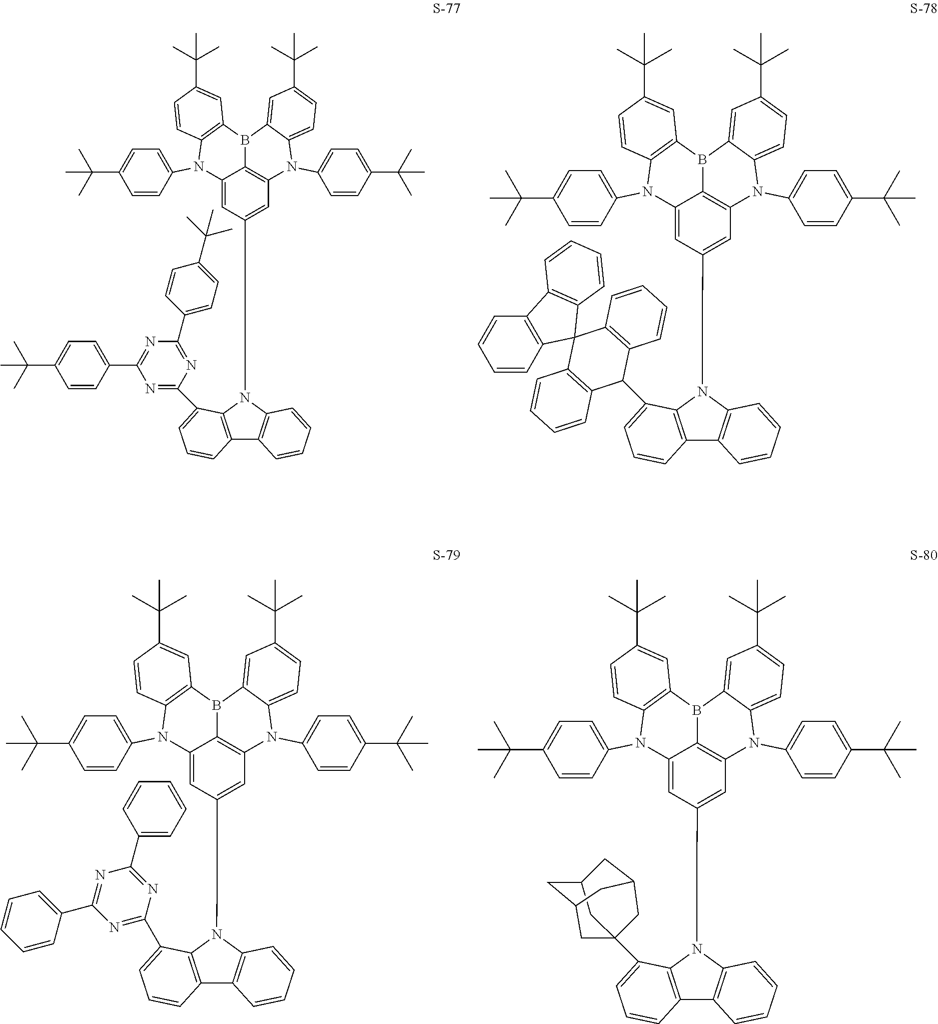

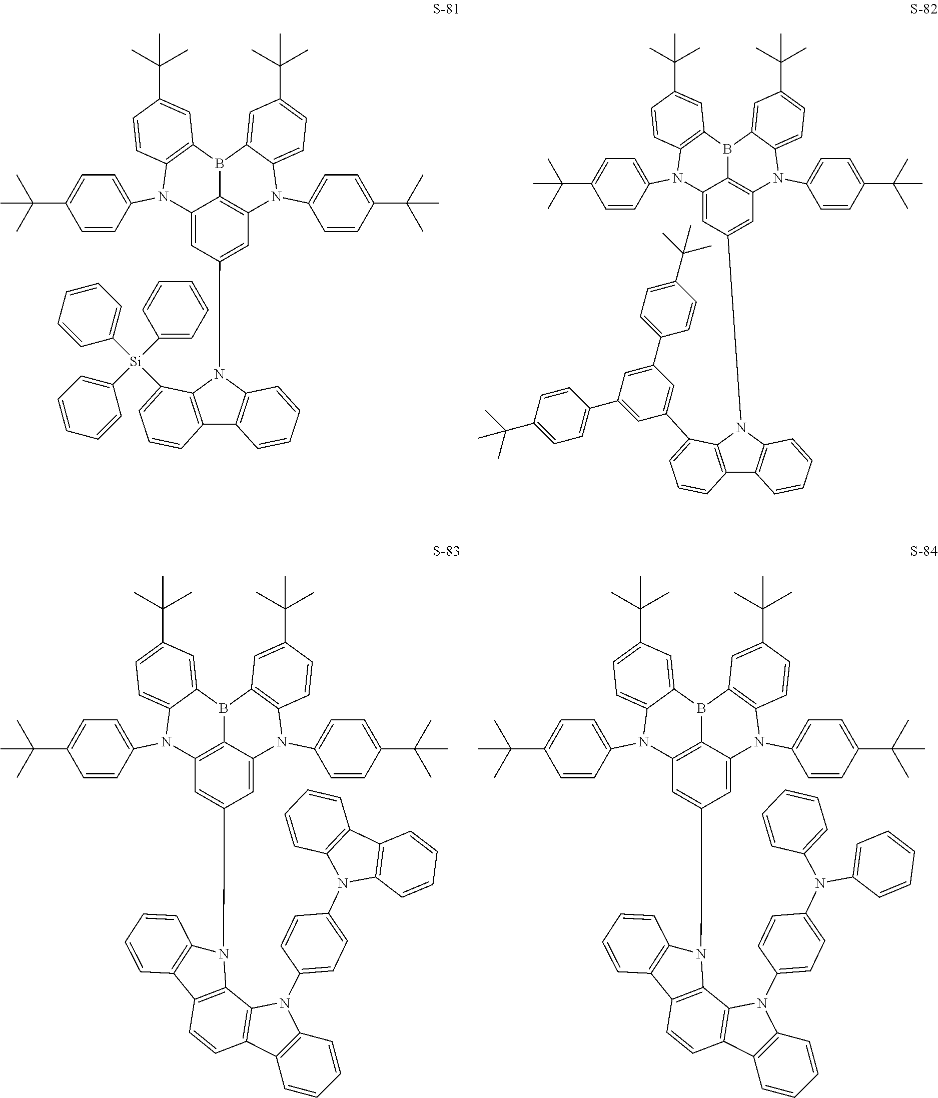

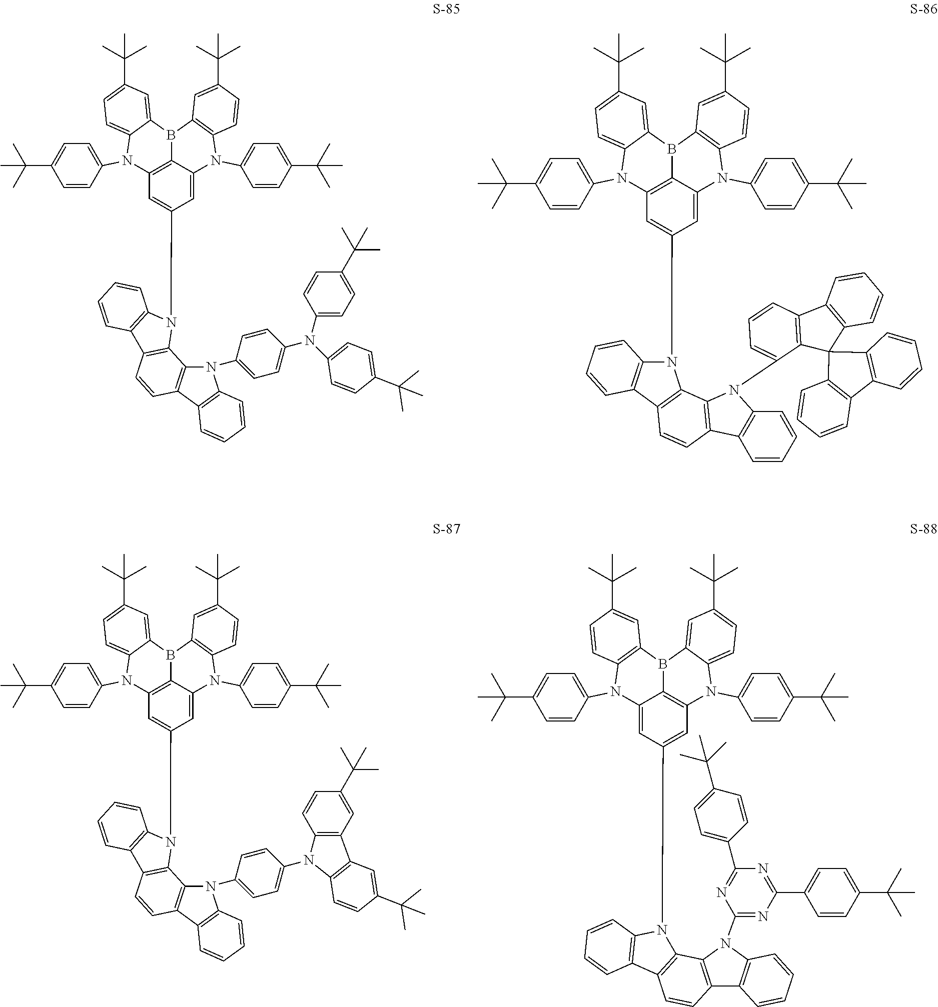

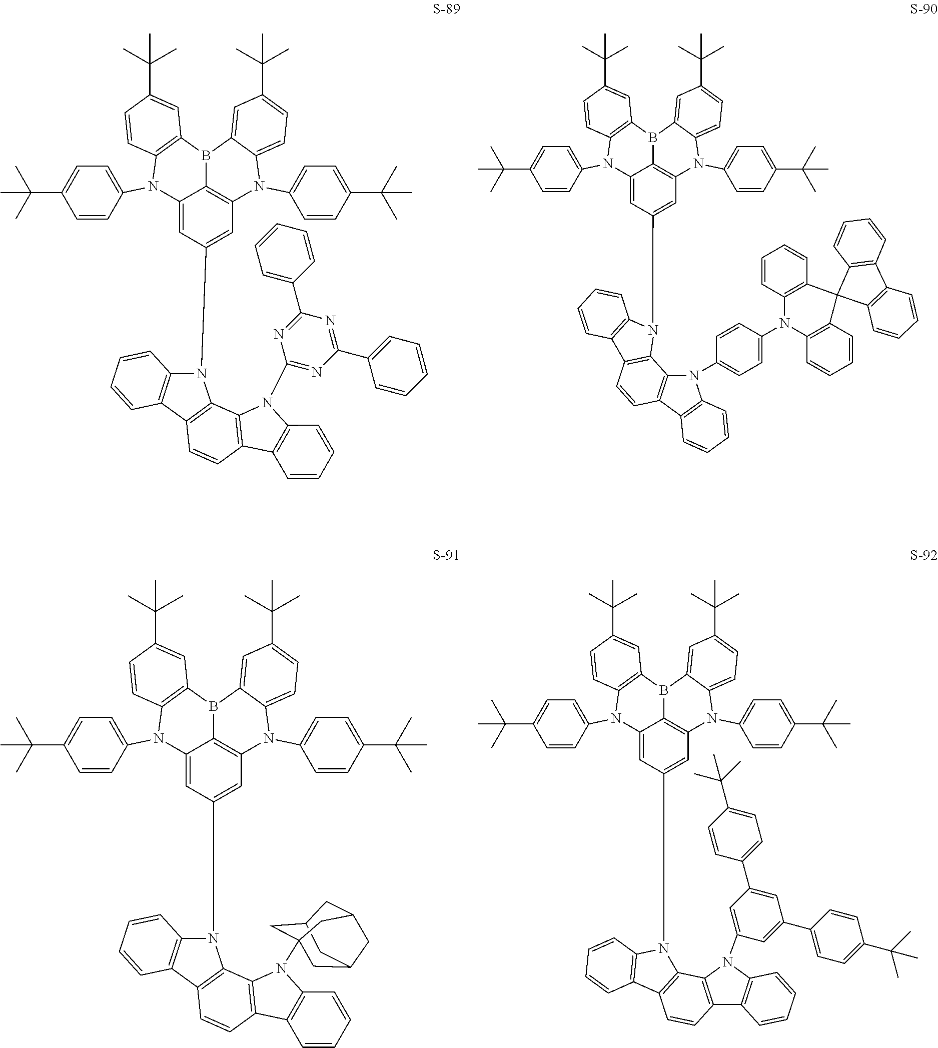

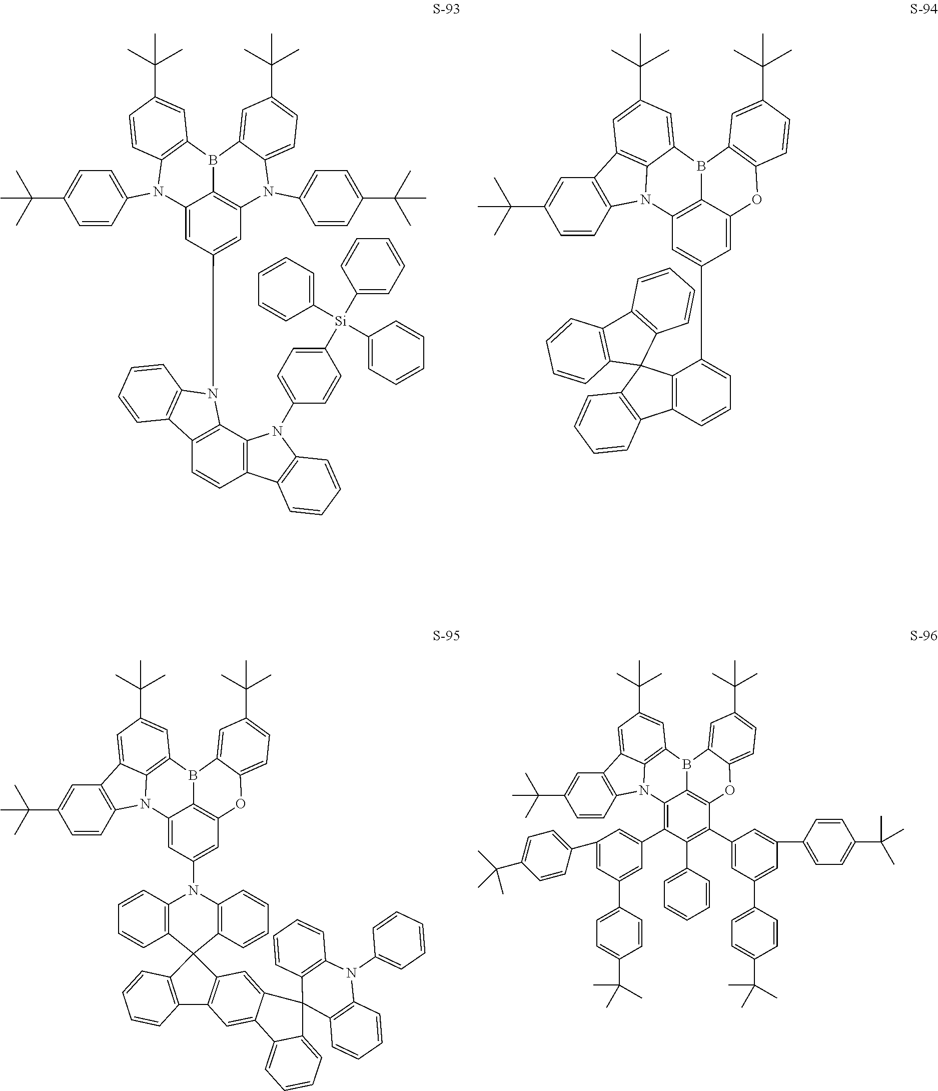

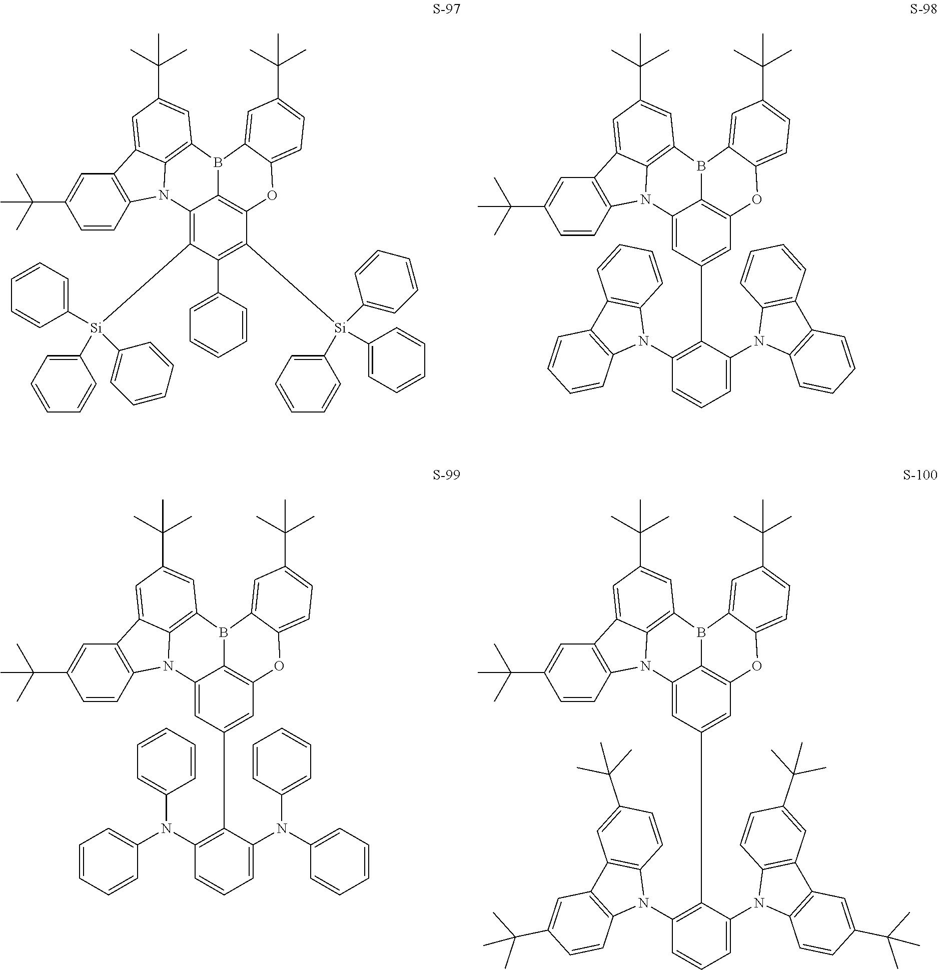

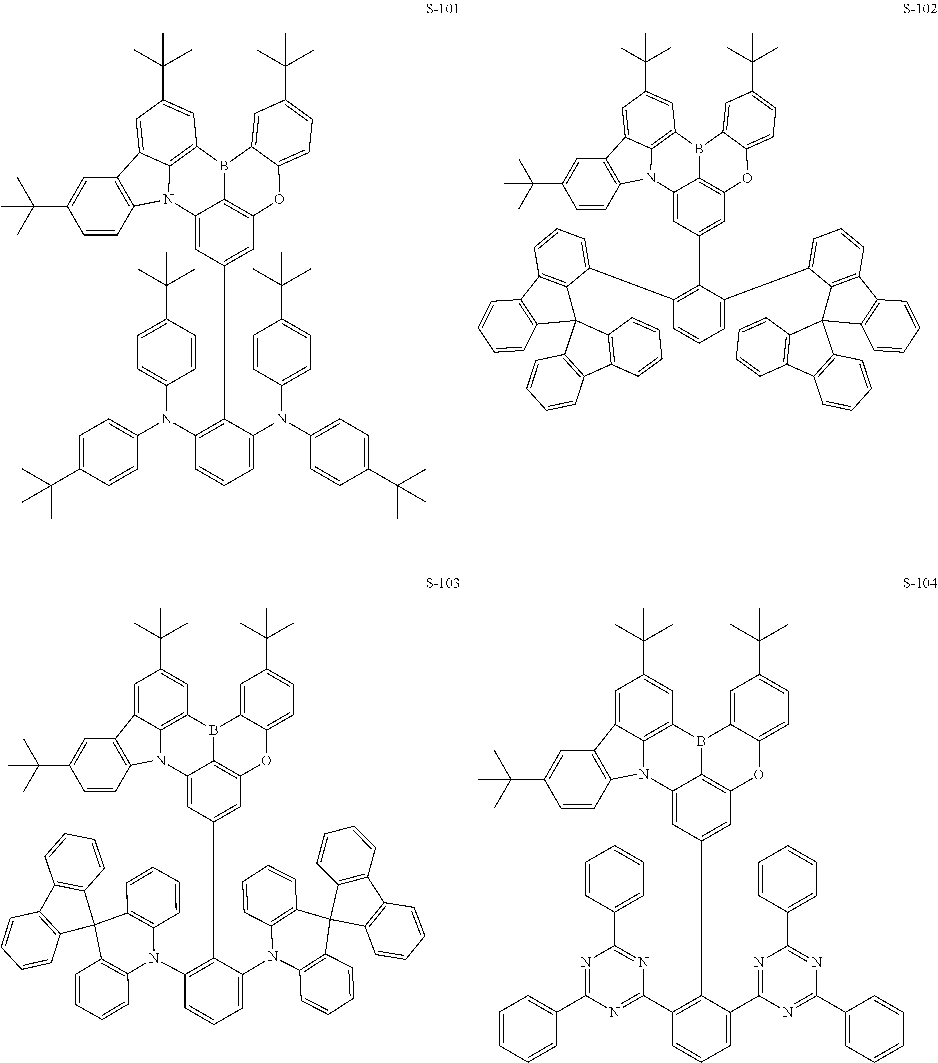

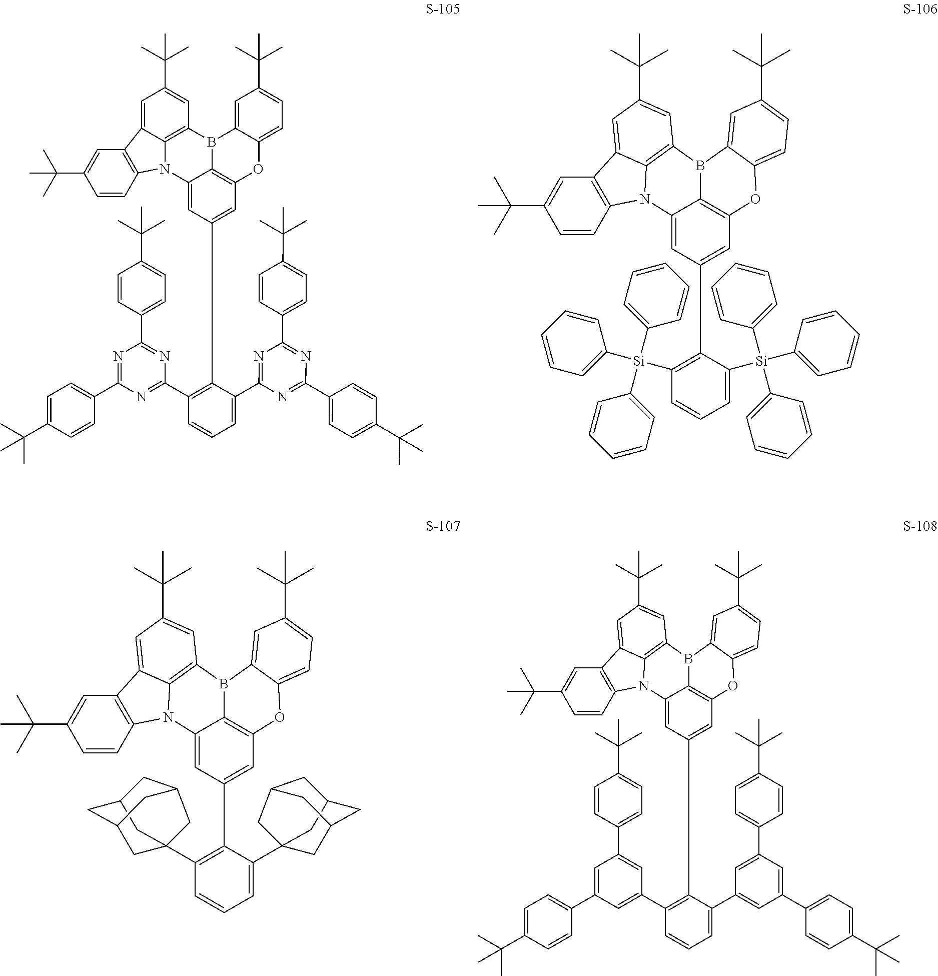

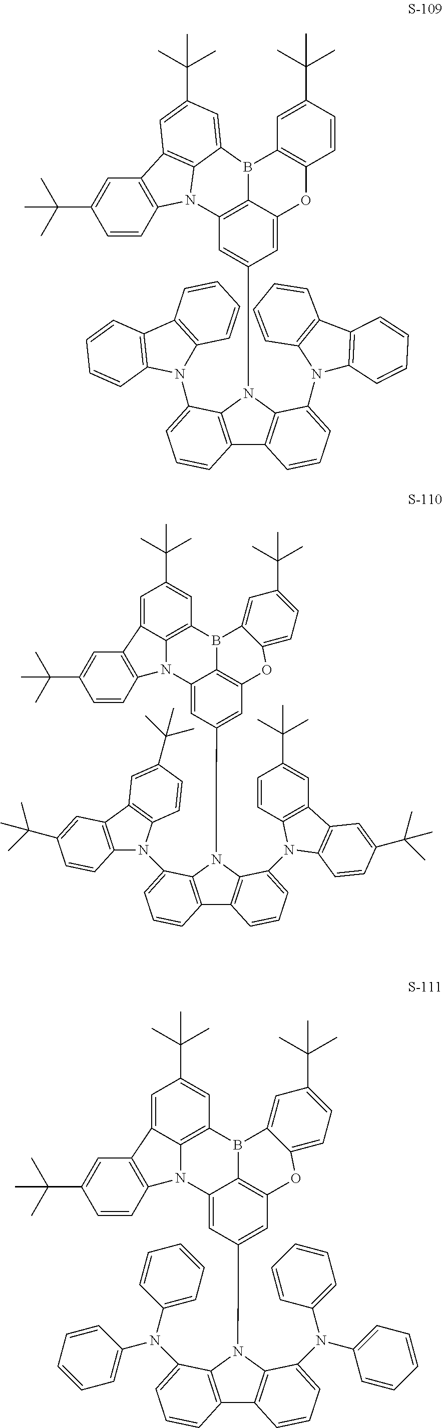

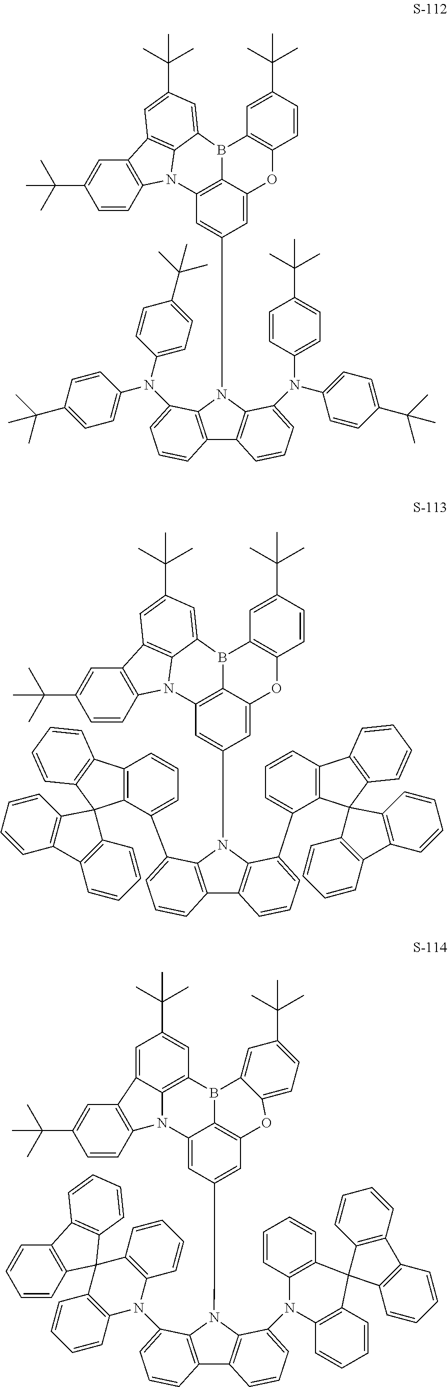

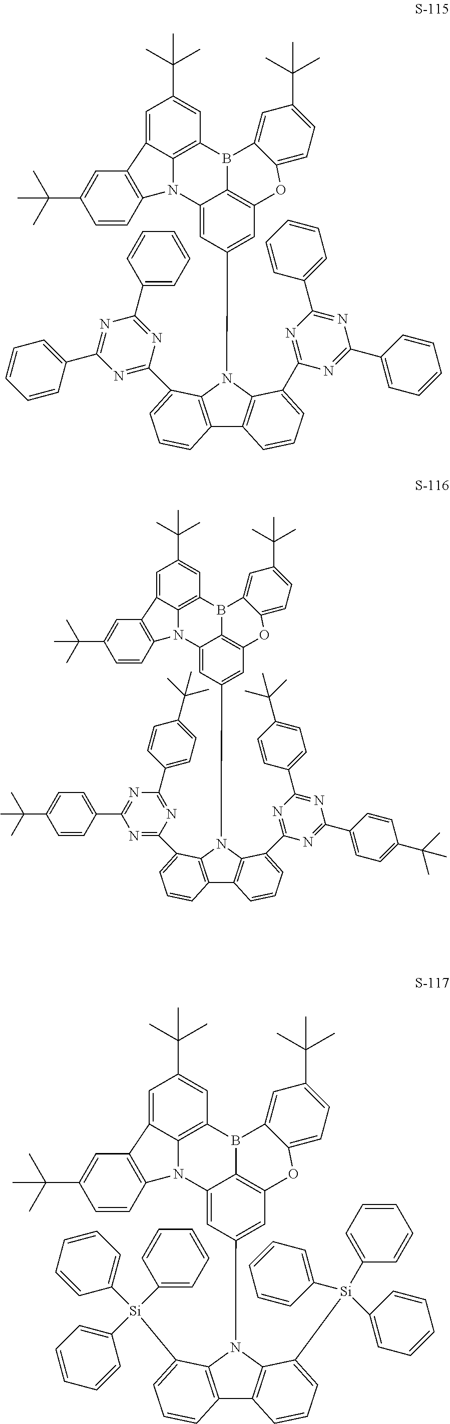

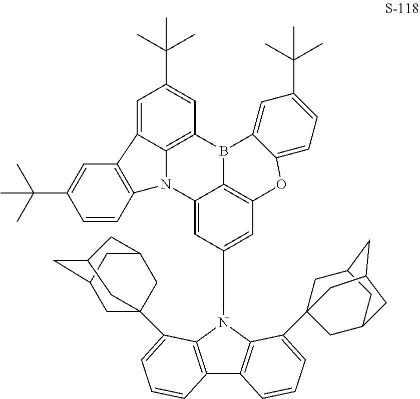 [SMILES: CC(C)(C)c1ccc2c(c1)B1c3c(cc(-n4c5c(C67CC8CC(CC(C8)C6)C7)cccc5c5cccc(C67CC8CC(CC(C8)C6)C7)c54)cc3-n3c4ccc(C(C)(C)C)cc4c4cc(C(C)(C)C)cc1c43)O2]

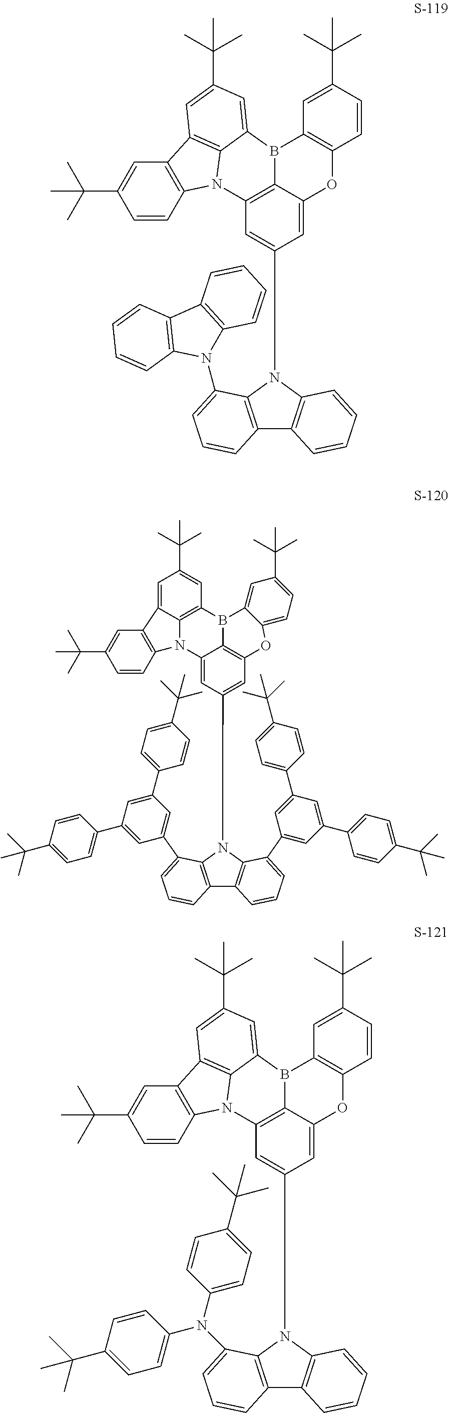

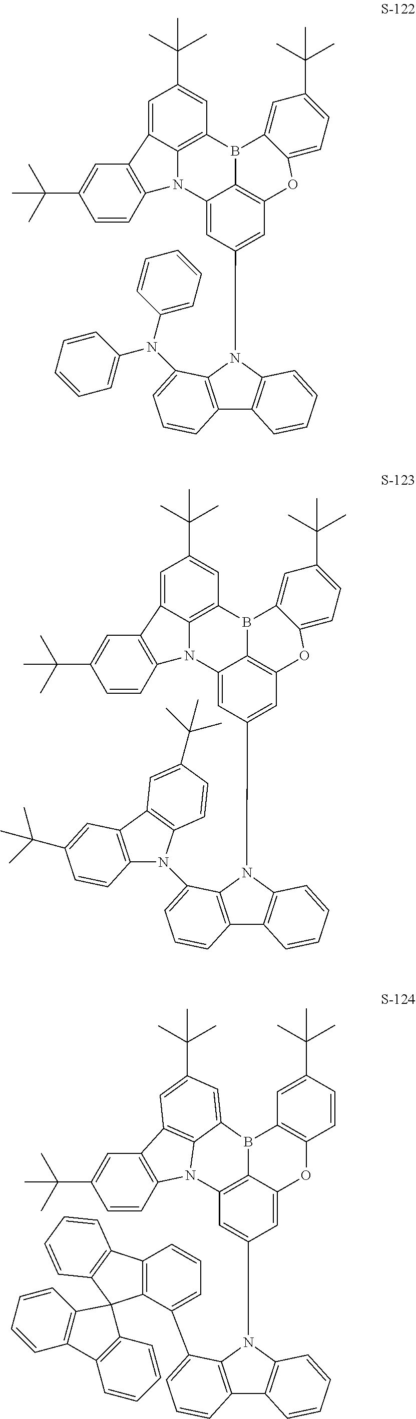

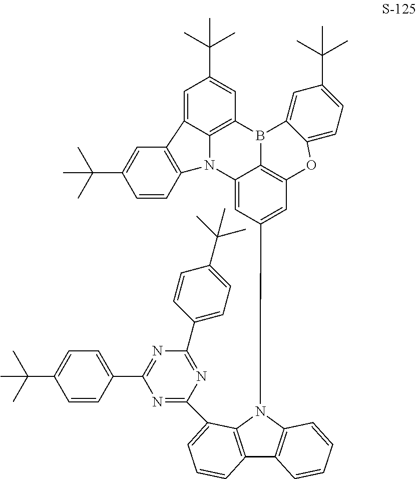 [SMILES: CC(C)(C)c1ccc(-c2nc(-c3ccc(C(C)(C)C)cc3)nc(-c3cccc4c5ccccc5n(-c5cc6c7c(c5)-n5c8ccc(C(C)(C)C)cc8c8cc(C(C)(C)C)cc(c85)B7c5cc(C(C)(C)C)ccc5O6)c34)n2)cc1]

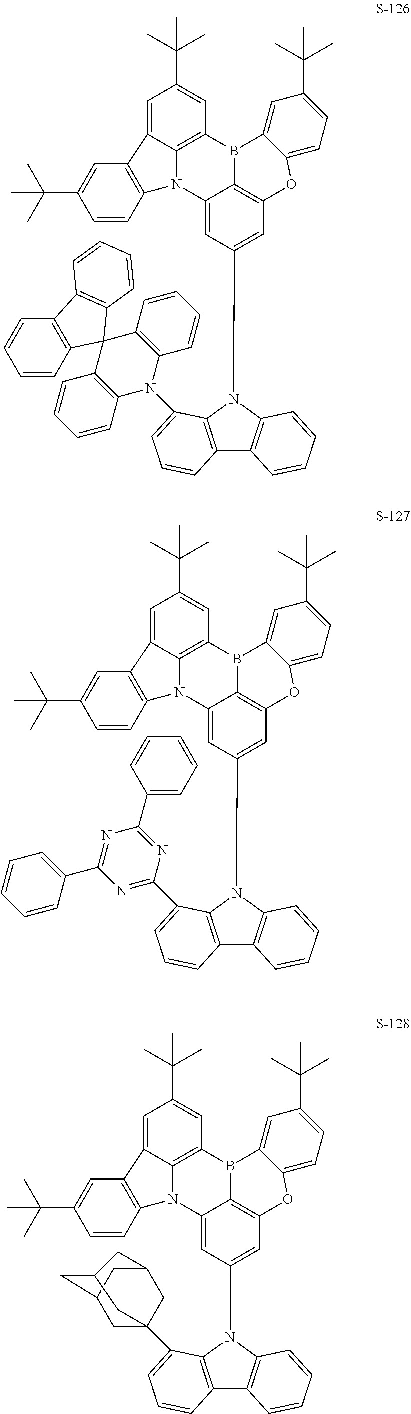

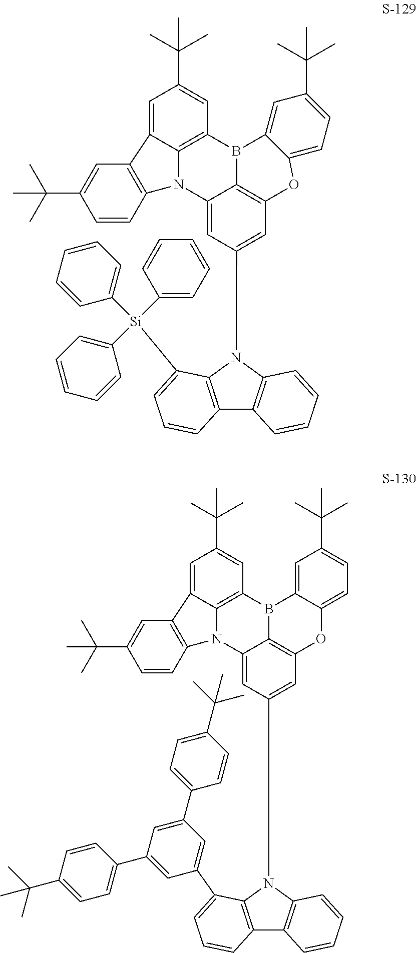

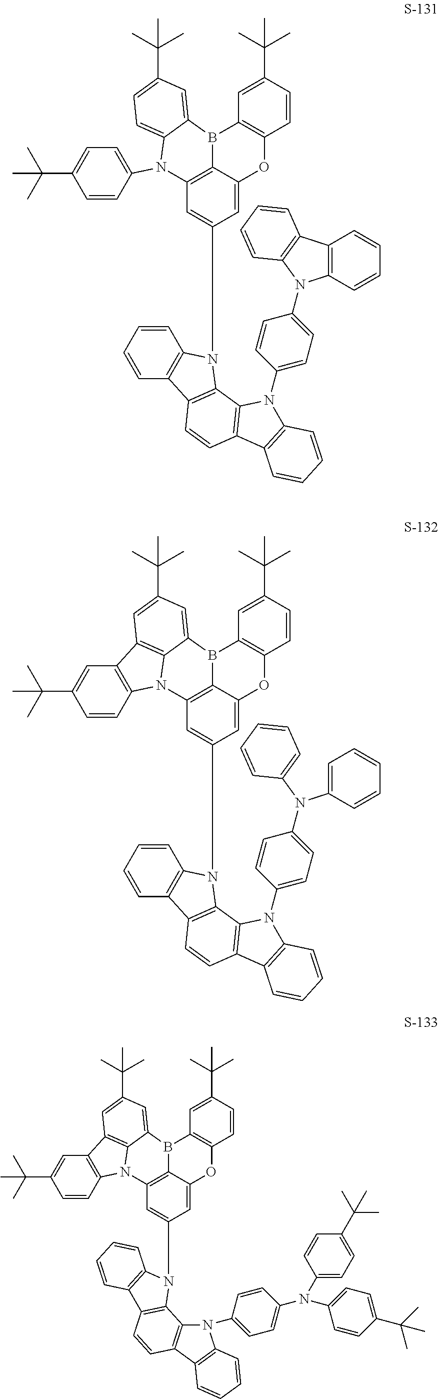 [SMILES: CC(C)(C)c1ccc(N2c3ccc(C(C)(C)C)cc3B3c4cc(C(C)(C)C)ccc4Oc4cc(-n5c6ccccc6c6ccc7c8ccccc8n(-c8ccc(-n9c%10ccccc%10c%10ccccc%109)cc8)c7c65)cc2c43)cc1 ; CC(C)(C)c1ccc2c(c1)B1c3c(cc(-n4c5ccccc5c5ccc6c7ccccc7n(-c7ccc(N(c8ccccc8)c8ccccc8)cc7)c6c54)cc3-n3c4ccc(C(C)(C)C)cc4c4cc(C(C)(C)C)cc1c43)O2 ; CC(C)(C)c1ccc(N(c2ccc(-n3c4ccccc4c4ccc5c6ccccc6n(-c6cc7c8c(c6)-n6c9ccc(C(C)(C)C)cc9c9cc(C(C)(C)C)cc(c96)B8c6cc(C(C)(C)C)ccc6O7)c5c43)cc2)c2ccc(C(C)(C)C)cc2)cc1]

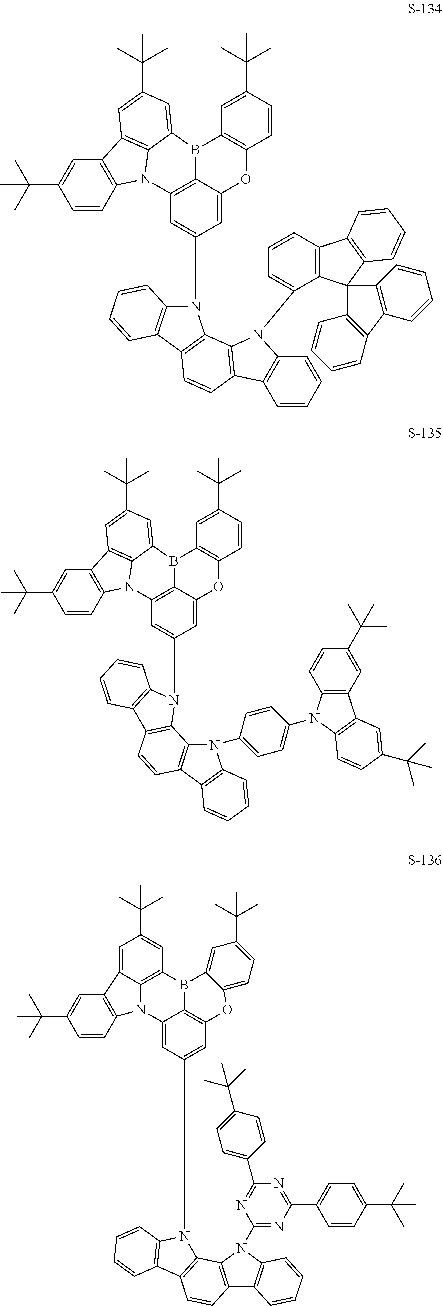

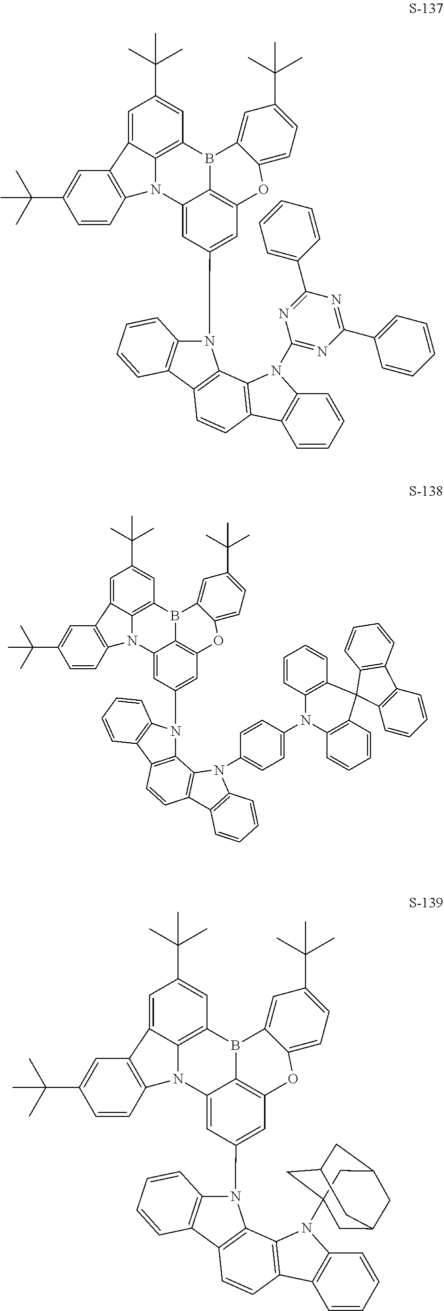 [SMILES: CC(C)(C)c1ccc2c(c1)B1c3c(cc(-n4c5ccccc5c5ccc6c7ccccc7n(-c7nc(-c8ccccc8)nc(-c8ccccc8)n7)c6c54)cc3-n3c4ccc(C(C)(C)C)cc4c4cc(C(C)(C)C)cc1c43)O2 ; CC(C)(C)c1ccc2c(c1)B1c3c(cc(-n4c5ccccc5c5ccc6c7ccccc7n(-c7ccc(N8c9ccccc9C9(c%10ccccc%10-c%10ccccc%109)c9ccccc98)cc7)c6c54)cc3-n3c4ccc(C(C)(C)C)cc4c4cc(C(C)(C)C)cc1c43)O2 ; CC(C)(C)c1ccc2c(c1)B1c3c(cc(-n4c5ccccc5c5ccc6c7ccccc7n(C78CC9CC(CC(C9)C7)C8)c6c54)cc3-n3c4ccc(C(C)(C)C)cc4c4cc(C(C)(C)C)cc1c43)O2]

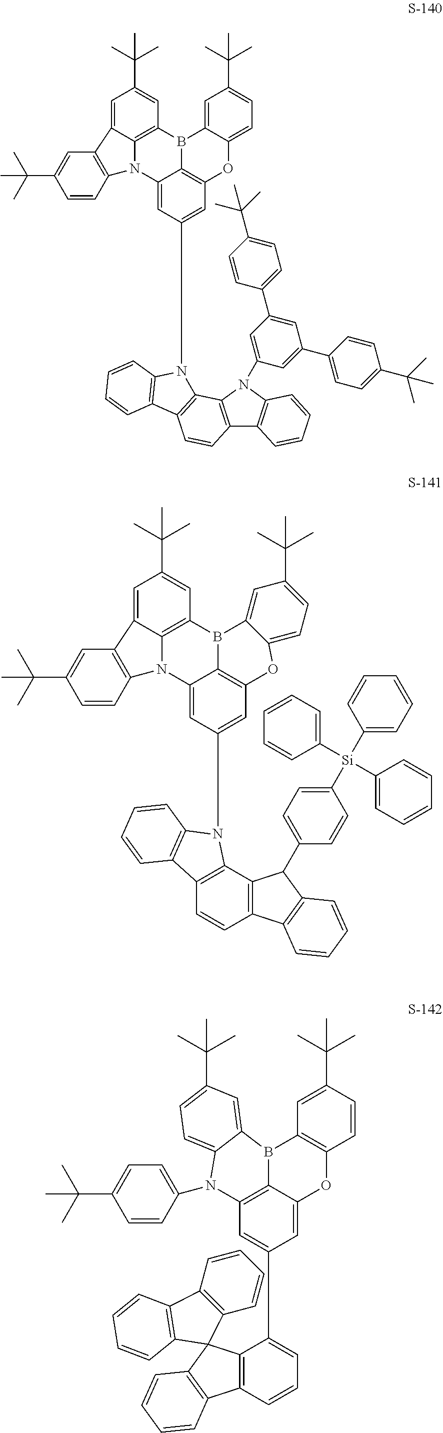

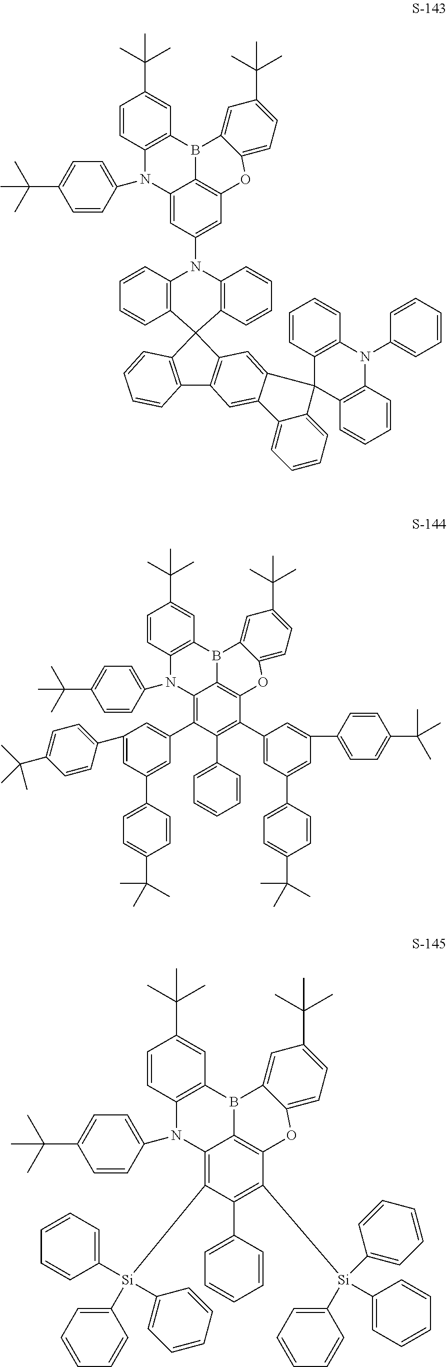

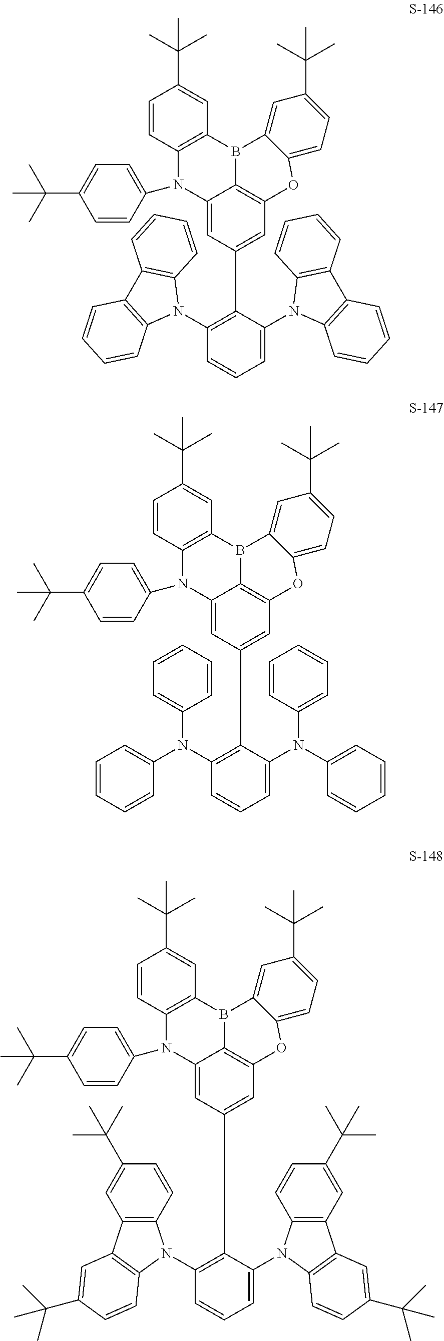 [SMILES: CC(C)(C)c1ccc(N2c3ccc(C(C)(C)C)cc3B3c4cc(C(C)(C)C)ccc4Oc4cc(-c5c(-n6c7ccccc7c7ccccc76)cccc5-n5c6ccccc6c6ccccc65)cc2c43)cc1 ; CC(C)(C)c1ccc(N2c3ccc(C(C)(C)C)cc3B3c4cc(C(C)(C)C)ccc4Oc4cc(-c5c(N(c6ccccc6)c6ccccc6)cccc5N(c5ccccc5)c5ccccc5)cc2c43)cc1 ; CC(C)(C)c1ccc(N2c3ccc(C(C)(C)C)cc3B3c4cc(C(C)(C)C)ccc4Oc4cc(-c5c(-n6c7ccc(C(C)(C)C)cc7c7cc(C(C)(C)C)ccc76)cccc5-n5c6ccc(C(C)(C)C)cc6c6cc(C(C)(C)C)ccc65)cc2c43)cc1]

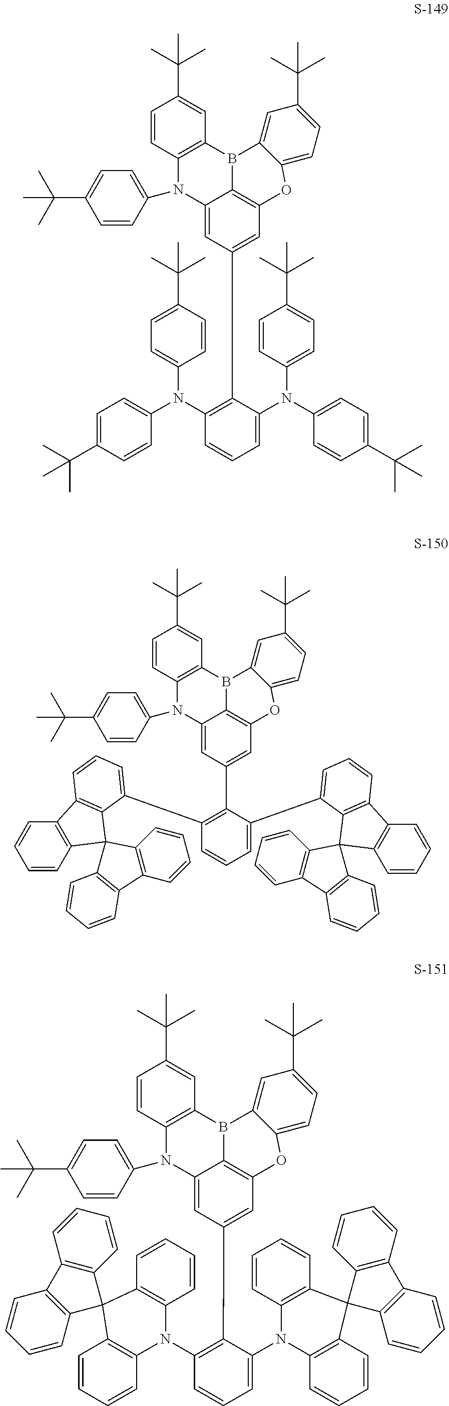

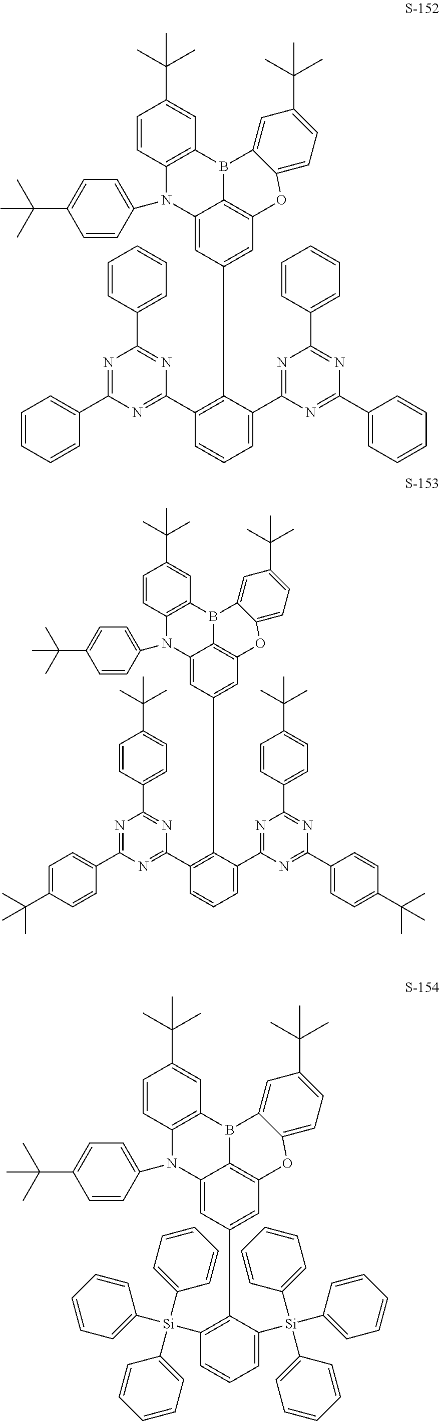

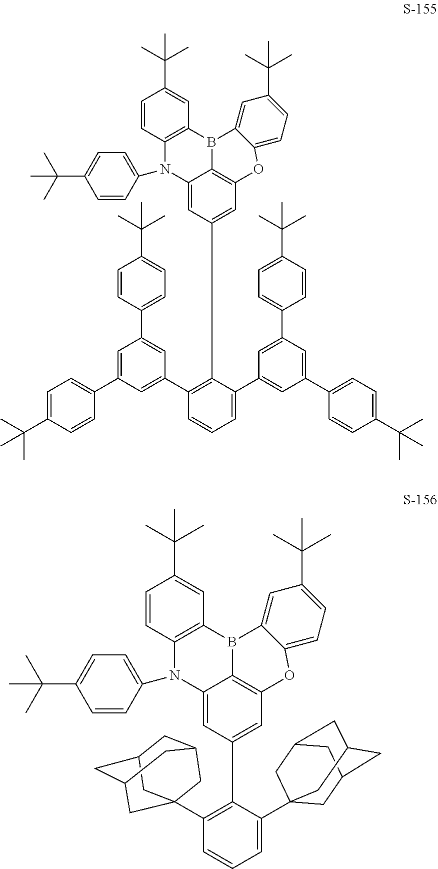

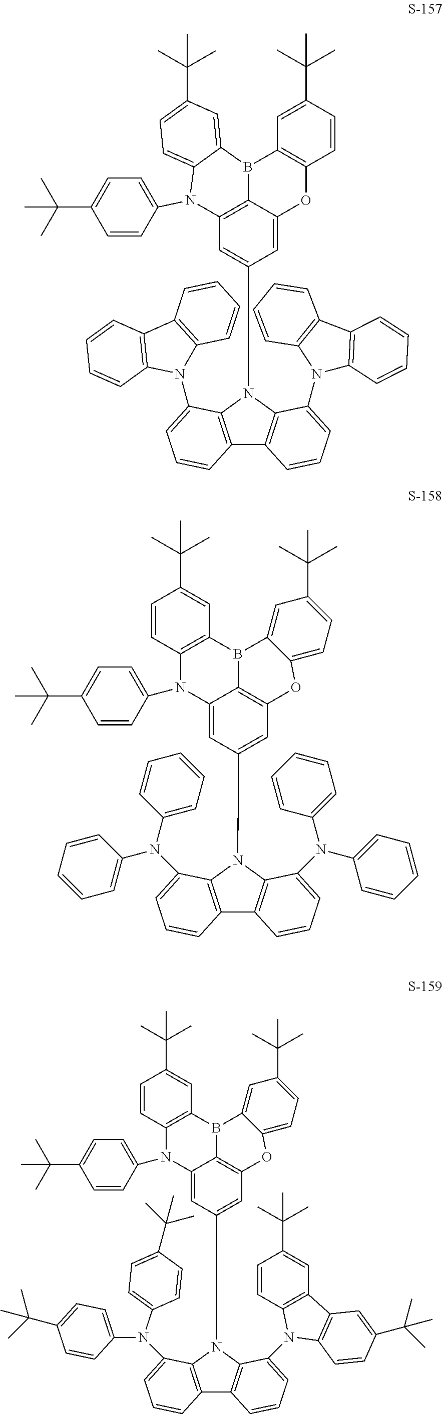 [SMILES: CC(C)(C)c1ccc(N2c3ccc(C(C)(C)C)cc3B3c4cc(C(C)(C)C)ccc4Oc4cc(-n5c6c(-n7c8ccccc8c8ccccc87)cccc6c6cccc(-n7c8ccccc8c8ccccc87)c65)cc2c43)cc1 ; CC(C)(C)c1ccc(N2c3ccc(C(C)(C)C)cc3B3c4cc(C(C)(C)C)ccc4Oc4cc(-n5c6c(N(c7ccccc7)c7ccccc7)cccc6c6cccc(N(c7ccccc7)c7ccccc7)c65)cc2c43)cc1 ; CC(C)(C)c1ccc(N2c3ccc(C(C)(C)C)cc3B3c4cc(C(C)(C)C)ccc4Oc4cc(-n5c6c(N(c7ccc(C(C)(C)C)cc7)c7ccc(C(C)(C)C)cc7)cccc6c6cccc(-n7c8ccc(C(C)(C)C)cc8c8cc(C(C)(C)C)ccc87)c65)cc2c43)cc1]

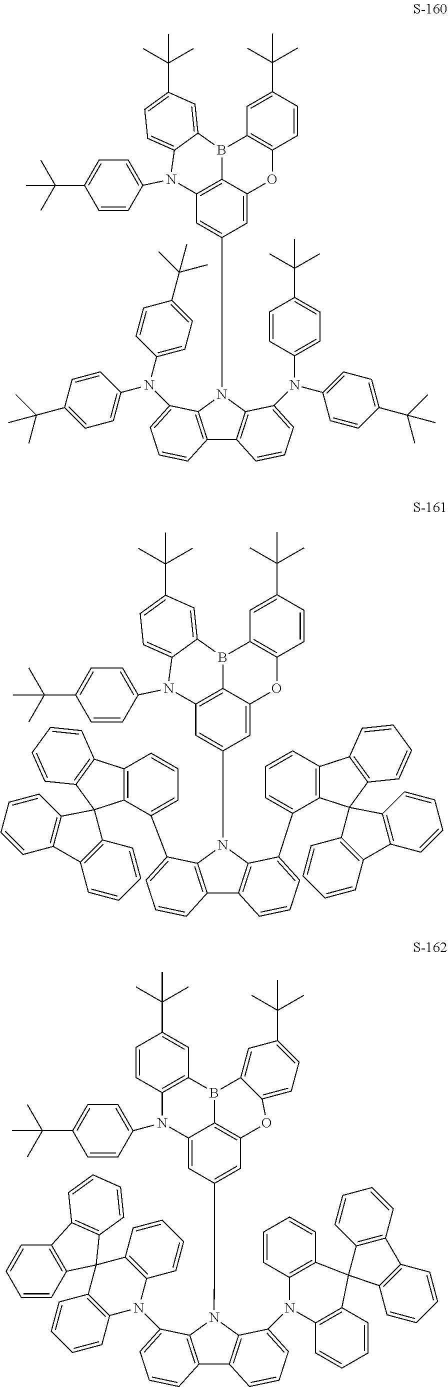

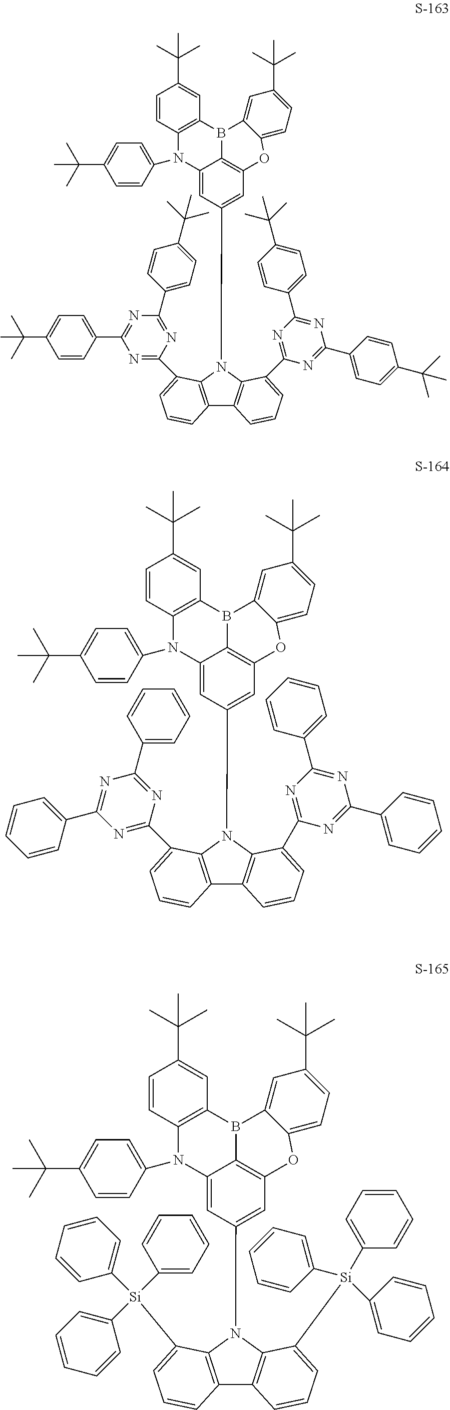

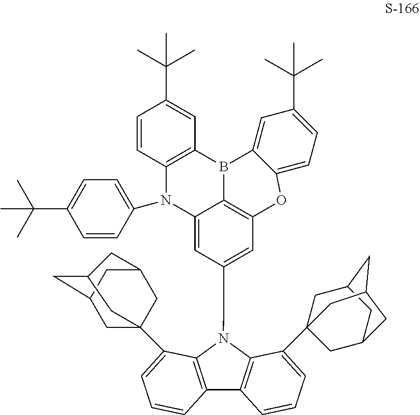 [SMILES: CC(C)(C)c1ccc(N2c3ccc(C(C)(C)C)cc3B3c4cc(C(C)(C)C)ccc4Oc4cc(-n5c6c(C78CC9CC(CC(C9)C7)C8)cccc6c6cccc(C78CC9CC(CC(C9)C7)C8)c65)cc2c43)cc1]

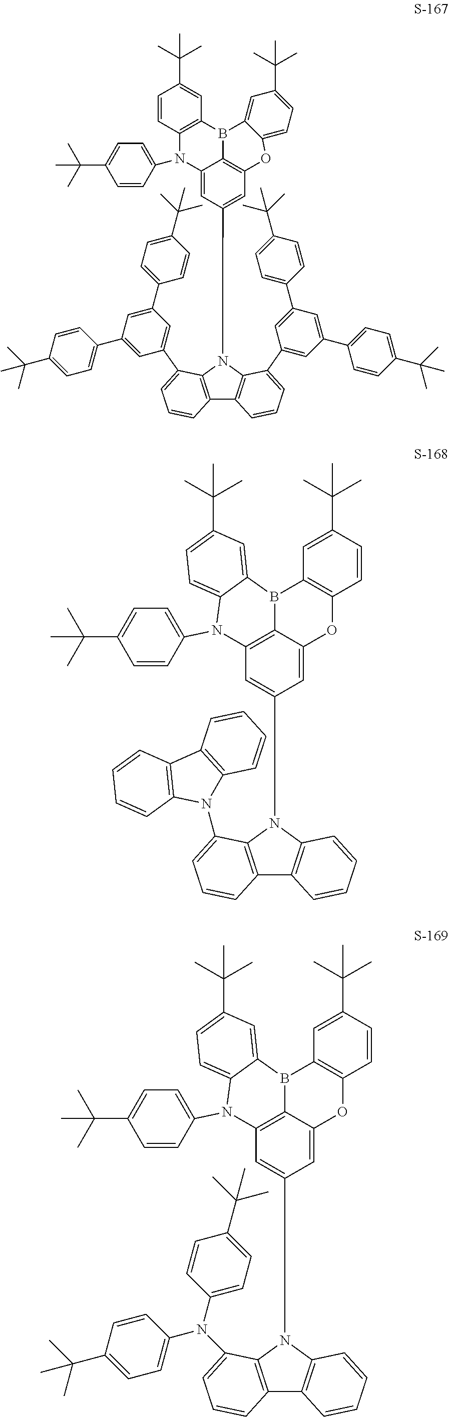

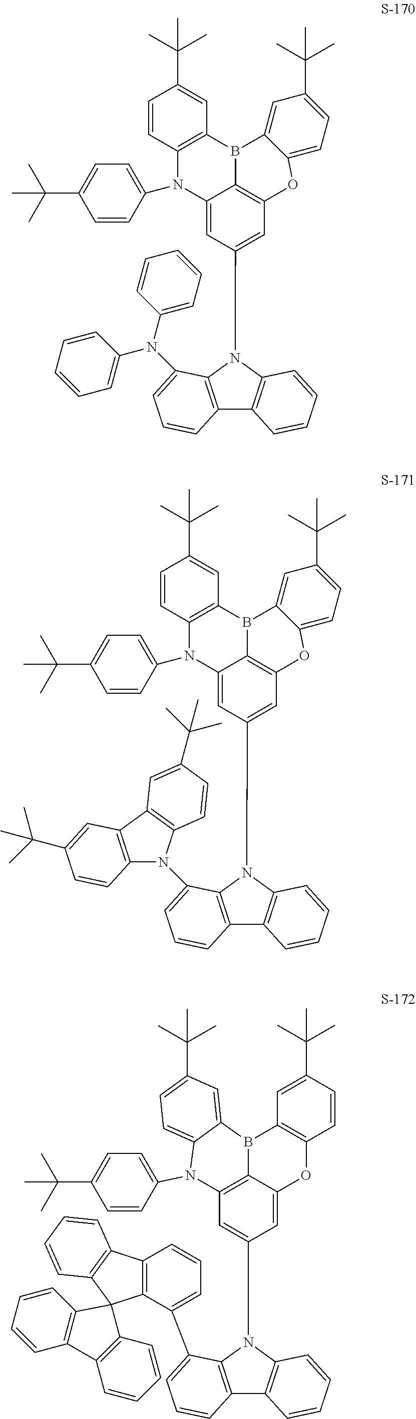

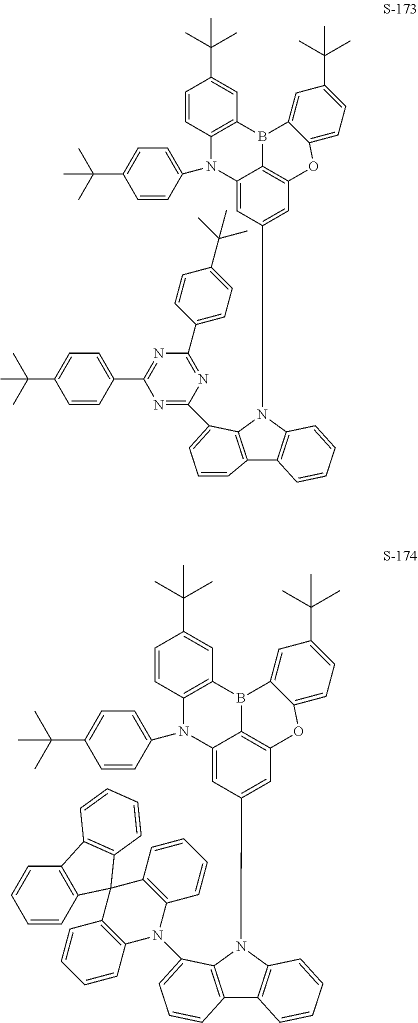 [SMILES: CC(C)(C)c1ccc(-c2nc(-c3ccc(C(C)(C)C)cc3)nc(-c3cccc4c5ccccc5n(-c5cc6c7c(c5)N(c5ccc(C(C)(C)C)cc5)c5ccc(C(C)(C)C)cc5B7c5cc(C(C)(C)C)ccc5O6)c34)n2)cc1 ; CC(C)(C)c1ccc(N2c3ccc(C(C)(C)C)cc3B3c4cc(C(C)(C)C)ccc4Oc4cc(-n5c6ccccc6c6cccc(N7c8ccccc8C8(c9ccccc9-c9ccccc98)c8ccccc87)c65)cc2c43)cc1]

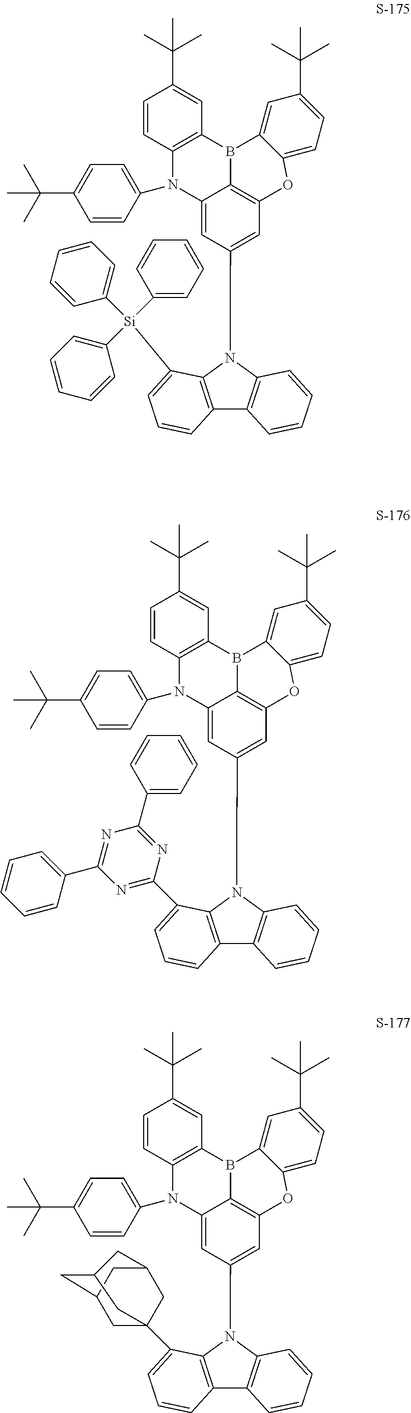

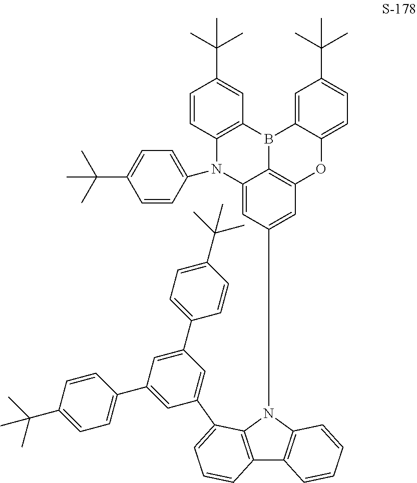 [SMILES: CC(C)(C)c1ccc(-c2cc(-c3ccc(C(C)(C)C)cc3)cc(-c3cccc4c5ccccc5n(-c5cc6c7c(c5)N(c5ccc(C(C)(C)C)cc5)c5ccc(C(C)(C)C)cc5B7c5cc(C(C)(C)C)ccc5O6)c34)c2)cc1]

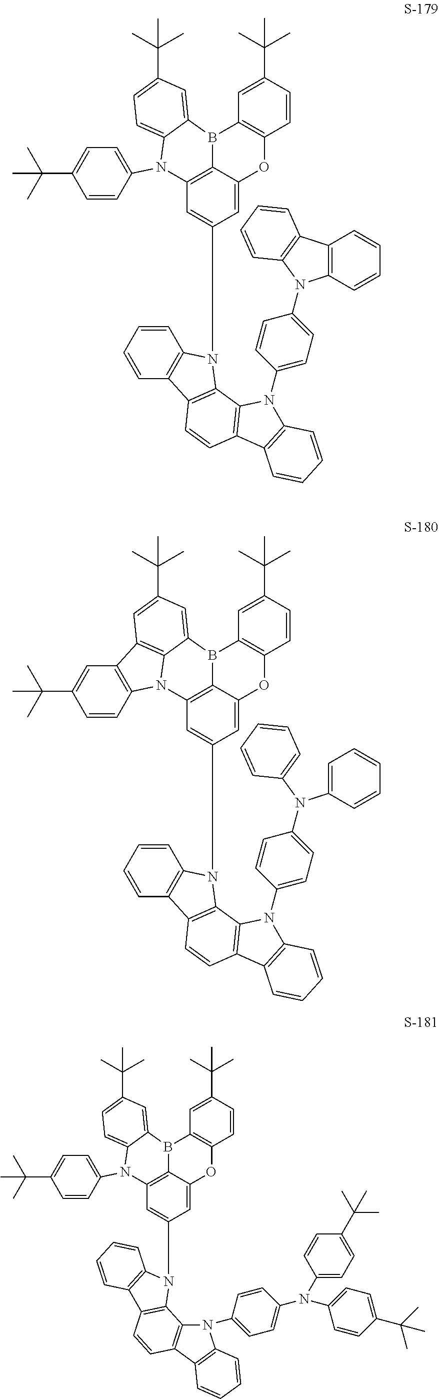 [SMILES: CC(C)(C)c1ccc(N2c3ccc(C(C)(C)C)cc3B3c4cc(C(C)(C)C)ccc4Oc4cc(-n5c6ccccc6c6ccc7c8ccccc8n(-c8ccc(-n9c%10ccccc%10c%10ccccc%109)cc8)c7c65)cc2c43)cc1 ; CC(C)(C)c1ccc2c(c1)B1c3c(cc(-n4c5ccccc5c5ccc6c7ccccc7n(-c7ccc(N(c8ccccc8)c8ccccc8)cc7)c6c54)cc3-n3c4ccc(C(C)(C)C)cc4c4cc(C(C)(C)C)cc1c43)O2 ; CC(C)(C)c1ccc(N(c2ccc(-n3c4ccccc4c4ccc5c6ccccc6n(-c6cc7c8c(c6)N(c6ccc(C(C)(C)C)cc6)c6ccc(C(C)(C)C)cc6B8c6cc(C(C)(C)C)ccc6O7)c5c43)cc2)c2ccc(C(C)(C)C)cc2)cc1]

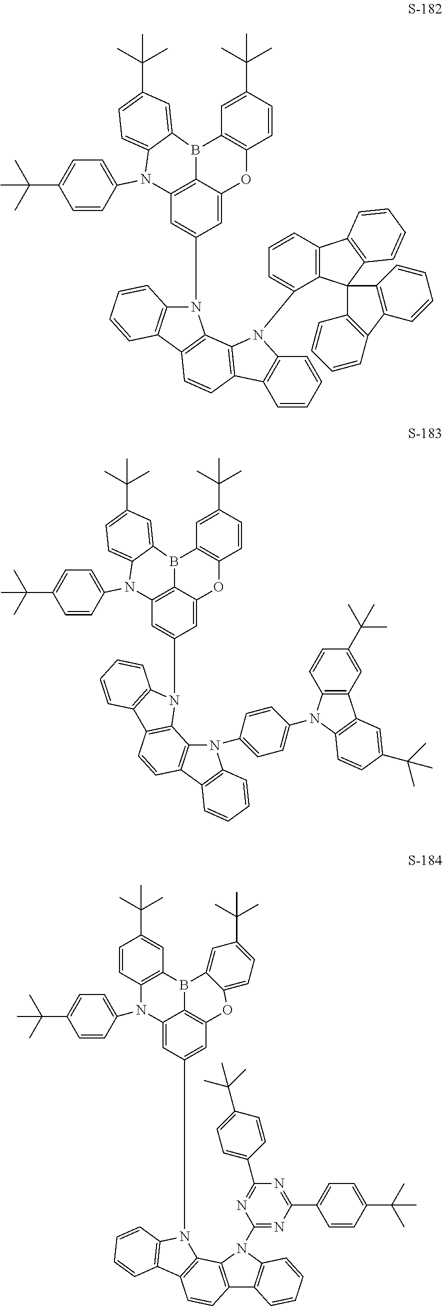

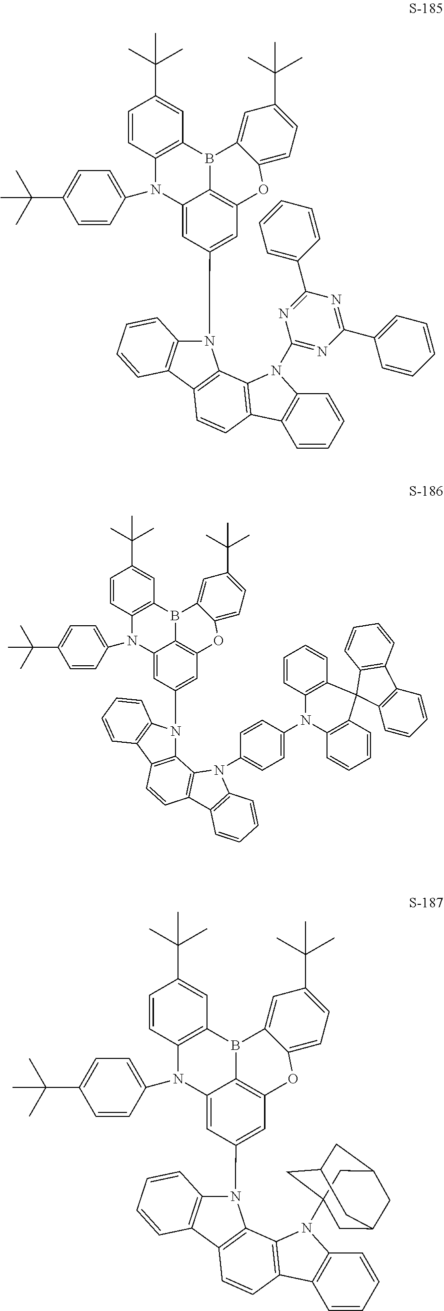 [SMILES: CC(C)(C)c1ccc(N2c3ccc(C(C)(C)C)cc3B3c4cc(C(C)(C)C)ccc4Oc4cc(-n5c6ccccc6c6ccc7c8ccccc8n(-c8nc(-c9ccccc9)nc(-c9ccccc9)n8)c7c65)cc2c43)cc1 ; CC(C)(C)c1ccc(N2c3ccc(C(C)(C)C)cc3B3c4cc(C(C)(C)C)ccc4Oc4cc(-n5c6ccccc6c6ccc7c8ccccc8n(-c8ccc(N9c%10ccccc%10C%10(c%11ccccc%11-c%11ccccc%11%10)c%10ccccc%109)cc8)c7c65)cc2c43)cc1 ; CC(C)(C)c1ccc(N2c3ccc(C(C)(C)C)cc3B3c4cc(C(C)(C)C)ccc4Oc4cc(-n5c6ccccc6c6ccc7c8ccccc8n(C89CC%10CC(CC(C%10)C8)C9)c7c65)cc2c43)cc1]

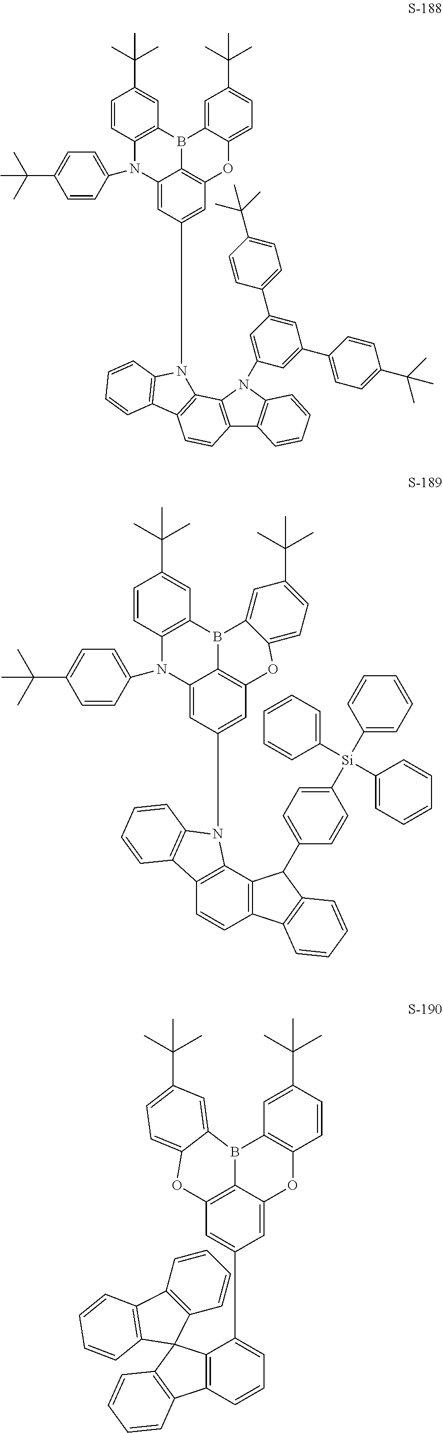

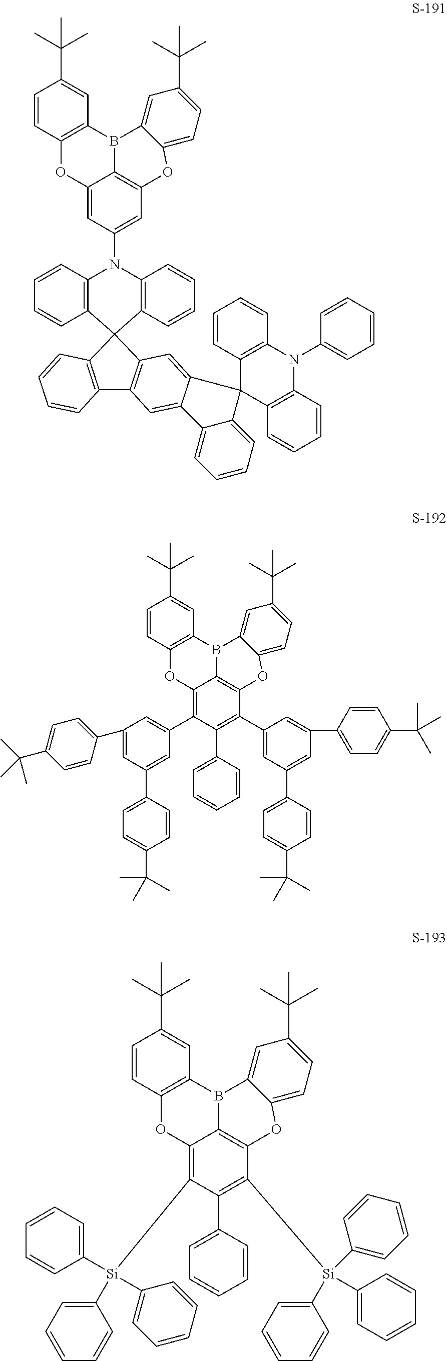 [SMILES: CC(C)(C)c1ccc2c(c1)B1c3cc(C(C)(C)C)ccc3Oc3cc(N4c5ccccc5C5(c6ccccc6-c6cc7c(cc65)C5(c6ccccc6-7)c6ccccc6N(c6ccccc6)c6ccccc65)c5ccccc54)cc(c31)O2 ; CC(C)(C)c1ccc(-c2cc(-c3ccc(C(C)(C)C)cc3)cc(-c3c4c5c(c(-c6cc(-c7ccc(C(C)(C)C)cc7)cc(-c7ccc(C(C)(C)C)cc7)c6)c3-c3ccccc3)Oc3ccc(C(C)(C)C)cc3B5c3cc(C(C)(C)C)ccc3O4)c2)cc1 ; CC(C)(C)c1ccc2c(c1)B1c3cc(C(C)(C)C)ccc3Oc3c1c(c([Si](c1ccccc1)(c1ccccc1)c1ccccc1)c(-c1ccccc1)c3[Si](c1ccccc1)(c1ccccc1)c1ccccc1)O2]

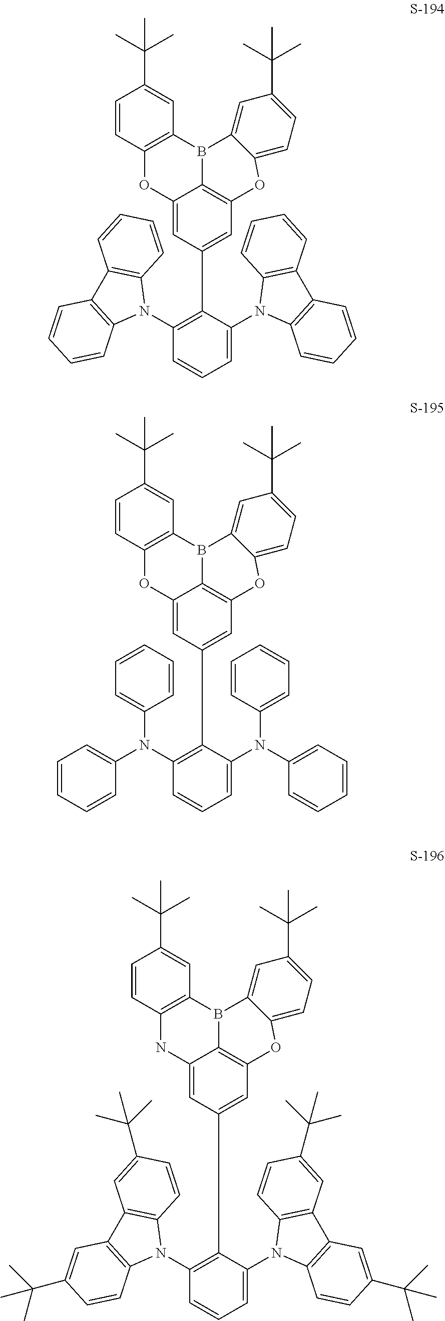 [SMILES: CC(C)(C)c1ccc2c(c1)B1c3cc(C(C)(C)C)ccc3Oc3cc(-c4c(-n5c6ccccc6c6ccccc65)cccc4-n4c5ccccc5c5ccccc54)cc(c31)O2 ; CC(C)(C)c1ccc2c(c1)B1c3cc(C(C)(C)C)ccc3Oc3cc(-c4c(N(c5ccccc5)c5ccccc5)cccc4N(c4ccccc4)c4ccccc4)cc(c31)O2 ; CC(C)(C)c1ccc2c(c1)B1c3cc(C(C)(C)C)ccc3Oc3cc(-c4c(-n5c6ccc(C(C)(C)C)cc6c6cc(C(C)(C)C)ccc65)cccc4-n4c5ccc(C(C)(C)C)cc5c5cc(C(C)(C)C)ccc54)cc(c31)N2]

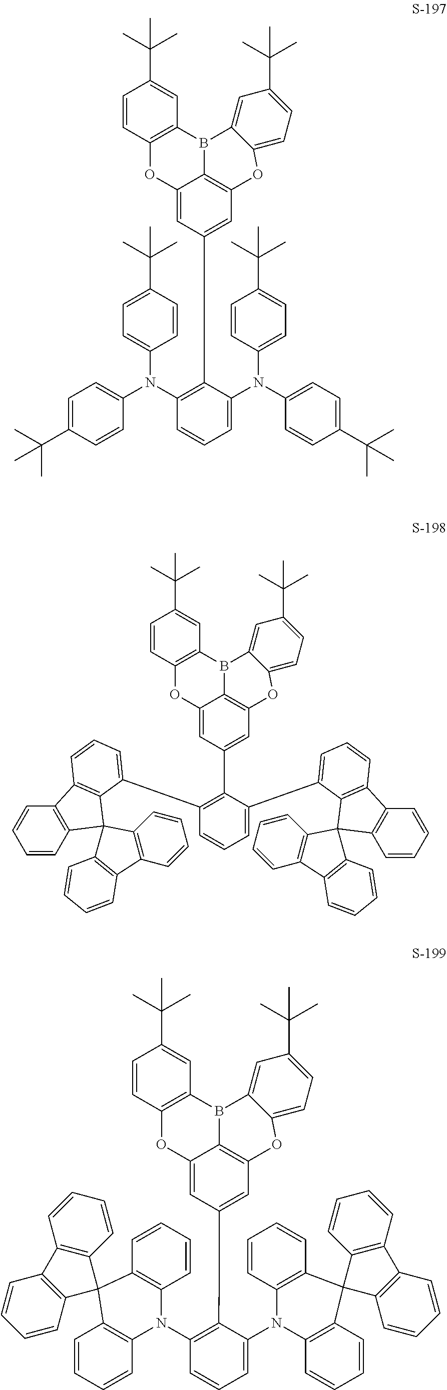 [SMILES: CC(C)(C)c1ccc(N(c2ccc(C(C)(C)C)cc2)c2cccc(N(c3ccc(C(C)(C)C)cc3)c3ccc(C(C)(C)C)cc3)c2-c2cc3c4c(c2)Oc2ccc(C(C)(C)C)cc2B4c2cc(C(C)(C)C)ccc2O3)cc1 ; CC(C)(C)c1ccc2c(c1)B1c3cc(C(C)(C)C)ccc3Oc3cc(-c4c(-c5cccc6c5C5(c7ccccc7-c7ccccc75)c5ccccc5-6)cccc4-c4cccc5c4C4(c6ccccc6-c6ccccc64)c4ccccc4-5)cc(c31)O2 ; CC(C)(C)c1ccc2c(c1)B1c3cc(C(C)(C)C)ccc3Oc3cc(-c4c(N5c6ccccc6C6(c7ccccc7-c7ccccc76)c6ccccc65)cccc4N4c5ccccc5C5(c6ccccc6-c6ccccc65)c5ccccc54)cc(c31)O2]

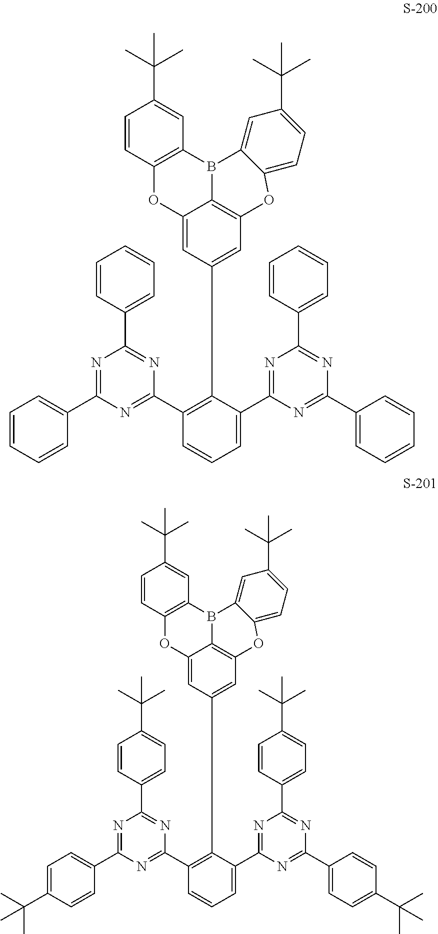 [SMILES: CC(C)(C)c1ccc2c(c1)B1c3cc(C(C)(C)C)ccc3Oc3cc(-c4c(-c5nc(-c6ccccc6)nc(-c6ccccc6)n5)cccc4-c4nc(-c5ccccc5)nc(-c5ccccc5)n4)cc(c31)O2 ; CC(C)(C)c1ccc(-c2nc(-c3ccc(C(C)(C)C)cc3)nc(-c3cccc(-c4nc(-c5ccc(C(C)(C)C)cc5)nc(-c5ccc(C(C)(C)C)cc5)n4)c3-c3cc4c5c(c3)Oc3ccc(C(C)(C)C)cc3B5c3cc(C(C)(C)C)ccc3O4)n2)cc1]

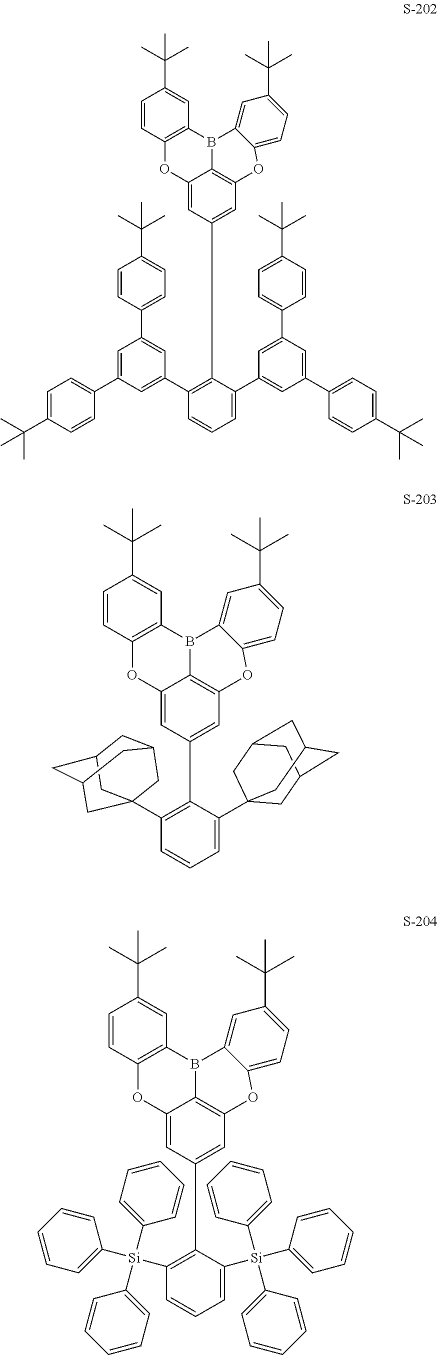 [SMILES: CC(C)(C)c1ccc(-c2cc(-c3ccc(C(C)(C)C)cc3)cc(-c3cccc(-c4cc(-c5ccc(C(C)(C)C)cc5)cc(-c5ccc(C(C)(C)C)cc5)c4)c3-c3cc4c5c(c3)Oc3ccc(C(C)(C)C)cc3B5c3cc(C(C)(C)C)ccc3O4)c2)cc1 ; CC(C)(C)c1ccc2c(c1)B1c3cc(C(C)(C)C)ccc3Oc3cc(-c4c(C56CC7CC(CC(C7)C5)C6)cccc4C45CC6CC(CC(C6)C4)C5)cc(c31)O2 ; CC(C)(C)c1ccc2c(c1)B1c3cc(C(C)(C)C)ccc3Oc3cc(-c4c([Si](c5ccccc5)(c5ccccc5)c5ccccc5)cccc4[Si](c4ccccc4)(c4ccccc4)c4ccccc4)cc(c31)O2]

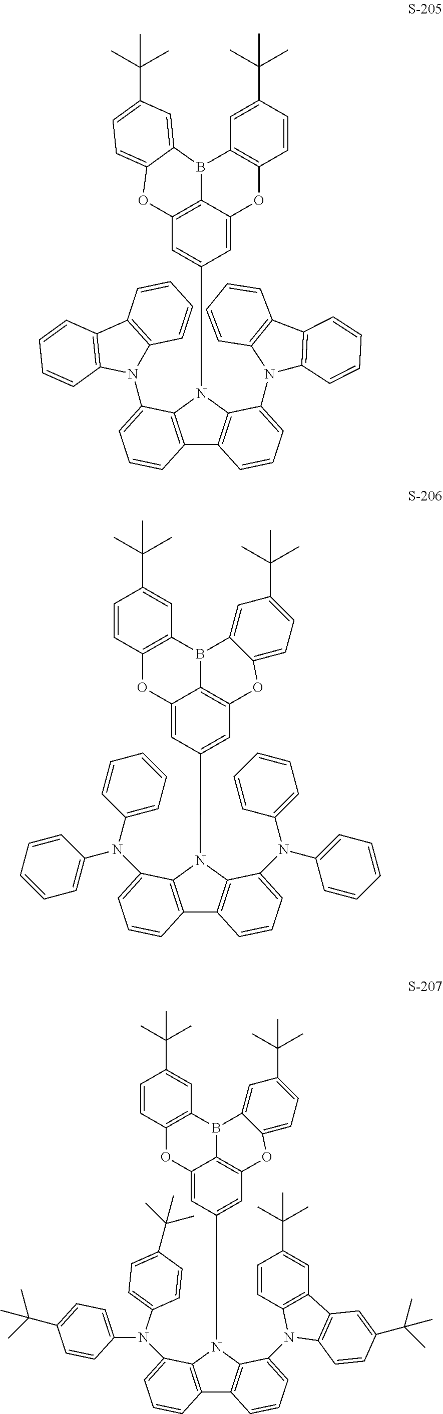 [SMILES: CC(C)(C)c1ccc2c(c1)B1c3cc(C(C)(C)C)ccc3Oc3cc(-n4c5c(-n6c7ccccc7c7ccccc76)cccc5c5cccc(-n6c7ccccc7c7ccccc76)c54)cc(c31)O2 ; CC(C)(C)c1ccc2c(c1)B1c3cc(C(C)(C)C)ccc3Oc3cc(-n4c5c(N(c6ccccc6)c6ccccc6)cccc5c5cccc(N(c6ccccc6)c6ccccc6)c54)cc(c31)O2 ; CC(C)(C)c1ccc(N(c2ccc(C(C)(C)C)cc2)c2cccc3c4cccc(-n5c6ccc(C(C)(C)C)cc6c6cc(C(C)(C)C)ccc65)c4n(-c4cc5c6c(c4)Oc4ccc(C(C)(C)C)cc4B6c4cc(C(C)(C)C)ccc4O5)c23)cc1]

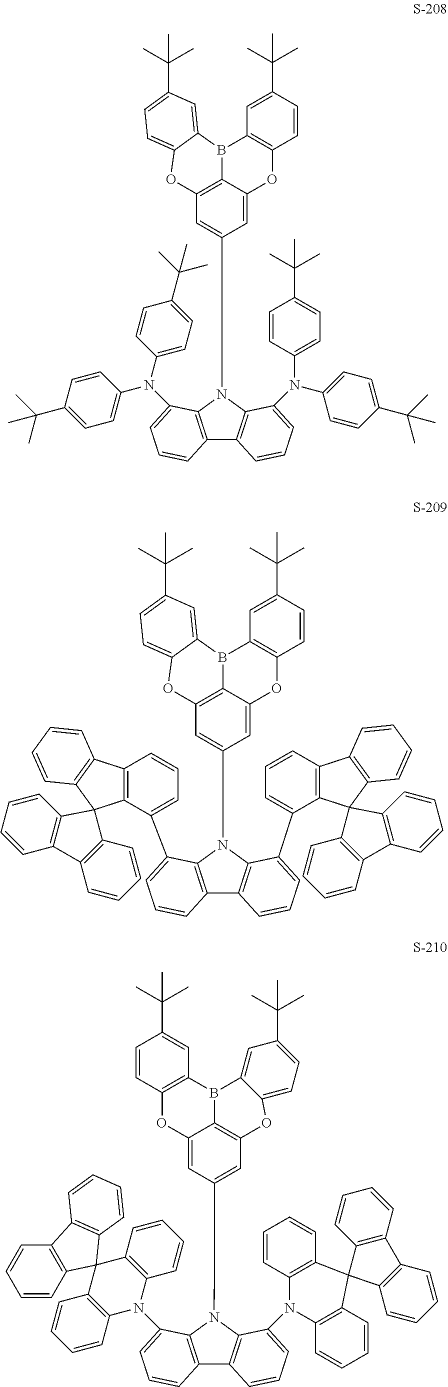 [SMILES: CC(C)(C)c1ccc(N(c2ccc(C(C)(C)C)cc2)c2cccc3c4cccc(N(c5ccc(C(C)(C)C)cc5)c5ccc(C(C)(C)C)cc5)c4n(-c4cc5c6c(c4)Oc4ccc(C(C)(C)C)cc4B6c4cc(C(C)(C)C)ccc4O5)c23)cc1 ; CC(C)(C)c1ccc2c(c1)B1c3cc(C(C)(C)C)ccc3Oc3cc(-n4c5c(-c6cccc7c6C6(c8ccccc8-c8ccccc86)c6ccccc6-7)cccc5c5cccc(-c6cccc7c6C6(c8ccccc8-c8ccccc86)c6ccccc6-7)c54)cc(c31)O2 ; CC(C)(C)c1ccc2c(c1)B1c3cc(C(C)(C)C)ccc3Oc3cc(-n4c5c(N6c7ccccc7C7(c8ccccc8-c8ccccc87)c7ccccc76)cccc5c5cccc(N6c7ccccc7C7(c8ccccc8-c8ccccc87)c7ccccc76)c54)cc(c31)O2]

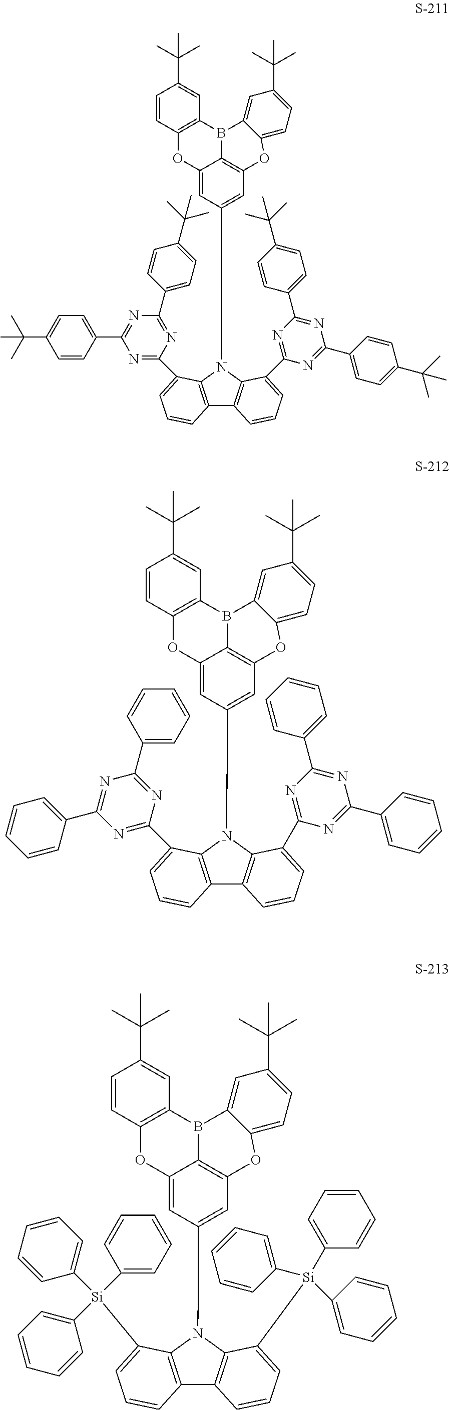 [SMILES: CC(C)(C)c1ccc(-c2nc(-c3ccc(C(C)(C)C)cc3)nc(-c3cccc4c5cccc(-c6nc(-c7ccc(C(C)(C)C)cc7)nc(-c7ccc(C(C)(C)C)cc7)n6)c5n(-c5cc6c7c(c5)Oc5ccc(C(C)(C)C)cc5B7c5cc(C(C)(C)C)ccc5O6)c34)n2)cc1 ; CC(C)(C)c1ccc2c(c1)B1c3cc(C(C)(C)C)ccc3Oc3cc(-n4c5c(-c6nc(-c7ccccc7)nc(-c7ccccc7)n6)cccc5c5cccc(-c6nc(-c7ccccc7)nc(-c7ccccc7)n6)c54)cc(c31)O2 ; CC(C)(C)c1ccc2c(c1)B1c3cc(C(C)(C)C)ccc3Oc3cc(-n4c5c([Si](c6ccccc6)(c6ccccc6)c6ccccc6)cccc5c5cccc([Si](c6ccccc6)(c6ccccc6)c6ccccc6)c54)cc(c31)O2]

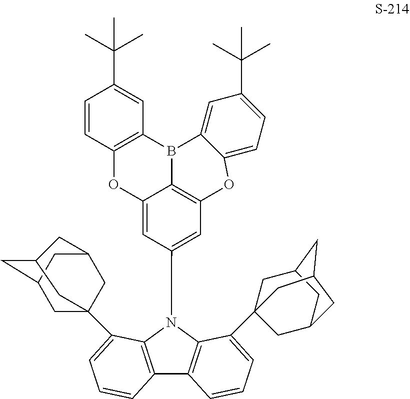 [SMILES: CC(C)(C)c1ccc2c(c1)B1c3cc(C(C)(C)C)ccc3Oc3cc(-n4c5c(C67CC8CC(CC(C8)C6)C7)cccc5c5cccc(C67CC8CC(CC(C8)C6)C7)c54)cc(c31)O2]

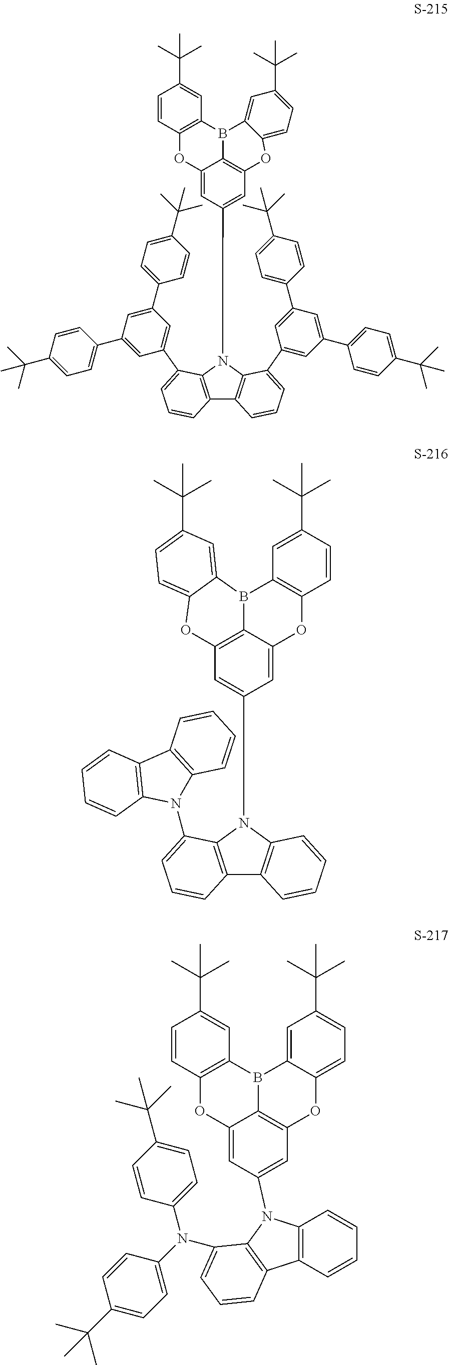

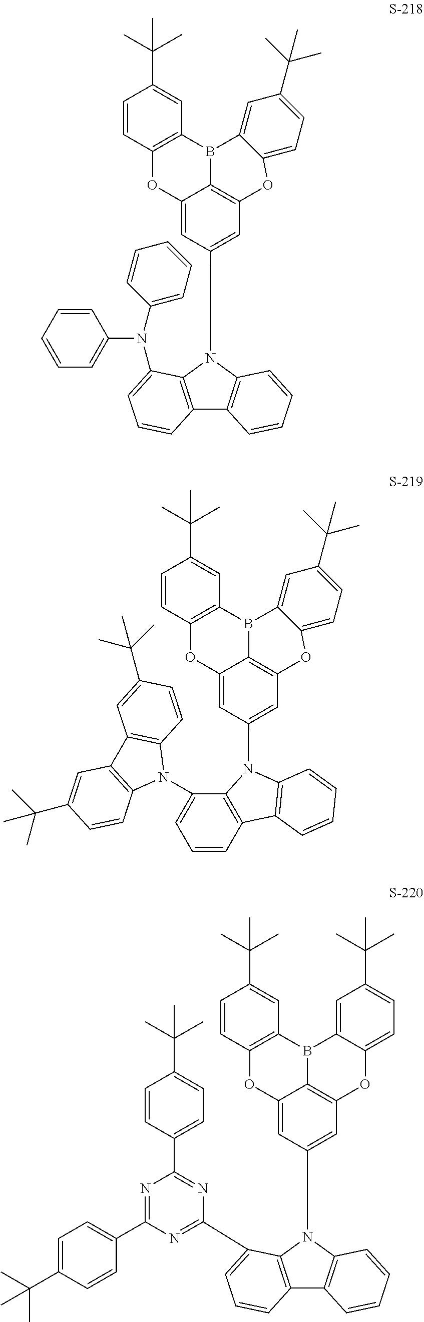

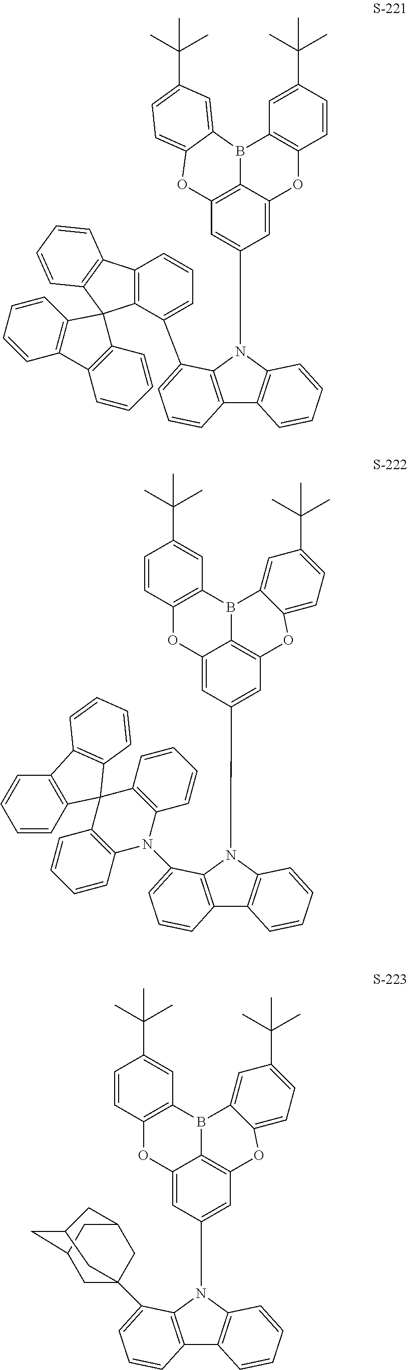 [SMILES: CC(C)(C)c1ccc2c(c1)B1c3cc(C(C)(C)C)ccc3Oc3cc(-n4c5ccccc5c5cccc(-c6cccc7c6C6(c8ccccc8-c8ccccc86)c6ccccc6-7)c54)cc(c31)O2 ; CC(C)(C)c1ccc2c(c1)B1c3cc(C(C)(C)C)ccc3Oc3cc(-n4c5ccccc5c5cccc(N6c7ccccc7C7(c8ccccc8-c8ccccc87)c7ccccc76)c54)cc(c31)O2 ; CC(C)(C)c1ccc2c(c1)B1c3cc(C(C)(C)C)ccc3Oc3cc(-n4c5ccccc5c5cccc(C67CC8CC(CC(C8)C6)C7)c54)cc(c31)O2]

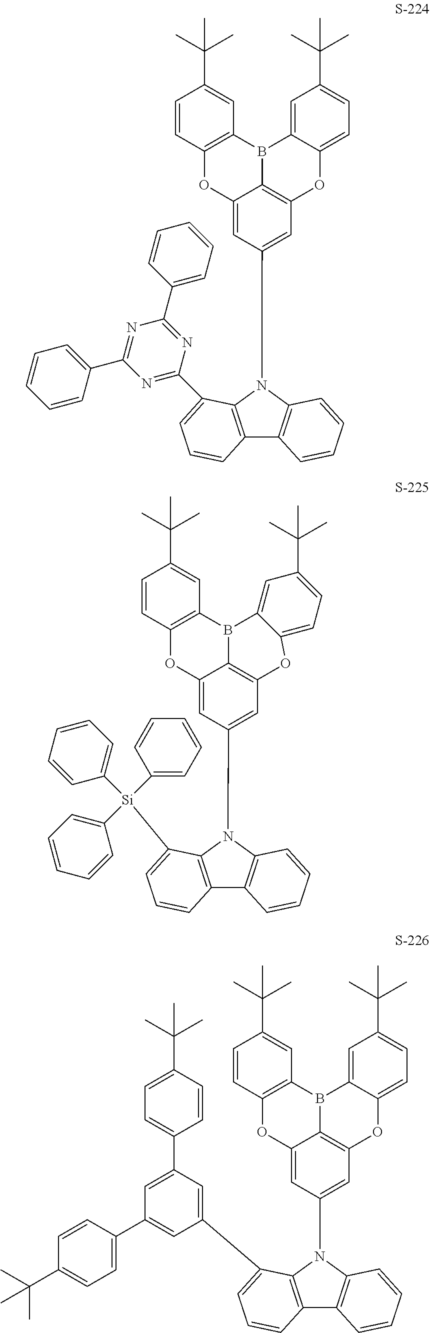

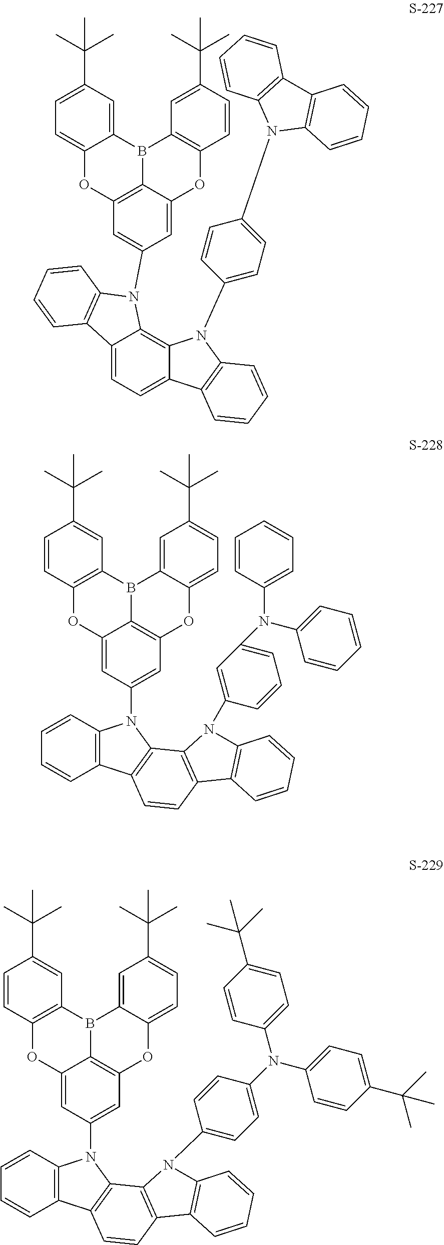

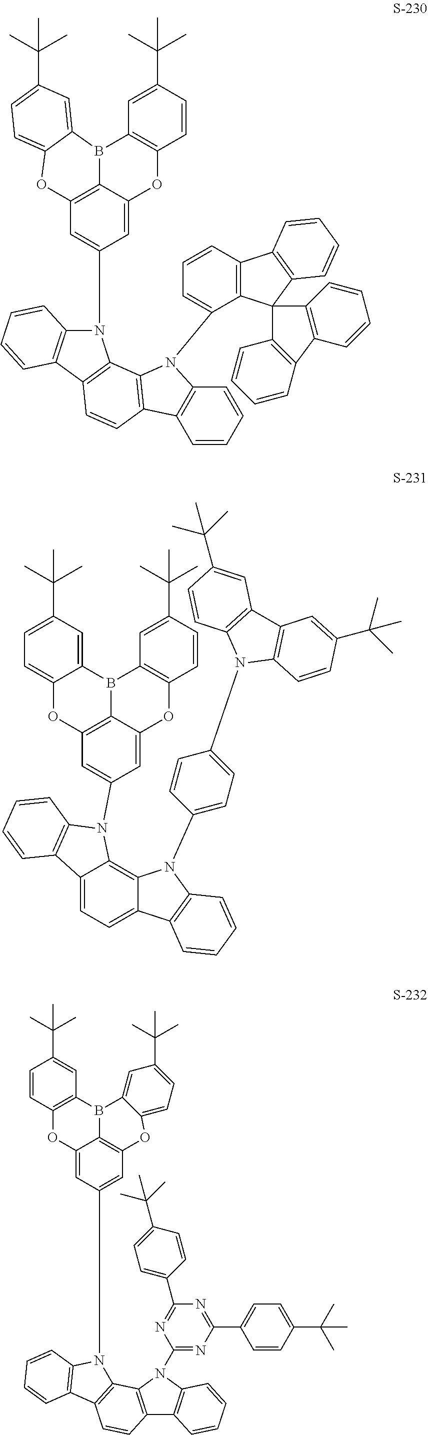 [SMILES: CC(C)(C)c1ccc2c(c1)B1c3cc(C(C)(C)C)ccc3Oc3cc(-n4c5ccccc5c5ccc6c7ccccc7n(-c7cccc8c7C7(c9ccccc9-c9ccccc97)c7ccccc7-8)c6c54)cc(c31)O2 ; CC(C)(C)c1ccc2c(c1)B1c3cc(C(C)(C)C)ccc3Oc3cc(-n4c5ccccc5c5ccc6c7ccccc7n(-c7ccc(-n8c9ccc(C(C)(C)C)cc9c9cc(C(C)(C)C)ccc98)cc7)c6c54)cc(c31)O2 ; CC(C)(C)c1ccc(-c2nc(-c3ccc(C(C)(C)C)cc3)nc(-n3c4ccccc4c4ccc5c6ccccc6n(-c6cc7c8c(c6)Oc6ccc(C(C)(C)C)cc6B8c6cc(C(C)(C)C)ccc6O7)c5c43)n2)cc1]

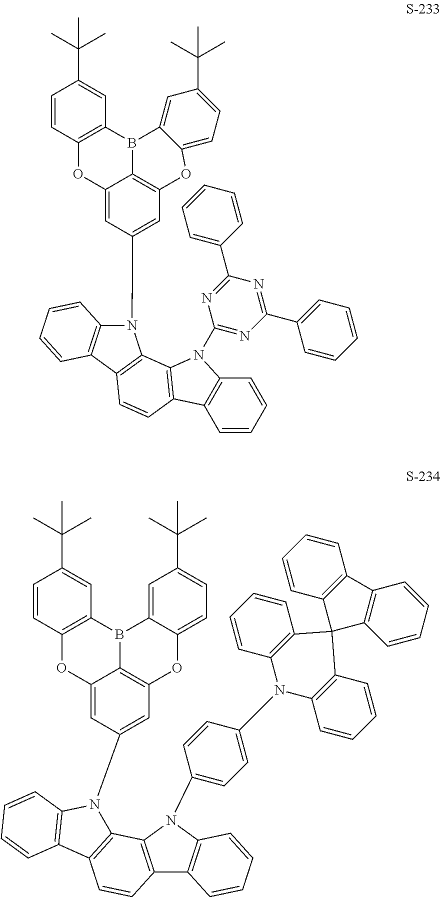 [SMILES: CC(C)(C)c1ccc2c(c1)B1c3cc(C(C)(C)C)ccc3Oc3cc(-n4c5ccccc5c5ccc6c7ccccc7n(-c7nc(-c8ccccc8)nc(-c8ccccc8)n7)c6c54)cc(c31)O2 ; CC(C)(C)c1ccc2c(c1)B1c3cc(C(C)(C)C)ccc3Oc3cc(-n4c5ccccc5c5ccc6c7ccccc7n(-c7ccc(N8c9ccccc9C9(c%10ccccc%10-c%10ccccc%109)c9ccccc98)cc7)c6c54)cc(c31)O2]

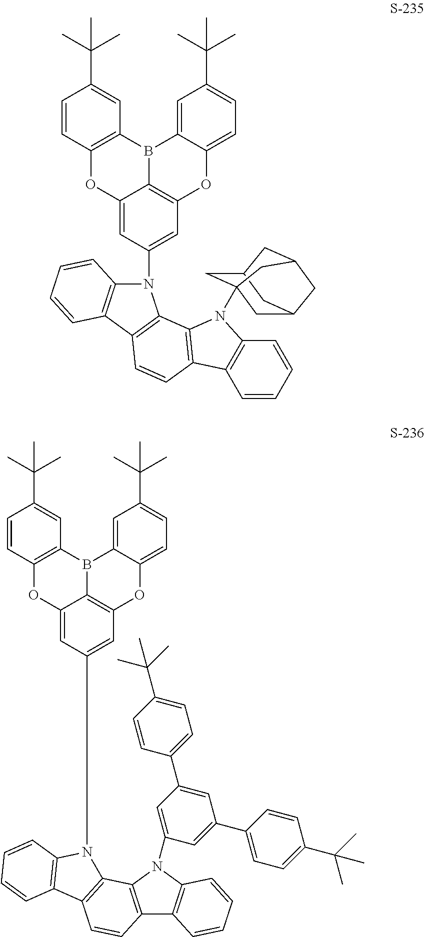 [SMILES: CC(C)(C)c1ccc2c(c1)B1c3cc(C(C)(C)C)ccc3Oc3cc(-n4c5ccccc5c5ccc6c7ccccc7n(C78CC9CC(CC(C9)C7)C8)c6c54)cc(c31)O2 ; CC(C)(C)c1ccc(-c2cc(-c3ccc(C(C)(C)C)cc3)cc(-n3c4ccccc4c4ccc5c6ccccc6n(-c6cc7c8c(c6)Oc6ccc(C(C)(C)C)cc6B8c6cc(C(C)(C)C)ccc6O7)c5c43)c2)cc1]

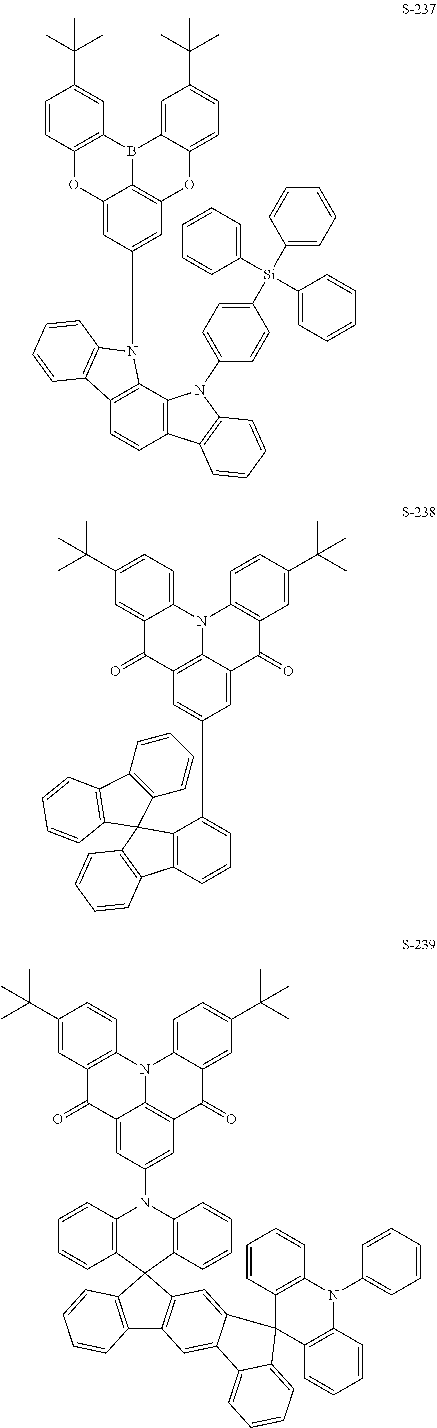

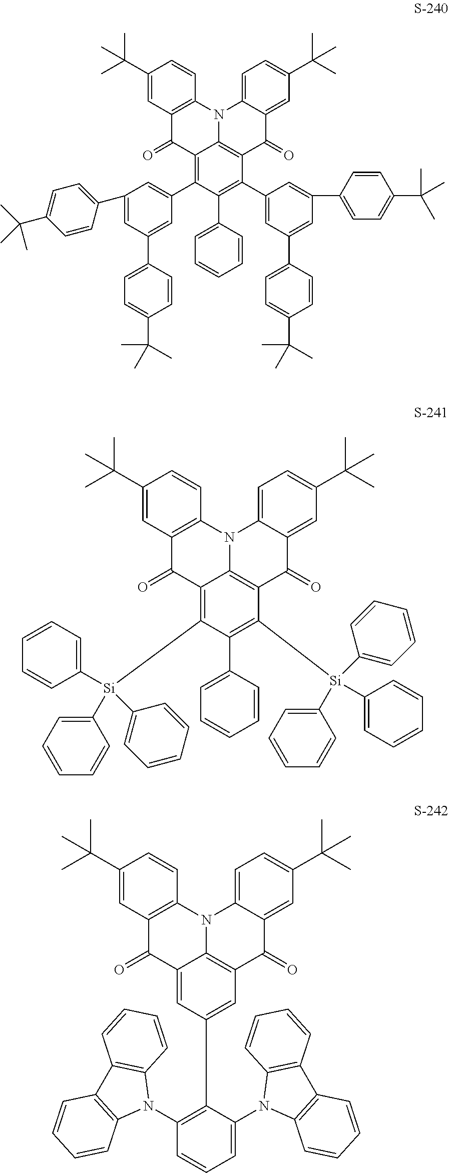

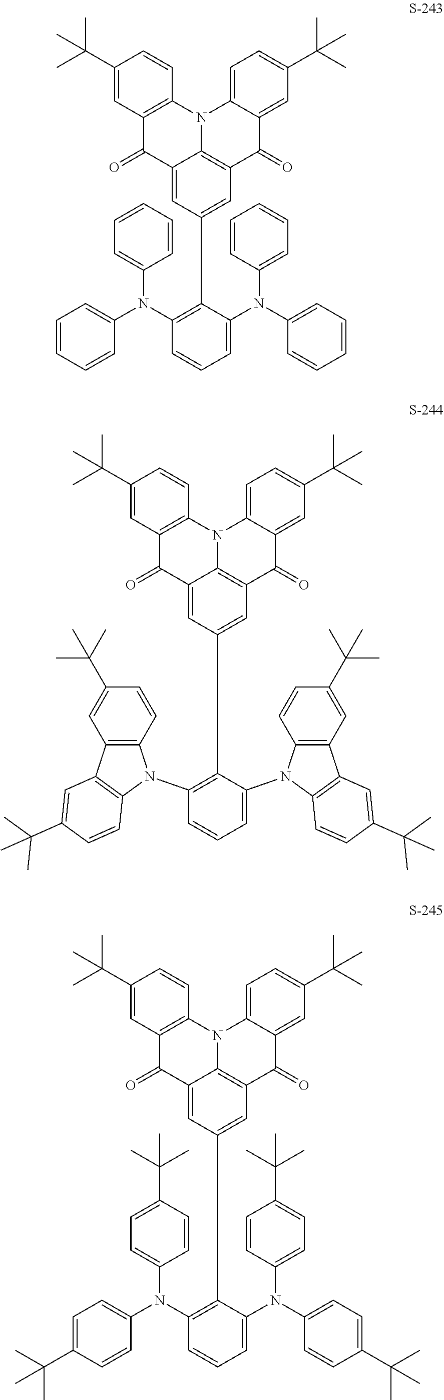

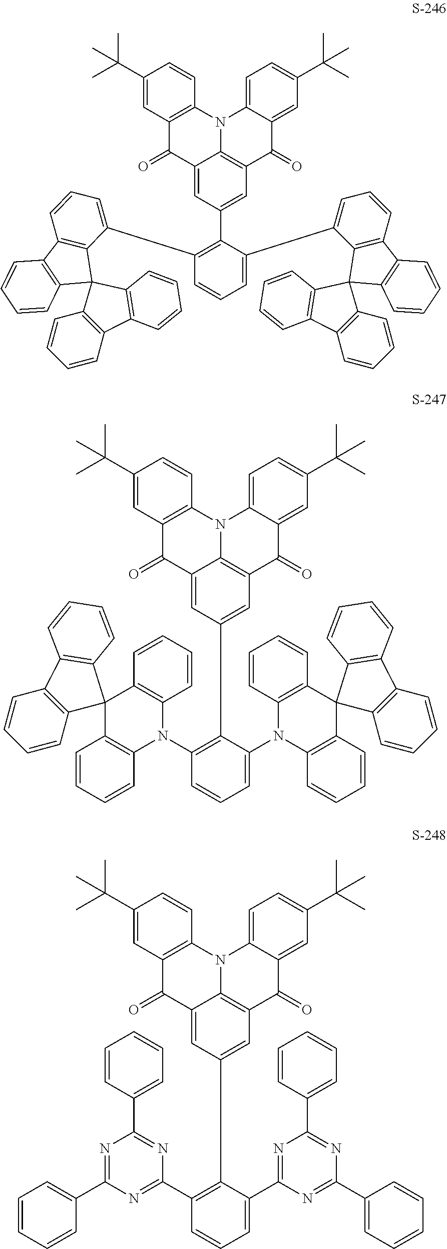 [SMILES: CC(C)(C)c1ccc2c(c1)c(=O)c1cc(-c3c(-c4cccc5c4C4(c6ccccc6-c6ccccc64)c4ccccc4-5)cccc3-c3cccc4c3C3(c5ccccc5-c5ccccc53)c3ccccc3-4)cc3c(=O)c4cc(C(C)(C)C)ccc4n2c13 ; CC(C)(C)c1ccc2c(c1)c(=O)c1cc(-c3c(N4c5ccccc5C5(c6ccccc6-c6ccccc65)c5ccccc54)cccc3N3c4ccccc4C4(c5ccccc5-c5ccccc54)c4ccccc43)cc3c(=O)c4cc(C(C)(C)C)ccc4n2c13 ; CC(C)(C)c1ccc2c(c1)c(=O)c1cc(-c3c(-c4nc(-c5ccccc5)nc(-c5ccccc5)n4)cccc3-c3nc(-c4ccccc4)nc(-c4ccccc4)n3)cc3c(=O)c4cc(C(C)(C)C)ccc4n2c13]

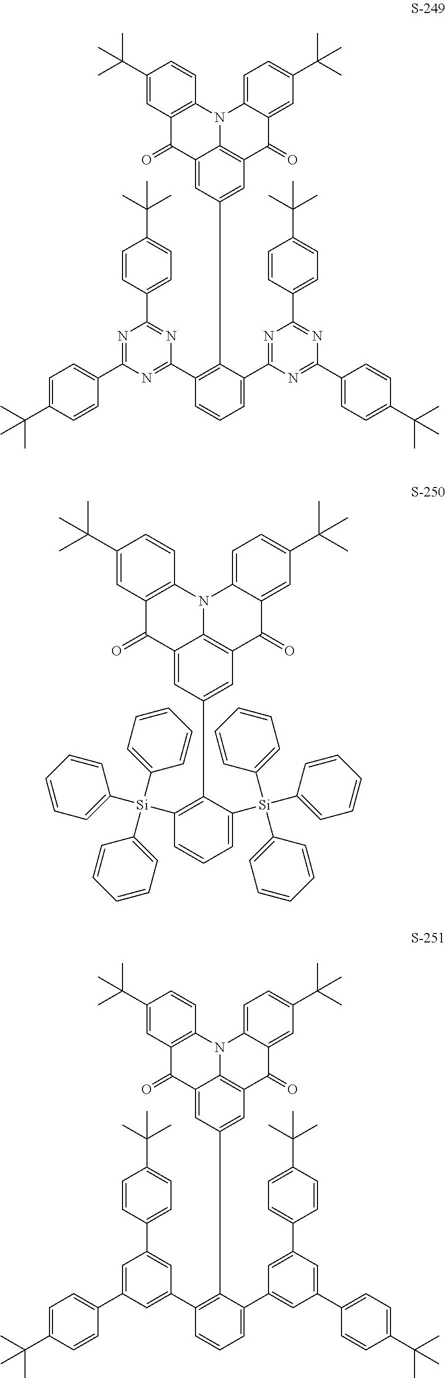

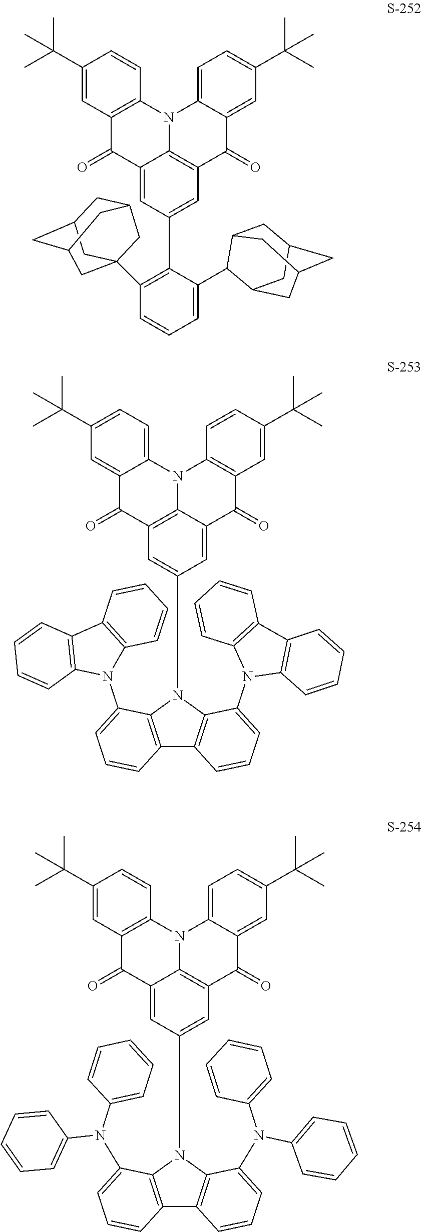 [SMILES: CC(C)(C)c1ccc2c(c1)c(=O)c1cc(-c3c(C4C5CC6CC(C5)CC4C6)cccc3C34CC5CC(CC(C5)C3)C4)cc3c(=O)c4cc(C(C)(C)C)ccc4n2c13 ; CC(C)(C)c1ccc2c(c1)c(=O)c1cc(-n3c4c(-n5c6ccccc6c6ccccc65)cccc4c4cccc(-n5c6ccccc6c6ccccc65)c43)cc3c(=O)c4cc(C(C)(C)C)ccc4n2c13 ; CC(C)(C)c1ccc2c(c1)c(=O)c1cc(-n3c4c(N(c5ccccc5)c5ccccc5)cccc4c4cccc(N(c5ccccc5)c5ccccc5)c43)cc3c(=O)c4cc(C(C)(C)C)ccc4n2c13]

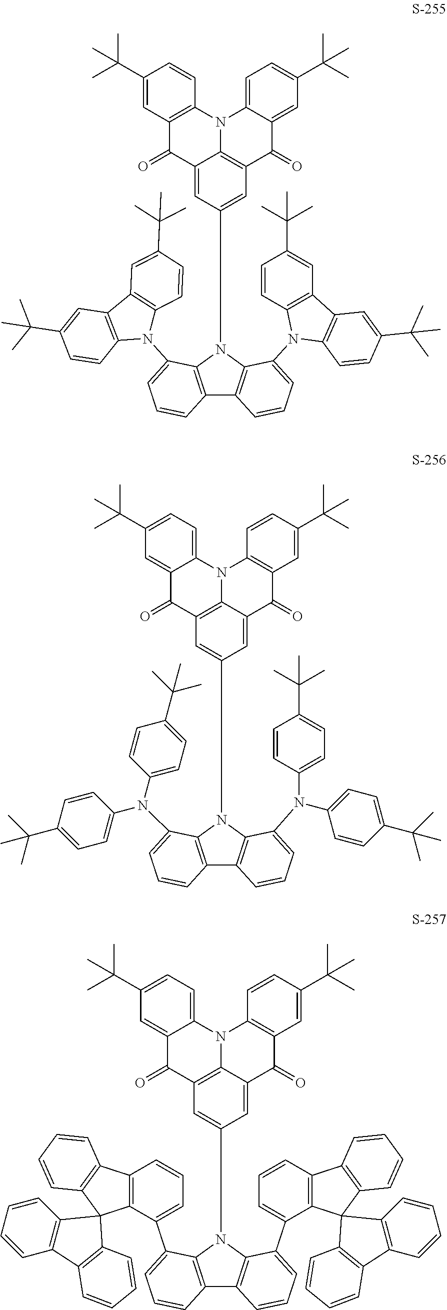

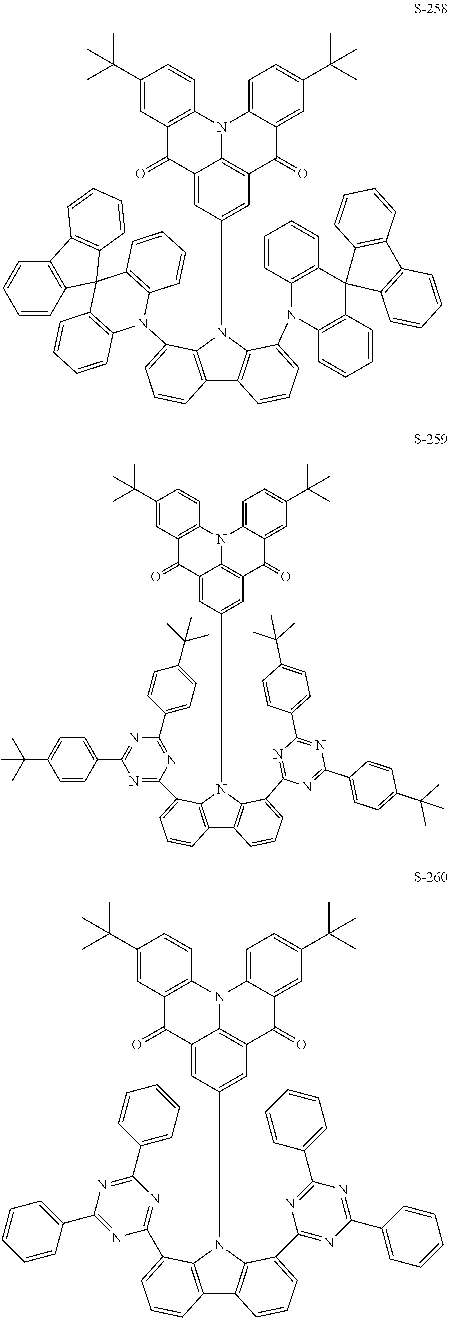

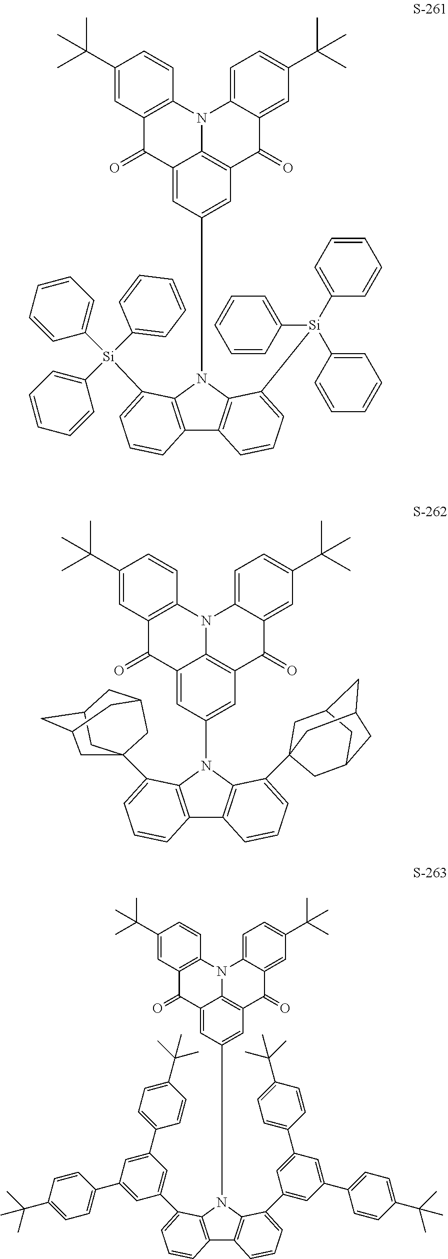 [SMILES: CC(C)(C)c1ccc2c(c1)c(=O)c1cc(-n3c4c([Si](c5ccccc5)(c5ccccc5)c5ccccc5)cccc4c4cccc([Si](c5ccccc5)(c5ccccc5)c5ccccc5)c43)cc3c(=O)c4cc(C(C)(C)C)ccc4n2c13 ; CC(C)(C)c1ccc2c(c1)c(=O)c1cc(-n3c4c(C56CC7CC(CC(C7)C5)C6)cccc4c4cccc(C56CC7CC(CC(C7)C5)C6)c43)cc3c(=O)c4cc(C(C)(C)C)ccc4n2c13 ; CC(C)(C)c1ccc(-c2cc(-c3ccc(C(C)(C)C)cc3)cc(-c3cccc4c5cccc(-c6cc(-c7ccc(C(C)(C)C)cc7)cc(-c7ccc(C(C)(C)C)cc7)c6)c5n(-c5cc6c(=O)c7cc(C(C)(C)C)ccc7n7c8ccc(C(C)(C)C)cc8c(=O)c(c5)c67)c34)c2)cc1]

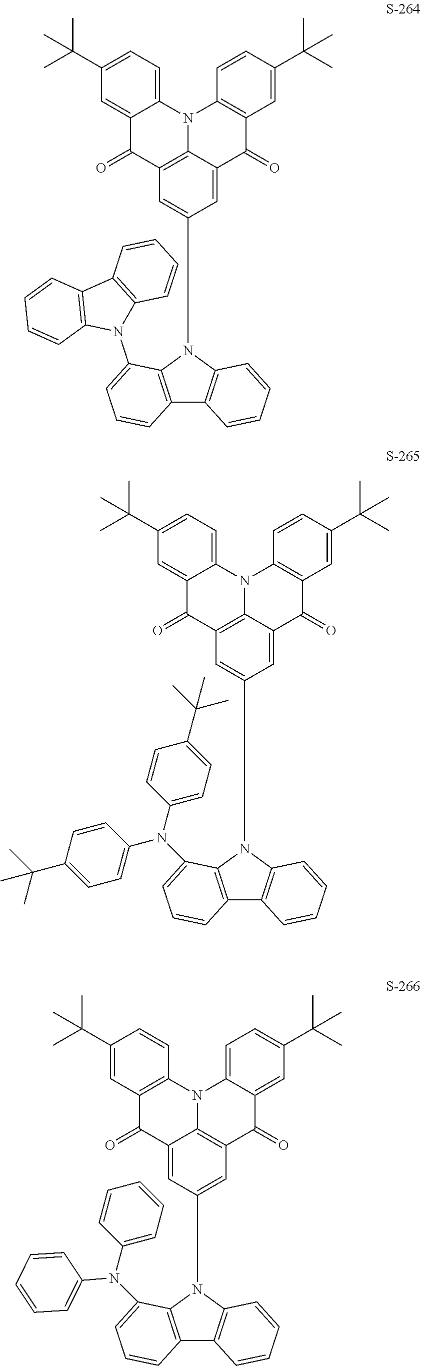

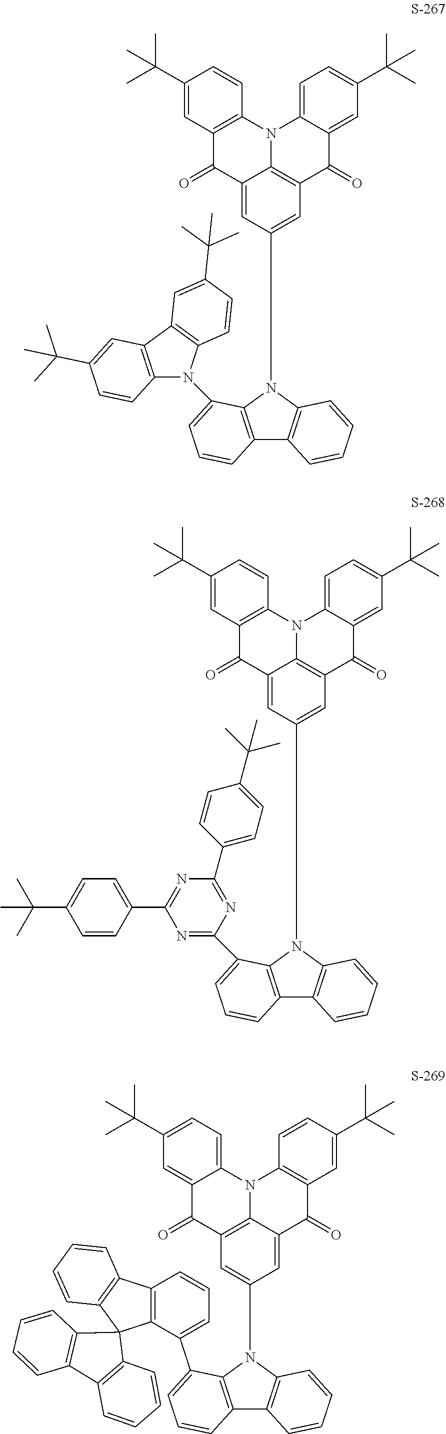

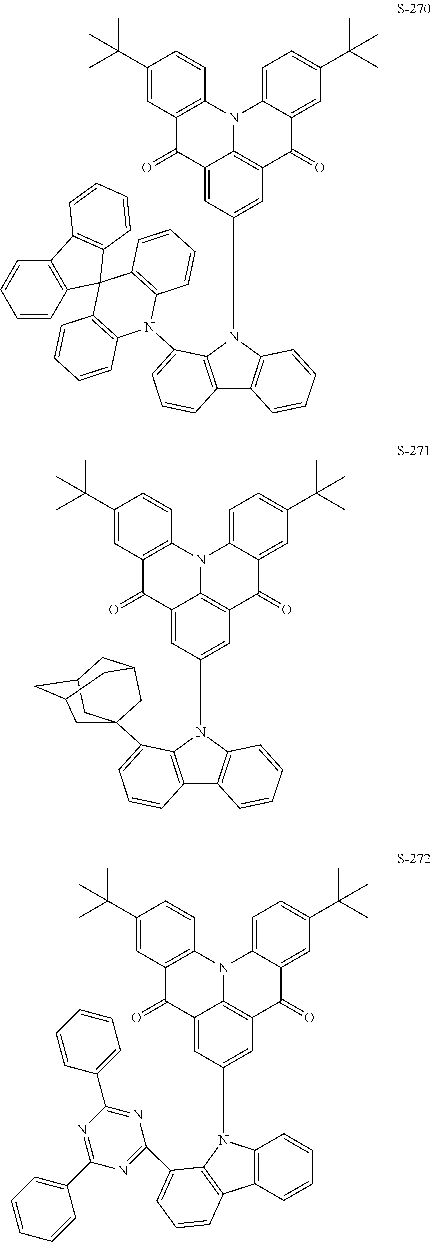 [SMILES: CC(C)(C)c1ccc2c(c1)c(=O)c1cc(-n3c4ccccc4c4cccc(N5c6ccccc6C6(c7ccccc7-c7ccccc76)c6ccccc65)c43)cc3c(=O)c4cc(C(C)(C)C)ccc4n2c13 ; CC(C)(C)c1ccc2c(c1)c(=O)c1cc(-n3c4ccccc4c4cccc(C56CC7CC(CC(C7)C5)C6)c43)cc3c(=O)c4cc(C(C)(C)C)ccc4n2c13 ; CC(C)(C)c1ccc2c(c1)c(=O)c1cc(-n3c4ccccc4c4cccc(-c5nc(-c6ccccc6)nc(-c6ccccc6)n5)c43)cc3c(=O)c4cc(C(C)(C)C)ccc4n2c13]

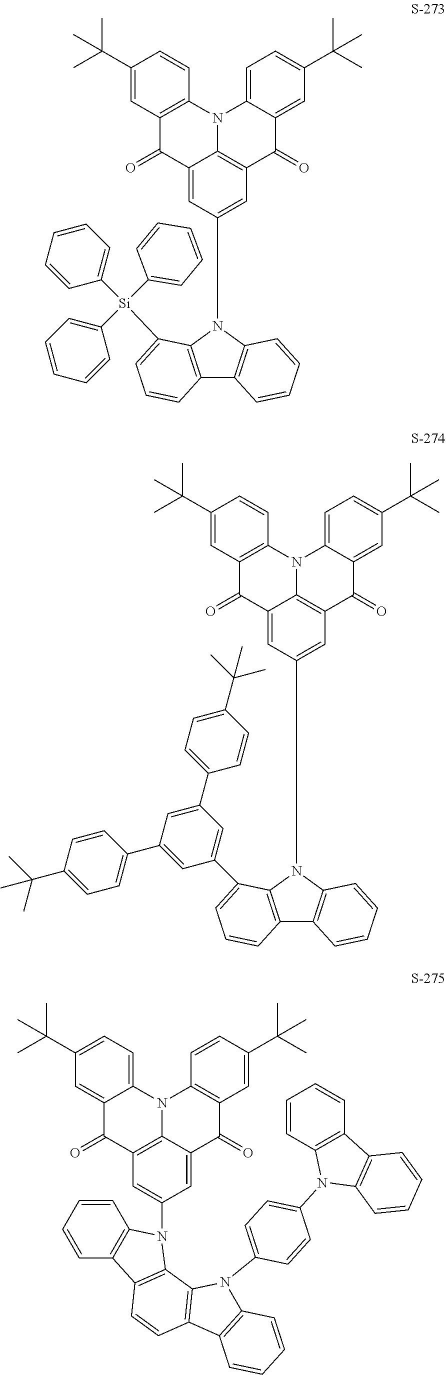

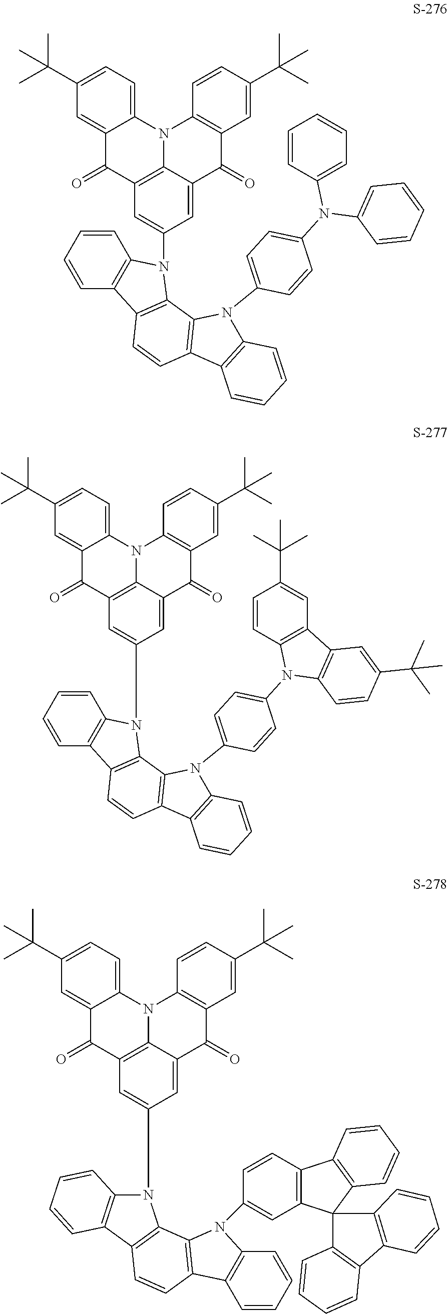

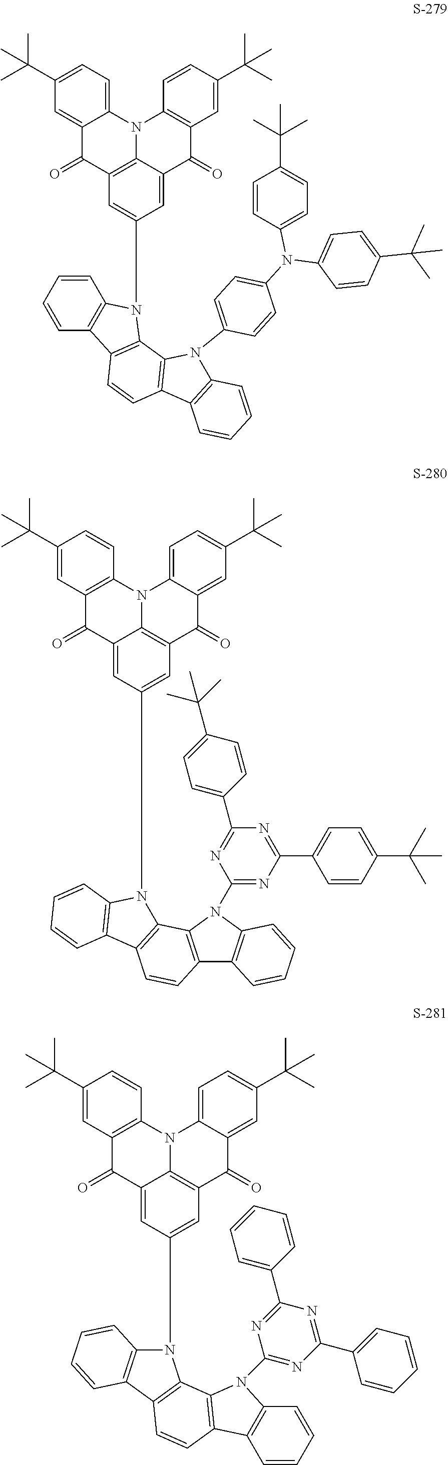

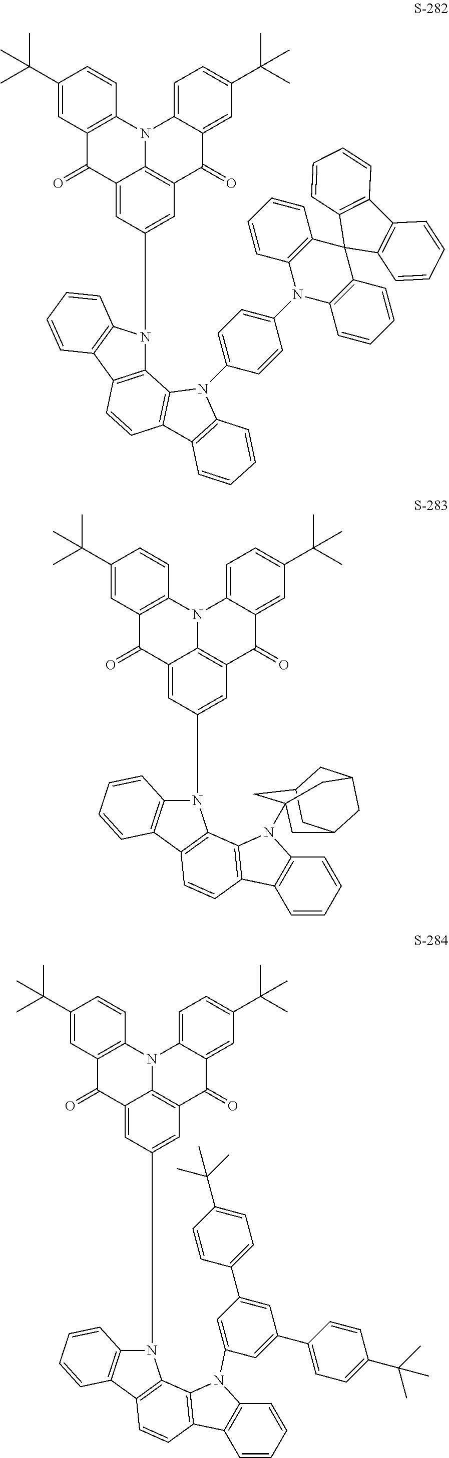 [SMILES: CC(C)(C)c1ccc2c(c1)c(=O)c1cc(-n3c4ccccc4c4ccc5c6ccccc6n(-c6ccc(N7c8ccccc8C8(c9ccccc9-c9ccccc98)c8ccccc87)cc6)c5c43)cc3c(=O)c4cc(C(C)(C)C)ccc4n2c13 ; CC(C)(C)c1ccc2c(c1)c(=O)c1cc(-n3c4ccccc4c4ccc5c6ccccc6n(C67CC8CC(CC(C8)C6)C7)c5c43)cc3c(=O)c4cc(C(C)(C)C)ccc4n2c13 ; CC(C)(C)c1ccc(-c2cc(-c3ccc(C(C)(C)C)cc3)cc(-n3c4ccccc4c4ccc5c6ccccc6n(-c6cc7c(=O)c8cc(C(C)(C)C)ccc8n8c9ccc(C(C)(C)C)cc9c(=O)c(c6)c78)c5c43)c2)cc1]

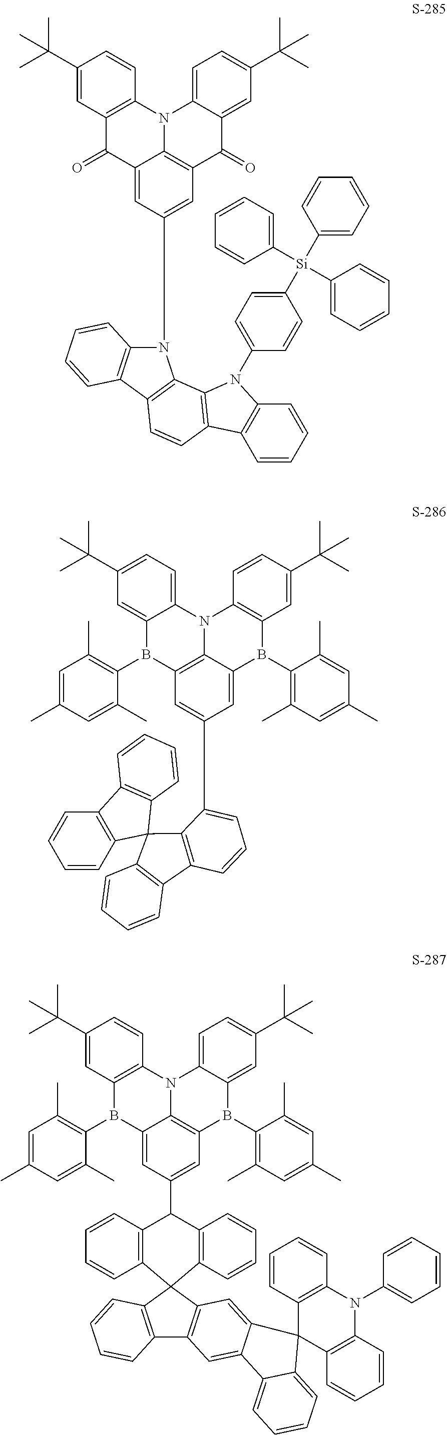

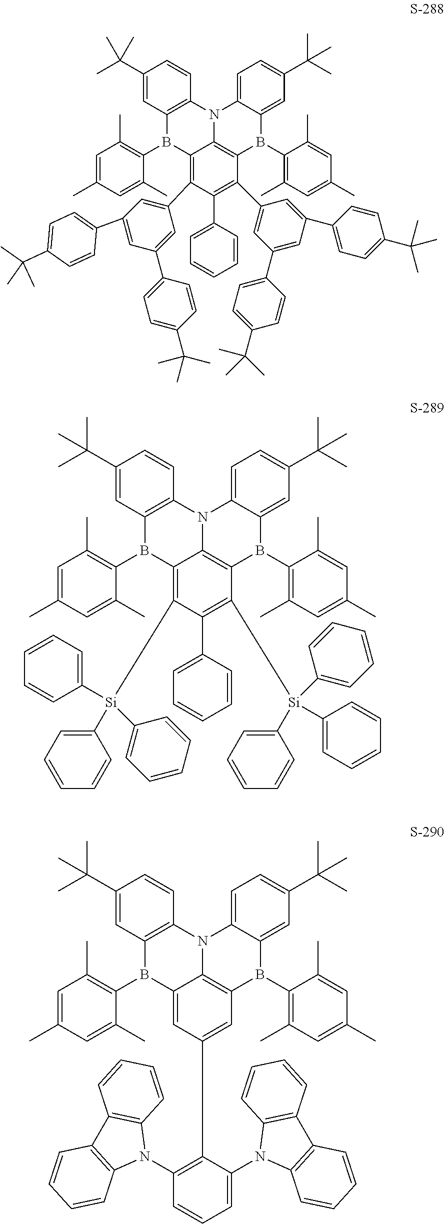

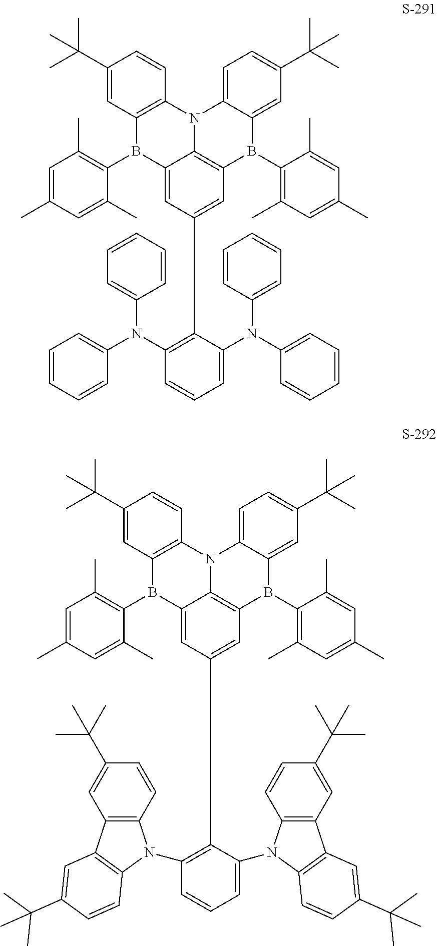

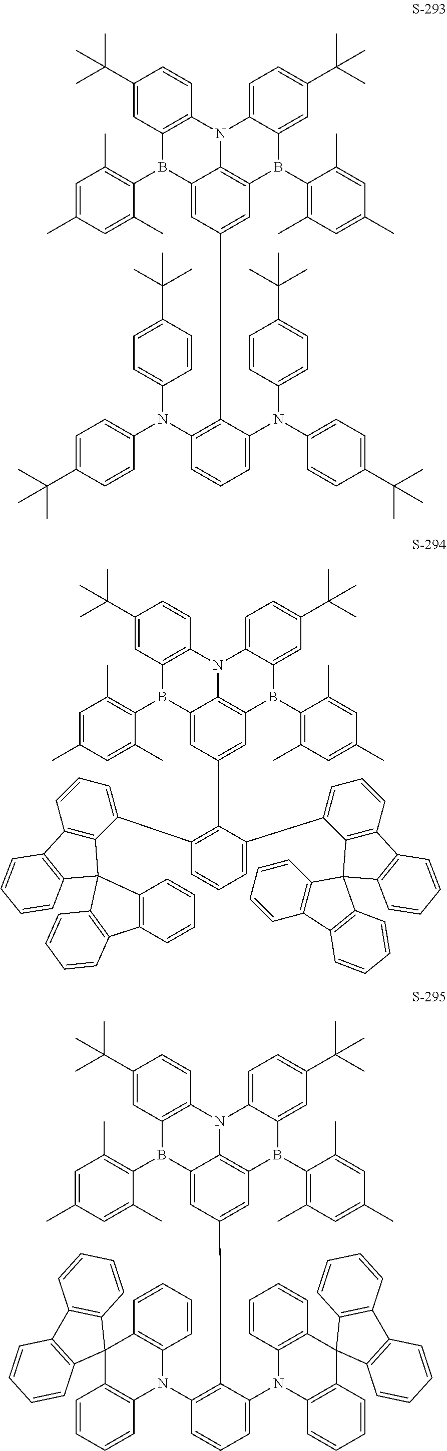

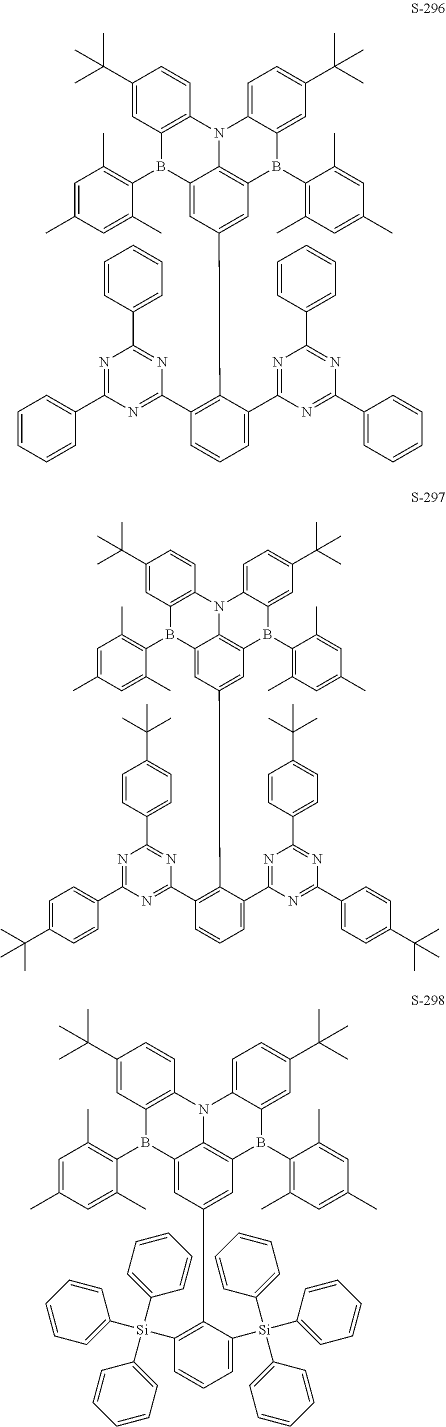

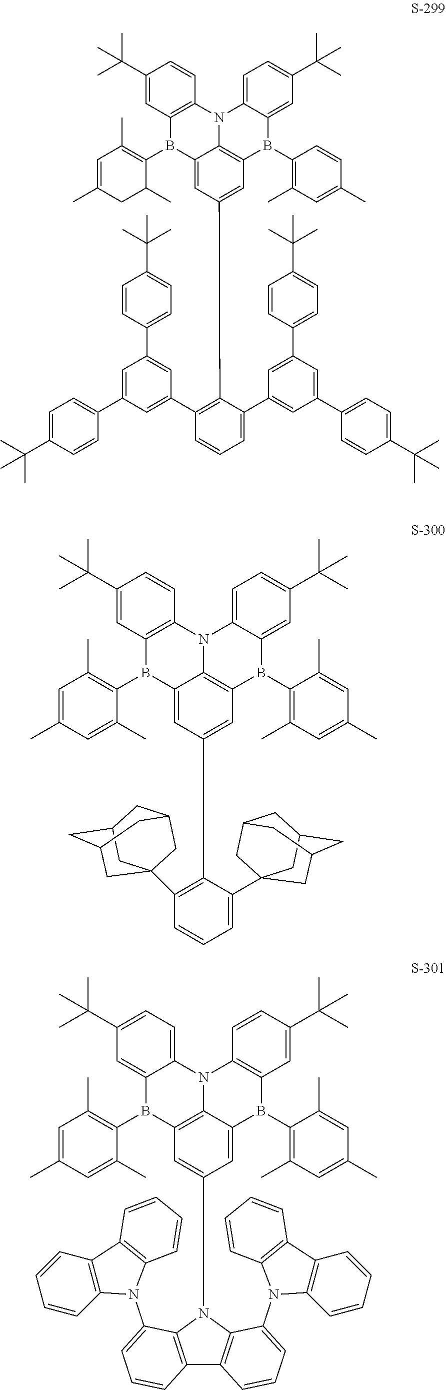

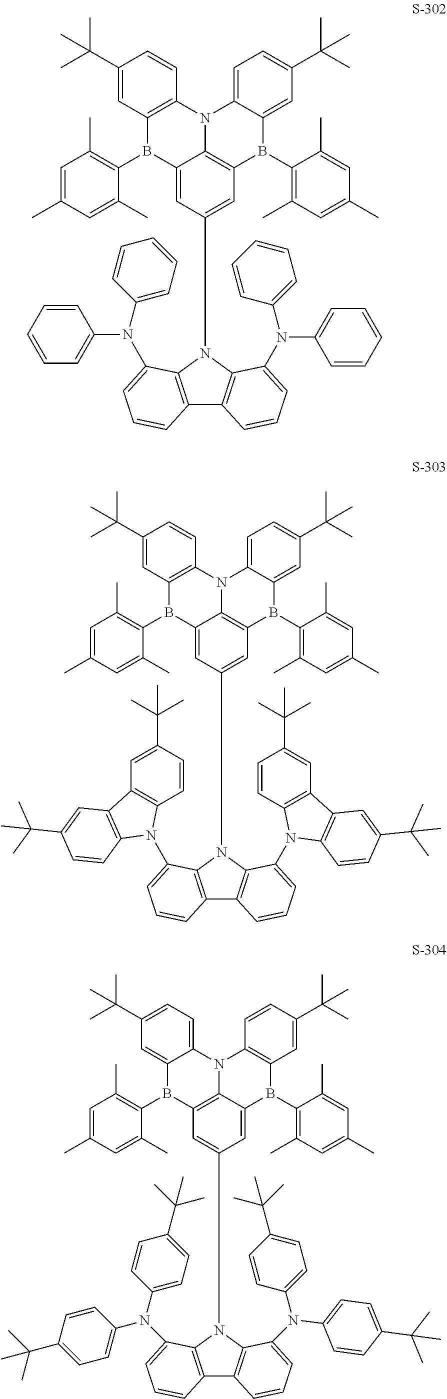

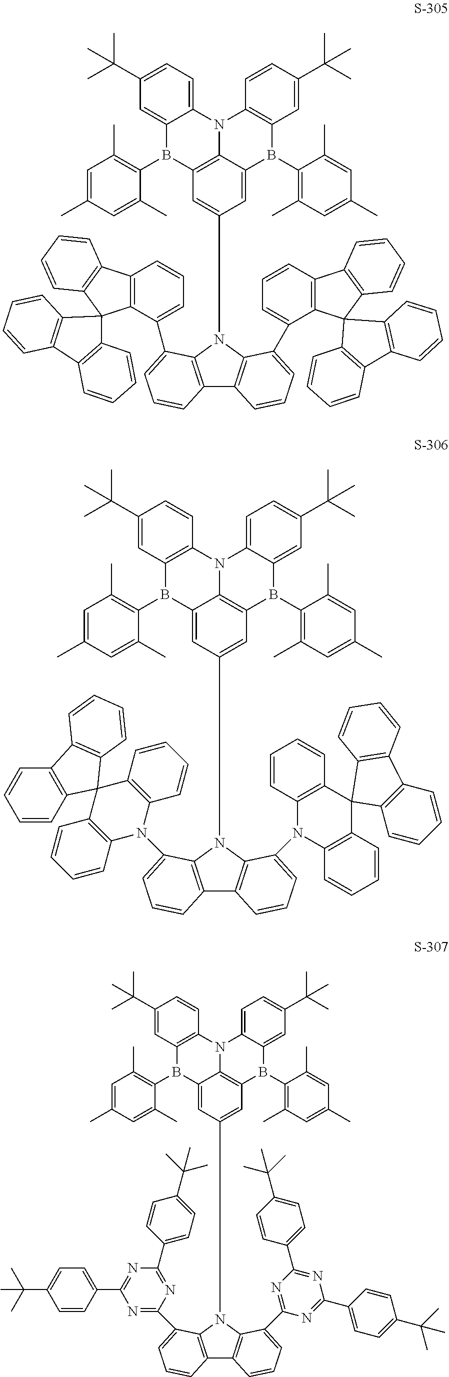

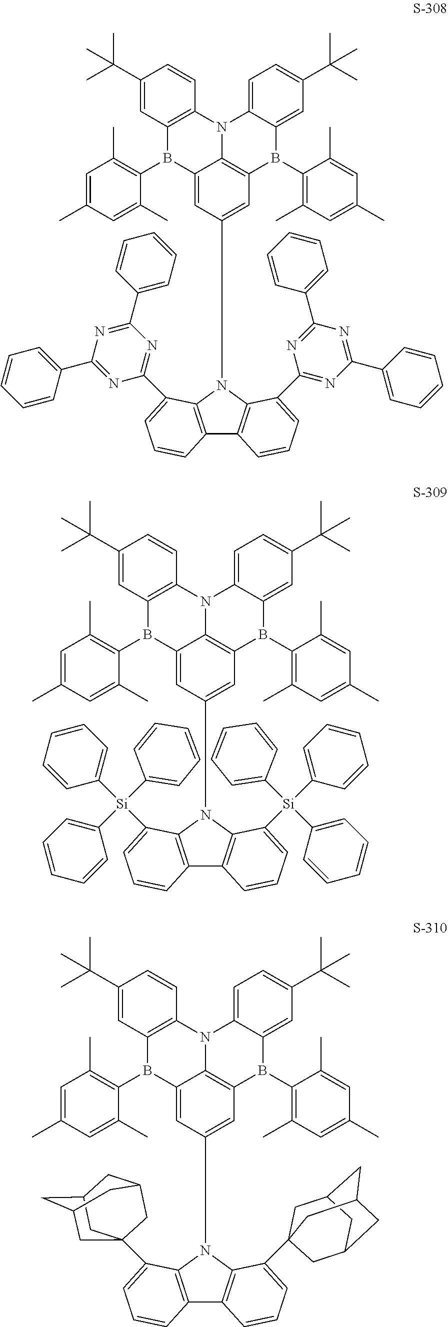

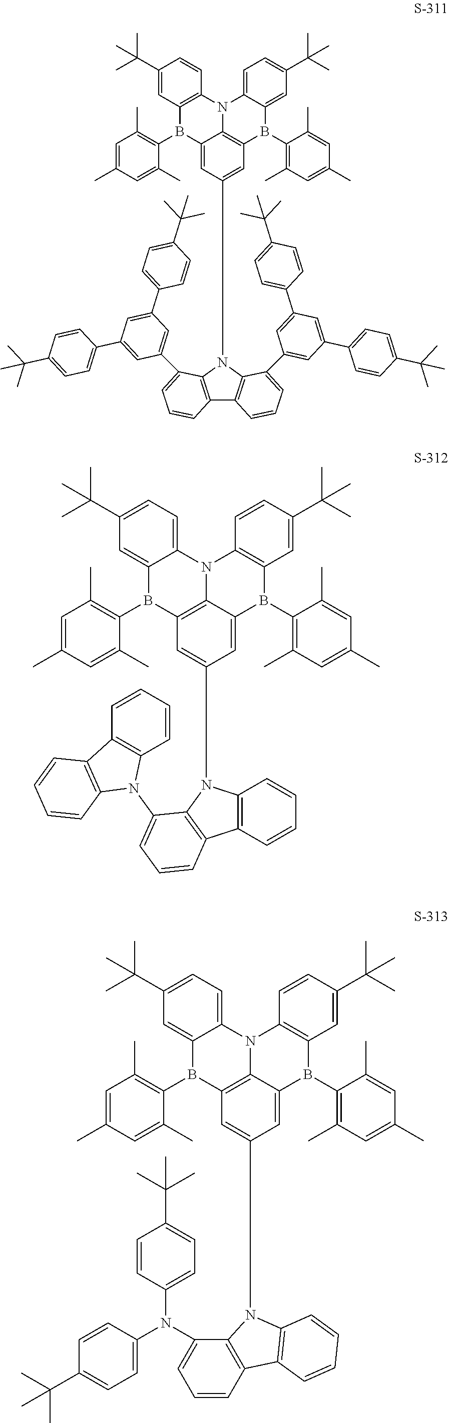

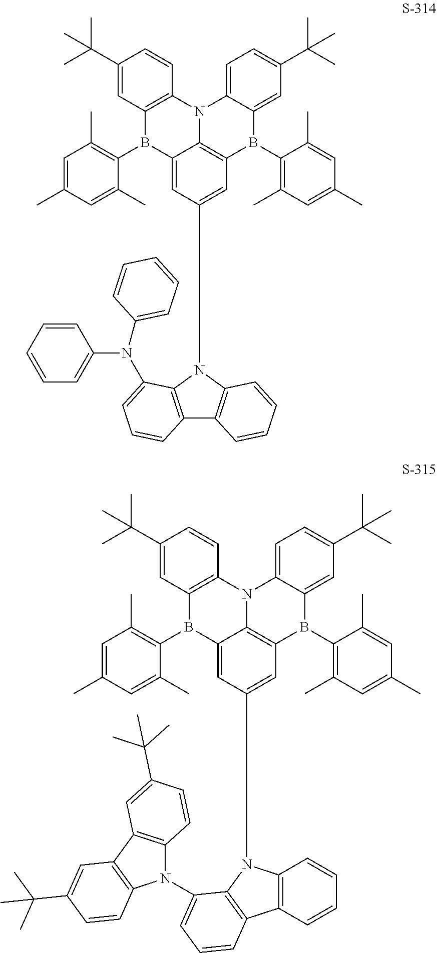

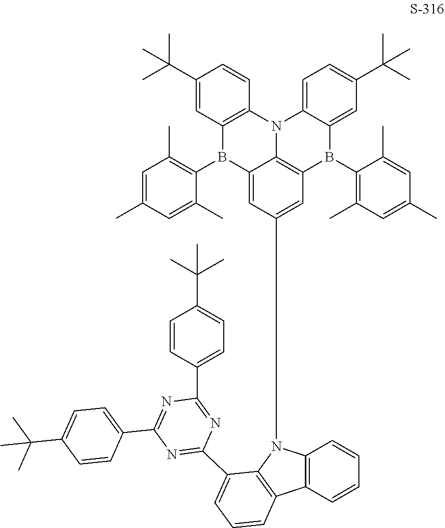 [SMILES: Cc1cc(C)c(B2c3cc(C(C)(C)C)ccc3N3c4ccc(C(C)(C)C)cc4B(c4c(C)cc(C)cc4C)c4cc(-n5c6ccccc6c6cccc(-c7nc(-c8ccc(C(C)(C)C)cc8)nc(-c8ccc(C(C)(C)C)cc8)n7)c65)cc2c43)c(C)c1]

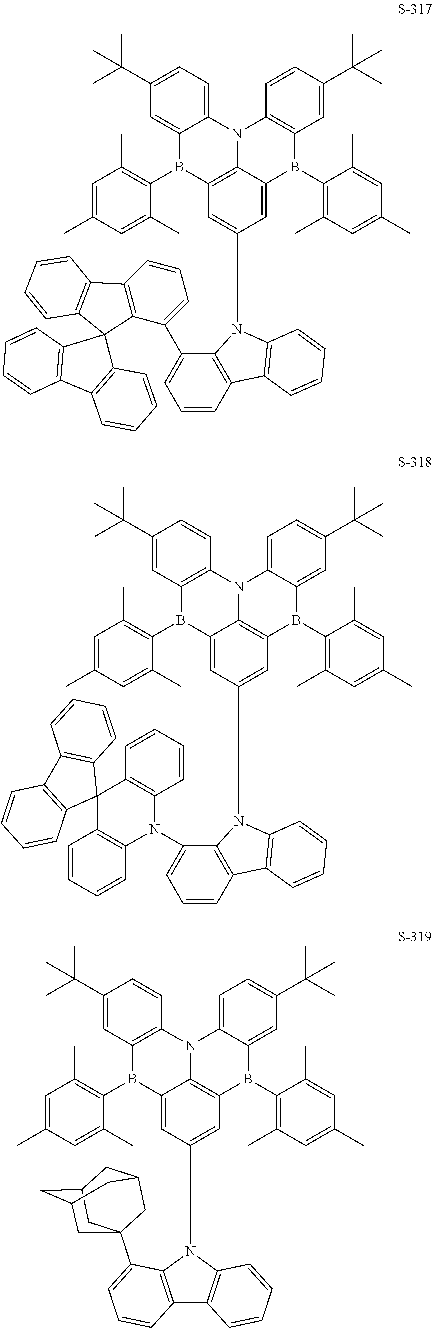

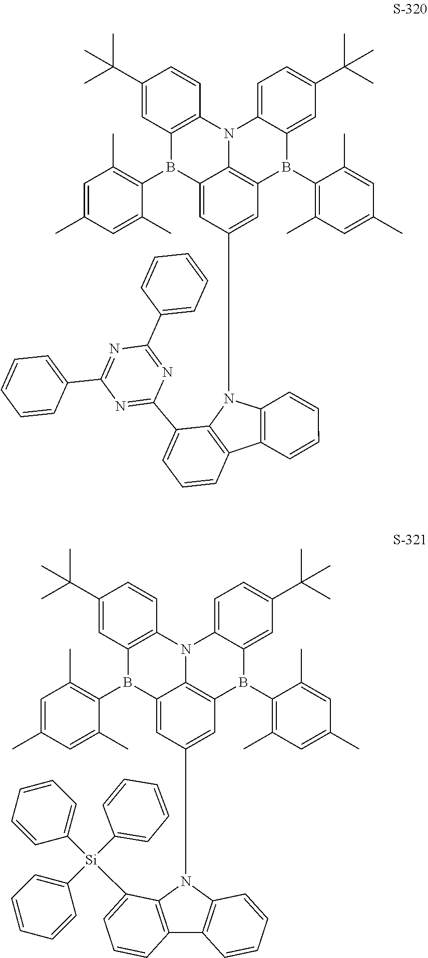 [SMILES: Cc1cc(C)c(B2c3cc(C(C)(C)C)ccc3N3c4ccc(C(C)(C)C)cc4B(c4c(C)cc(C)cc4C)c4cc(-n5c6ccccc6c6cccc(-c7nc(-c8ccccc8)nc(-c8ccccc8)n7)c65)cc2c43)c(C)c1 ; Cc1cc(C)c(B2c3cc(C(C)(C)C)ccc3N3c4ccc(C(C)(C)C)cc4B(c4c(C)cc(C)cc4C)c4cc(-n5c6ccccc6c6cccc([Si](c7ccccc7)(c7ccccc7)c7ccccc7)c65)cc2c43)c(C)c1]

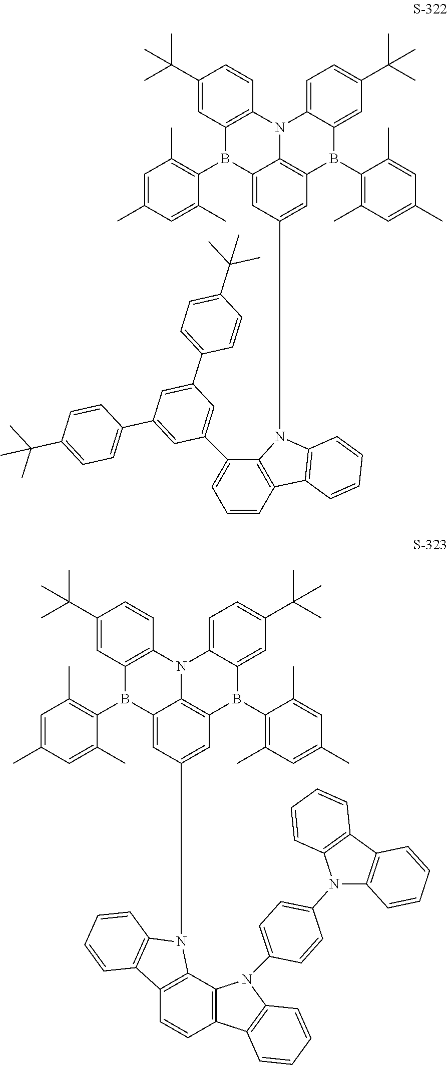

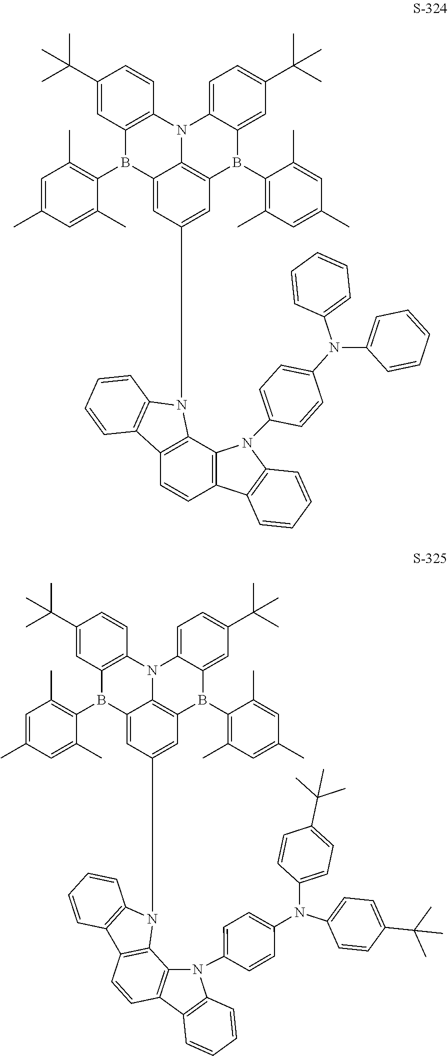 [SMILES: Cc1cc(C)c(B2c3cc(C(C)(C)C)ccc3N3c4ccc(C(C)(C)C)cc4B(c4c(C)cc(C)cc4C)c4cc(-n5c6ccccc6c6ccc7c8ccccc8n(-c8ccc(N(c9ccccc9)c9ccccc9)cc8)c7c65)cc2c43)c(C)c1 ; Cc1cc(C)c(B2c3cc(C(C)(C)C)ccc3N3c4ccc(C(C)(C)C)cc4B(c4c(C)cc(C)cc4C)c4cc(-n5c6ccccc6c6ccc7c8ccccc8n(-c8ccc(N(c9ccc(C(C)(C)C)cc9)c9ccc(C(C)(C)C)cc9)cc8)c7c65)cc2c43)c(C)c1]

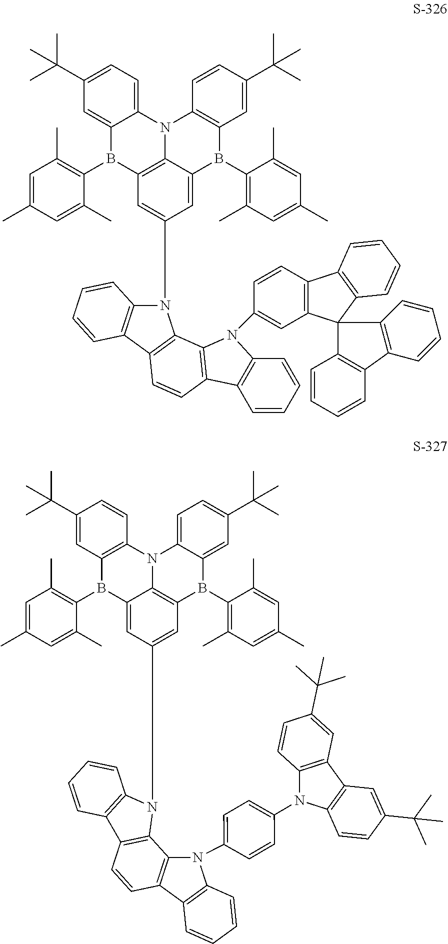 [SMILES: Cc1cc(C)c(B2c3cc(C(C)(C)C)ccc3N3c4ccc(C(C)(C)C)cc4B(c4c(C)cc(C)cc4C)c4cc(-n5c6ccccc6c6ccc7c8ccccc8n(-c8ccc9c(c8)C8(c%10ccccc%10-c%10ccccc%108)c8ccccc8-9)c7c65)cc2c43)c(C)c1 ; Cc1cc(C)c(B2c3cc(C(C)(C)C)ccc3N3c4ccc(C(C)(C)C)cc4B(c4c(C)cc(C)cc4C)c4cc(-n5c6ccccc6c6ccc7c8ccccc8n(-c8ccc(-n9c%10ccc(C(C)(C)C)cc%10c%10cc(C(C)(C)C)ccc%109)cc8)c7c65)cc2c43)c(C)c1]

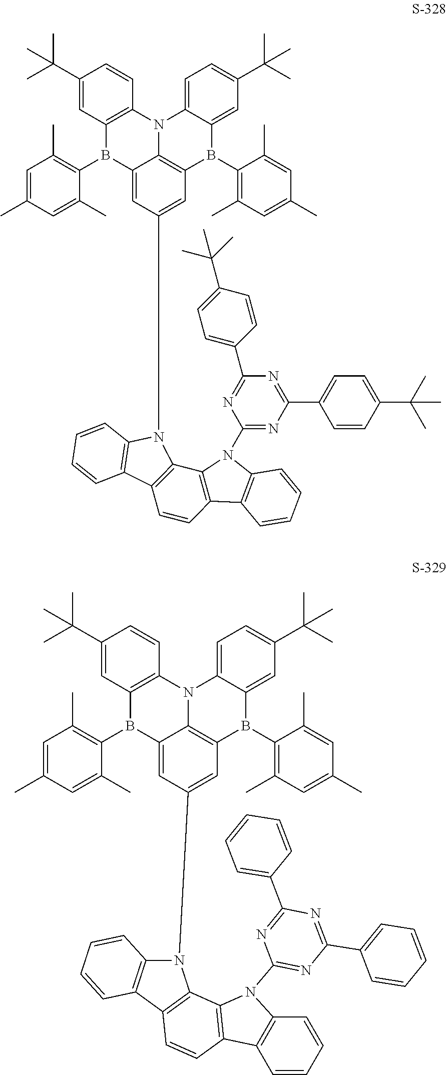

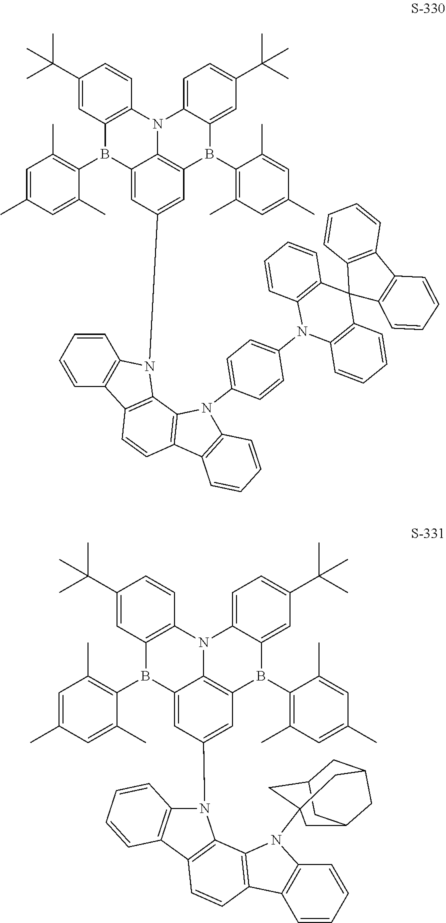

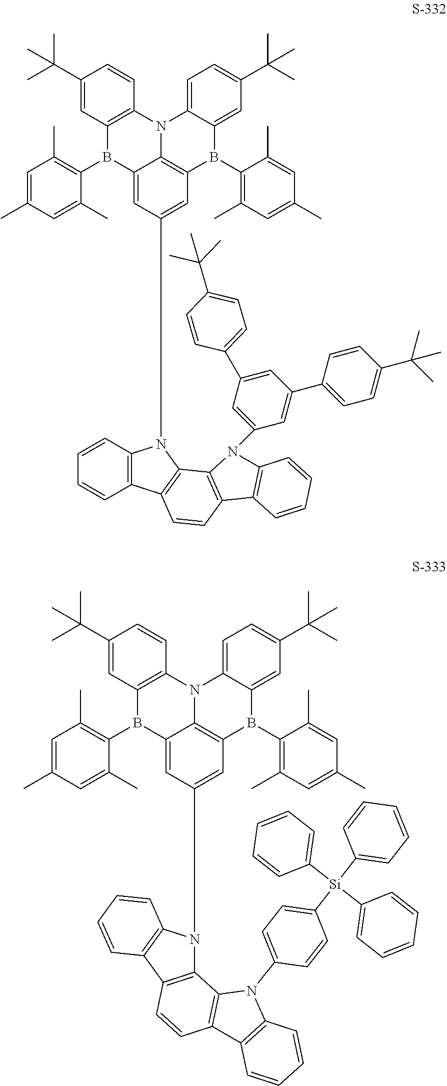

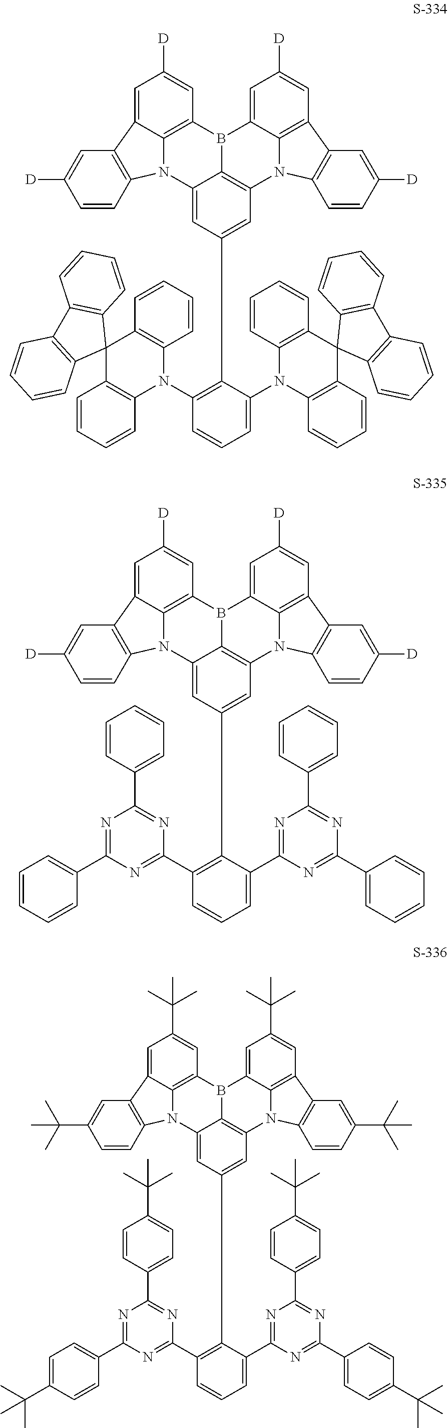

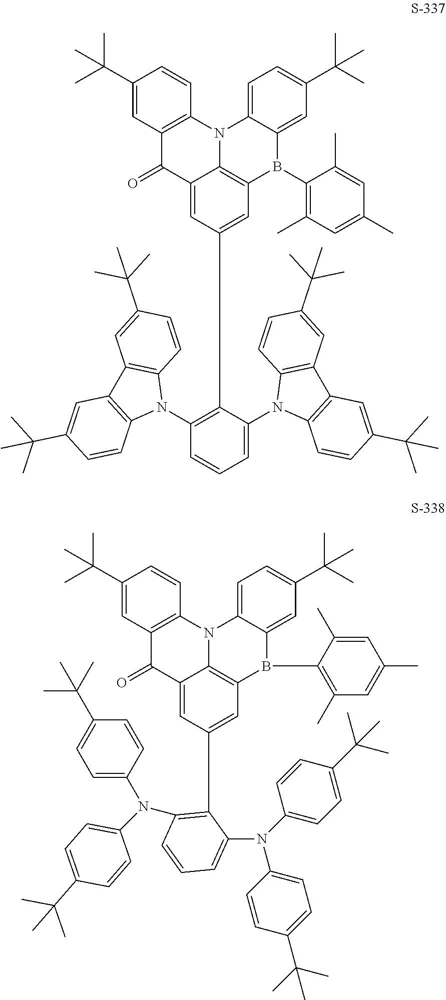

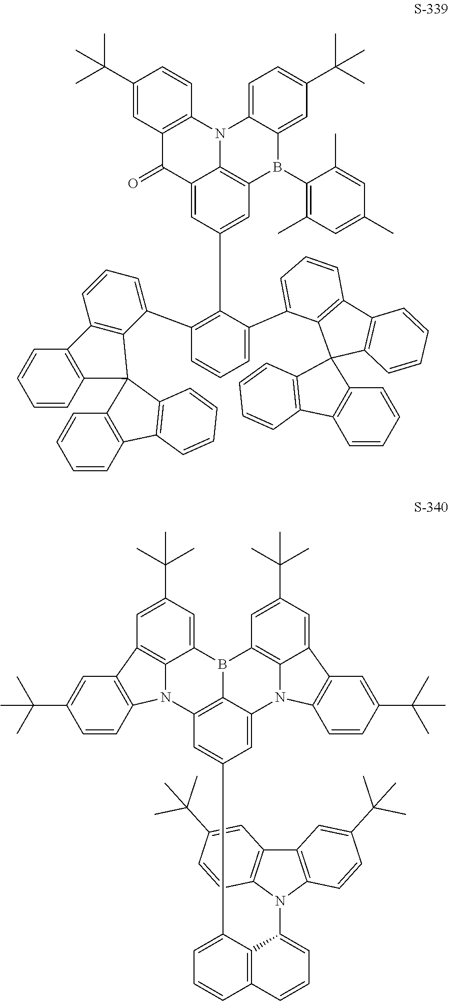

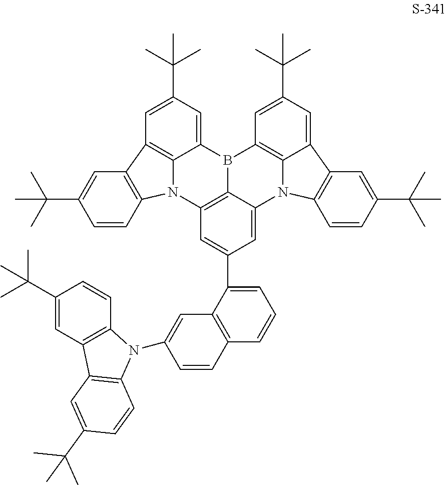

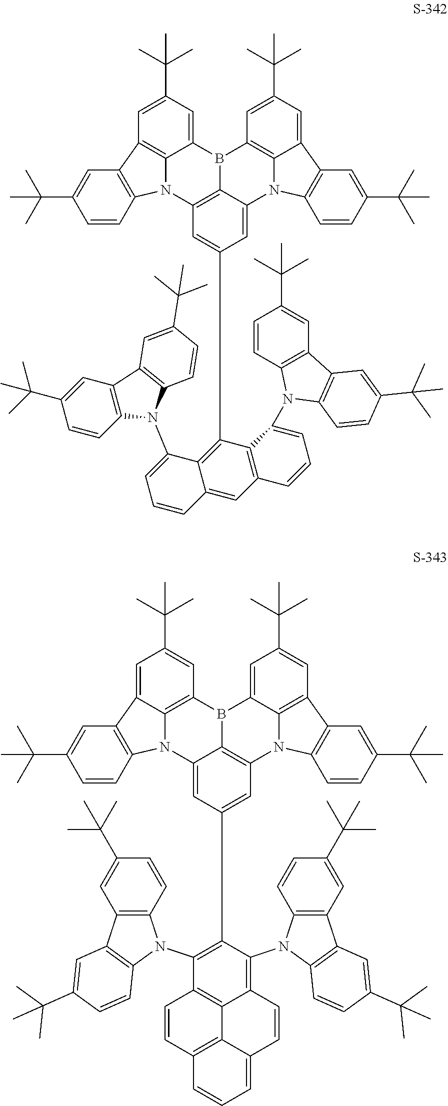

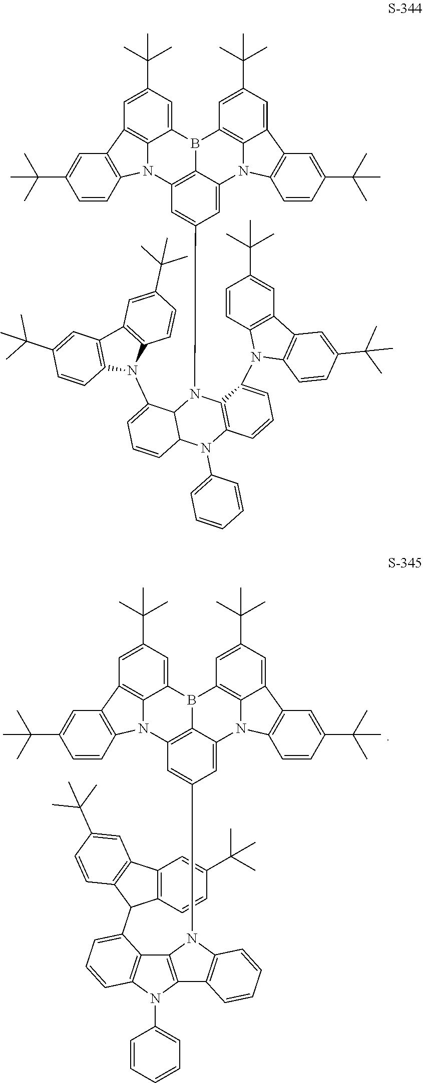

In the present application, there is no special limitation to the TTA host material in the light-emitting layer. In an implementation, when the TTA host material is selected from at least one compound represented by BFH-1 to BFH-25, the performance of the organic electroluminescent device is improved more significantly.

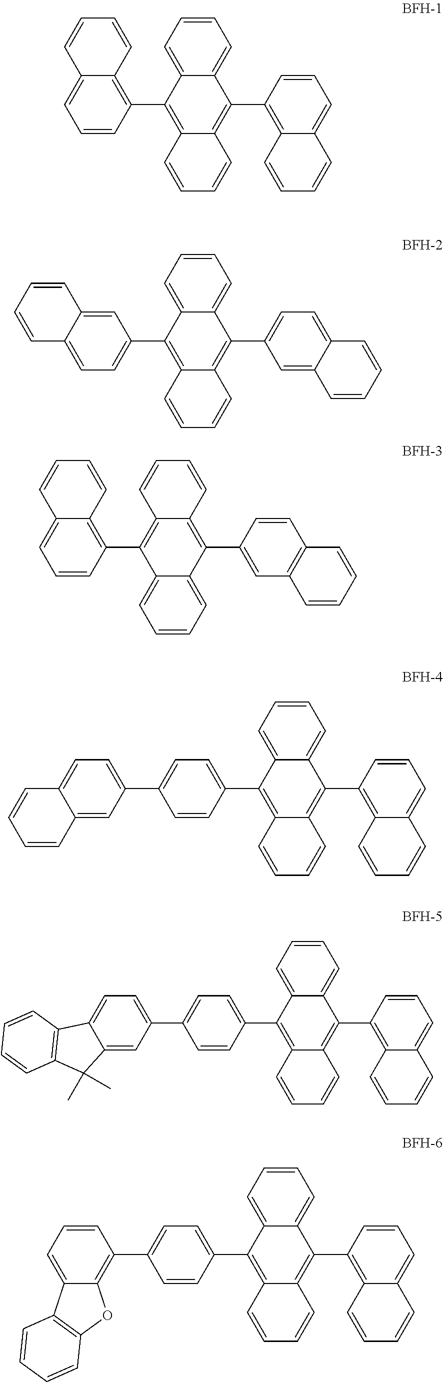

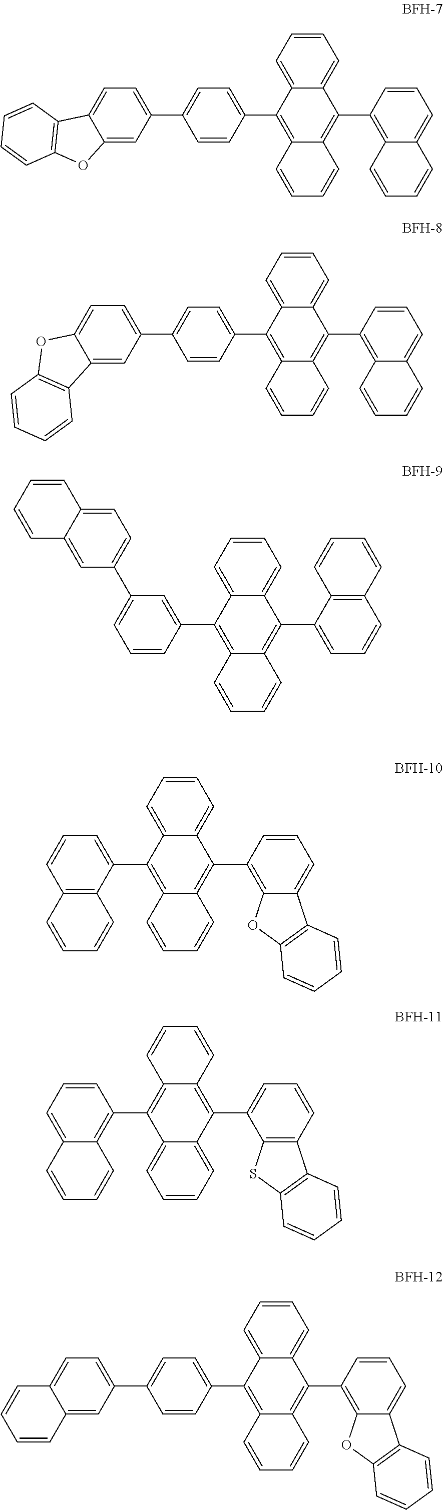

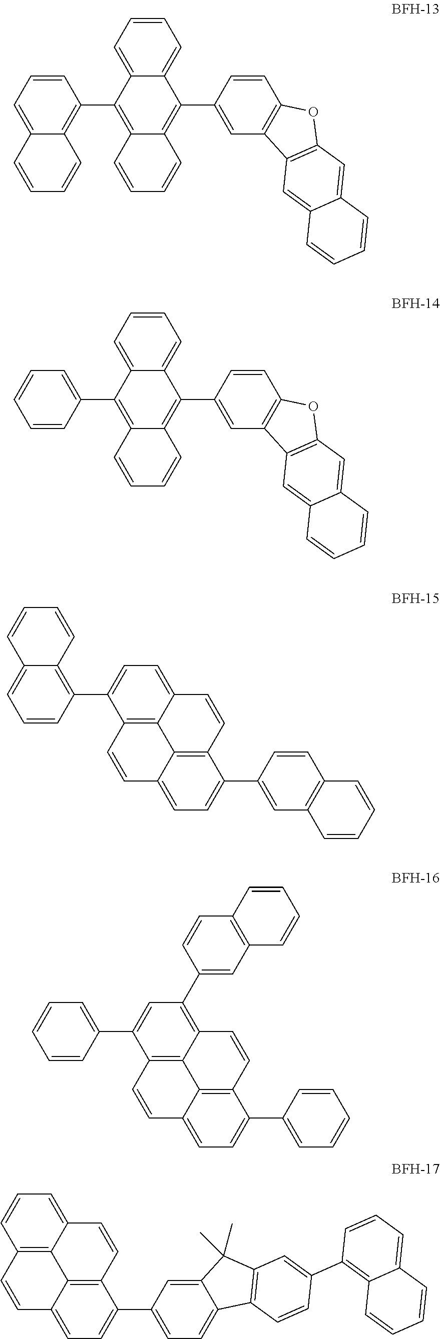

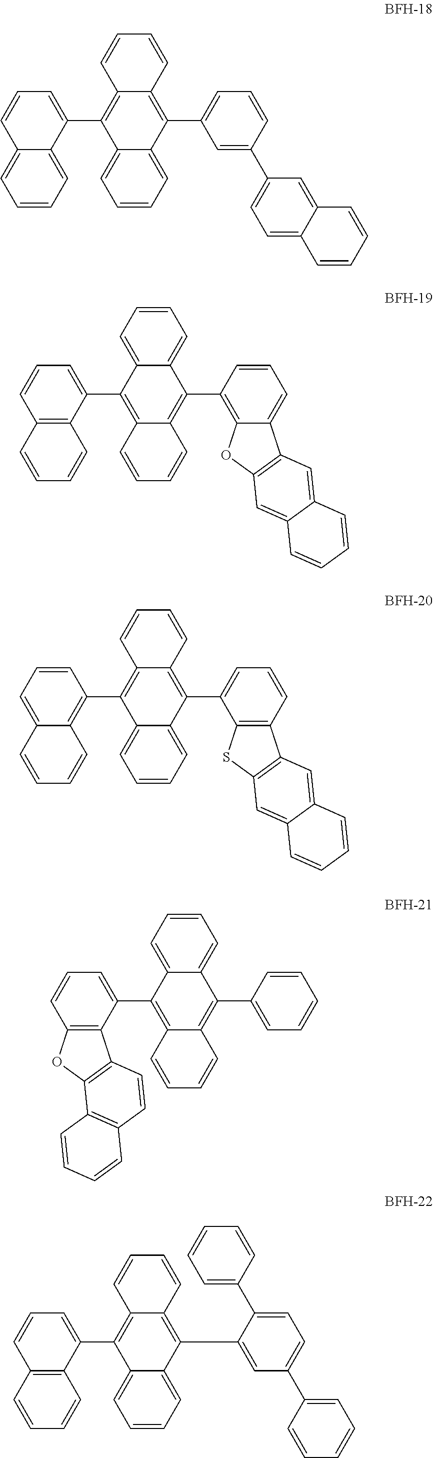

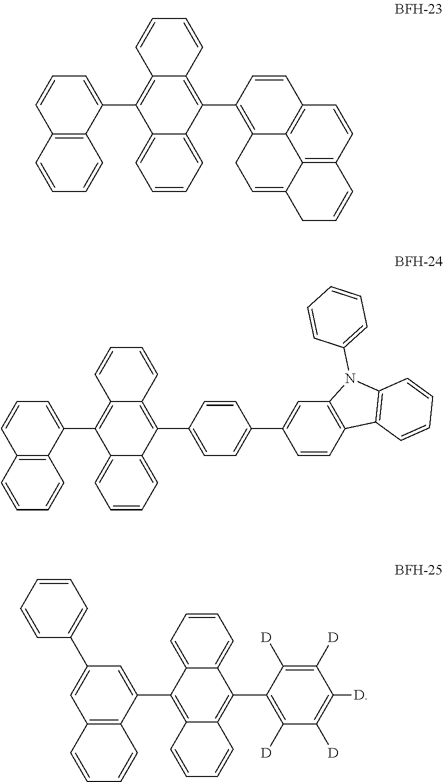

In a specific implementation of the present application, a mass proportion of the fluorescent dye in the light-emitting layer is generally controlled to be 0.1% to 50%. Reasonable controlling the doping amount of the dye in the light-emitting layer is beneficial for further improving the luminescence efficiency of the device. Of course, different host materials and dyes in the light-emitting layer of the organic electroluminescent device in the present application will affect the performance of the device. Therefore, in general, for different host materials and dyes, when the mass proportion of dyes in the light-emitting layer is controlled to 0.5%-20%, it can be basically ensured that the device has excellent luminescence efficiency.

The organic electroluminescent device of the present application has no special limitation to the thickness of the light-emitting layer, which is consistent with the thickness of the light-emitting layer of the existing device in the art, for example, 10-60 nm.

Besides the light-emitting layer, the organic electroluminescent device of the present application further includes an anode located on one side of the light-emitting layer and a cathode located on the other side of the light-emitting layer, that is, the light-emitting layer is disposed between the cathode and the anode. The anode and cathode may employ materials commonly used in the art. For example, transparent conductive materials such as indium tin oxide (ITO), indium zinc oxide (IZO), stannic oxide (SnO₂), zinc oxide (ZnO) and other oxide and any combination thereof are used as the material for anode; metals or alloys such as magnesium (Mg), silver (Ag), aluminum (Al), aluminum-lithium (Al—Li), calcium (Ca), magnesium-indium (Mg—In), magnesium-silver (Mg—Ag) and any combination thereof are used as the material for cathode. Specifically, the cathode or anode can be formed by sputtering or depositing on a substrate as a corresponding material, the substrate is a glass or a polymer material with excellent mechanical strength, thermal stability, waterproofing effect, and transparency. In addition, thin film transistors (TFT) may also be provided on the substrate that is used for a display apparatus.

Further, in addition to the cathode, light-emitting layer and anode, the organic electroluminescent device of the present application further includes other auxiliary functional area that is conducive to inject and recombine carriers. For example, a hole transmission area is disposed between the anode and the light-emitting layer, and an electron transmission area is disposed between the cathode and the light-emitting layer.

Specifically, the hole transmission area can be a hole transmission layer (HTL) having a single layer structure, including a single-layer hole transmission layer containing only one compound and a single-layer hole transmission layer containing multiple compounds. In a direction of the anode pointing towards the light-emitting layer, the hole transmission area may also be a multi-layer structure that sequentially includes at least two layers of the hole injection layer (HIL), the hole transmission layer (HTL), and the electron blocking layer (EBL).

The material in the hole transmission area (including HIL, HTL and EBL) is selected from, but not limited to, a phthalocyanine derivative such as CuPc, a conductive polymer or a polymer containing a conductive dopant, such as polyphenylenevinylene, polyaniline/dodecylbenzene sulfonic acid (Pani/DBSA), poly (3,4-ethylenedioxythiophene)/poly (4-styrene sulfonate)(PEDOT/PSS), polyaniline/camphorsulfonic acid (Pani/CSA), polyaniline/poly (4-styrene sulfonate)(Pani/PSS), and aromatic amine derivative.

Where, if the material of the hole transmission auxiliary layer is an aromatic amine derivative, it is one or more of the compounds represented by HT-1 to HT-34.

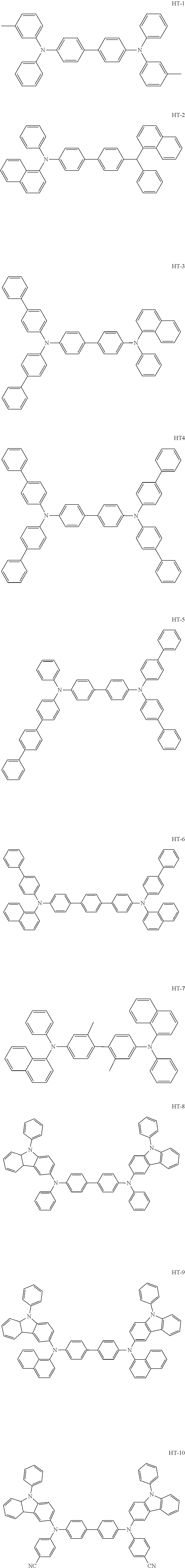

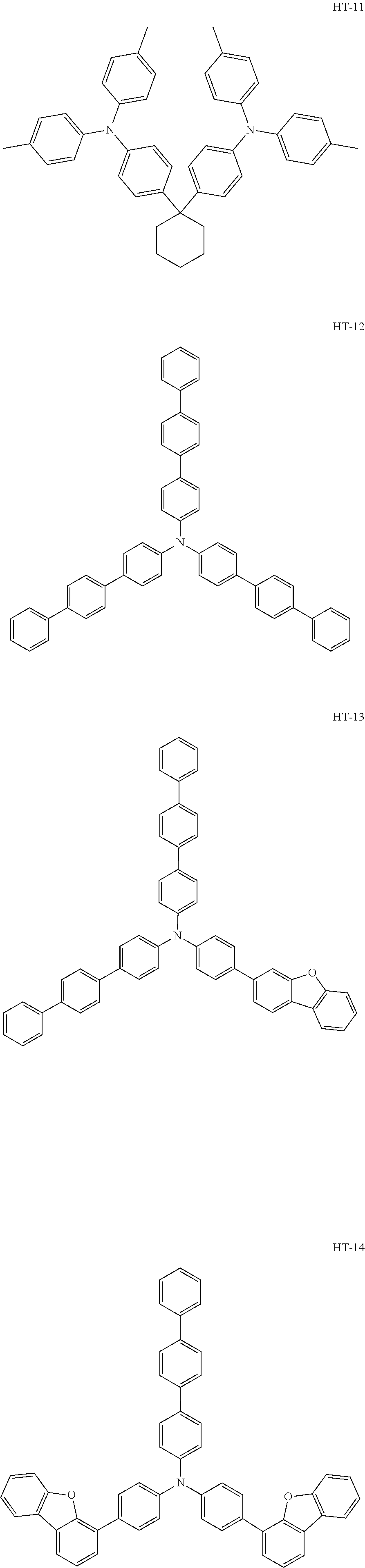

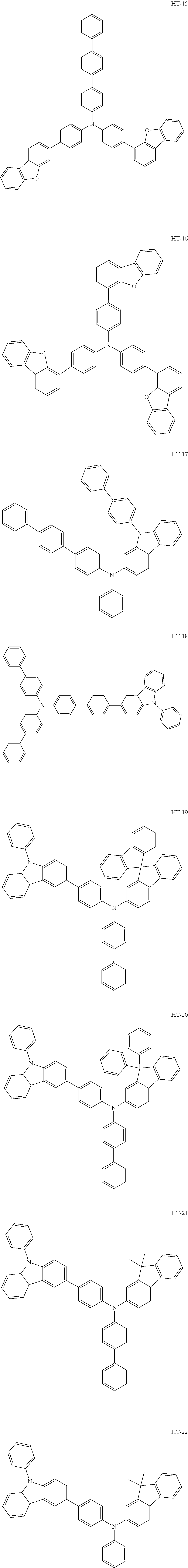

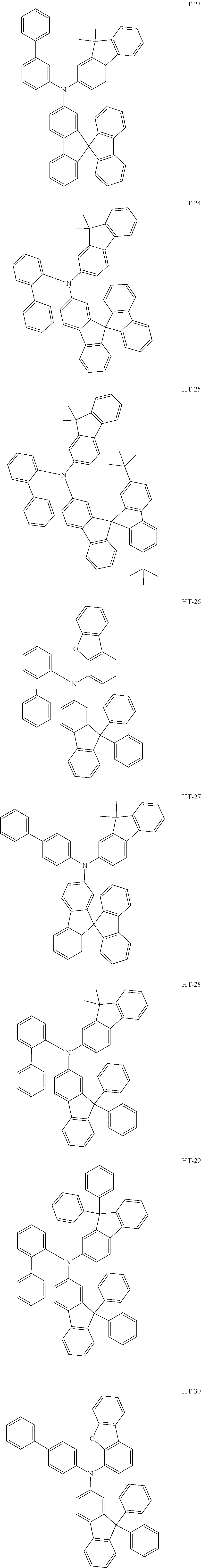

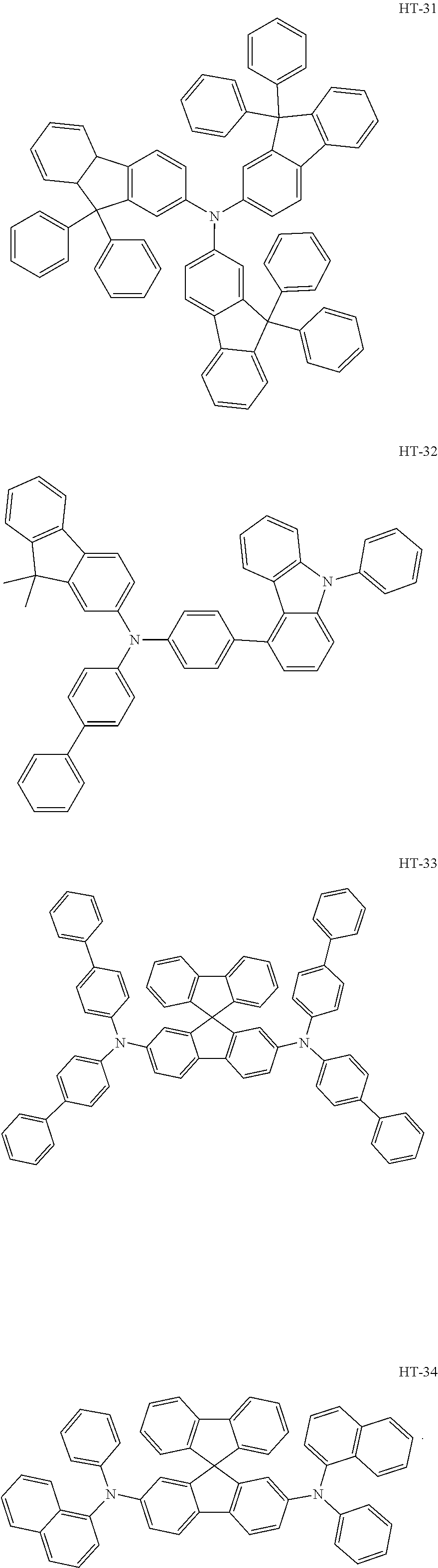 [SMILES: C1=CC2c3ccc(N(c4ccc5c(c4)C(c4ccccc4)(c4ccccc4)c4ccccc4-5)c4ccc5c(c4)C(c4ccccc4)(c4ccccc4)c4ccccc4-5)cc3C(c3ccccc3)(c3ccccc3)C2C=C1 ; CC1(C)c2ccccc2-c2ccc(N(c3ccc(-c4ccccc4)cc3)c3ccc(-c4cccc5c4c4ccccc4n5-c4ccccc4)cc3)cc21 ; c1ccc(-c2ccc(N(c3ccc(-c4ccccc4)cc3)c3ccc4c(c3)C3(c5ccccc5-c5ccccc53)c3cc(N(c5ccc(-c6ccccc6)cc5)c5ccc(-c6ccccc6)cc5)ccc3-4)cc2)cc1 ; c1ccc(N(c2ccc3c(c2)C2(c4ccccc4-c4ccccc42)c2cc(N(c4ccccc4)c4cccc5ccccc45)ccc2-3)c2cccc3ccccc23)cc1]

The hole injection layer is located between the anode and the hole transmission layer. The hole injection layer is a single compound material or a combination of multiple compounds. For example, the hole injection layer may employ one or more compounds of HT-1 to HT-34, or one or more compounds of HI1 to HI3; or one or more compounds of HT-1 to HT-34 that are doped with one or more of the following compounds of HI1 to HI3.

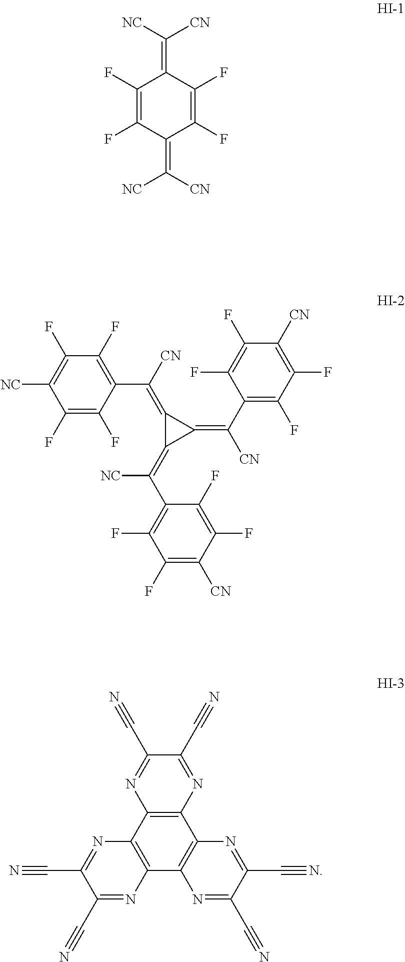 [SMILES: N#CC(C#N)=c1c(F)c(F)c(=C(C#N)C#N)c(F)c1F ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; N#Cc1nc2c3nc(C#N)c(C#N)nc3c3nc(C#N)c(C#N)nc3c2nc1C#N]

The electron transmission area is an electron transmission layer (ETL) with a single-layer structure, including a single-layer electron transmission layer containing only one compound and a single-layer electron transmission layer containing multiple compounds. In a direction of the cathode pointing towards the light-emitting layer, the electron transmission area may also be a multi-layer structure including at least two of the electron injection layer (EIL), the electron transmission layer (ETL), and the hole blocking layer (HBL).

The material of the electronic transmission layer is selected from, but not limited to, one or more from the following ET-1 to ET-73.

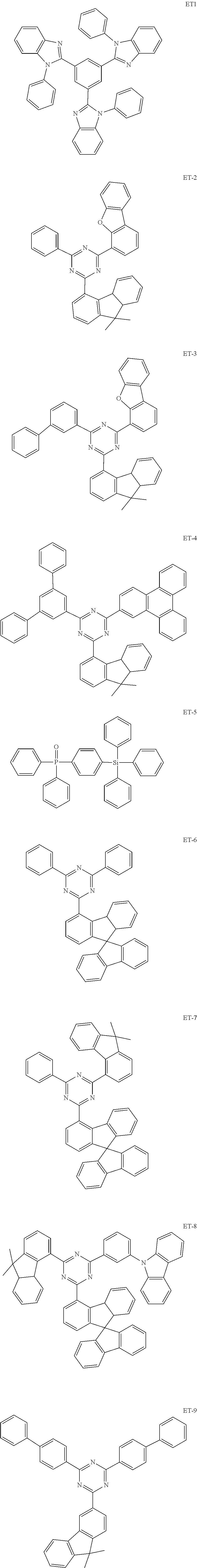

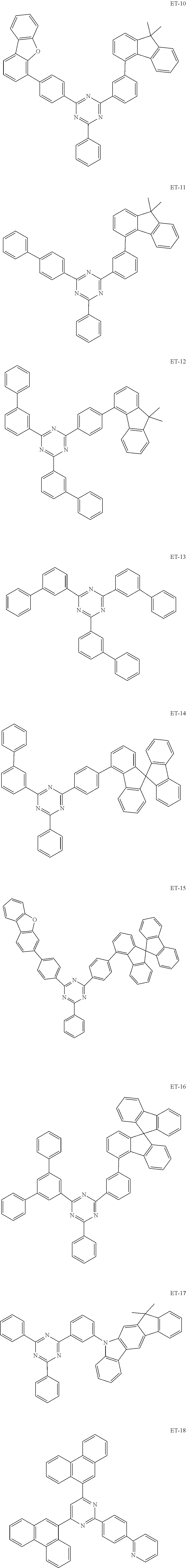

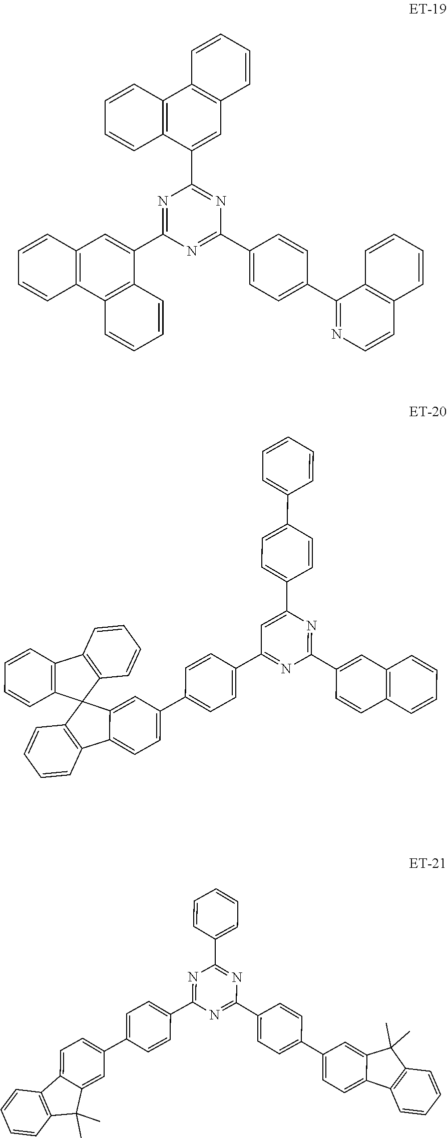 [SMILES: c1ccc2c(-c3ccc(-c4nc(-c5cc6ccccc6c6ccccc56)nc(-c5cc6ccccc6c6ccccc56)n4)cc3)nccc2c1 ; c1ccc(-c2ccc(-c3cc(-c4ccc(-c5ccc6c(c5)C5(c7ccccc7-c7ccccc75)c5ccccc5-6)cc4)nc(-c4ccc5ccccc5c4)n3)cc2)cc1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

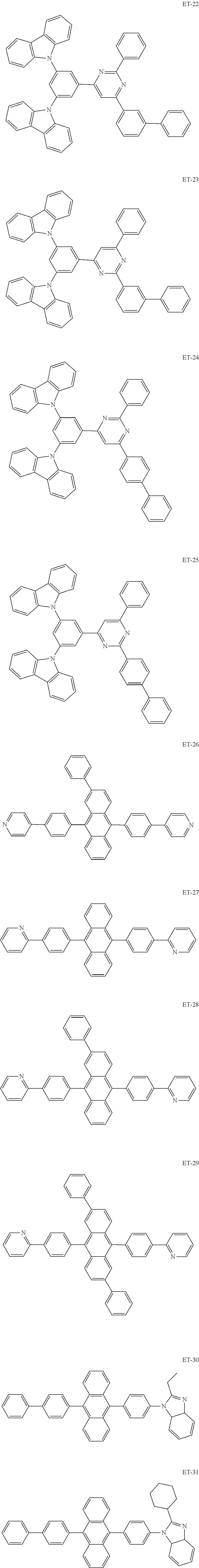

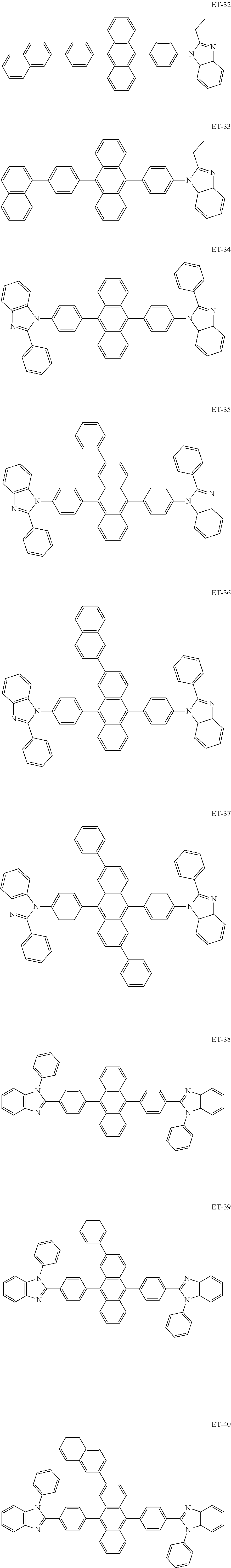

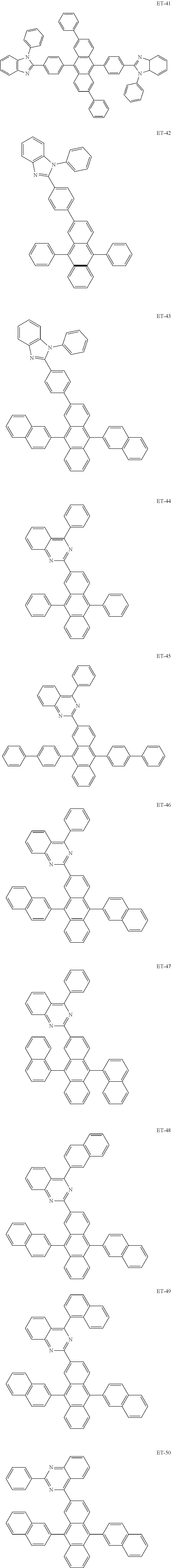

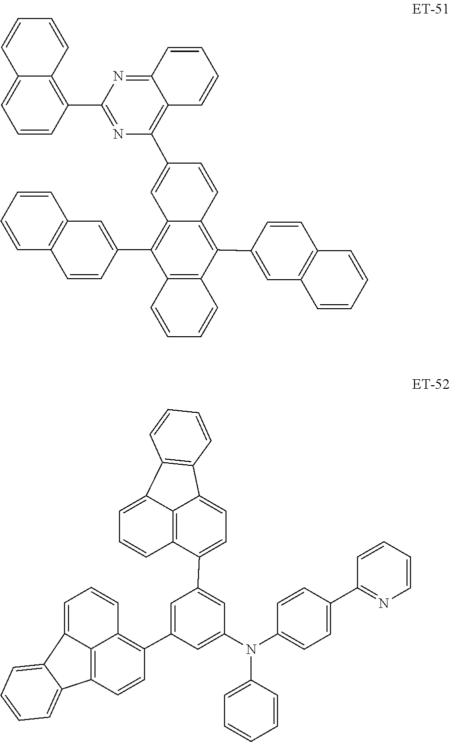 [SMILES: c1ccc2cc(-c3c4ccccc4c(-c4ccc5ccccc5c4)c4cc(-c5nc(-c6cccc7ccccc67)nc6ccccc56)ccc34)ccc2c1 ; c1ccc(N(c2ccc(-c3ccccn3)cc2)c2cc(-c3ccc4c5c(cccc35)-c3ccccc3-4)cc(-c3ccc4c5c(cccc35)-c3ccccc3-4)c2)cc1]

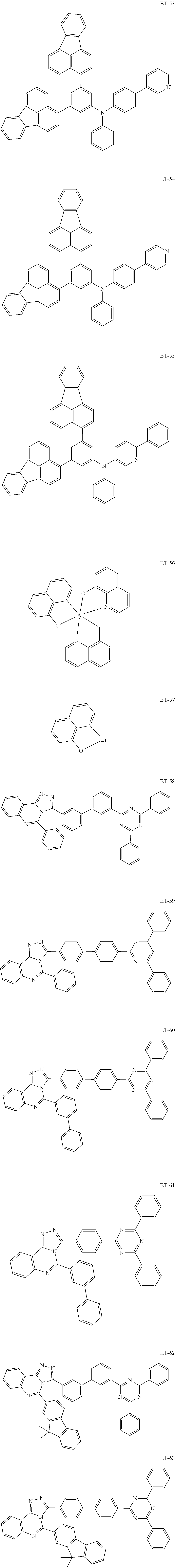

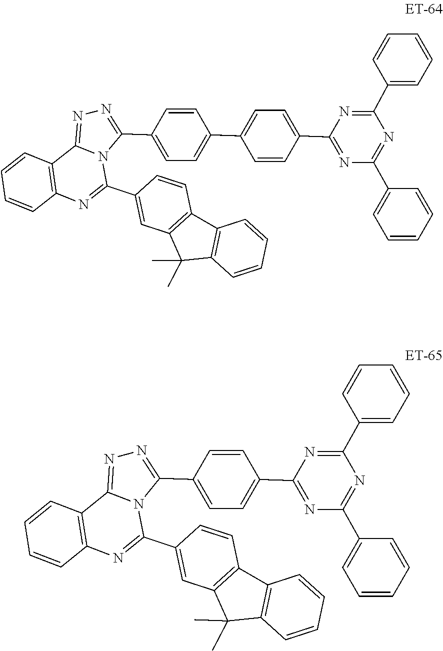 [SMILES: CC1(C)c2ccccc2-c2ccc(-c3nc4ccccc4c4nnc(-c5ccc(-c6ccc(-c7nc(-c8ccccc8)nc(-c8ccccc8)n7)cc6)cc5)n34)cc21 ; CC1(C)c2ccccc2-c2ccc(-c3nc4ccccc4c4nnc(-c5ccc(-c6nc(-c7ccccc7)nc(-c7ccccc7)n6)cc5)n34)cc21]

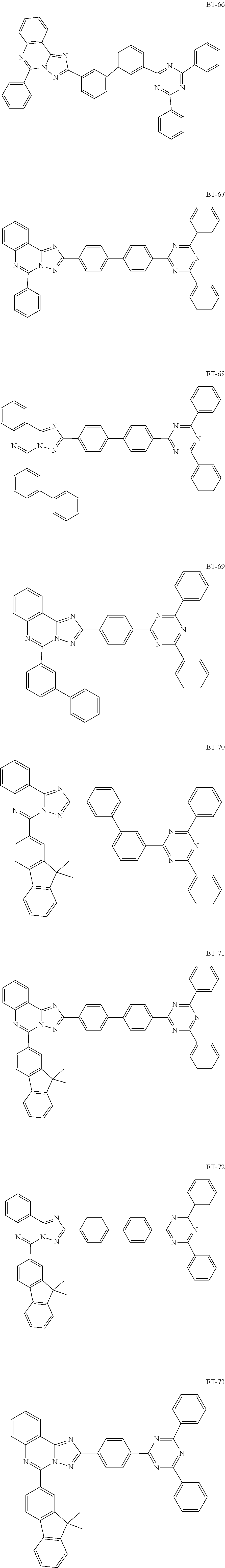

The hole barrier layer (HBL) is located between the electron transmission layer and the light-emitting layer. The hole blocking layer can use, but is not limited to, one or more compounds of the above ET-1 to ET-73.

The electron injection material in the electron injection layer includes any one or at least two from the following compounds: Liq, LiF, CaCl, CsF, Li₂O, Cs₂CO₃, BaO, Na, Li, Ca, Mg, Ag, and Yb.

A capping layer (SPL layer) is deposited via evaporation on the cathode of the device to improve the efficiency of the device and adjust the optical microcavity, etc.

The thickness of the above respective layers can be the conventional thickness of these layers in the art.

The present application further provides a preparation method of the organic electroluminescent device, including depositing an anode, a hole transmission area, a light-emitting layer, an electron transmission area, and a cathode on a substrate in sequence, and then sealing. In the preparation of the light-emitting layer, the evaporation speed of the host material and the evaporation speed of the fluorescent dye are adjusted by a method of multi-source co-evaporation to make the fluorescent dye reach a preset doping ratio, and the light-emitting layer is formed by a method of co-evaporation of the triplet-triplet annihilation material source and any one of the fluorescent dye sources mentioned above. The deposition methods of the anode, hole transmission area, electron transmission area, and cathode are the same as those existing methods in the art.

The organic electroluminescent device of the present application has the advantages of low driving voltage and high efficiency through the matching of specific materials of the light-emitting layer and the selection of special fluorescent dye.

A second aspect of the present application further provides a display apparatus, including the above organic electroluminescent device. The display apparatus may specifically include an OLED display and other display device, as well as any product or component with a displaying function such as televisions, digital cameras, mobile phones, tablets, etc. that includes the display apparatus. The display apparatus has the same advantages as the above organic electroluminescent device compared with the prior art, which will not be described here.

The following will take multiple synthesis Examples as examples to elaborate the preparation method of a specific compound of the fluorescent dye of the present application, but the preparation method of the dye of the present application is not limited to these synthesis Examples.

Various chemicals used in the synthesis process such as petroleum ether, tert-butylbenzene, ethyl acetate, sodium sulfate, toluene, dichloromethane, potassium carbonate, boron tribromide, N,N-diisopropylethylamine, reaction intermediate and other basic chemical raw materials are purchased from Shanghai Titan Technology Co., Ltd. (Titan) and Xilong Chemical Co., Ltd. The mass spectrometer used to determine the following compounds is ZAB-HS mass spectrometer (manufactured by UK Micromass company).

The synthetic method of the dye compound in the present application is briefly described below. Firstly, hydrogen and Cl atoms between/on X₁, X₂, X₃ and X₄ are ortho-metallized with N-butyl lithium or tert-butyl lithium. Then, after boron tribromide is added for lithium-boron metal exchange, the Bronsted base such as N, N-diisopropylethylamine is added, and the Tandem Bora-Friedel-Crafts Reaction is performed to obtain a target.

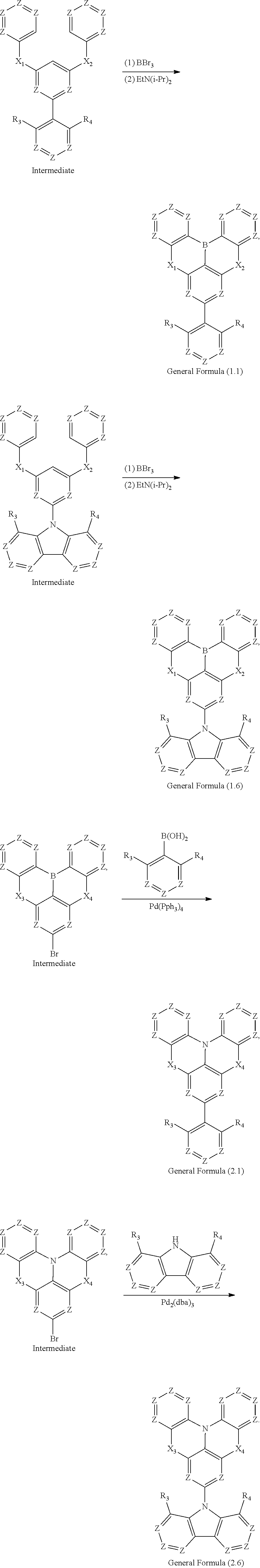

More specifically, a synthesis method for representative and specific fluorescent dye compounds in the present application is provided below.

### Synthesis Example 1: Synthesis of Compound S-7

1. Synthesis of compound S-7-2: in a three-necked bottle, under nitrogen protection, 0.01 mol of S-7-1, 0.025 mol of 3,6-di-tert-butylcarbazole and 150 ml of toluene were added and mixed under stirring, and then 5×10⁻⁵ mol Pd₂(dba)₃ and 0.03 mol sodium tert-butoxide were added for reflux reaction for 12 hours, followed by sampling and dropping on a plate, when if there was no bromine residue, the reaction was complete; the obtained reaction product was naturally cooled to room temperature and filtered, the filtrate was spin-evaporated to no fraction, and then purified through a neutral silica gel column (developing agent: dichloromethane and petroleum ether) to give a target compound S-7-2 (9.22 g; yield: 73%; HPLC analysis purity: 99.56%) as a white powder.

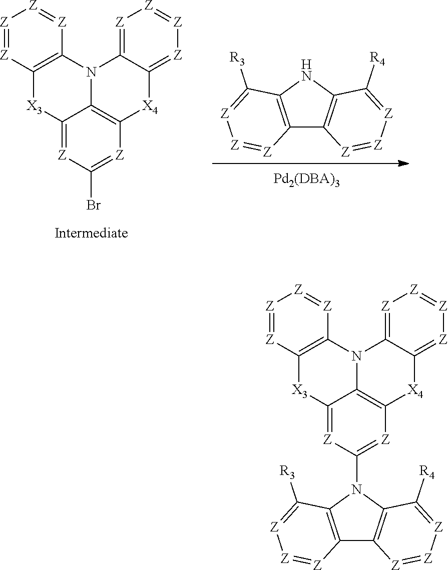

2. Synthesis of compound S-7: under nitrogen atmosphere, 0.03 mol of BBr₃ was added into a solution of 0.01 mol of S-7-2 in o-dichlorobenzene (100 mL), and subjected to reaction at 190° C. for 24 hours. The solvent after reaction was spin-dried under vacuum, followed by purification through a silica gel column (developing agent, ethyl acetate: petroleum ether=50:1), giving a target compound S-7 (0.64 g; yield: 5%; HPLC analysis purity: 99.42%) as a green solid. MALDI-TOF-MS result: molecular-ion peak: 1271.55, element analysis result: theoretical value (%): C, 86.90; H, 7.85; B, 0.85; N, 4.41; experimental value (%): C, 86.80; H, 7.85; B. 0.85; N, 4.51.

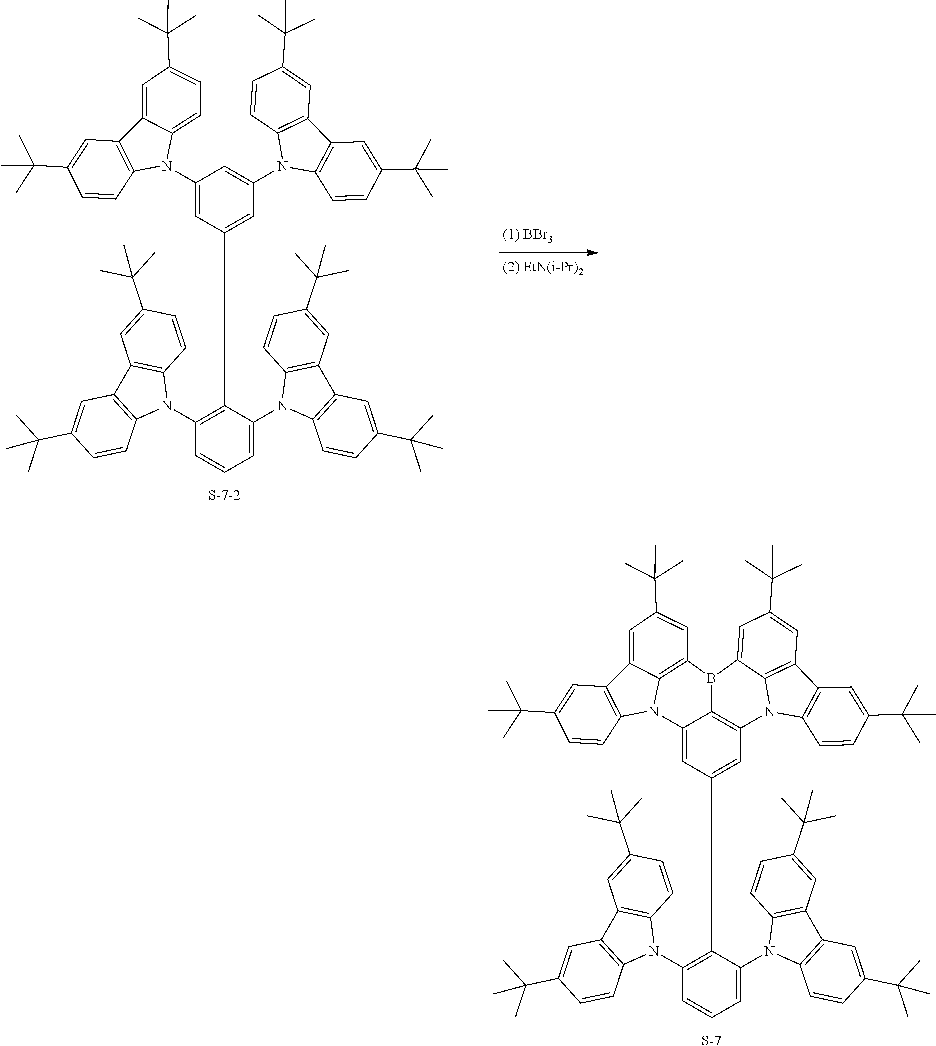

### Synthesis Example 2: Synthesis of Compound S-13

1. Synthesis of compound S-13-2: this Example was basically the same as the that of compound S-7-2, except that in this Example, S-7-1 was replaced by S-13-1 with an equal amount of substance. A target compound S-13-2 (10.38 g; yield: 92%; HPLC analysis purity: 99.37%) as a white solid was obtained.

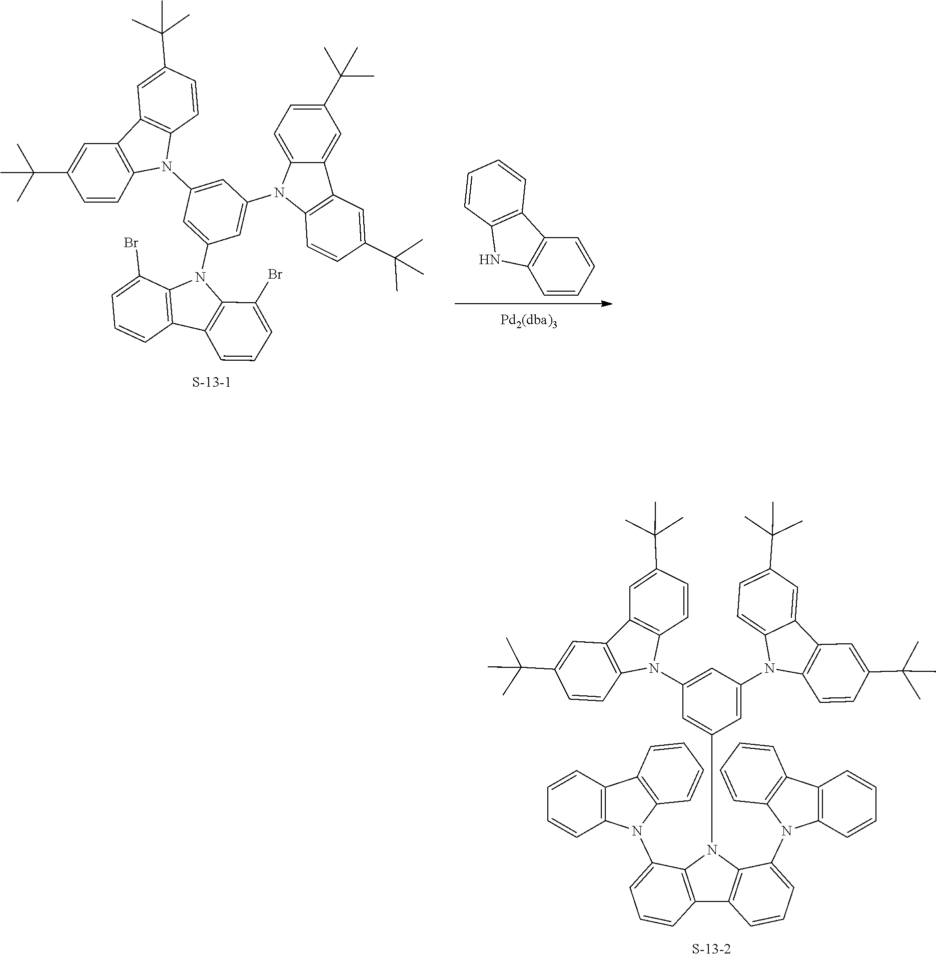 [SMILES: CC(C)(C)c1ccc2c(c1)c1cc(C(C)(C)C)ccc1n2-c1cc(-n2c3ccc(C(C)(C)C)cc3c3cc(C(C)(C)C)ccc32)cc(-n2c3c(Br)cccc3c3cccc(Br)c32)c1 ; c1ccc2c(c1)[nH]c1ccccc12 ; CC(C)(C)c1ccc2c(c1)c1cc(C(C)(C)C)ccc1n2-c1cc(-n2c3ccc(C(C)(C)C)cc3c3cc(C(C)(C)C)ccc32)cc(-n2c3c(-n4c5ccccc5c5ccccc54)cccc3c3cccc(-n4c5ccccc5c5ccccc54)c32)c1]

2. Synthesis of compound S-13: this Example was basically the same as that of compound S-7, except that in this Example, S-7-2 was replaced by S-13-2 with an equal amount of substance. A target compound S-13 (2.38 g; yield: 21%; HPLC analysis purity: 99.33%) as a green solid was obtained. MALDI-TOF-MS result: molecular-ion peak: 1135.62, element analysis result: theoretical value (%): C, 86.68; H, 6.21; B, 0.95; N, 6.16; experimental value (%): C, 86.78; H, 6.31; B, 0.96; N, 6.15.

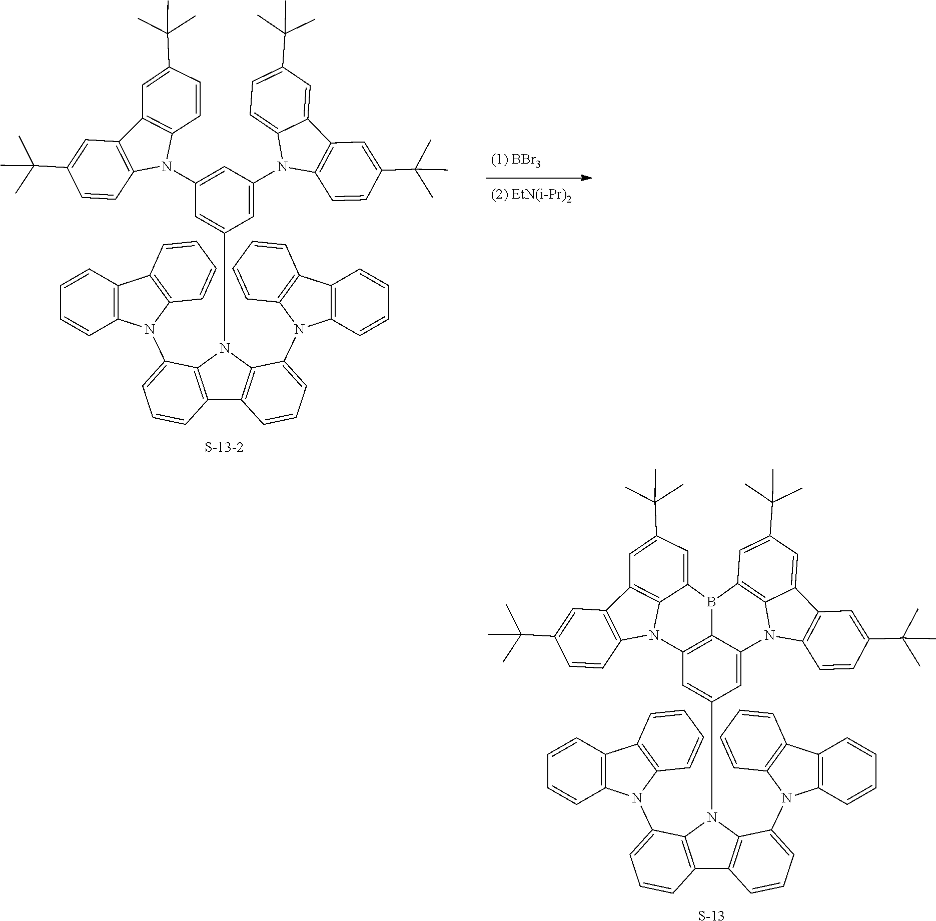

### Synthesis Example 3: Synthesis of Compound S-52

1. Synthesis of compound S-52-2: this Example was basically the same as that of compound S-7-2, except that S-7-1 was replaced by S-52-1 with an equivalent amount of substance. A target compound S-52-2 (10.89 g; yield: 86%; HPLC analysis purity: 99.53%) as a white solid was obtained.

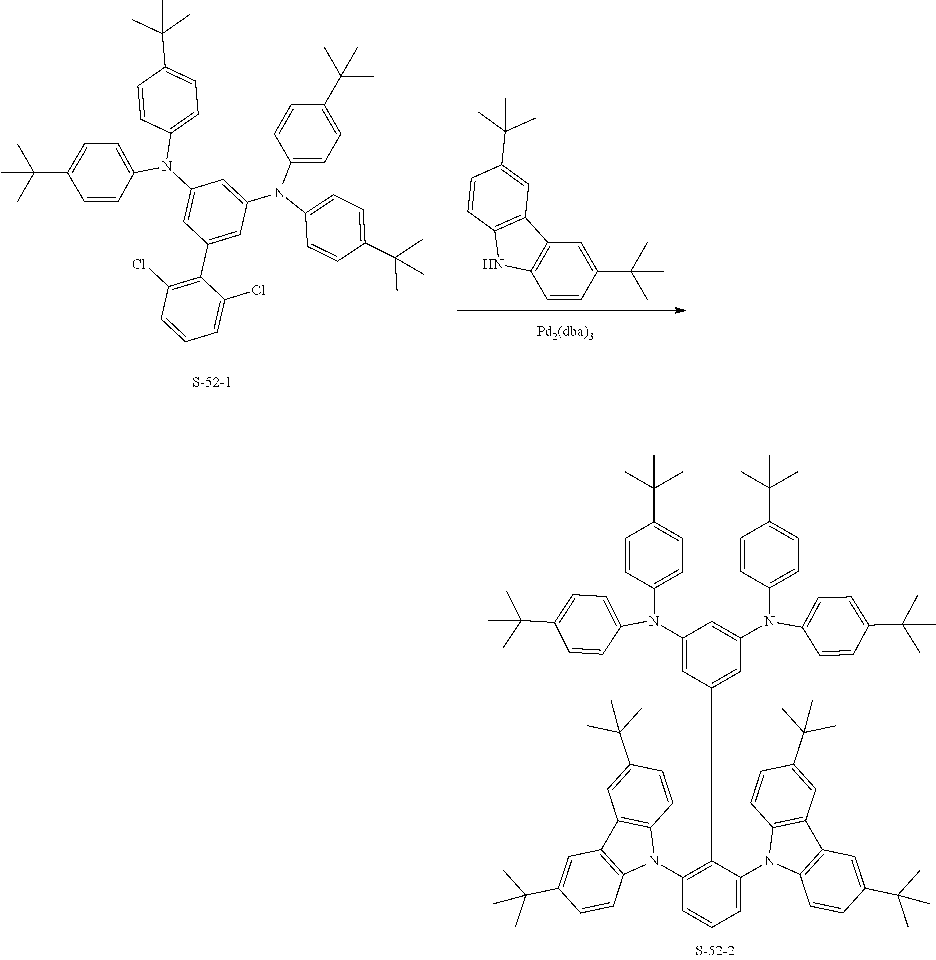 [SMILES: CC(C)(C)c1ccc(N(c2ccc(C(C)(C)C)cc2)c2cc(-c3c(Cl)cccc3Cl)cc(N(c3ccc(C(C)(C)C)cc3)c3ccc(C(C)(C)C)cc3)c2)cc1 ; CC(C)(C)c1ccc2[nH]c3ccc(C(C)(C)C)cc3c2c1 ; CC(C)(C)c1ccc(N(c2ccc(C(C)(C)C)cc2)c2cc(-c3c(-n4c5ccc(C(C)(C)C)cc5c5cc(C(C)(C)C)ccc54)cccc3-n3c4ccc(C(C)(C)C)cc4c4cc(C(C)(C)C)ccc43)cc(N(c3ccc(C(C)(C)C)cc3)c3ccc(C(C)(C)C)cc3)c2)cc1]

2. Synthesis of compound S-52: this Example was basically the same as the synthesis of compound S-7, except that in this example, S-7-2 was replaced by S-52-2 with an equivalent amount. A target compound S-52 (4.59 g; yield: 36%; HPLC analysis purity: 99.23%) as a green solid was obtained. MALDI-TOF-MS result: molecular-ion peak: 1275.02, element analysis result: theoretical value (%): C, 86.62; H, 8.14; B, 0.85; N, 4.39; experimental value (%): C, 86.52; H, 8.24; B, 0.86; N, 4.38.

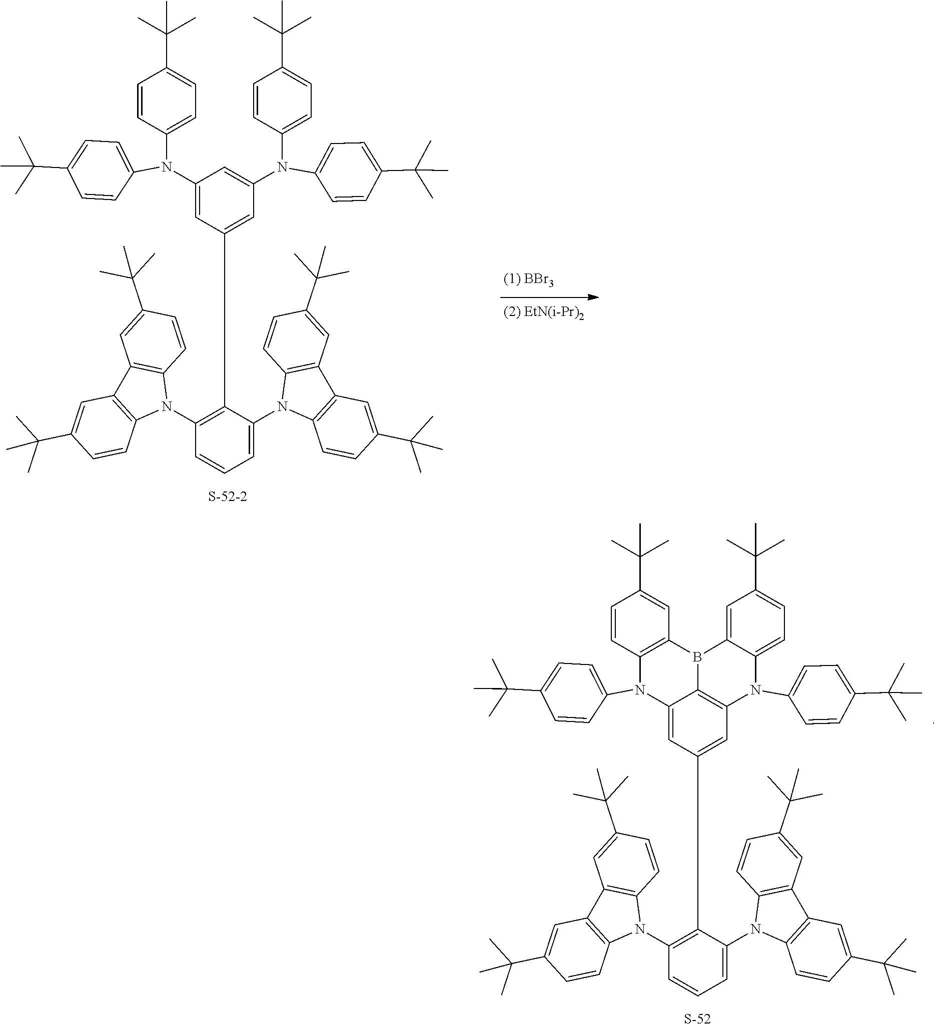

### Synthesis Example 4: Synthesis of Compound S-244

1. This Example was basically the same as the synthesis of compound S-7-2, except that in this Example, S-7-1 was replaced by S-244-1 with an equivalent amount of substance. A target compound S-244 (3.43 g, yield: 33%, purity: 99.39% by HPLC analysis) was a green solid. MALDI-TOF-MS result: molecular-ion peak: 1039.62, element analysis result: theoretical value (%): C, 85.43; H, 7.46; N, 4.04; O, 3.08; experimental value (%): C, 85.53; H, 7.36; N, 4.06; O, 3.06.

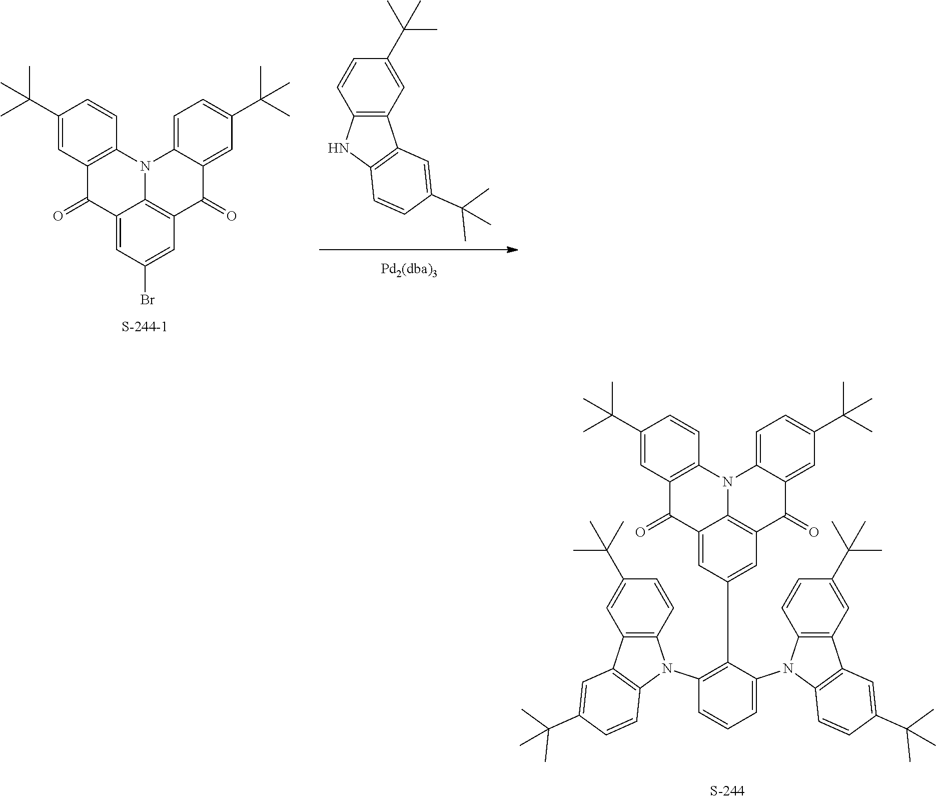 [SMILES: CC(C)(C)c1ccc2c(c1)c(=O)c1cc(Br)cc3c(=O)c4cc(C(C)(C)C)ccc4n2c13 ; CC(C)(C)c1ccc2[nH]c3ccc(C(C)(C)C)cc3c2c1 ; CC(C)(C)c1ccc2c(c1)c(=O)c1cc(-c3c(-n4c5ccc(C(C)(C)C)cc5c5cc(C(C)(C)C)ccc54)cccc3-n3c4ccc(C(C)(C)C)cc4c4cc(C(C)(C)C)ccc43)cc3c(=O)c4cc(C(C)(C)C)ccc4n2c13]

In addition, other obtained fluorescent dyes in the present application were also characterized through mass spectrometry (MALDI-TOF-MS molecular ion peak), as shown in Table 1 below.

TABLE 1

Mass spectrum

| | Theoretical value | Experimental value |
|---|---|---|
| S-24 | 970.51 | 970.75 |
| S-49 | 1236.63 | 1236.71 |
| S-50 | 1050.57 | 1050.69 |
| S-58 | 1236.63 | 1236.77 |
| S-69 | 1325.66 | 1325.71 |
| S-96 | 1267.77 | 1267.89 |
| S-108 | 1267.77 | 1267.81 |
| S-146 | 919.46 | 919.56 |
| S-252 | 753.45 | 753.51 |

The organic electroluminescent device of the present application is further introduced below through specific embodiments.

### Examples 1-29

Examples 1-29 respectively provided organic electroluminescent devices, the structure of the device successively included an anode, a hole injection layer (HIL), a hole transmission layer (HTL), an electron blocking layer (EBL), a light-emitting layer (EML), a hole blocking layer (HBL), an electron transmission layer (ETL), an electron injection layer (EIL), a cathode, and a capping layer (CPL). Taking Example 1 as an example, the specific preparation method was as following:
(1) A glass plate coated with a conductive layer of ITO/Ag/ITO was ultrasonically treated in a commercial cleaning agent, rinsed in deionized water, ultrasonically degreased in a mixed solvent of acetone and ethanol, baked in a clean environment until the water was completely removed, cleaned with ultraviolet light and ozone, and then subjected to a surface bombardment with a low energy cation beam;
(2) The above glass plate with the anode was placed in a vacuum chamber and vacuumized to less than 1×10⁻⁵ Pa, HT-24 and HI-2 as the hole injection layer were deposited on the anode layer film via co-evaporation, where the ratio of HI-2 was 3%, an evaporation speed of HT-24 was 0.1 nm/s, and an evaporation film had a thickness of 10 nm; (3) The hole transmission layer HT-24 was deposited on the hole injection layer via vacuum evaporation, with an evaporation speed of 0.1 nm/s and a total thickness of evaporation film of 110 nm;
(4) The electron blocking layer EB-1 was deposited on the hole transmission layer via vacuum evaporation, with an evaporation speed of 0.1 nm/s and a total thickness of evaporation film of 5 nm;
(5) The light-emitting layer was deposited on the electron blocking layer via vacuum co-evaporation, the light-emitting layer included the host material BFH-4 and fluorescent dye S-7, a multi-source co-evaporation method was used, with a doping ratio of the dye being 2% for evaporation, an evaporation speed of the host being 0.1 nm/s, and an evaporation film thickness being 20 nm;
(6) The hole blocking layer HB-1 was deposited on the light-emitting layer via vacuum evaporation, with an evaporation speed of 0.1 nm/s and a total thickness of evaporation film of 5 nm;
(7) ET-57 and ET-69 (with a mass ratio of 1:1) as the electron transmission layer were deposited on the hole blocking layer via vacuum evaporation, the evaporation speeds of ET-57 and ET-69 were both 0.1 nm/s, and a total thickness of evaporation film was 30 nm; (8) Yb with a thickness of 1 nm was deposited via vacuum evaporation on the electron transmission layer as the electron injection layer;
(9) An magnesium-silver (Mg—Ag) alloy layer with a thickness of 15 nm was deposited via evaporation on the electron injection layer as the cathode of the device, with a mass ratio of Mg:Ag=1:9;
(10) C-1 layer with a thickness of 65 nm was deposited via evaporation on the cathode as the capping layer of the device.

Specifically, the device is a top-emitting structure that includes an anode, a hole injection layer, a hole transmission layer, an electron blocking layer, a light-emitting layer, a hole blocking layer, an electron transmission layer, an electron injection layer, a cathode, and a capping layer from bottom to top.

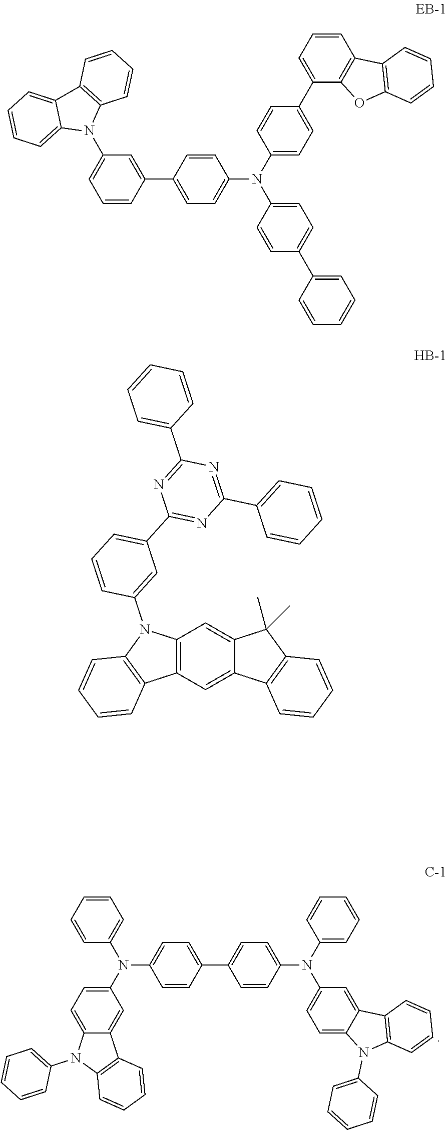 [SMILES: c1ccc(-c2ccc(N(c3ccc(-c4cccc(-n5c6ccccc6c6ccccc65)c4)cc3)c3ccc(-c4cccc5c4oc4ccccc45)cc3)cc2)cc1 ; CC1(C)c2ccccc2-c2cc3c4ccccc4n(-c4cccc(-c5nc(-c6ccccc6)nc(-c6ccccc6)n5)c4)c3cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

In the organic electroluminescent devices provided by Examples 2-29, the specific preparation methods are similar to that of Example 1, except for the specific selection of the host material and fluorescent dye, and the mass proportion of fluorescent dye in the light-emitting layer. The relevant characterizations of fluorescent dye in some devices in the Examples are shown in Table 2 below.

### Comparative Examples 1-8

Comparative Examples of 1-8 provide organic electroluminescent devices. The device structures are consistent with those of Examples 1-29, and the parameters of the corresponding functional layers are basically consistent with those of Examples 1-29. The difference is only that the host material and dye of the light-emitting layer are inconsistent with the materials or the doping concentration used in the Examples.

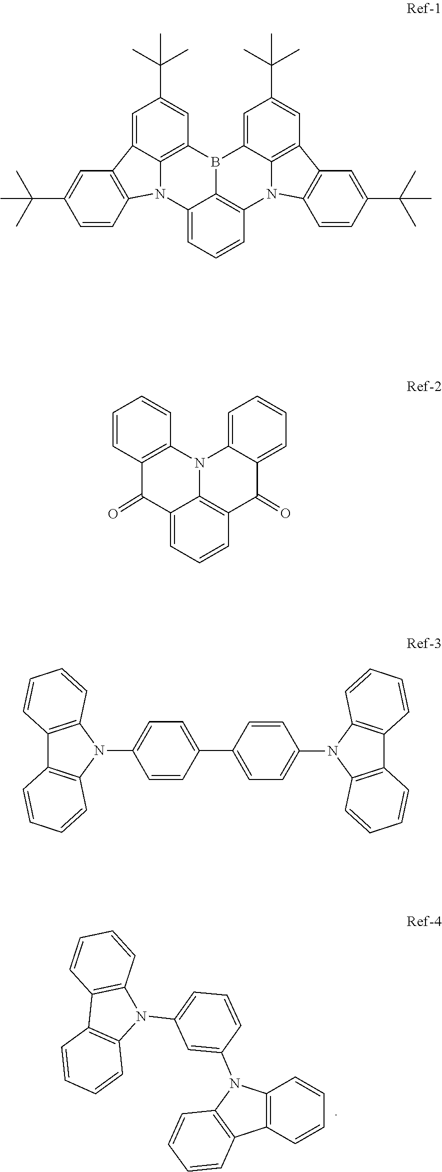 [SMILES: CC(C)(C)c1ccc2c(c1)c1cc(C(C)(C)C)cc3c1n2-c1cccc2c1B3c1cc(C(C)(C)C)cc3c4cc(C(C)(C)C)ccc4n-2c13 ; O=c1c2ccccc2n2c3ccccc3c(=O)c3cccc1c32 ; c1ccc2c(c1)c1ccccc1n2-c1ccc(-c2ccc(-n3c4ccccc4c4ccccc43)cc2)cc1 ; c1cc(-n2c3ccccc3c3ccccc32)cc(-n2c3ccccc3c3ccccc32)c1]

The specific composition of organic electroluminescent devices for Examples 1-29 and Comparative Examples 1-8 is shown in Table 2.

The following tests were conducted on the devices of Examples and Comparative Examples, and the test results are shown in Table 2.

Under the same brightness, Keithley K 2400 digital source meter and PR 655 spectral scanning luminance meter are used to measure the driving voltage and BI value of the organic electroluminescent devices prepared in Examples 1-29 and Comparative Examples 1-8. Specifically, the voltage is increased at a speed of 0.1V per second, and when the brightness of the organic electroluminescent device reaches 1000 cd/m², the measured voltage is the driving voltage and the current density is measured at this time, and the ratio of brightness to current density is the current efficiency; BI value of the device at 1000 cd/m² is derived from the current efficiency at 1000 cd/m² by dividing the CIEy value of the spectrum of the device at this time.

TABLE 2

| | | | Voltage (V) | BI |
|---|---|---|---|---|
| | Host | Dyes and mass | under | value(CE/CIEy) |
| | material | proportion | 1000 cd/m² | under1000 cd/m² |
| Example 1 | BFH-4 | S-7, 2% | 3.98 V | 176 |
| Example 2 | BFH-4 | S-7, 10% | 3.99 V | 174 |
| Example 3 | BFH-4 | S-7, 15% | 4.02 V | 170 |
| Example 4 | BFH-4 | S-7, 20% | 4.02 V | 169 |
| Example 5 | BFH-4 | S-7, 30% | 4.05 V | 166 |
| Example 6 | BFH-14 | S-7, 2% | 3.51 V | 177 |
| Example 7 | BFH-14 | S-7, 15% | 3.58 V | 171 |
| Example 8 | BFH-14 | S-7, 20% | 3.59 V | 170 |
| Example 9 | BFH-14 | S-7, 30% | 3.77 V | 167 |
| Example 10 | BFH-4 | S-13, 2% | 3.97 V | 178 |
| Example 11 | BFH-4 | S-13, 15% | 4.04 V | 173 |
| Example 12 | BFH-4 | S-24, 2% | 4.03 V | 169 |
| Example 13 | BFH-4 | S-24, 15% | 4.07 V | 165 |
| Example 14 | BFH-4 | S-50, 2% | 3.87 V | 180 |
| Example 15 | BFH-4 | S-50, 15% | 3.90 V | 175 |
| Example 16 | BFH-4 | S-52, 2% | 3.88 V | 183 |
| Example 17 | BFH-4 | S-52, 15% | 3.92 V | 180 |
| Example 18 | BFH-4 | S-69, 2% | 3.95 V | 181 |
| Example 19 | BFH-4 | S-69, 15% | 3.98 V | 179 |
| Example 20 | BFH-4 | S-146, 2% | 4.05 V | 166 |
| Example 21 | BFH-4 | S-146, 15% | 4.09 V | 162 |
| Example 22 | BFH-4 | S-244, 2% | 4.06 V | 171 |
| Example 23 | BFH-4 | S-244, 15% | 4.11 V | 164 |
| Example 24 | BFH-4 | S-252, 2% | 4.11 V | 170 |
| Example 25 | BFH-4 | S-252, 15% | 4.13 V | 163 |
| Example 26 | BFH-21 | S-49, 2% | 3.61 V | 172 |
| Example 27 | BFH-21 | S-58, 2% | 3.58 V | 176 |
| Example 28 | BFH-18 | S-96, 2% | 3.99 V | 173 |
| Example 29 | BFH-18 | S-108, 2% | 3.96 V | 178 |
| Comparative | BFH-4 | Ref-1, 2% | 4.26 V | 152 |
| Example 1 | | | | |
| Comparative | BFH-4 | Ref-1, 15% | 4.39 V | 121 |
| Example 2 | | | | |
| Comparative | BFH-4 | Ref-2, 2% | 4.33 V | 135 |
| Example 3 | | | | |
| Comparative | BFH-4 | Ref-2, 15% | 4.44 V | 87 |
| Example 4 | | | | |
| Comparative | Ref-3 | S-7, 2% | 4.62 V | 103 |
| Example 5 | | | | |
| Comparative | Ref-3 | S-7, 15% | 4.67 V | 92 |
| Example 6 | | | | |
| Comparative | Ref-4 | S-7, 2% | 4.55 V | 95 |
| Example 7 | | | | |
| Comparative | Ref-4 | S-7, 15% | 4.59 V | 83 |
| Example 8 | | | | |

According to Table 2, it can be seen that:
1. Compared with the Comparative Examples, organic electroluminescent devices in Examples 1-29 of the present application can effectively reduce the driving voltage and improve the luminescence efficiency by introducing carbon ring groups or heterocyclic groups represented by A and coated with a group with large steric effect in the molecular structure;
2. Compared with Comparative Examples 1-4, organic electroluminescent devices using the fluorescent dye represented by Formula (1) or Formula (2) in Examples 1-29 of the present application have less dependence on the mass proportion of the fluorescent dye; with the change of the mass proportion of the fluorescent dye, the fluctuation of the driving voltage and the luminescence efficiency of the device is not significant; further, from Examples 1-5 and 6-9, it can be seen that the performance of the device is more excellent when the mass proportion of the fluorescent dye is 0.5-20%;
3. From Examples 26-27 and 28-29, it can be seen that when Z₉ and Z₁₀ of the fluorescent dye represented by Formula (1) or (2) are each CH, it is more beneficial to reduce the driving voltage of the device and improve the luminescence efficiency thereof;
4. For Comparative Examples 1-4, when molecules represented by Ref-1 and Ref-2 are used as the dyes, the driving voltage and luminescence efficiency of the device are not as good as those in the Examples; when the doping concentration of dye is changed by changing Ref1 and Ref2 at the same time, the luminescence efficiency and driving voltage of the device have significant changes; for Comparative Examples 5-8, other types of non-triplet-triplet quenching host (ref-3 and ref-4 molecules) are used, the driving voltage of the device is significantly increased compared with Examples, and the luminescence efficiency of the device is reduced. Therefore, the present application can achieve better device performance by matching a class of triplet-triplet annihilation hosts, which can meet the requirements of current panel manufacturers for high-performance materials, and has good application prospects.

Finally, it should be noted that the above embodiments are only used to illustrate the technical solution of the present application and not to limit it; although the present application has been described in detail with reference to the aforementioned embodiments, ordinary technical personnel in the art should understand that they can still make modification on the technical solutions recorded in the aforementioned embodiments, or make an equivalent substitution some or all of the technical features; and these modification or substitute do not make the essence of the corresponding technical solutions deviate from the scope of the technical solutions of the various embodiments of the present application.

What is claimed is:

1. An organic electroluminescent device, comprising: a light-emitting layer, the light-emitting layer comprises a triplet-triplet annihilation type host and a fluorescent dye, wherein the fluorescent dye has a structure represented by the following Formula (1) or Formula (2): 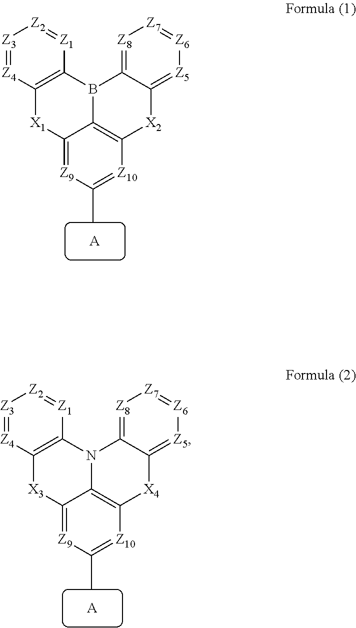
in Formula (1), X₁ and X₂ are each independently represented as O, S, or N(R₁);
in Formula (2), X₃ and X₄ are each independently represented as B(R₂) or C(═O);
in Formula (1) or (2), A represents one of a substituted or unsubstituted C₆-C₆₀ carbocyclic group, and a substituted or unsubstituted C₃-C₆₀ heterocyclyl; a substituted group in A is one or a combination of at least two selected from deuterium, tritium, cyano, halogen, C₁-C₁₀ alkyl, C₃-C₁₀ cycloalkyl, silyl, C₆-C₃₀ arylamino, C₆-C₃₀ aryl, and C₂-C₃₀ heteroaryl, the substituted group is independently connected to a connected aromatic ring or heteroaromatic ring to form a ring or not to form a ring;
in Formula (1) or (2), Z₁-Z₁₀ are each independently represented as N or CR, with R being the same or different each time, two adjacent R can bond to each other to form a ring;
R₁ is connected to adjacent R through a single bond, while R₂ is connected to adjacent R through a single bond;
R₁ represents one of a substituted or unsubstituted C₁-C₁₀ alkyl, a substituted or unsubstituted C₃-C₁₀ cycloalkyl, a substituted or unsubstituted C₆-C₃₀ aryl, and a substituted or unsubstituted C₂-C₃₀ heteroaryl;
R₂ represents one of a substituted or unsubstituted C₆-C₃₀ aryl, and a substituted or unsubstituted C₂-C₃₀ heteroaryl;
R represents one of hydrogen, deuterium, tritium, cyano, halogen, a substituted or unsubstituted C₁-C₁₀ alkyl, a substituted or unsubstituted C₃-C₁₀ cycloalkyl, a substituted or unsubstituted C₁-C₁₀ alkoxy, a substituted or unsubstituted C₆-C₃₀ aryloxy, a substituted or unsubstituted C₆-C₃₀ arylamino, a substituted or unsubstituted C₆-C₃₀ aryl, and a substituted or unsubstituted C₂-C₃₀ heteroaryl; and
a substituted group in the each substituted R₁, R₂, and R is one or a combination of at least two selected from deuterium, tritium, cyano, halogen, C₁-C₁₀ alkyl, C₃-C₁₀ cycloalkyl, silyl, C₆-C₃₀ arylamino, C₆-C₃₀ aryl, and C₂-C₃₀ heteroaryl, and the substituted group is independently connected to the connected aromatic ring or heteroaromatic ring to form a ring or not to form a ring.

2. The organic electroluminescent device according to claim 1, wherein the fluorescent dye has a structure represented by any one of the following (1-1), (1-2), (1-3), (1-4), (1-5), (1-6), (2-1), (2-2) or (2-3): 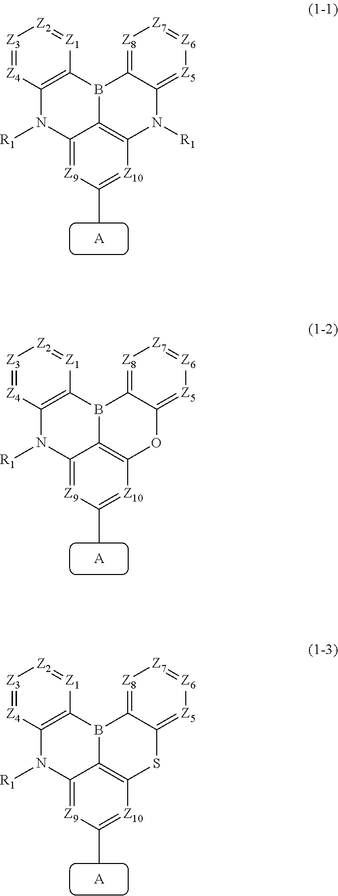 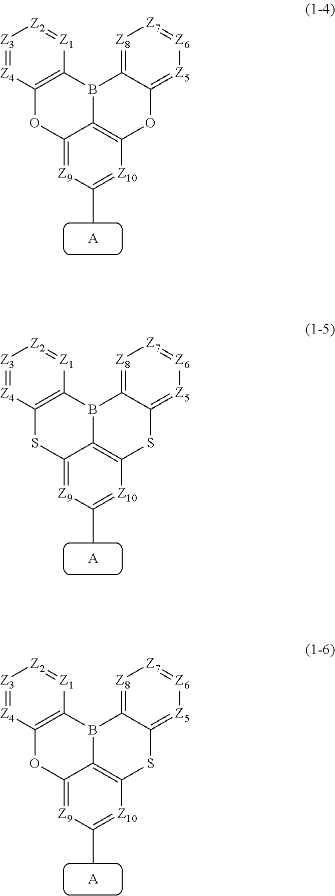 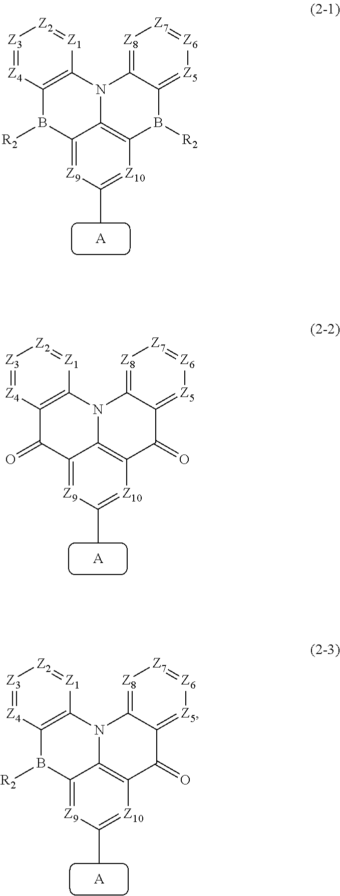
wherein, Z₁-Z₁₀ are each independently represented as CR.

3. The organic electroluminescent device according to claim 2, wherein R₁ is connected to adjacent R through a single bond, and R₂ is connected to adjacent R through a single bond.

4. The organic electroluminescent device according to claim 1, wherein A represents a substituted group represented by any one of the following (3-1), (3-2), (3-3), (3-4), (3-5), (3-6), (3-7), (3-8) or (3-9): 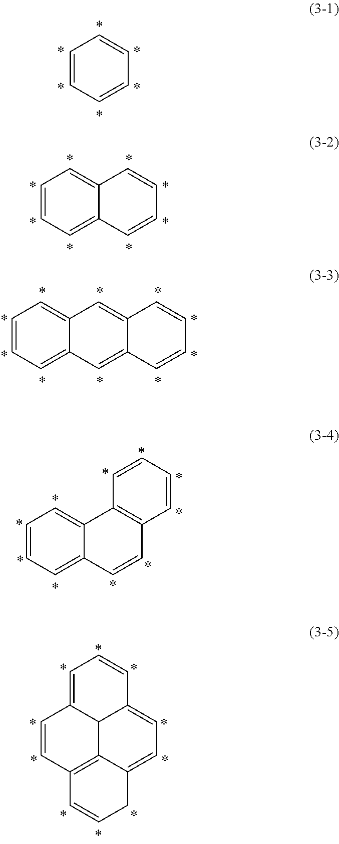 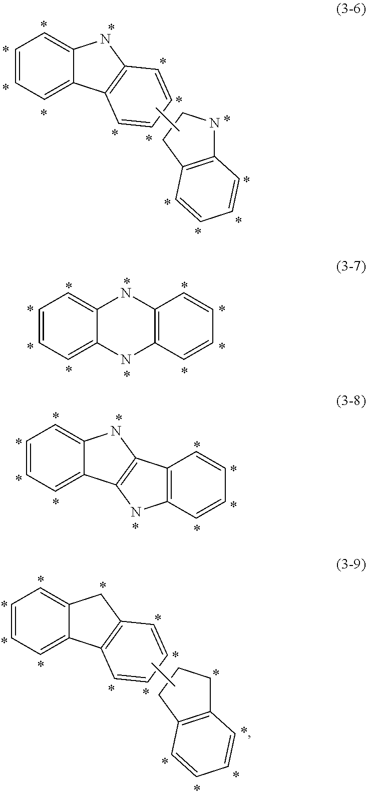
an asterisk in the Formulas denotes a connectable site, this connection denotes connection to a parent nucleus and/or connection with a substituted group; an expression of a ring structure streaked by “-” denotes any position on the ring structure where the connectable site can form a bond; a dashed line in the Formulas represents being connected or being unconnected;
wherein a substituted group in A is one or a combination of at least two selecting from deuterium, tritium, cyano, halogen, C₁-C₁₀ alkyl, C₃-C₁₀ cycloalkyl, C₆-C₃₀ arylamino, C₆-C₃₀ aryl, and C₂-C₃₀ heteroaryl, and the substituted group is independently connected to a connected aromatic ring or heteroaromatic ring to form a ring or not to form a ring.

5. The organic electroluminescent device according to claim 1, wherein A is represented by any one of the following structural formulas: 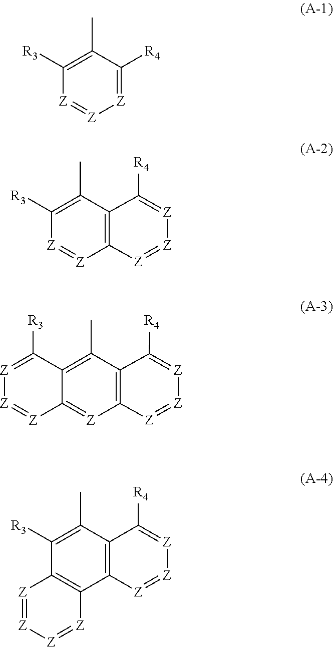 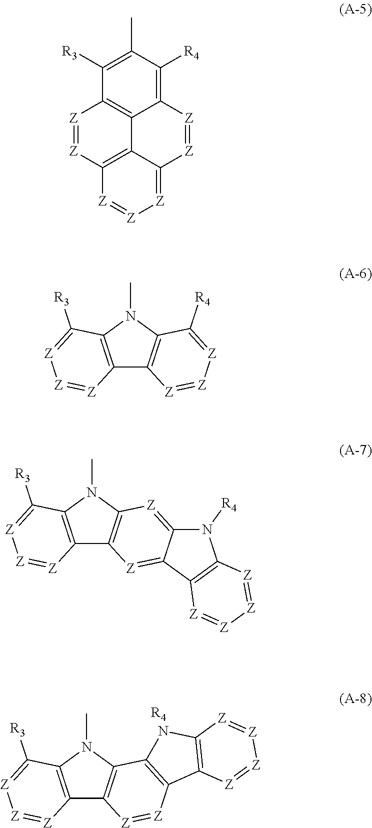 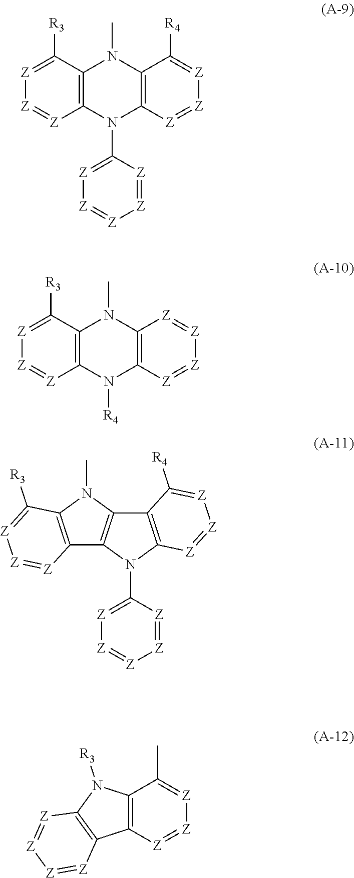
wherein, R₃ and R₄ are each independently represented as any one of hydrogen, deuterium, tritium, a substituted or unsubstituted C₁-C₃₀ alkyl, a substituted or unsubstituted C₃-C₃₀ cycloalkyl, a silyl, a substituted or unsubstituted C₁-C₃₀ alkoxy, a substituted or unsubstituted C₆-C₆₀ aryloxy, a substituted or unsubstituted C₆-C₆₀ arylamino, a substituted or unsubstituted C₆-C₆₀ aryl, and a substituted or unsubstituted C₂-C₆₀ heteroaryl;
Z is independently represented as N or CR₅, with R₅ being the same or different each time, and two adjacent R₅ are bond with each other to form a ring; R₅ is represented as one of hydrogen, deuterium, tritium, cyano, halogen, a substituted or unsubstituted C₁-C₁₀ alkyl, a substituted or unsubstituted C₃-C₁₀ cycloalkyl, a substituted or unsubstituted C₁-C₁₀ alkoxy, a substituted or unsubstituted C₆-C₃₀ aryloxy, a substituted or unsubstituted C₆-C₃₀ arylamino, a substituted or unsubstituted C₆-C₃₀ aryl, or a substituted or unsubstituted C₂-C₃₀ heteroaryl;
a substituted group in the each substituted R₃, R₄, and R₅ is one or a combination of at least two selected from deuterium, tritium, cyano, halogen, C₁-C₁₀ alkyl, C₃-C₁₀ cycloalkyl, silyl, C₆-C₃₀ arylamino, C₆-C₃₀ aryl, and C₂-C₃₀ heteroaryl, which is independently connected to the connected aromatic ring or heteroaromatic ring to form a ring or not to form a ring.

6. The organic electroluminescent device according to claim 5, wherein the R₃ and R₄ are each independently represented as any one of a substituted or unsubstituted C₁-C₃₀ alkyl, a substituted or unsubstituted C₃-C₃₀ cycloalkyl, a substituted or unsubstituted C₆-C₆₀ aryl, and a substituted or unsubstituted C₂-C₆₀ heteroaryl.

7. The organic electroluminescent device according to claim 6, wherein at least one of the R₃ and R₄ is selected from one of the following groups with large steric effect: terphenyl, trimericphenyl, tetraphenyl, fluorenyl, spirodifluorenyl, dihydrophenanthryl, dihydropyrenyl, tetrahydropyrenyl, cis or trans indenofluorenyl, trimericindenyl, isotrimericindenyl, spiro-trimericindenyl, spiro-isotrimericindenyl, furyl, benzofuryl, isobenzofuryl, dibenzofuryl, benzothienyl, isobenzothienyl, dibenzothienyl, isoindolyl, carbazolyl, indenocarbazolyl, isoquinolyl, acridinyl, phenanthridinyl, benzo-5,6-quinolyl, benzo-6,7-quinolyl, benzo-7,8-quinolyl, benzimidazolyl, naphthoimidazolyl, phenanthroimidazolyl, pyridino imidazolyl, pyrazino imidazolyl, quinoxalino imidazolyl, oxazolyl, benzo-oxazolyl, naphtho-oxazolyl, anthro-oxazolyl, phenanthro-oxazolyl, 1,2-thiazolyl, 1,3-thiazolyl, benzothiazolyl, pyridazinyl, benzopyridazinyl, 1,5-diaza anthryl, 2,7-diaza pyrenyl, 2,3-diaza pyrenyl, 1,6-diaza pyrenyl, 1,8-diaza pyrenyl, 4,5-diaza pyrenyl, 4,5,9,10-tetraaza perylenyl, phenazinyl, phenothiazinyl, azacarbazolyl, benzocarbolinyl, phenanthrolinyl, 1,2,3-triazolyl, 1,2,4-triazolyl, benzotriazolyl, 1,2,3-oxadiazolyl, 1,2,4-oxadiazolyl, 1,2,5-oxadiazolyl, 1,2,3-thiadiazolyl, 1,2,4-thiadiazolyl, 1,2,5-thiadiazolyl, 1,3,4-thiadiazolyl, 1,3,5-triazinyl, 1,2,4-triazinyl, 1,2,3-triazinyl, tetrazolyl, 1,2,4,5-tetrazinyl, 1,2,3,4-tetrazinyl, 1,2,3,5-tetrazinyl, purinyl, pteridyl, indozinyl, benzothiadiazolyl, 9,9-dimethylacridinyl, diphenylamindo, adamantyl, fluorophenyl, methylphenyl, trimethylphenyl, cyanophenyl, silyl; or at least one of R₃ or R₄ is selected from a combination of two or more from the aforementioned groups with large steric effect.

8. The organic electroluminescent device according to claim 1, wherein the R denotes one of hydrogen, deuterium, tritium, fluorine atom, cyano, methyl, deuterated methyl, tritiated methyl, ethyl, deuterated ethyl, tritiated ethyl, isopropyl, deuterated isopropyl, tritiated isopropyl, tert-butyl, deuterated tert-buty, tritiated tert-butyl, deuterated cyclopentyl, tritiated cyclopentyl, cyclohexyl, cyclopentyl, adamantyl, phenyl, deuterated phenyl, tritiated phenyl, biphenyl, deuterated biphenyl, tritiated biphenyl, deuterated terphenyl, tritiated terphenyl, terphenyl, naphthyl, anthryl, phenanthryl, pyridyl, quinolyl, furyl, thienyl, dibenzofuryl, dibenzothienyl, carbazolyl, N-phenylcarbazolyl, 9,9-dimethylfluorenyl, 9,9-diphenylfluorenyl, spirofluorenyl, methyl-substituted phenyl, ethyl-substituted phenyl, isopropyl-substituted phenyl, tert-butyl-substituted phenyl, methyl-substituted biphenyl, ethyl-substituted biphenyl, isopropyl-substituted biphenyl, tert-butyl-substituted biphenyl, deuterated methyl-substituted phenyl, deuterated ethyl-substituted phenyl, deuterated isopropyl-substituted phenyl, deuterated tert-butyl-substituted phenyl, deuterated methyl-substituted biphenyl, deuterated ethyl-substituted biphenyl, deuterated isopropyl-substituted biphenyl, deuterated tert-butyl-substituted diphenyl, tritiated methyl-substituted phenyl, tritiated ethyl-substituted phenyl, tritiated isopropyl-substituted phenyl, tritiated tert-butyl-substituted phenyl, tritiated methyl-substituted diphenyl, tritiated ethyl-substituted diphenyl, tritiated isopropyl-substituted diphenyl, tritiated tert-butyl-substituted diphenyl, diphenylamido, di-biphenylamido, and triphenylamino.

9. The organic electroluminescent device according to claim 1, wherein R₁ represents one of methyl, deuterated methyl, tritiated methyl, ethyl, deuterated ethyl, tritiated ethyl, isopropyl, deuterated isopropyl, tritiated isopropyl, tert-butyl, deuterated tert-butyl, tritiated tert-butyl, deuterated cyclopentyl, tritiated cyclopentyl, cyclopentyl, adamantyl, phenyl, deuterated phenyl, tritiated phenyl, biphenyl, deuterated biphenyl, tritiated biphenyl, deuterated terphenyl, tritiated terphenyl, terphenyl, naphthyl, anthryl, phenanthryl, pyridyl, quinolyl, furyl, thienyl, dibenzofuryl, dibenzothienyl, carbazolyl, N-phenylcarbazolyl, 9,9-dimethylfluorenyl, 9,9-diphenylfluorenyl, spirofluorenyl, methyl-substituted phenyl, ethyl-substituted phenyl, isopropyl-substituted phenyl, tert-butyl-substituted phenyl, methyl-substituted diphenyl, ethyl-substituted diphenyl, isopropyl-substituted diphenyl, tert-butyl-substituted diphenyl, deuterated methyl-substituted phenyl, deuterated ethyl-substituted phenyl, deuterated isopropyl-substituted phenyl, deuterated tert-butyl-substituted phenyl, deuterated methyl-substituted diphenyl, deuterated ethyl-substituted diphenyl, deuterated isopropyl-substituted diphenyl, deuterated tert-butyl-substituted diphenyl, tritiated methyl-substituted phenyl, tritiated ethyl-substituted phenyl, tritiated isopropyl-substituted phenyl, tritiated tert-butyl-substituted phenyl, tritiated methyl-substituted diphenyl, tritiated ethyl-substituted diphenyl, tritiated isopropyl-substituted diphenyl, and tritiated tert-butyl-substituted diphenyl.

10. The organic electroluminescent device according to claim 1, wherein the R₂ represents one of phenyl, deuterated phenyl, tritiated phenyl, biphenyl, deuterated biphenyl, tritiated biphenyl, deuterated terphenyl, tritiated terphenyl, terphenyl, naphthyl, anthryl, phenanthryl, pyridyl, quinolyl, dibenzofuryl, dibenzothienyl, N-phenylcarbazolyl, methyl-substituted phenyl, amidine, ethyl-substituted phenyl, isopropyl-substituted phenyl, tert-butyl-substituted phenyl, methyl-substituted biphenyl, ethyl-substituted biphenyl, isopropyl-substituted biphenyl, tert-butyl-substituted biphenyl, deuterated methyl-substituted phenyl, deuterated ethyl-substituted phenyl, deuterated isopropyl-substituted phenyl, deuterated tert-butyl-substituted phenyl, deuterated methyl-substituted biphenyl, deuterated ethyl-substituted biphenyl, deuterated isopropyl-substituted biphenyl, deuterated tert-butyl-substituted biphenyl, tritiated methyl-substituted phenyl, tritiated ethyl-substituted phenyl, tritiated isopropyl-substituted phenyl, tritiated tert-butyl-substituted phenyl, tritiated methyl-substituted diphenyl, tritiated ethyl-substituted diphenyl, tritiated isopropyl-substituted diphenyl, and tritiated tert-butyl-substituted diphenyl.

11. The organic electroluminescent device according to claim 8, wherein in Formula (1) or Formula (2), the Z₉ and Z₁₀ are each CH.

12. The organic electroluminescent device according to claim 8, wherein in Formula (1) or Formula (2), the Z₂ and Z₇ are each CC(CH₃)₃, and Z₁, Z₃-Z₆, and Z₈-Z₁₀ are each CH.

13. The organic electroluminescent device according to claim 1, wherein the fluorescent dye is selected from a compound represented by the following specific structural formulas:

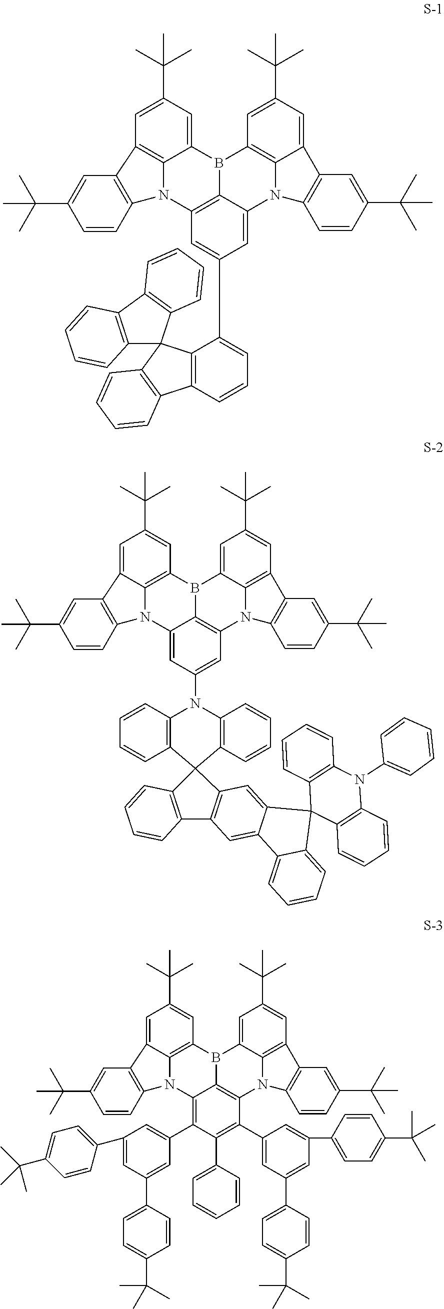

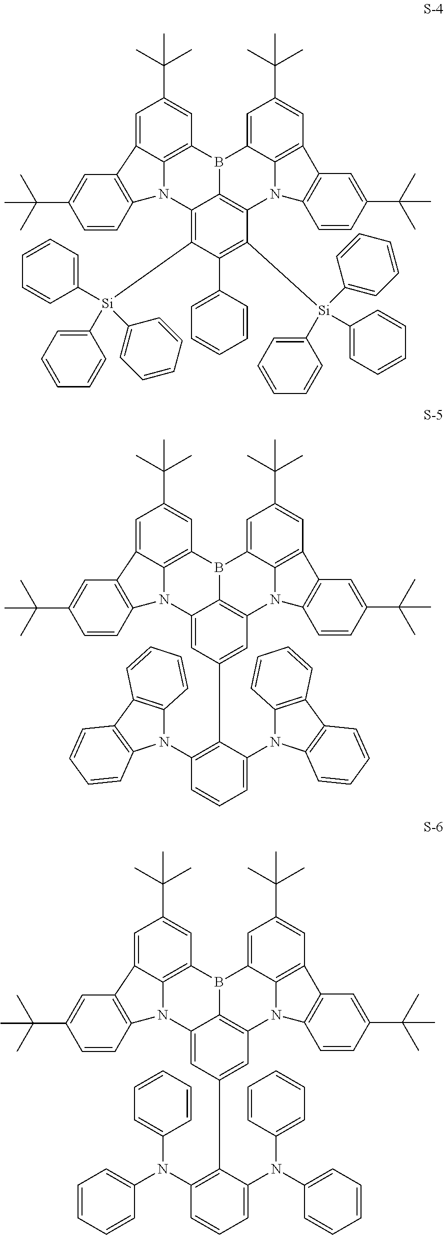

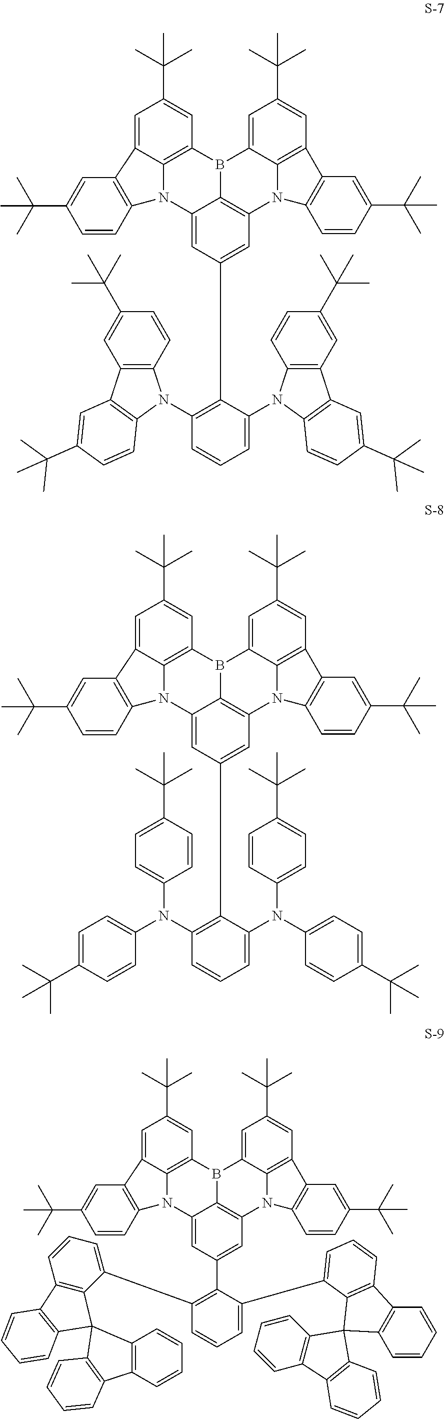

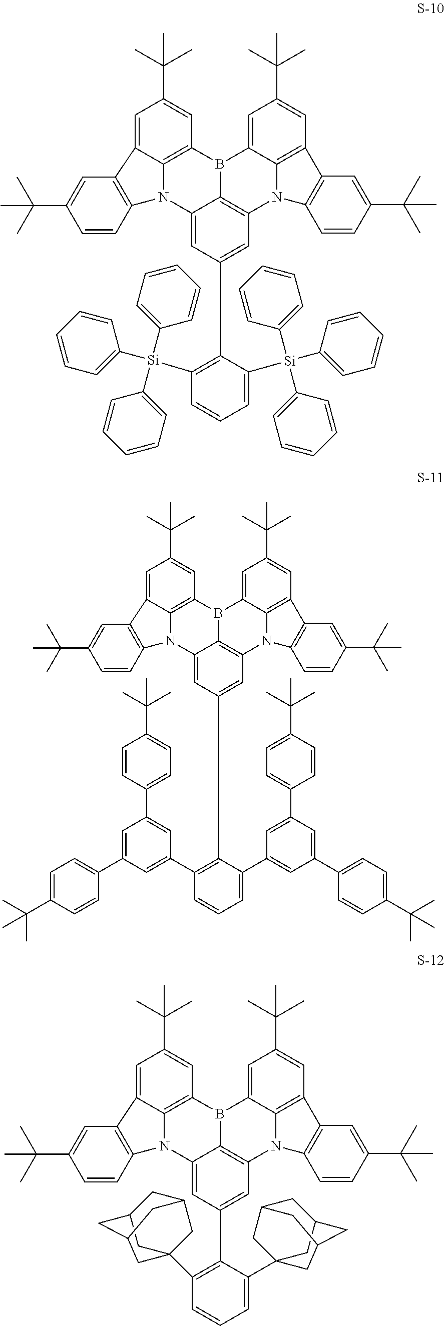

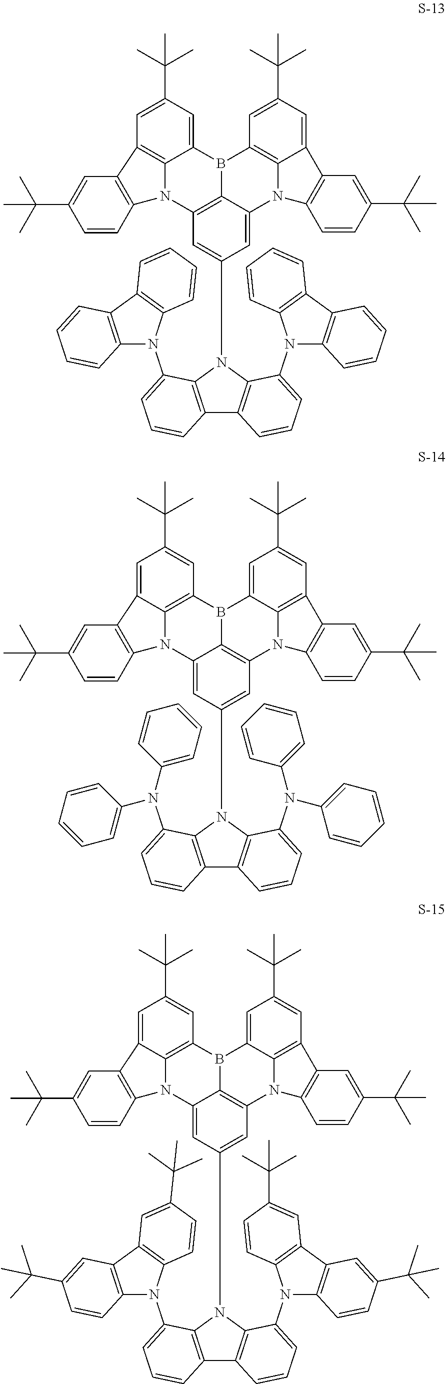

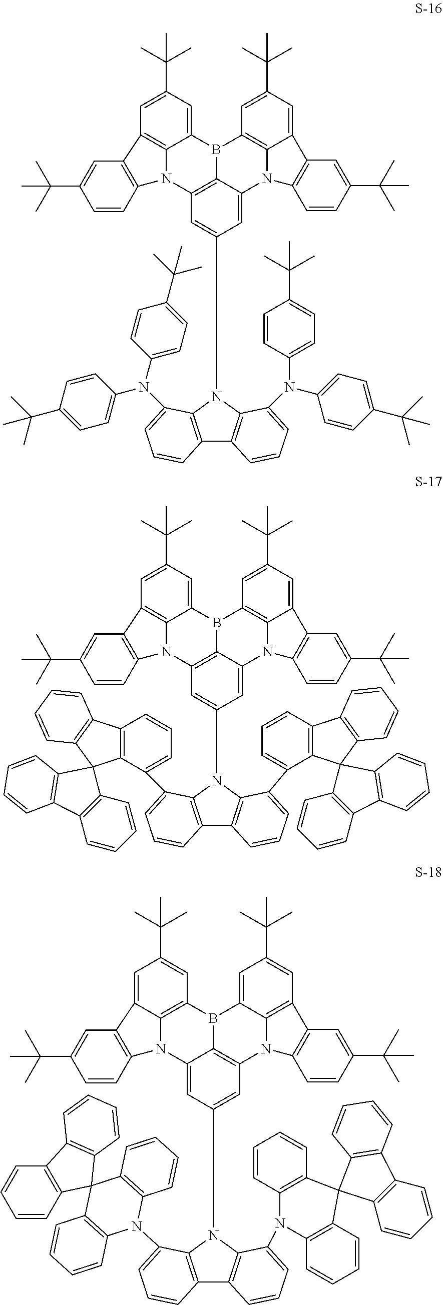

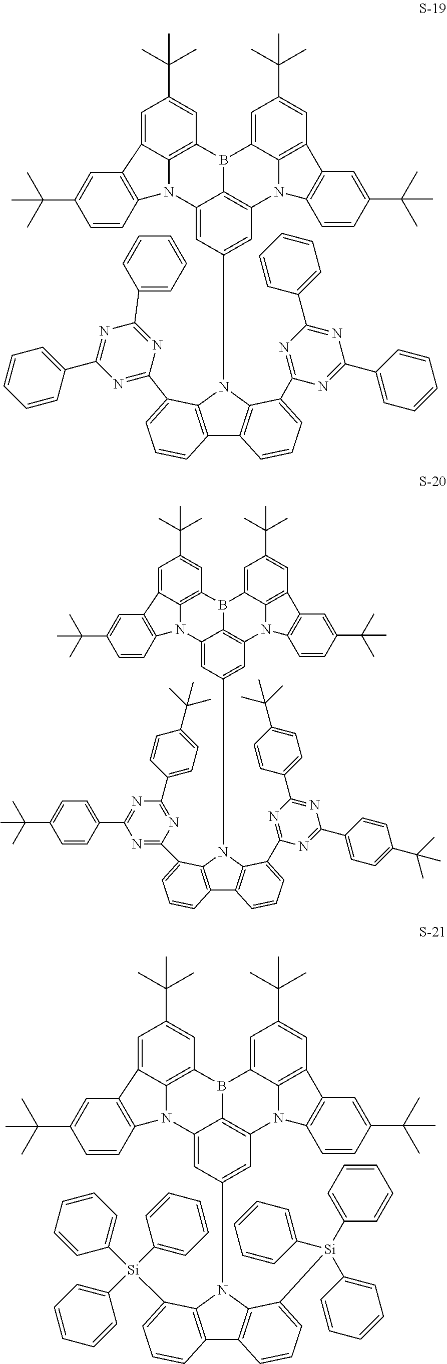

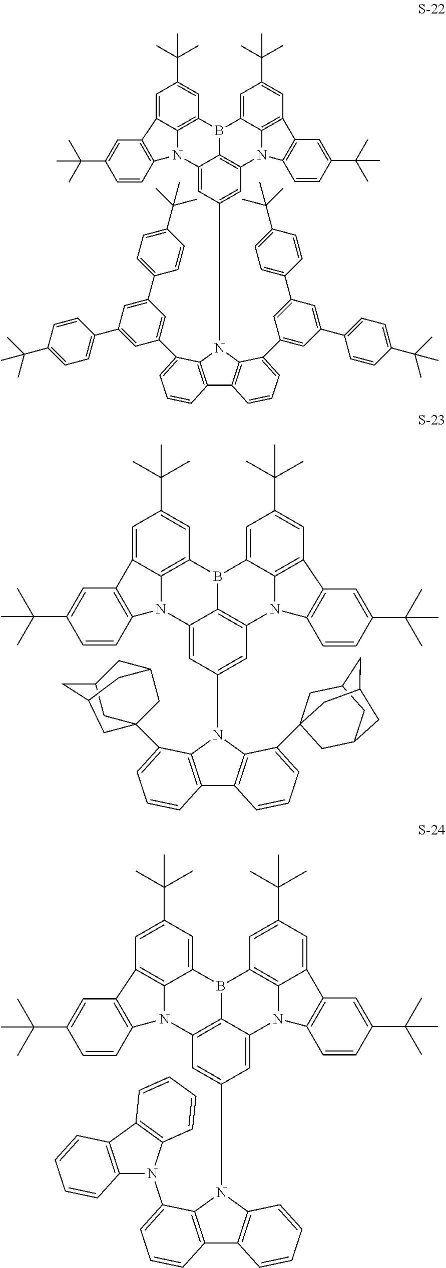

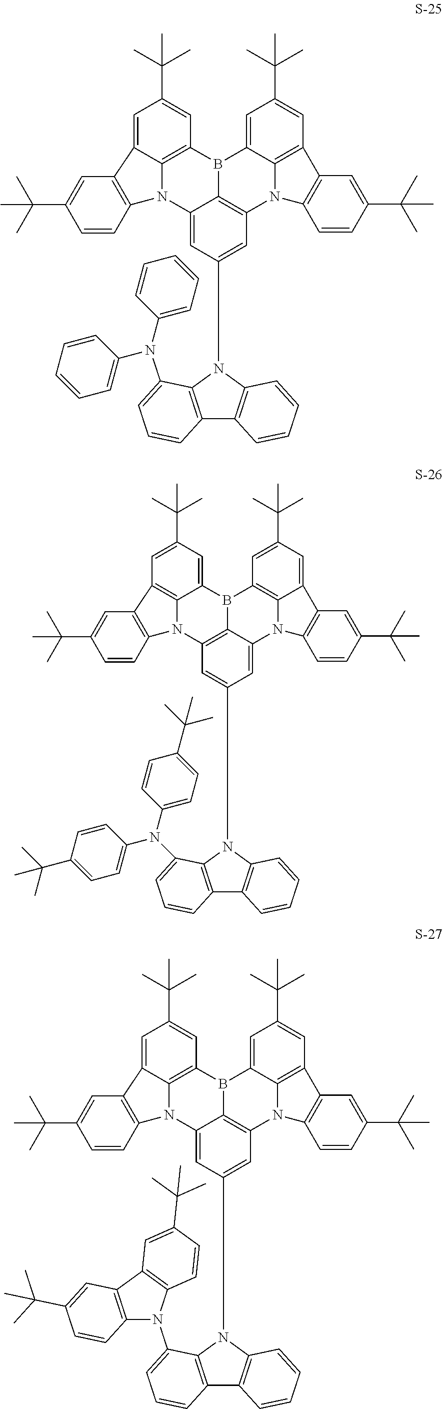

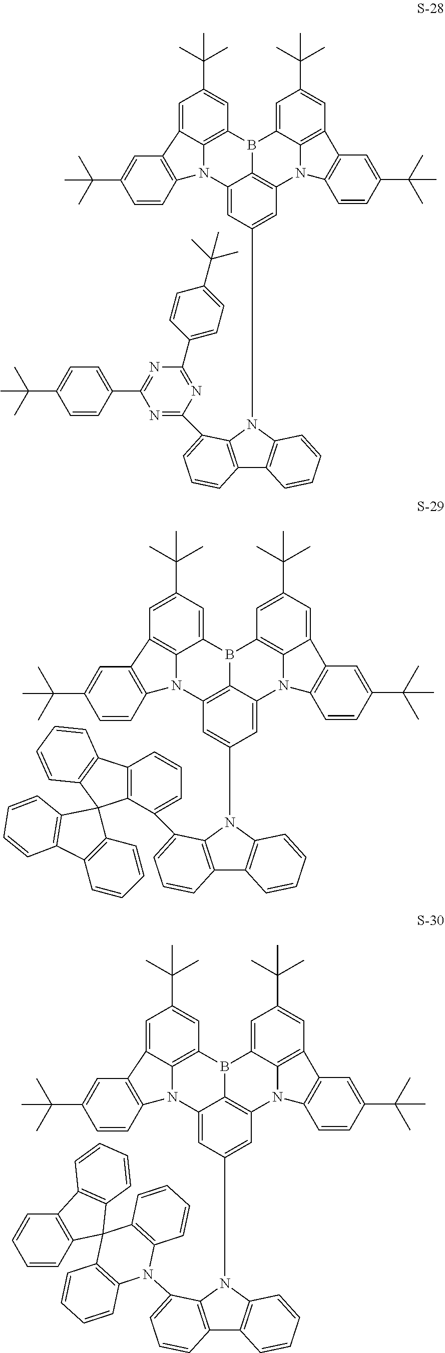 [SMILES: CC(C)(C)c1ccc(-c2nc(-c3ccc(C(C)(C)C)cc3)nc(-c3cccc4c5ccccc5n(-c5cc6c7c(c5)-n5c8ccc(C(C)(C)C)cc8c8cc(C(C)(C)C)cc(c85)B7c5cc(C(C)(C)C)cc7c8cc(C(C)(C)C)ccc8n-6c57)c34)n2)cc1 ; CC(C)(C)c1ccc2c(c1)c1cc(C(C)(C)C)cc3c1n2-c1cc(-n2c4ccccc4c4cccc(-c5cccc6c5C5(c7ccccc7-c7ccccc75)c5ccccc5-6)c42)cc2c1B3c1cc(C(C)(C)C)cc3c4cc(C(C)(C)C)ccc4n-2c13 ; CC(C)(C)c1ccc2c(c1)c1cc(C(C)(C)C)cc3c1n2-c1cc(-n2c4ccccc4c4cccc(N5c6ccccc6C6(c7ccccc7-c7ccccc76)c6ccccc65)c42)cc2c1B3c1cc(C(C)(C)C)cc3c4cc(C(C)(C)C)ccc4n-2c13]

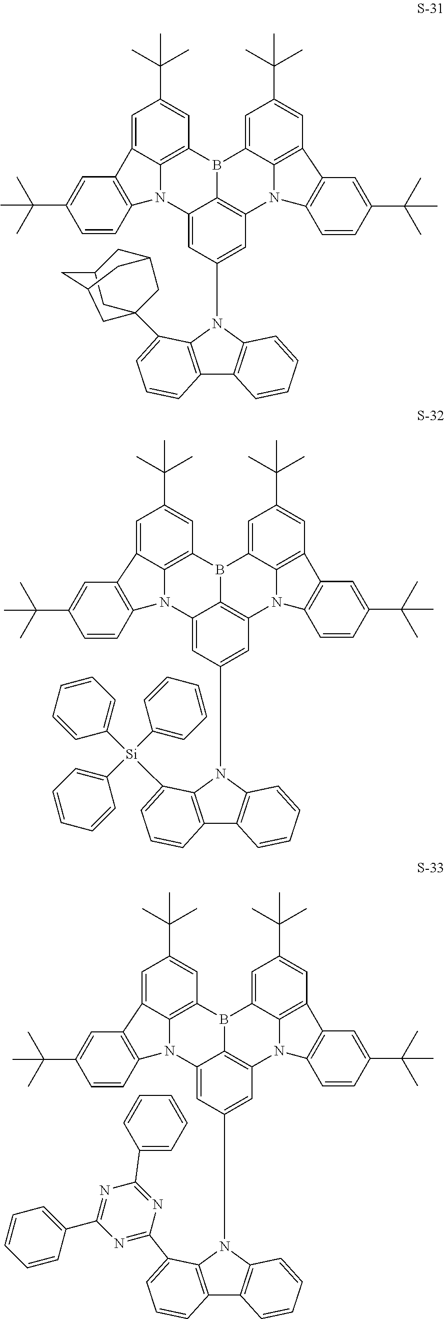

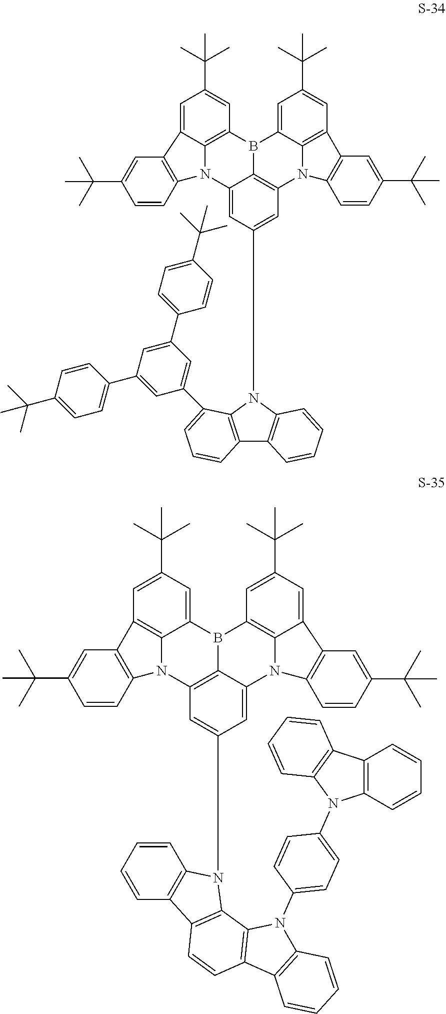

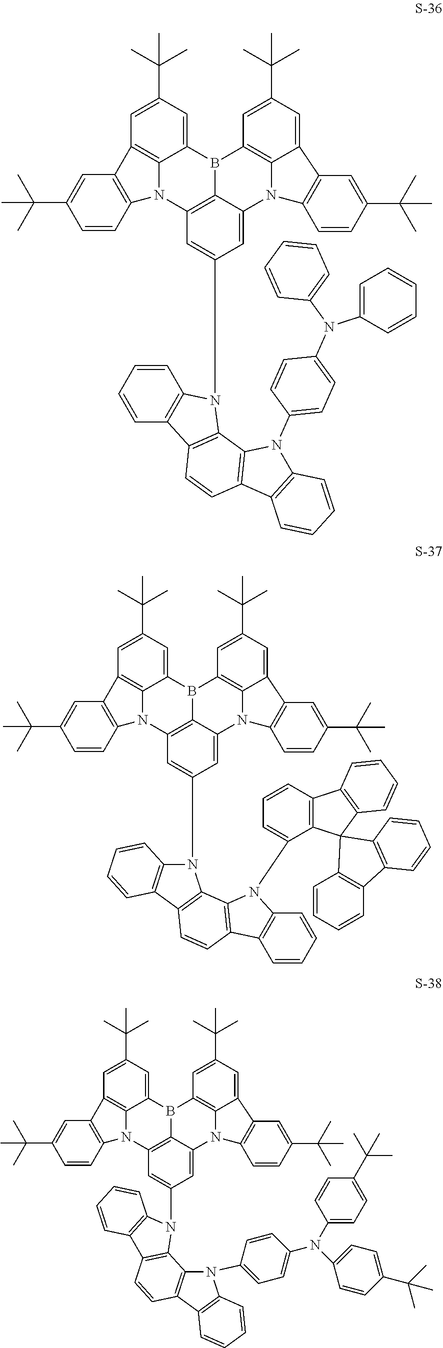

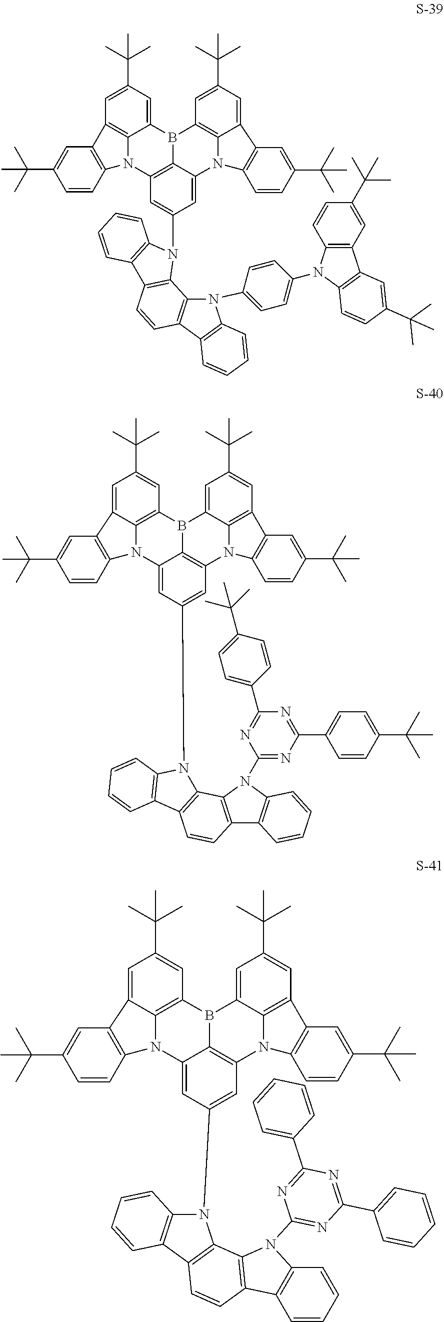

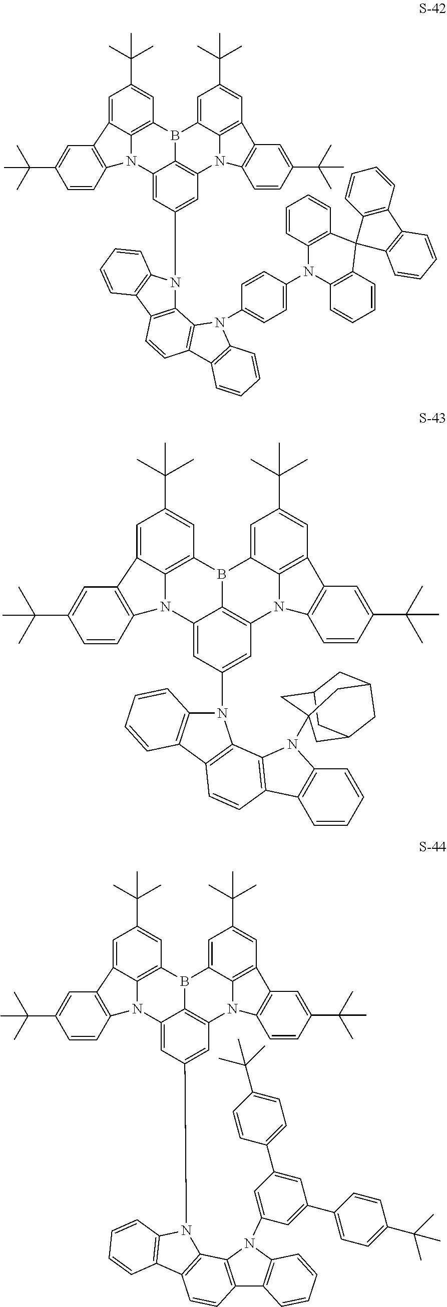

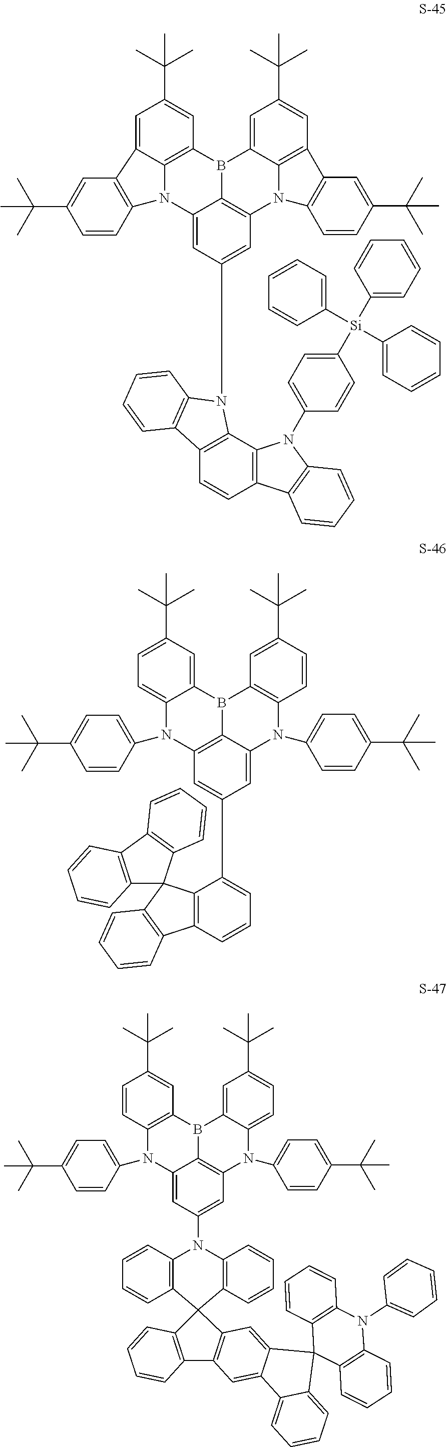

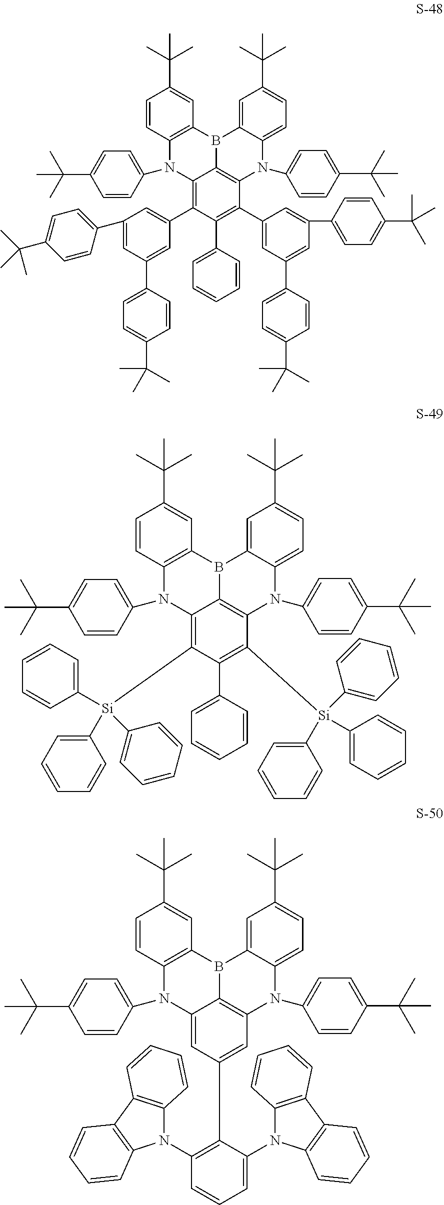

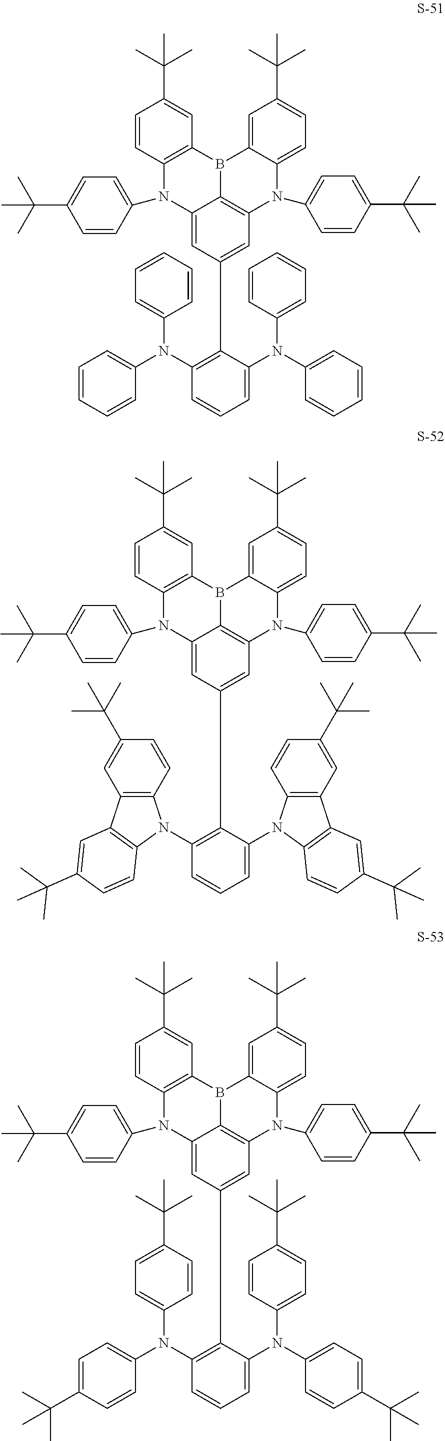

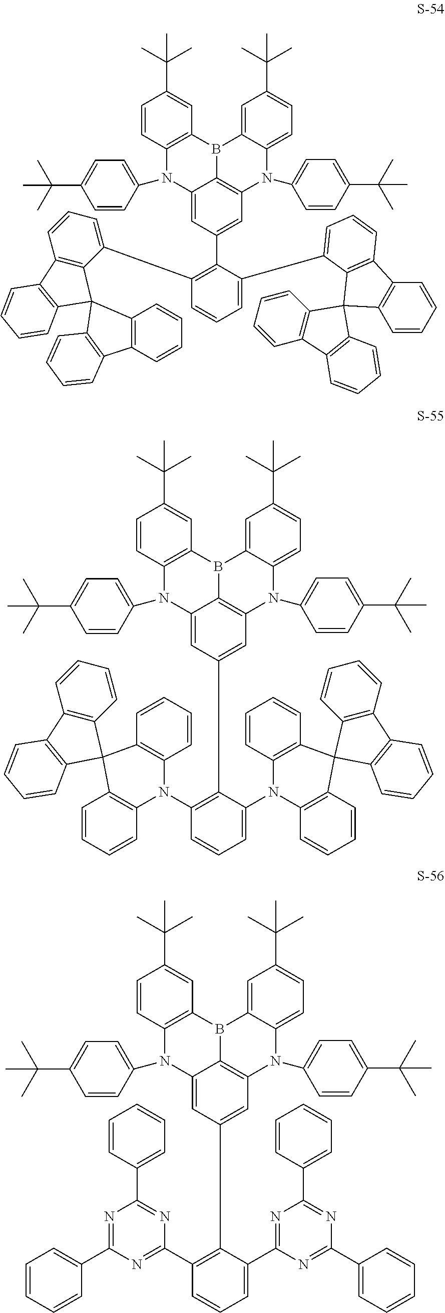

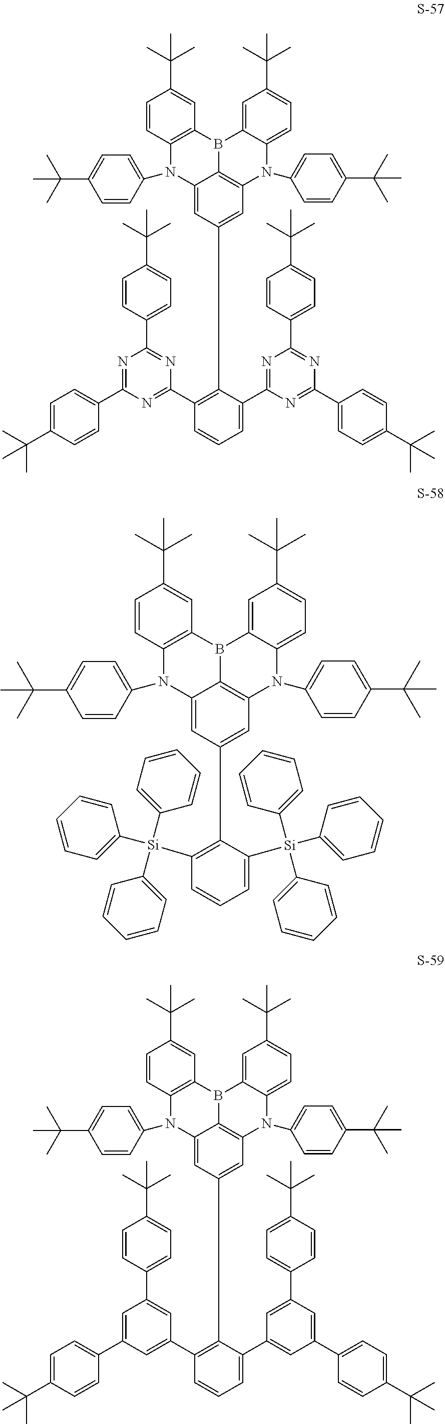

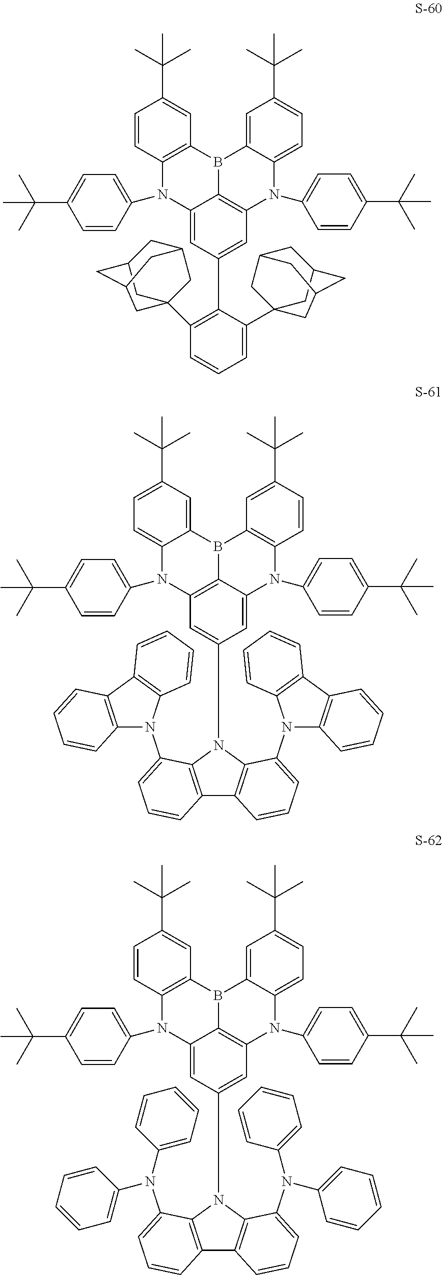

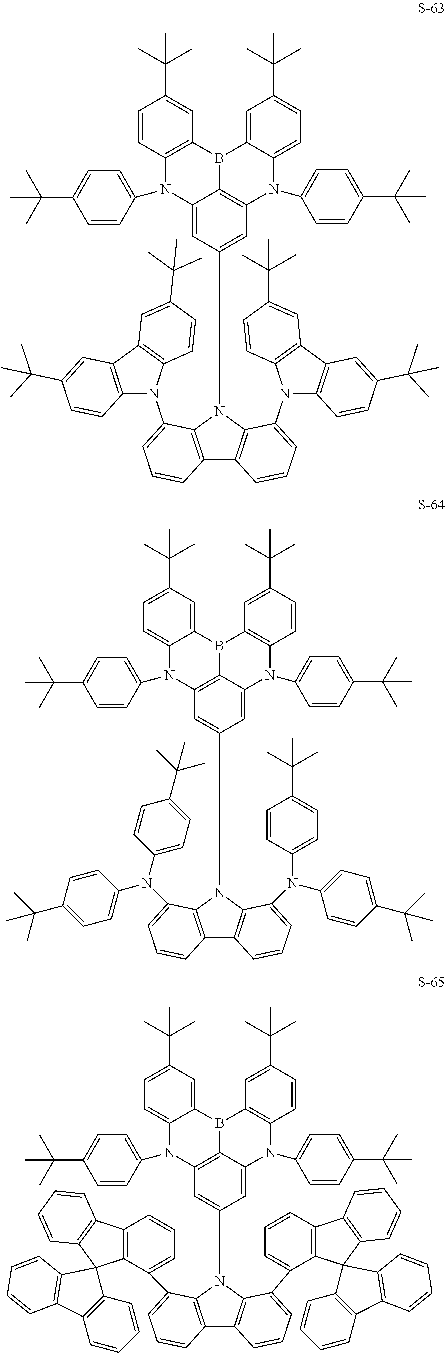

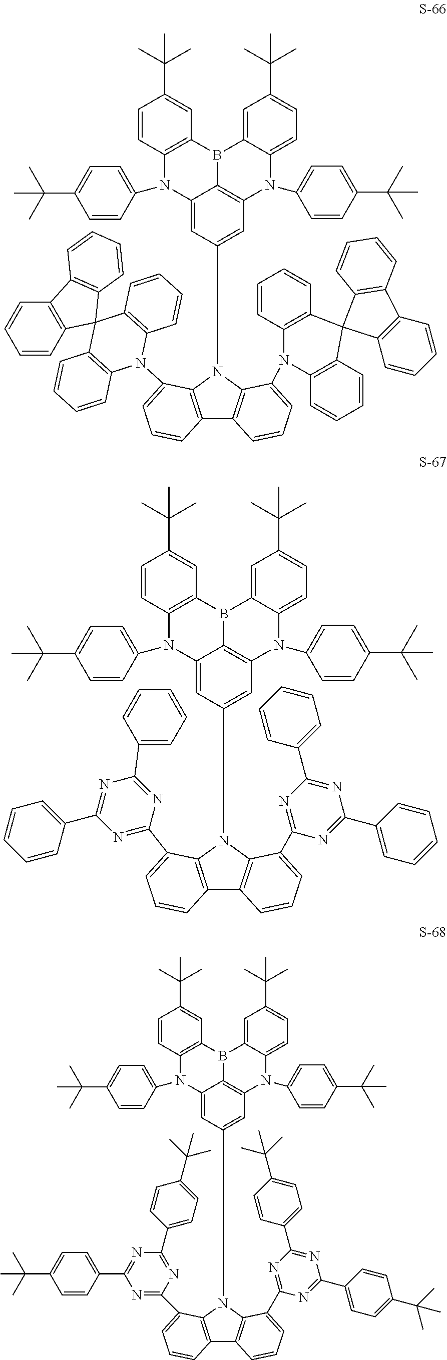

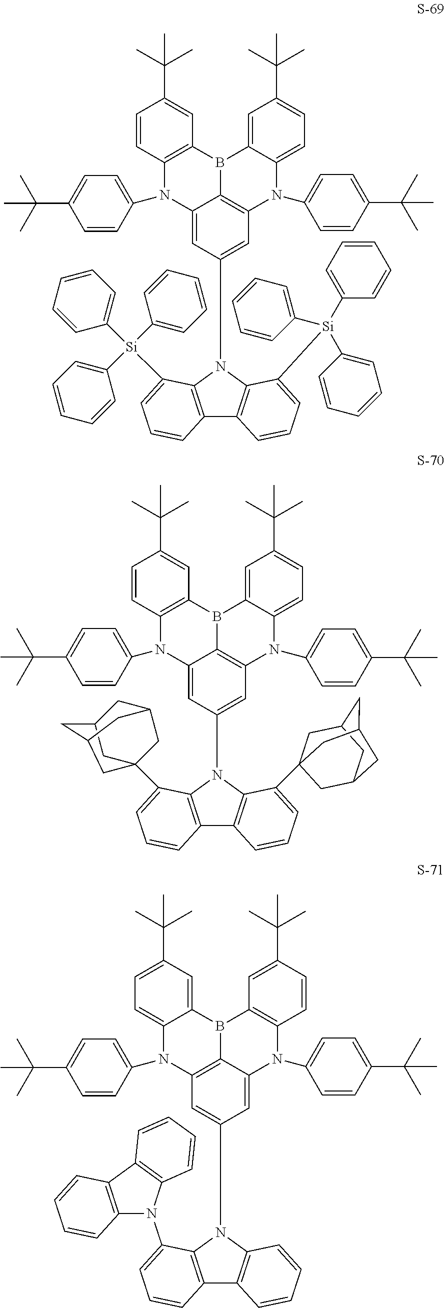

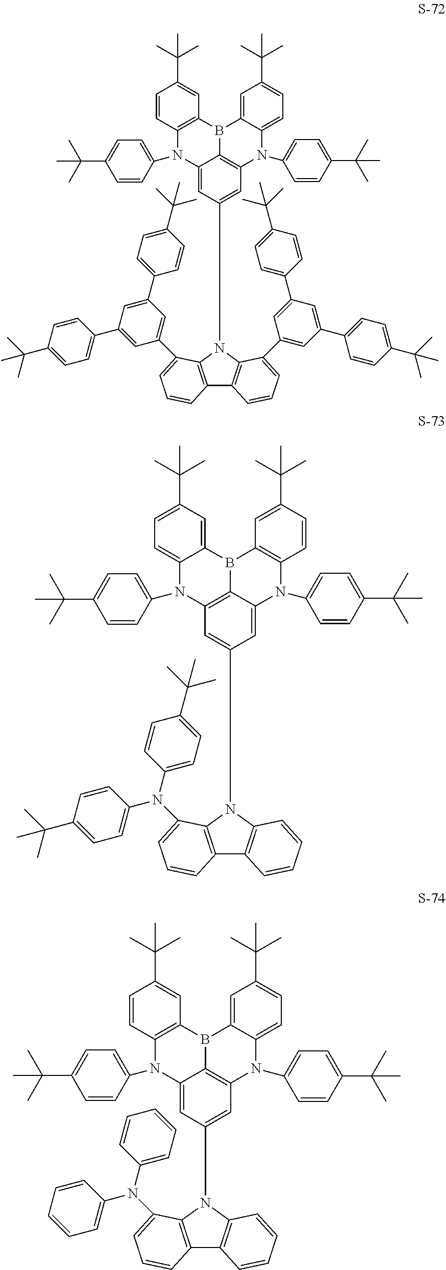

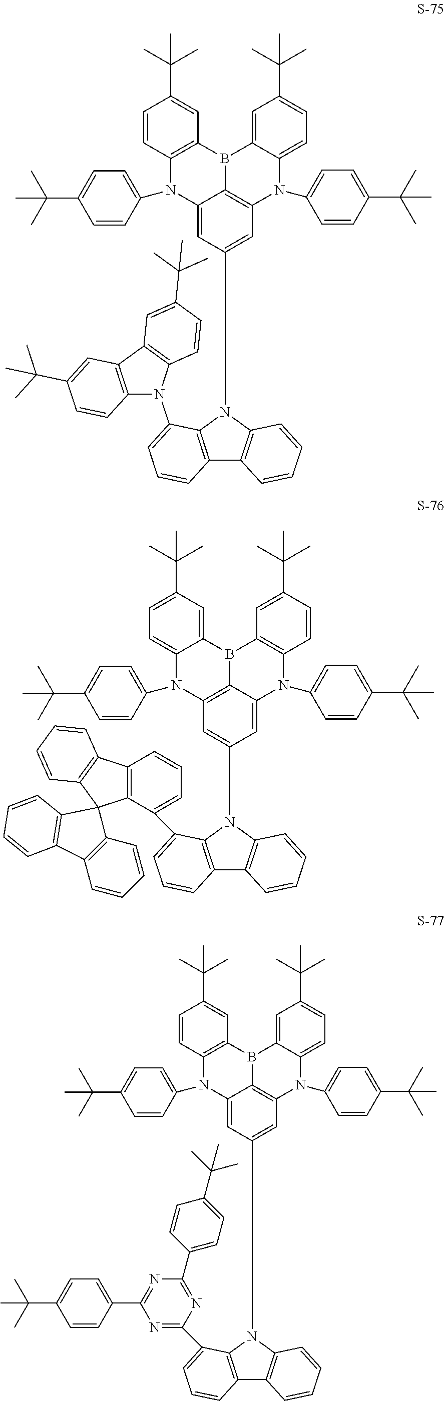

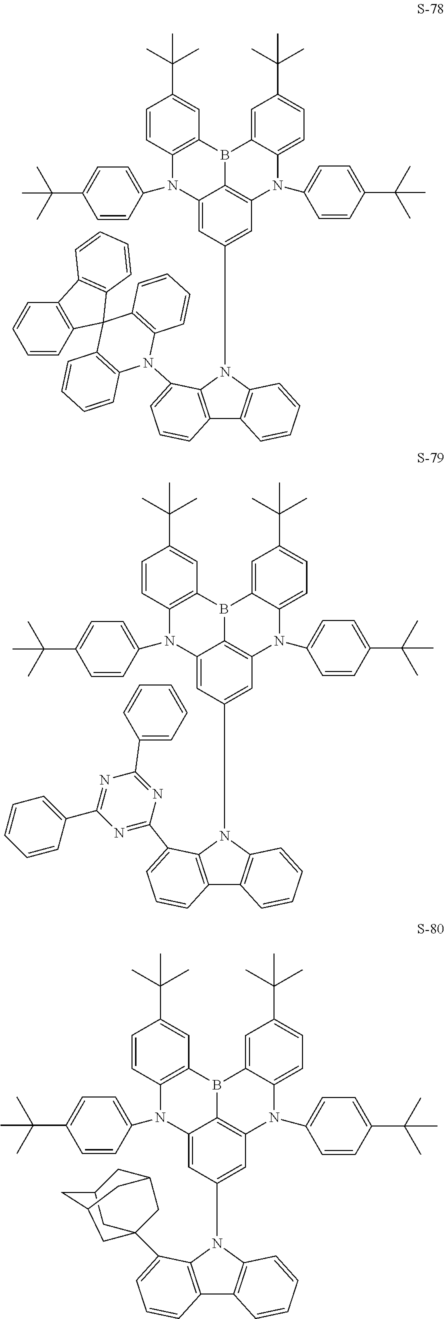

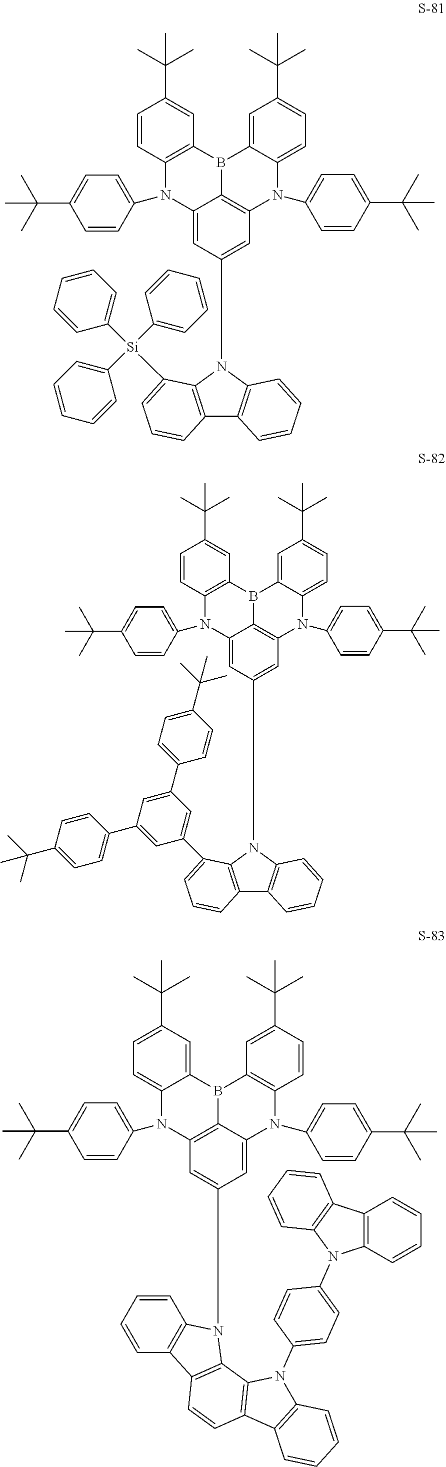

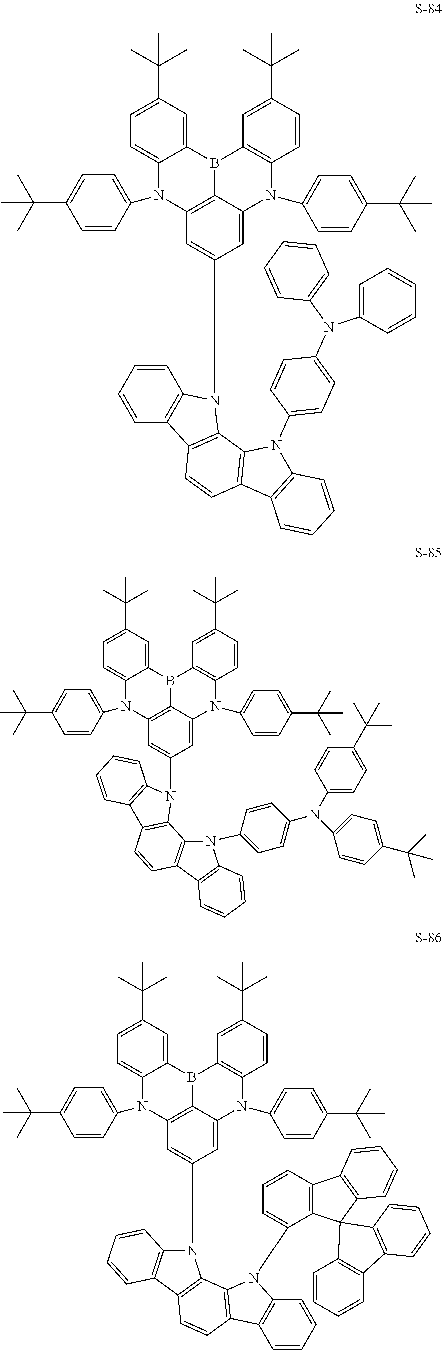

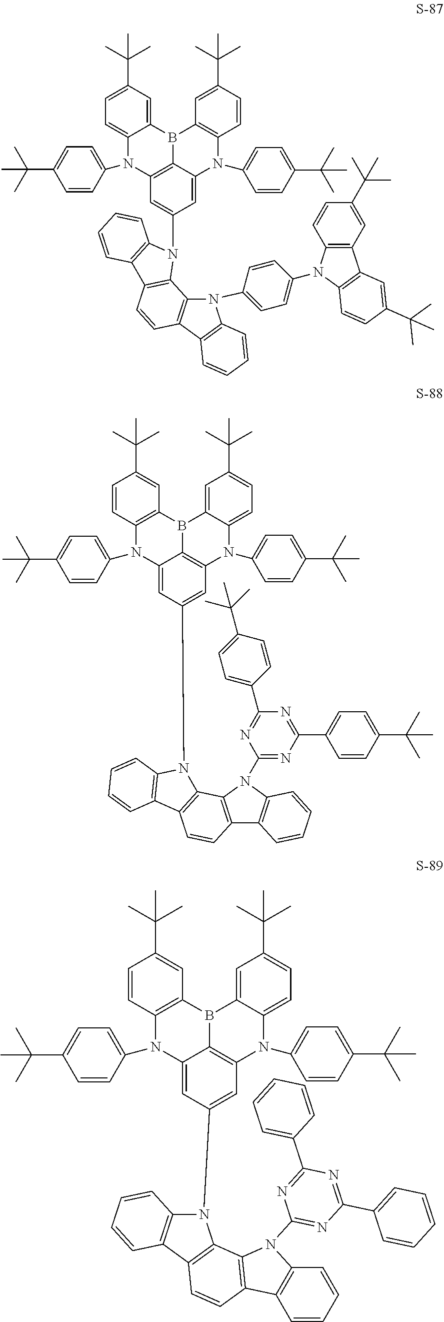

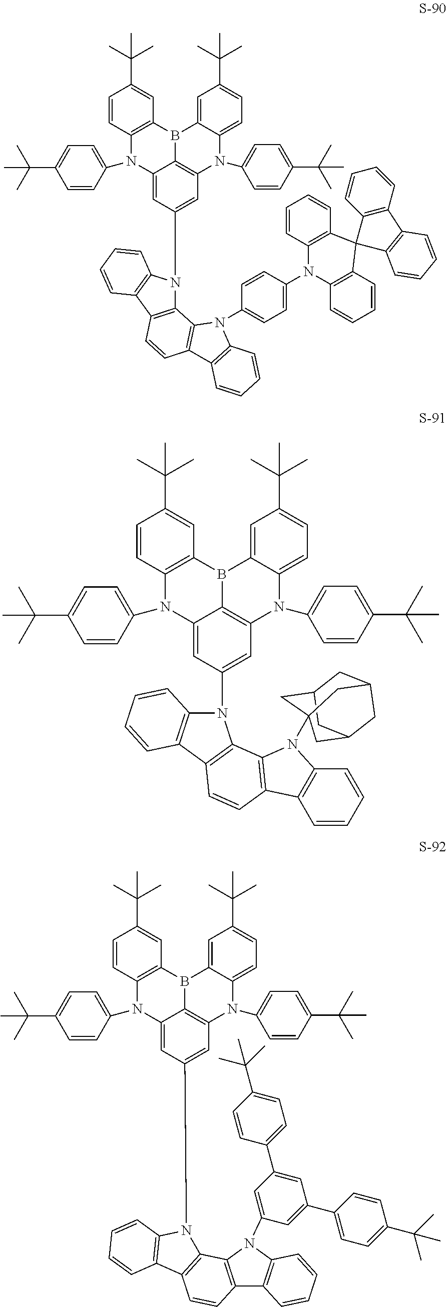

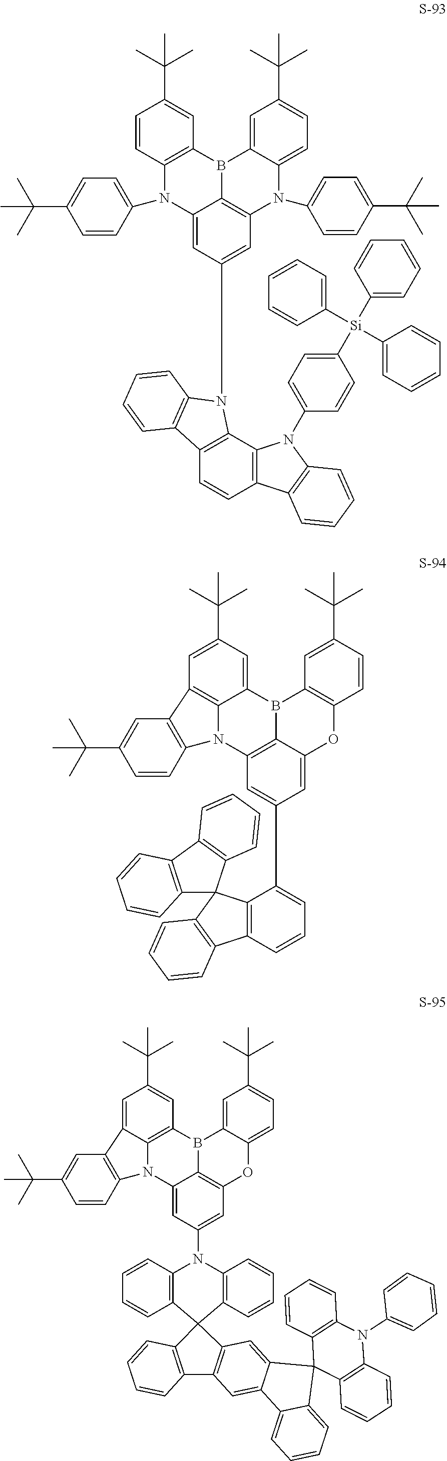

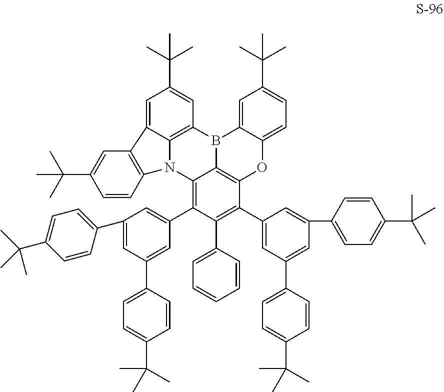 [SMILES: CC(C)(C)c1ccc(-c2cc(-c3ccc(C(C)(C)C)cc3)cc(-c3c4c5c(c(-c6cc(-c7ccc(C(C)(C)C)cc7)cc(-c7ccc(C(C)(C)C)cc7)c6)c3-c3ccccc3)-n3c6ccc(C(C)(C)C)cc6c6cc(C(C)(C)C)cc(c63)B5c3cc(C(C)(C)C)ccc3O4)c2)cc1]

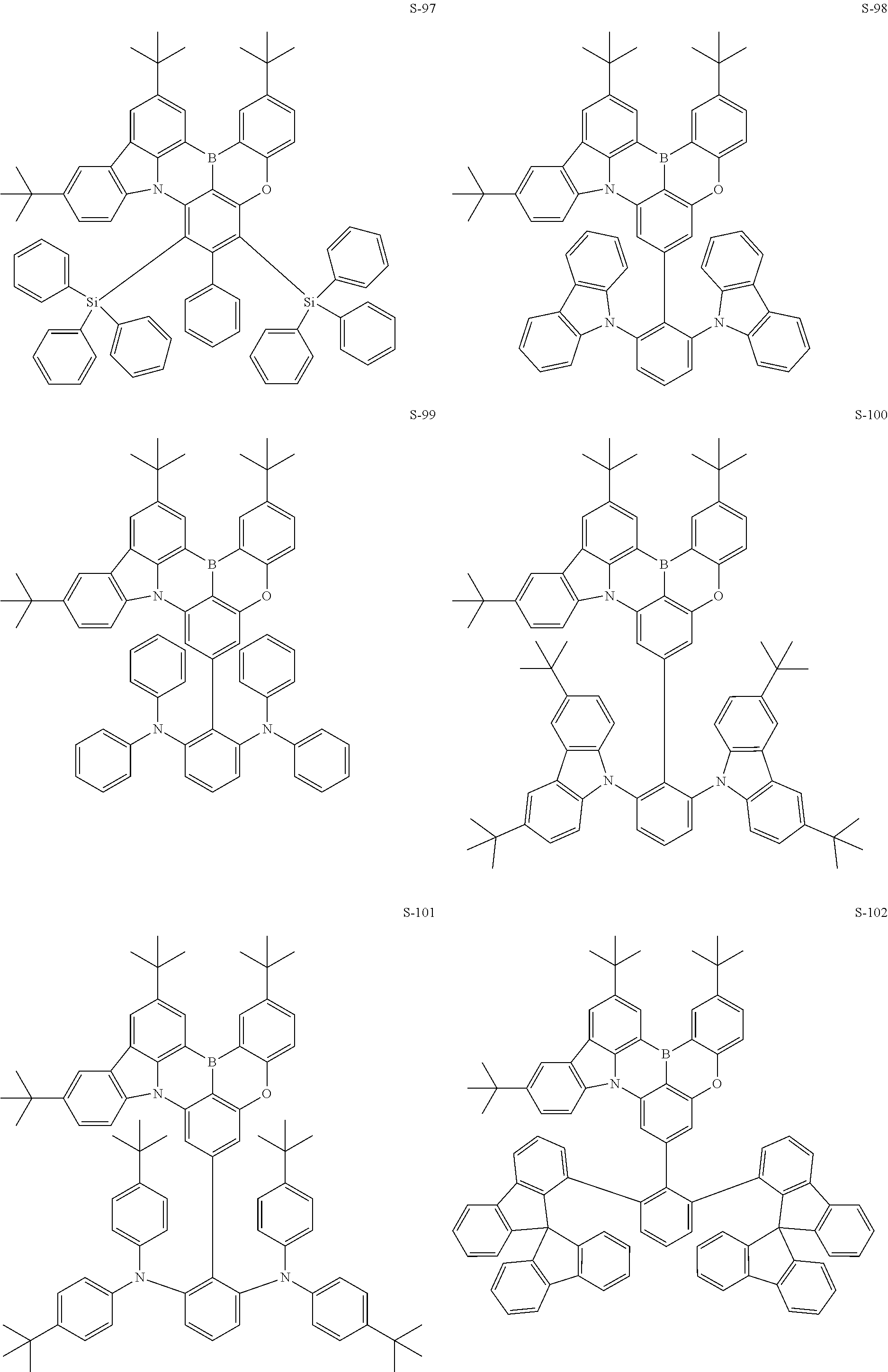

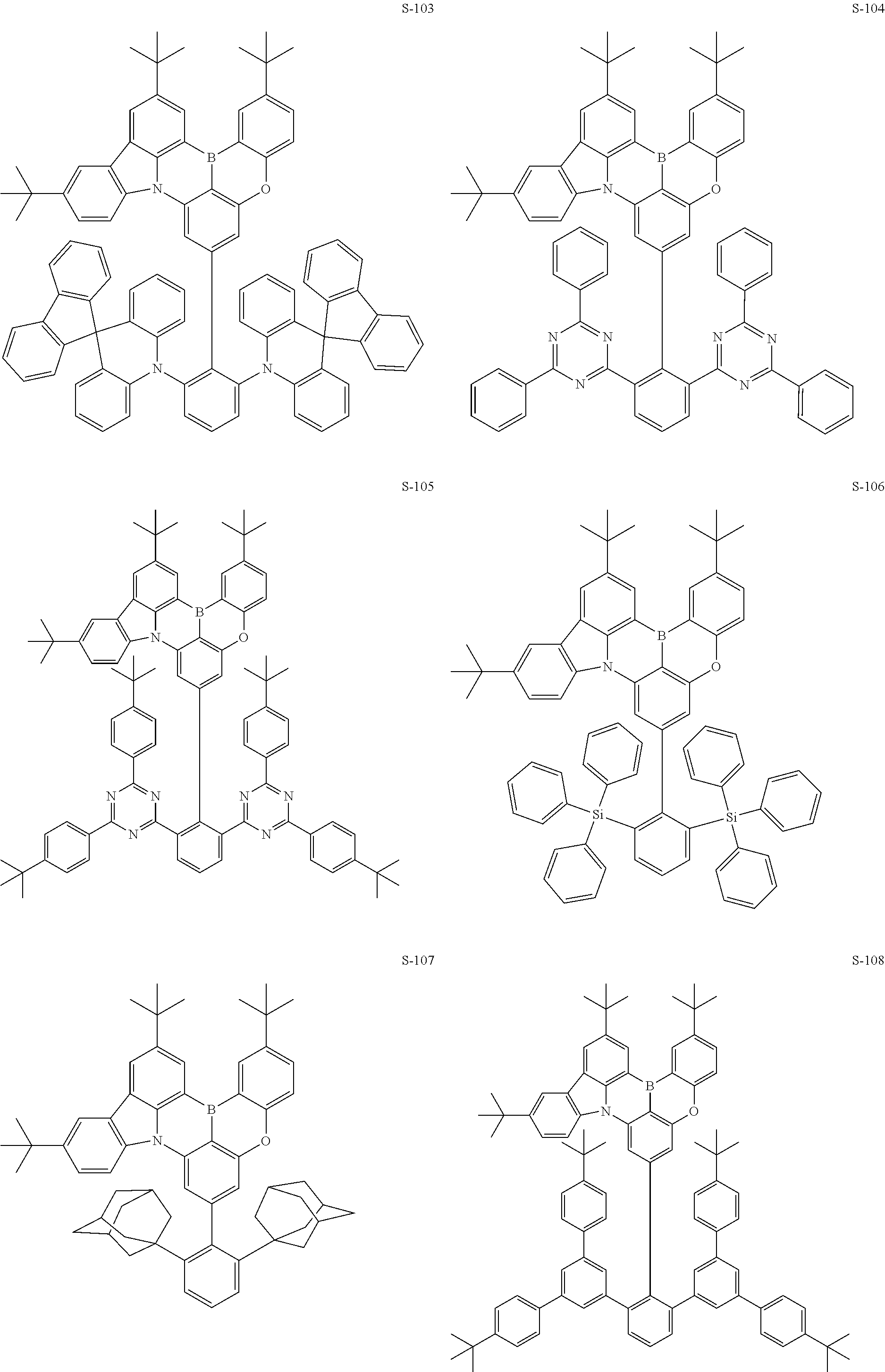

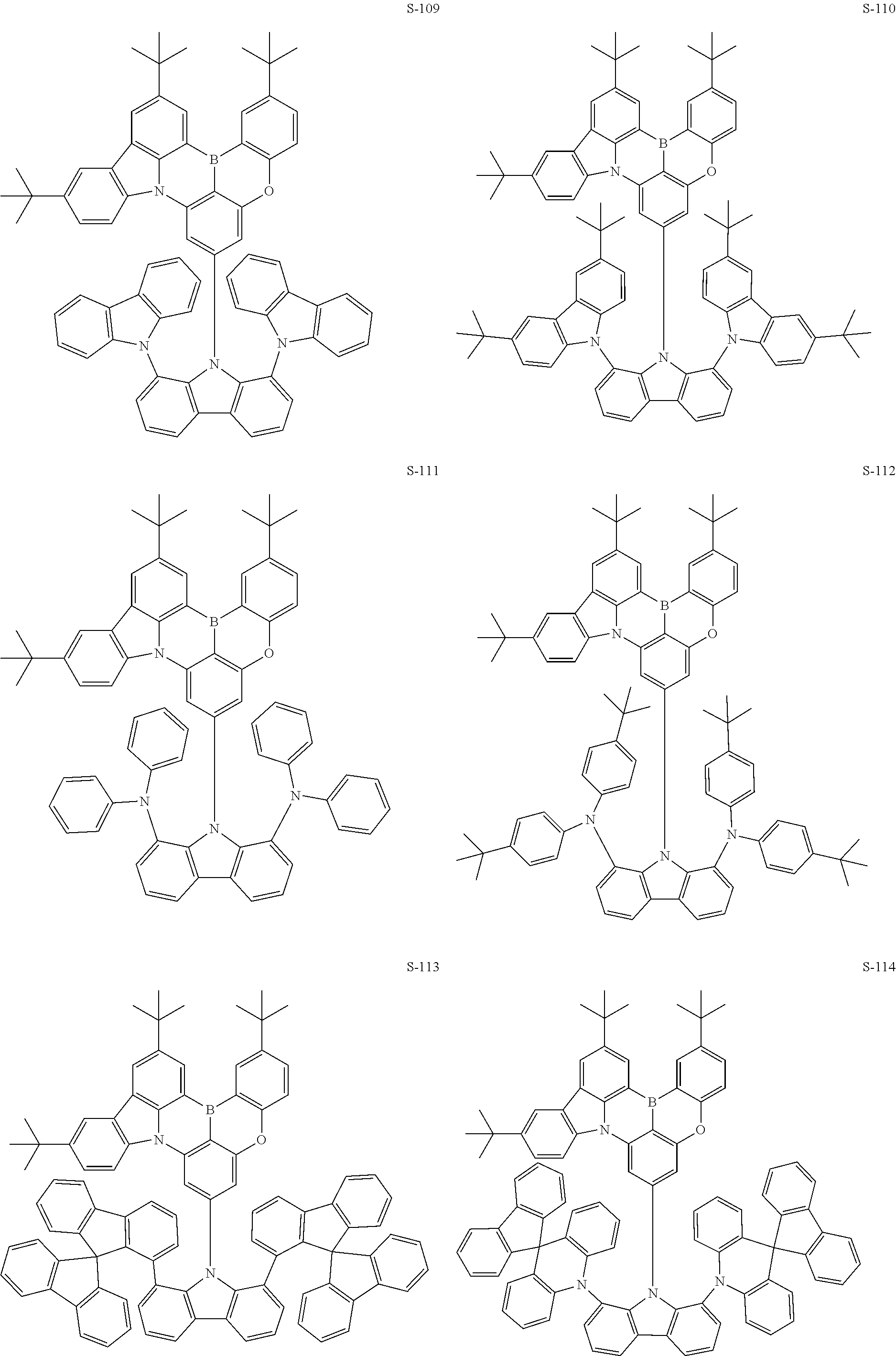

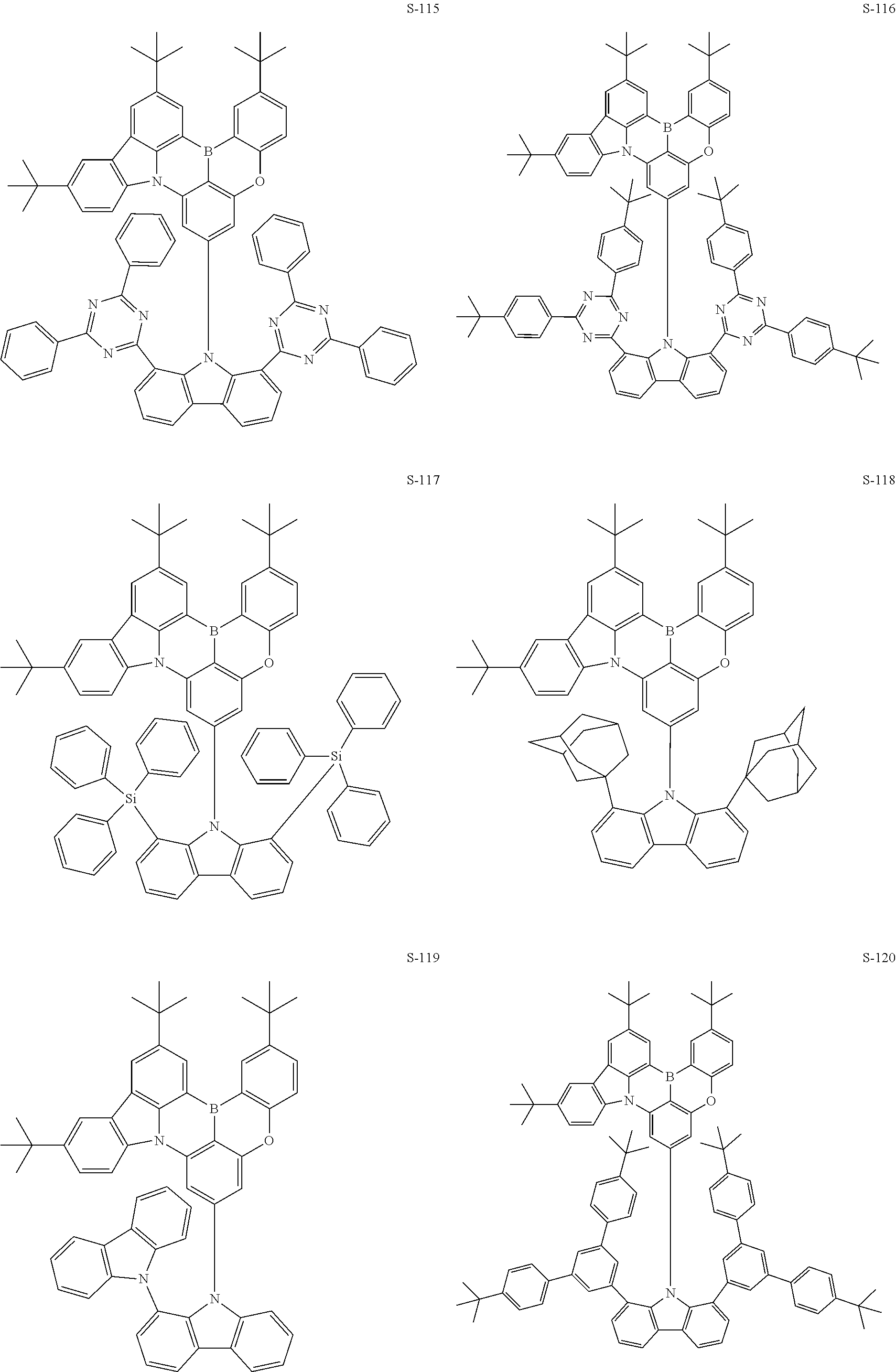

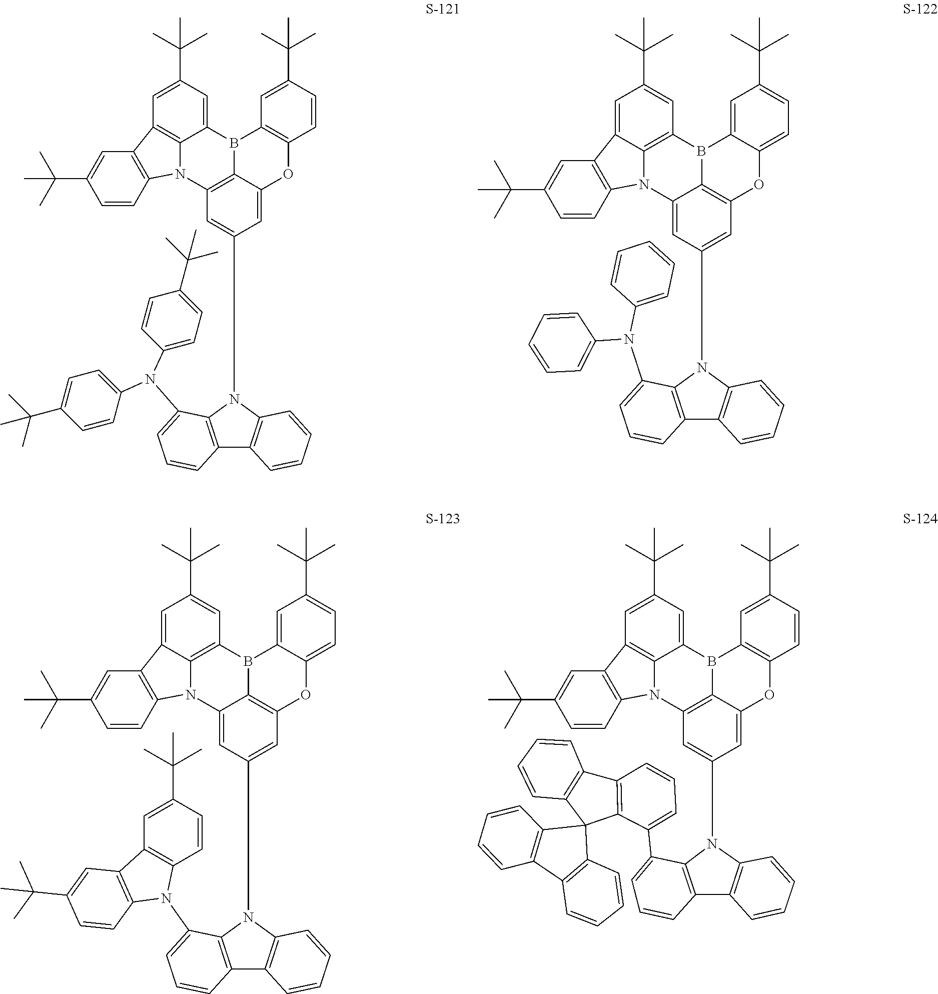

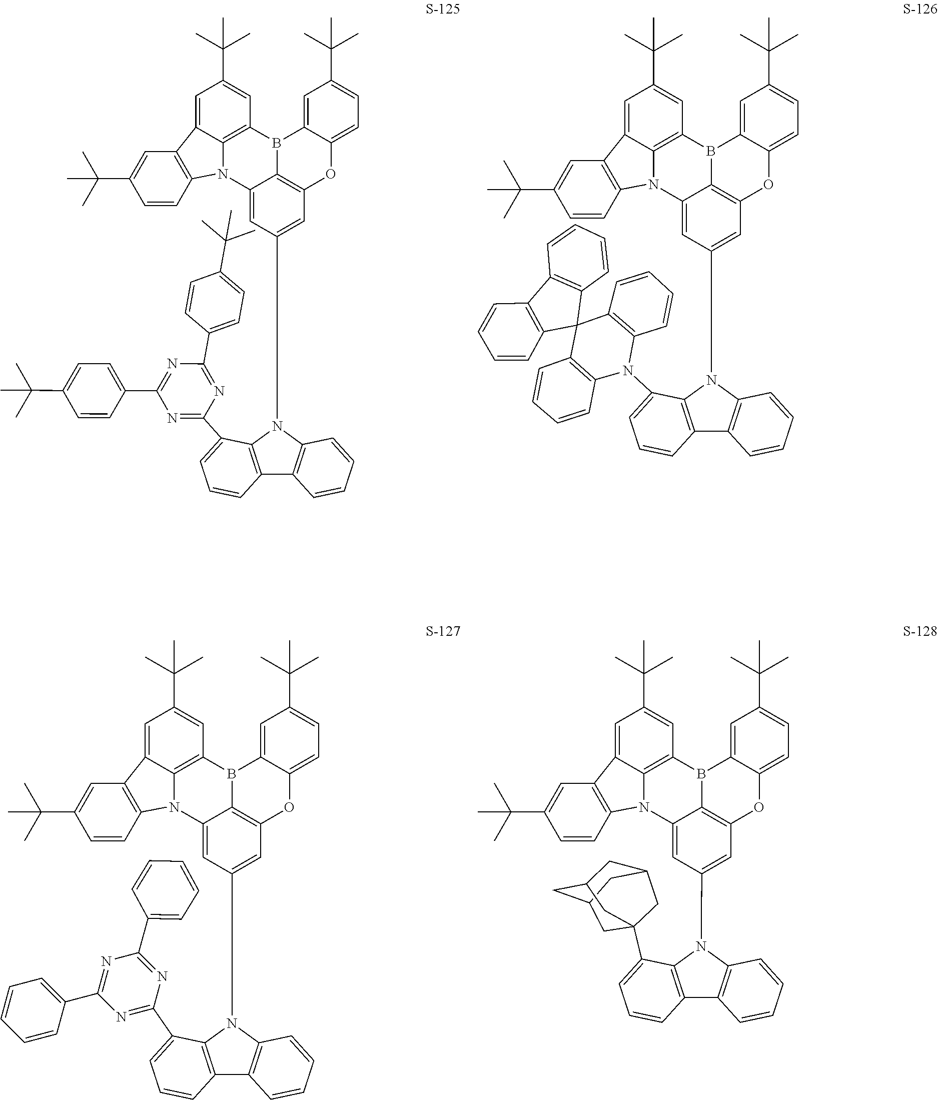

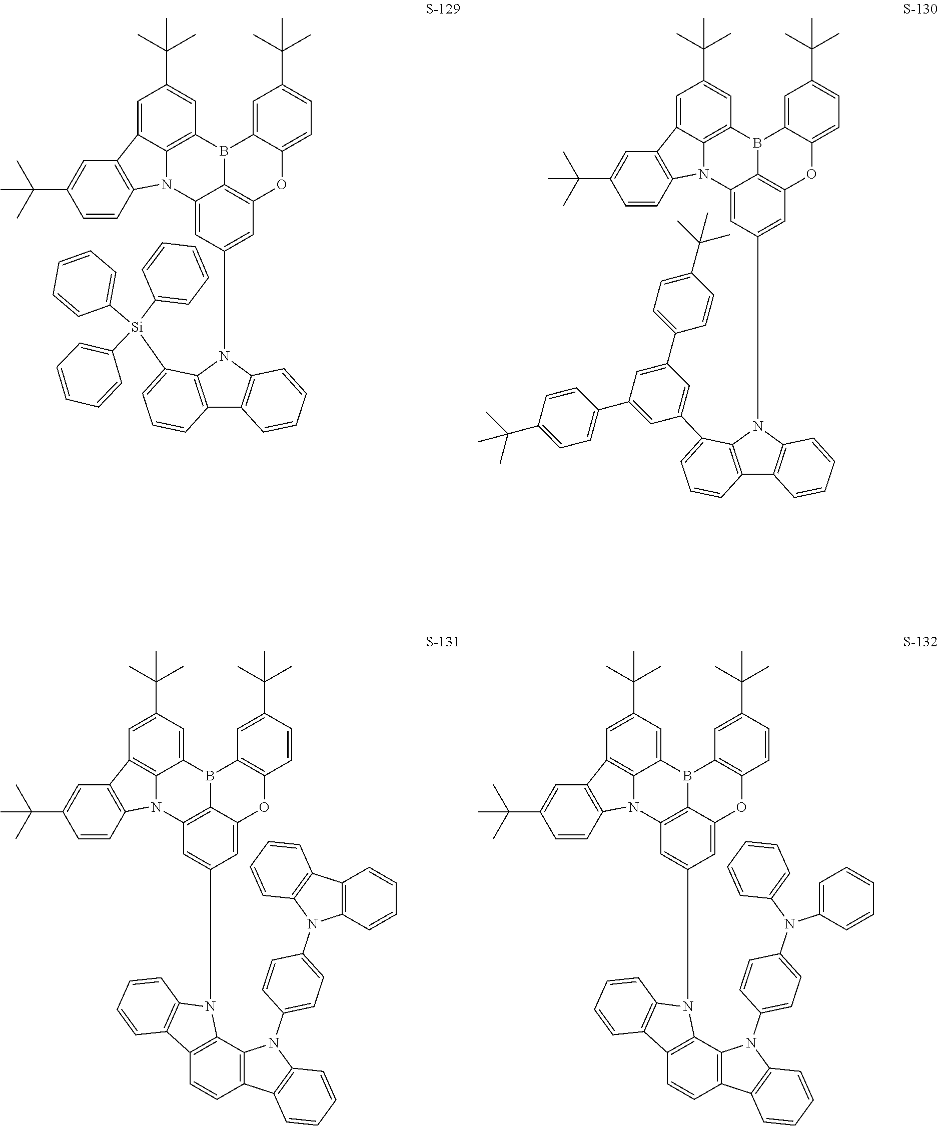

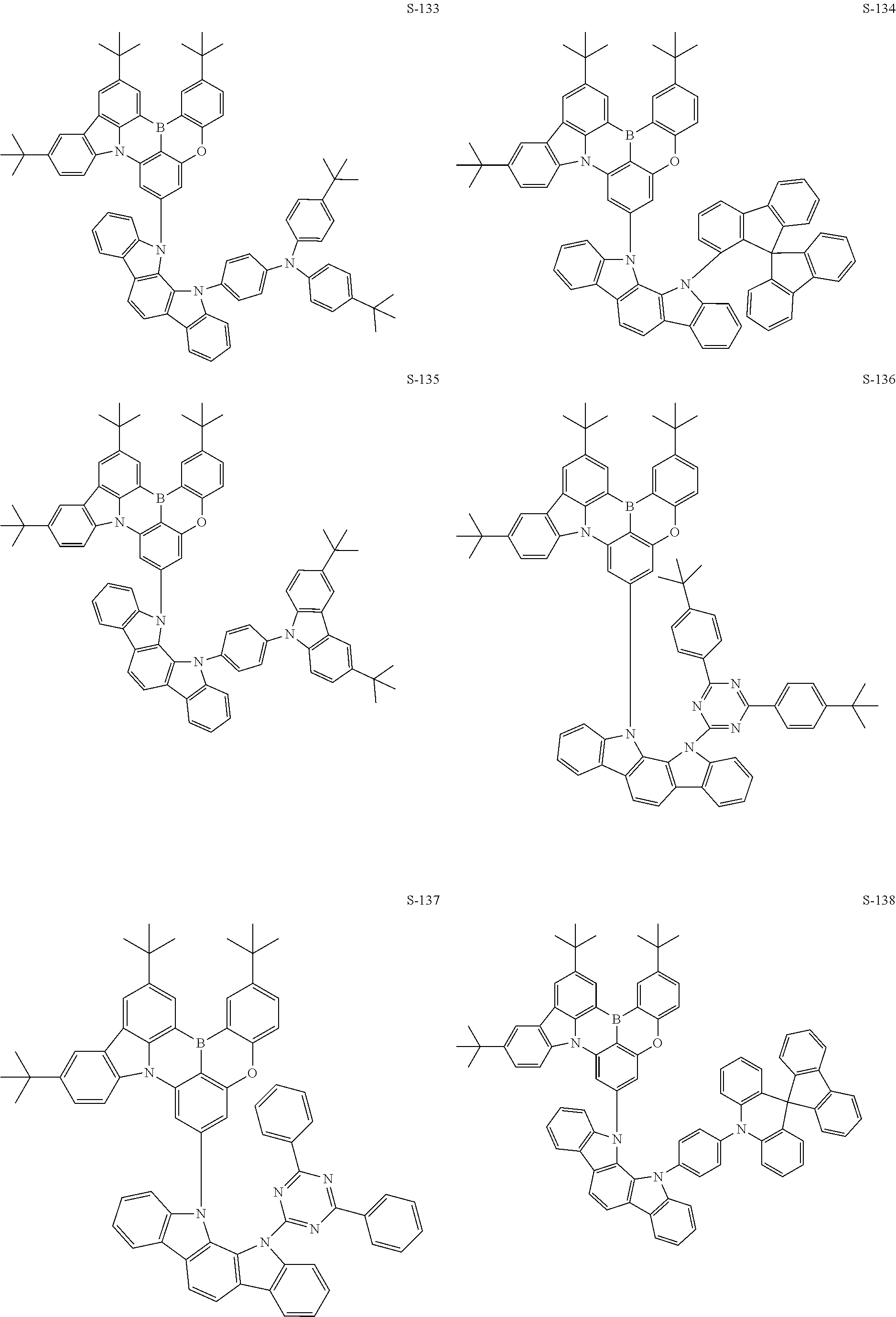

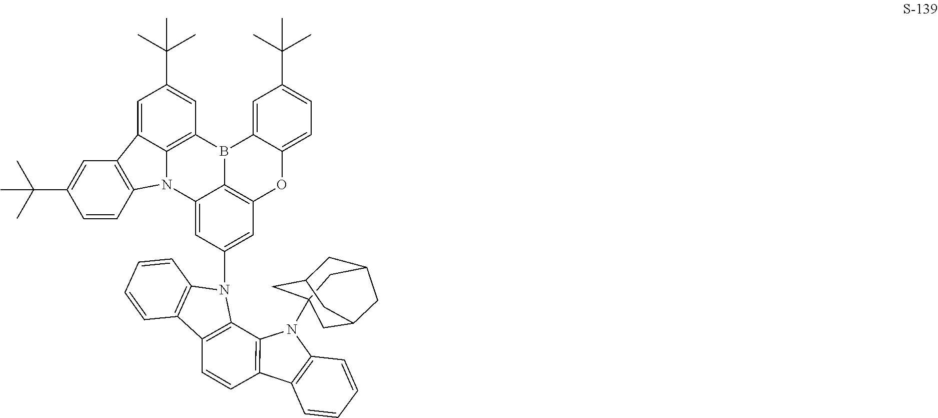 [SMILES: CC(C)(C)c1ccc2c(c1)B1c3c(cc(-n4c5ccccc5c5ccc6c7ccccc7n(C78CC9CC(CC(C9)C7)C8)c6c54)cc3-n3c4ccc(C(C)(C)C)cc4c4cc(C(C)(C)C)cc1c43)O2]

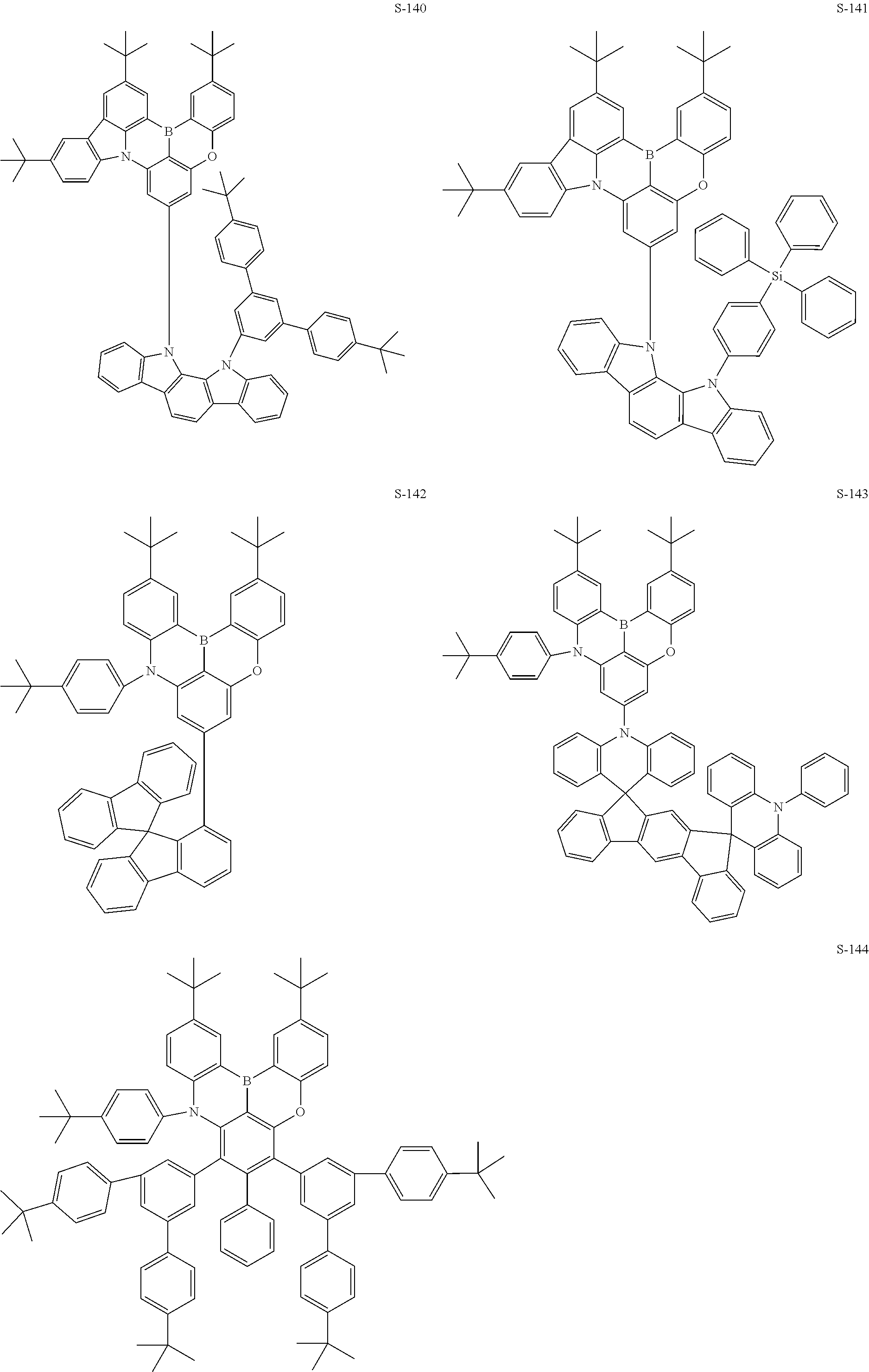

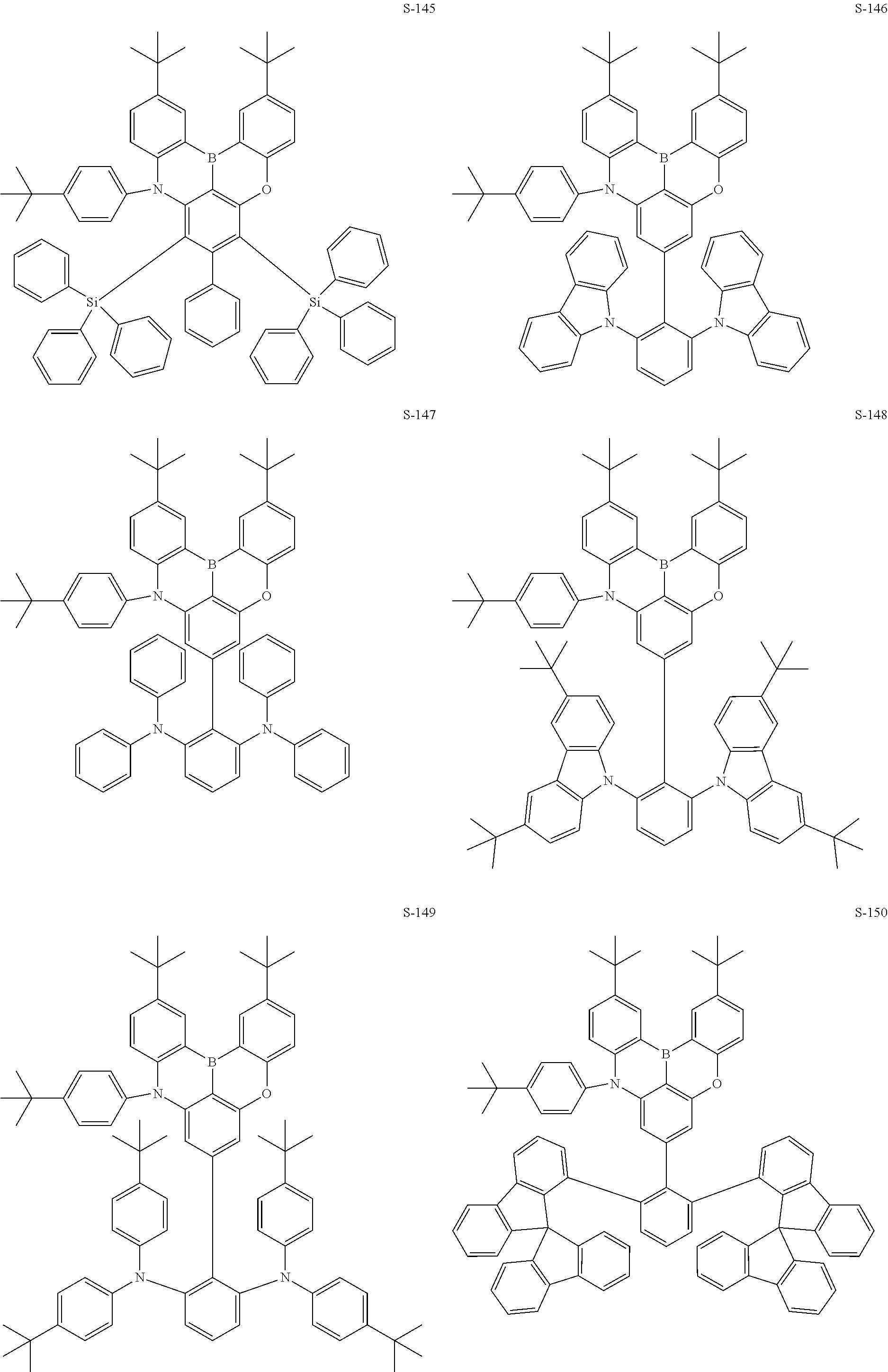

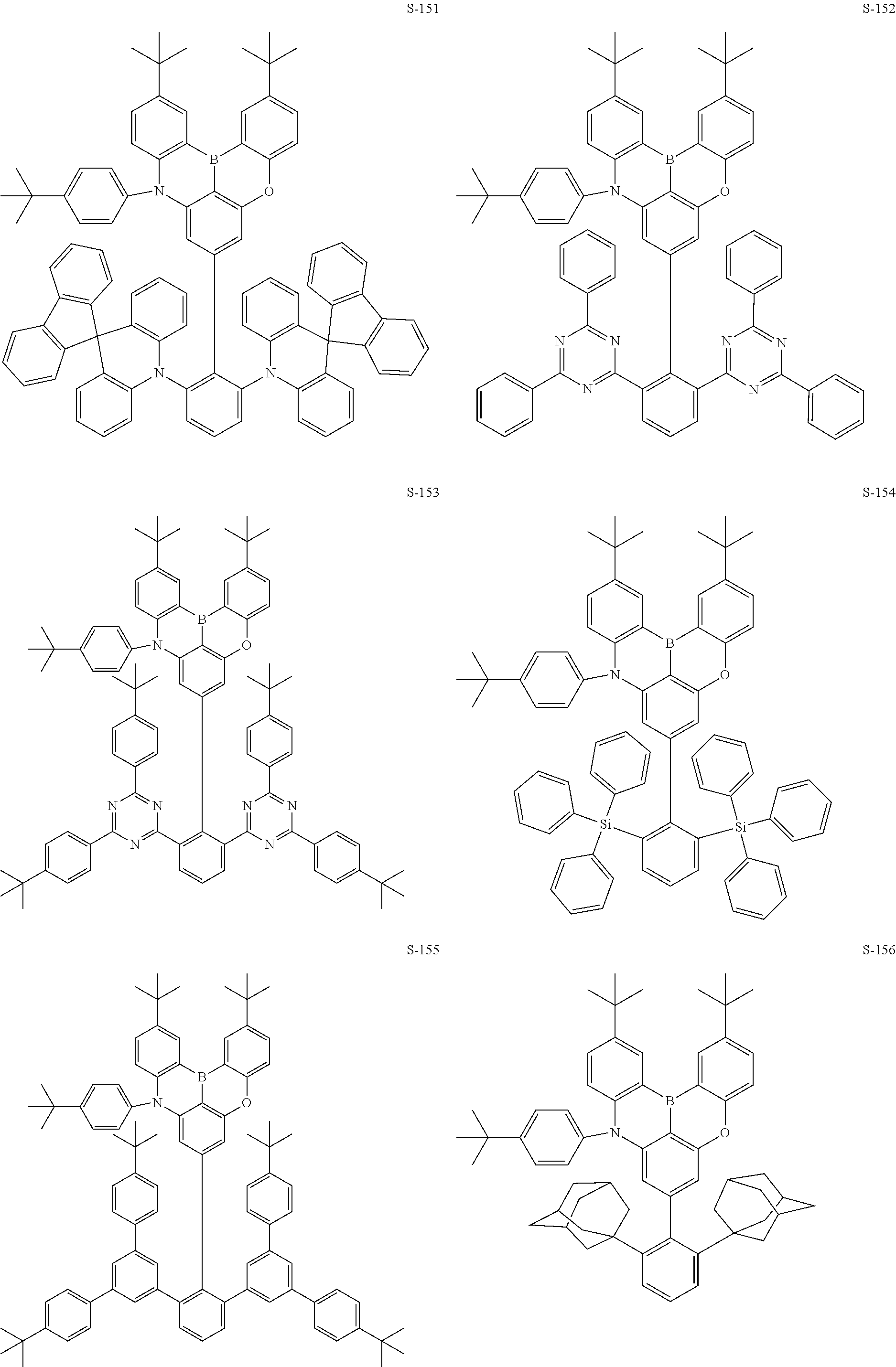

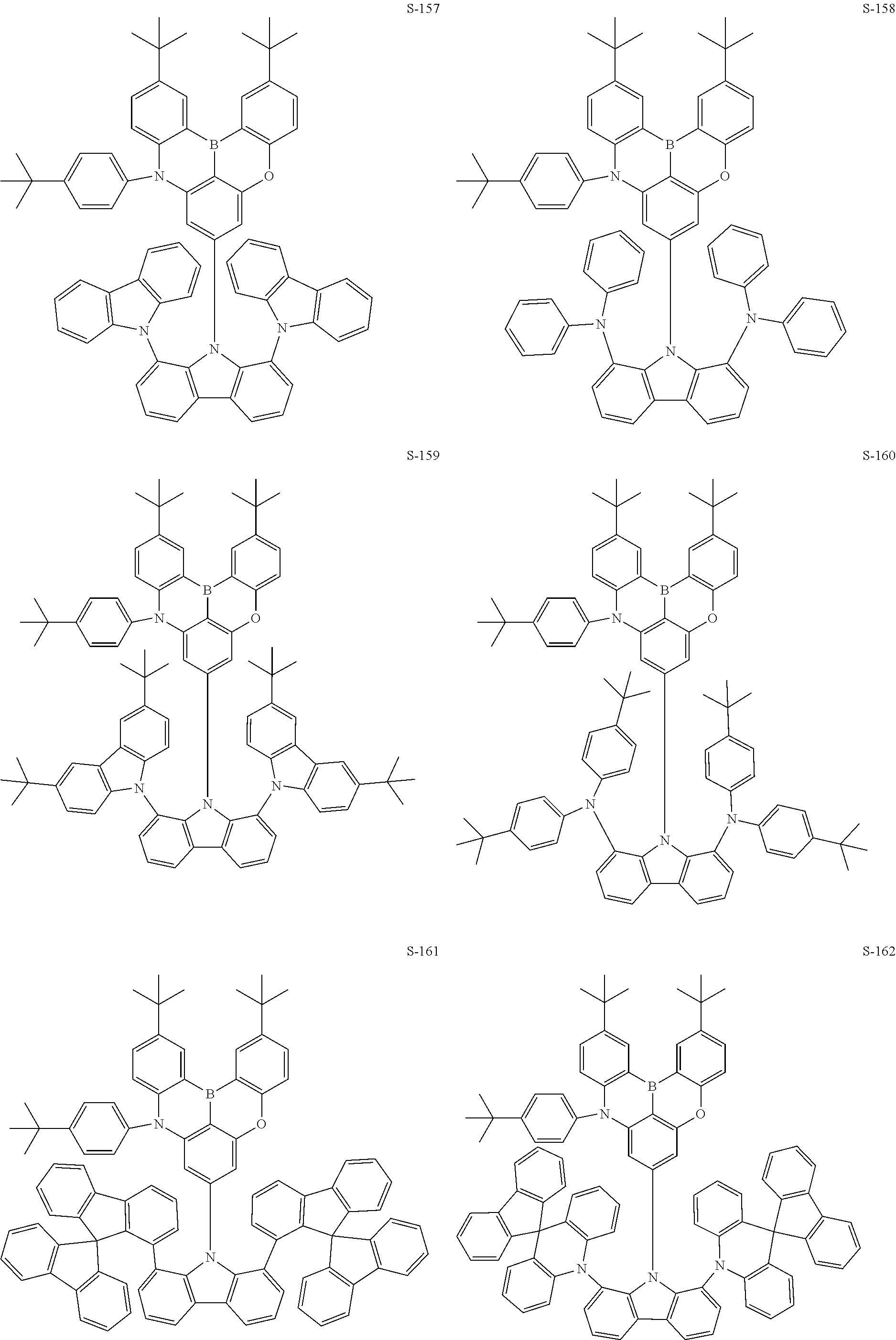

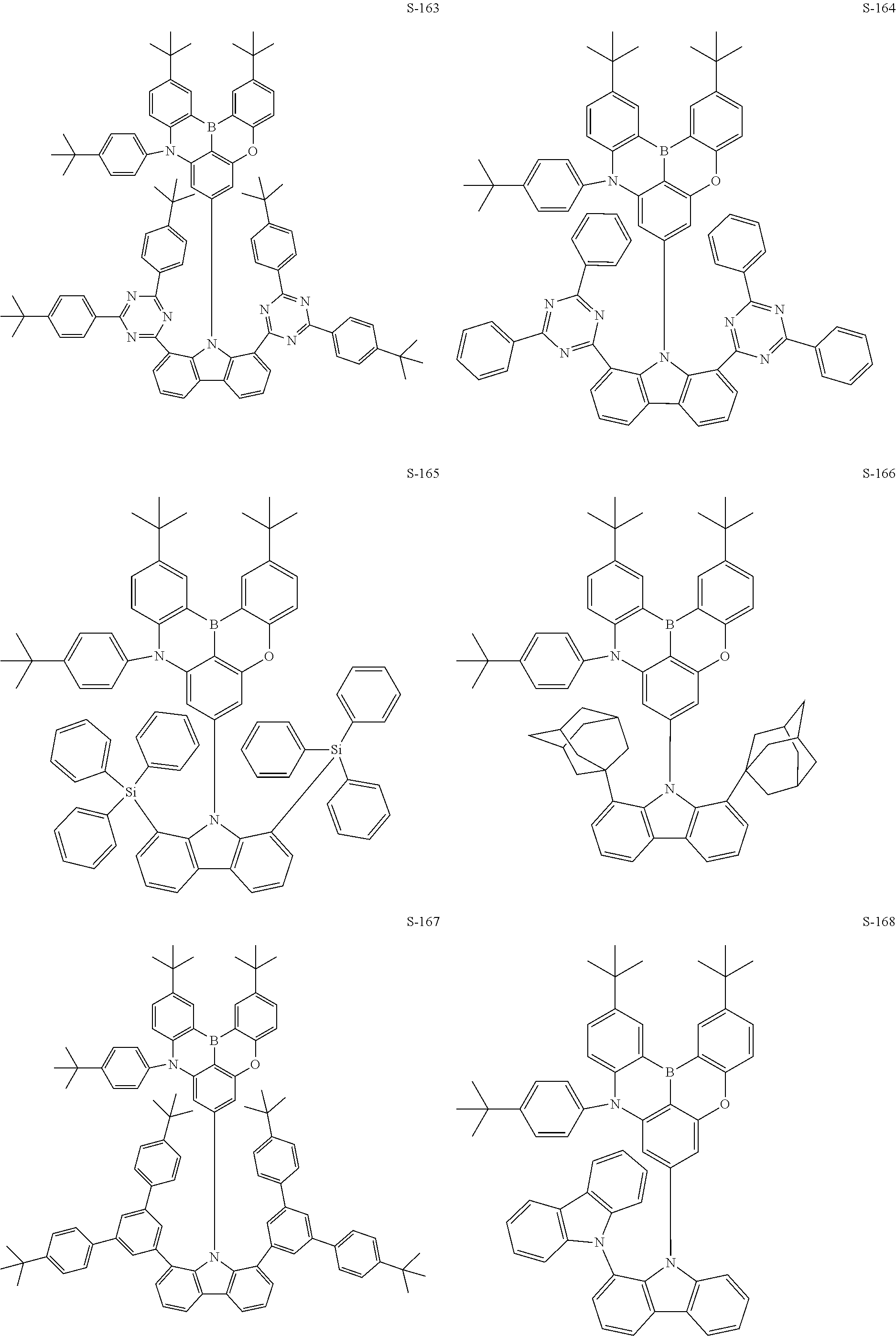

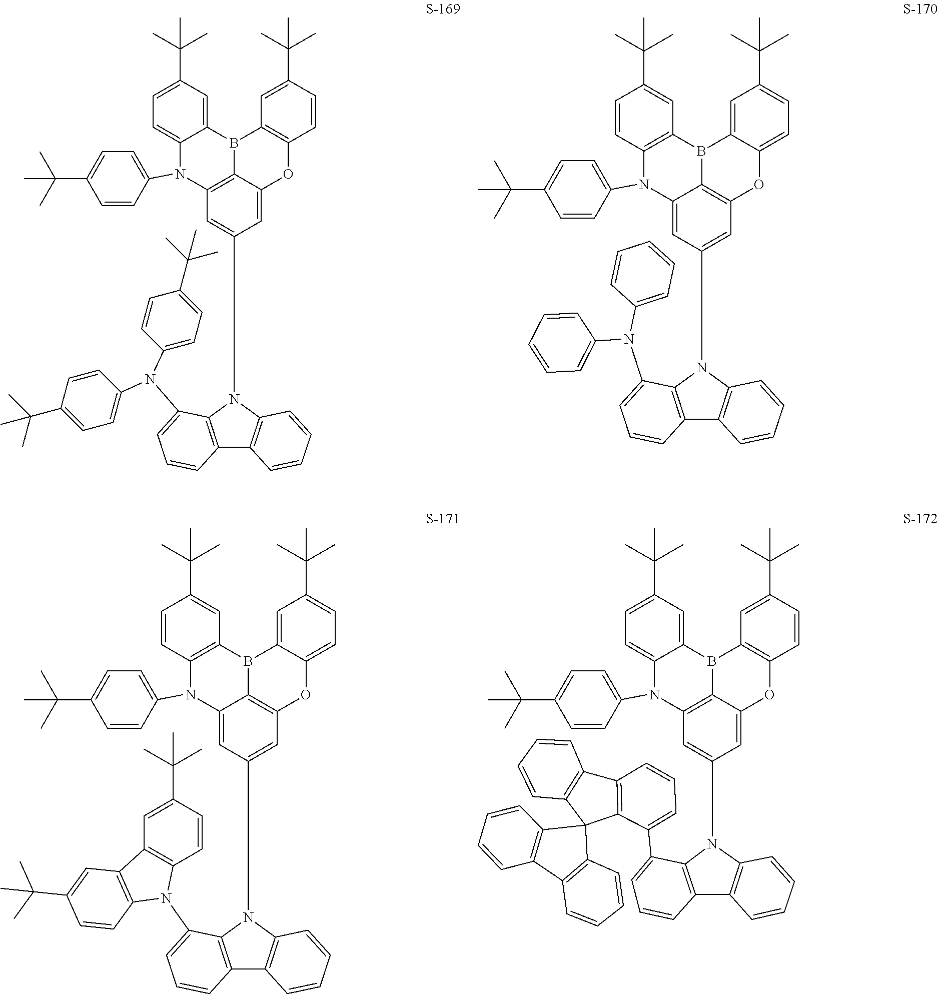

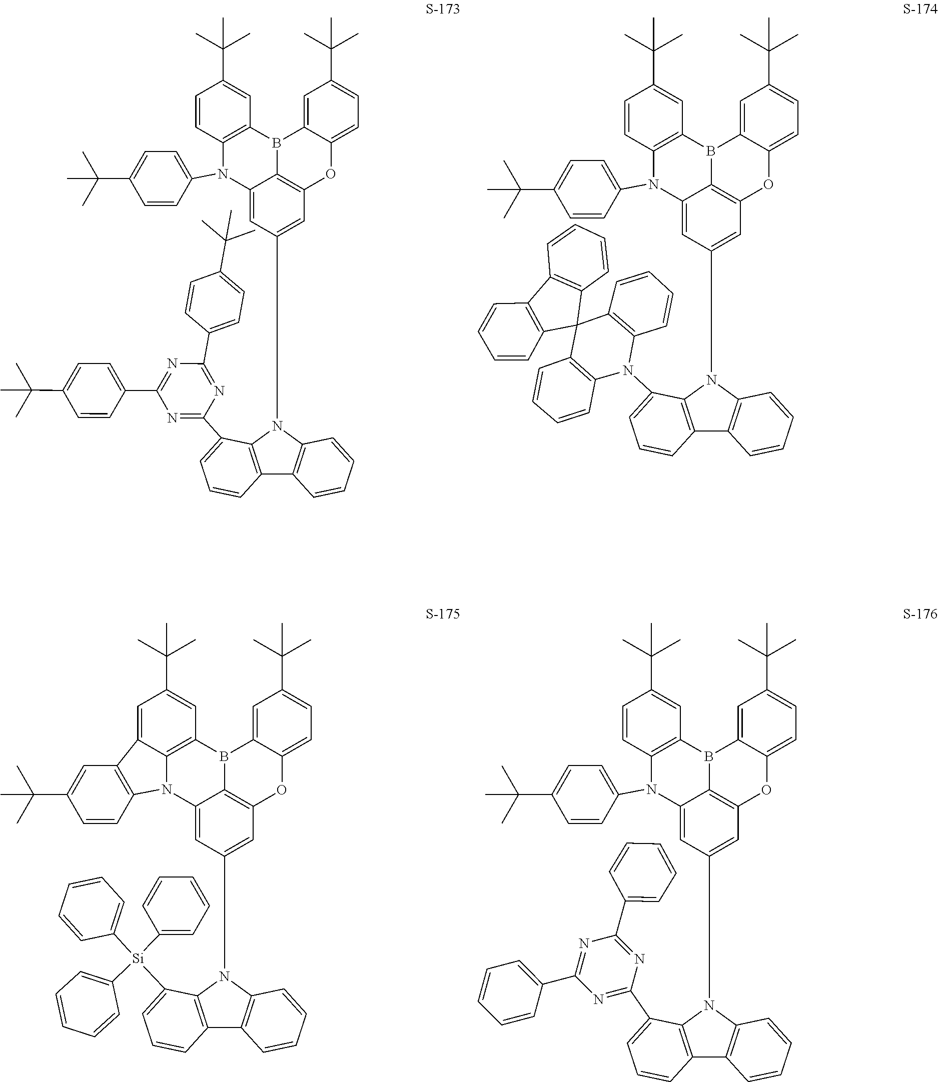

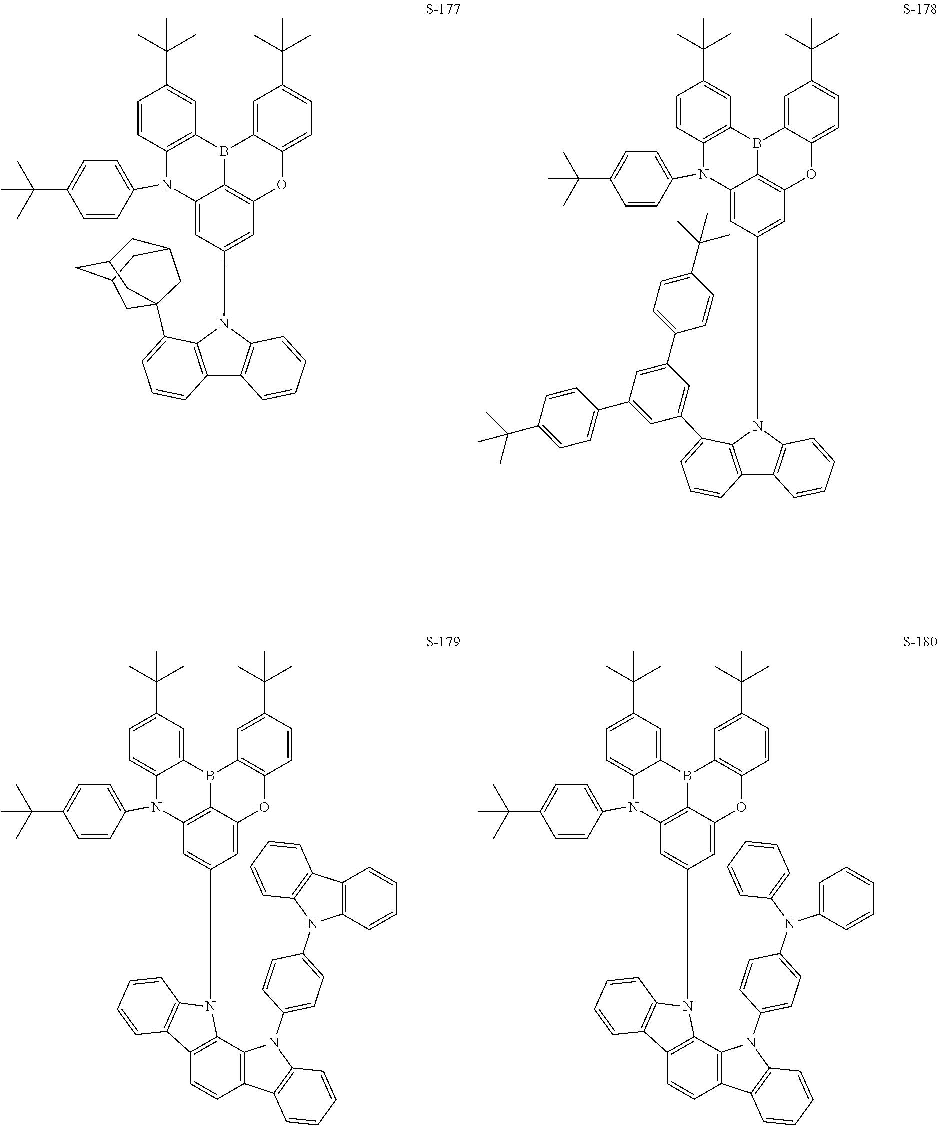

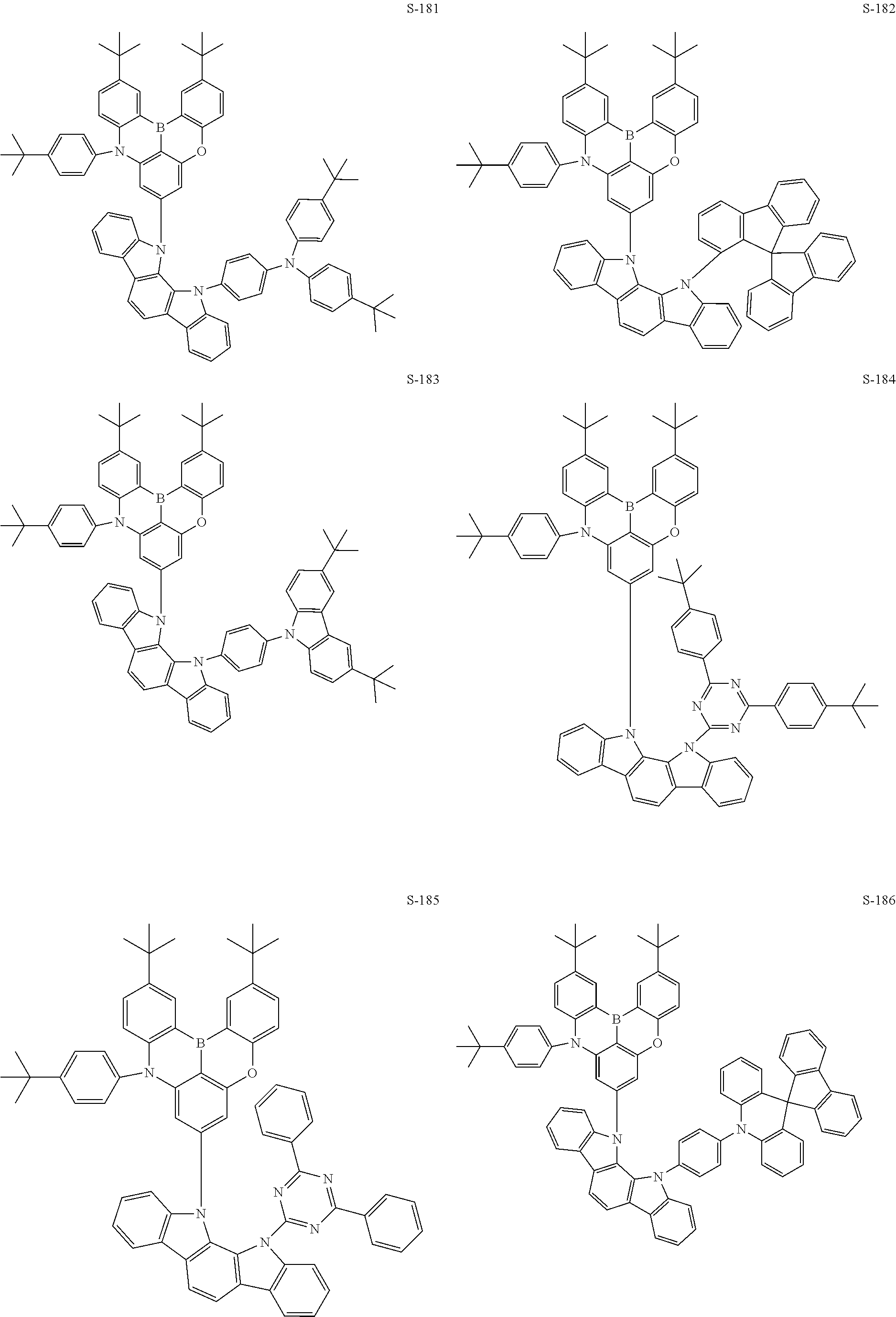

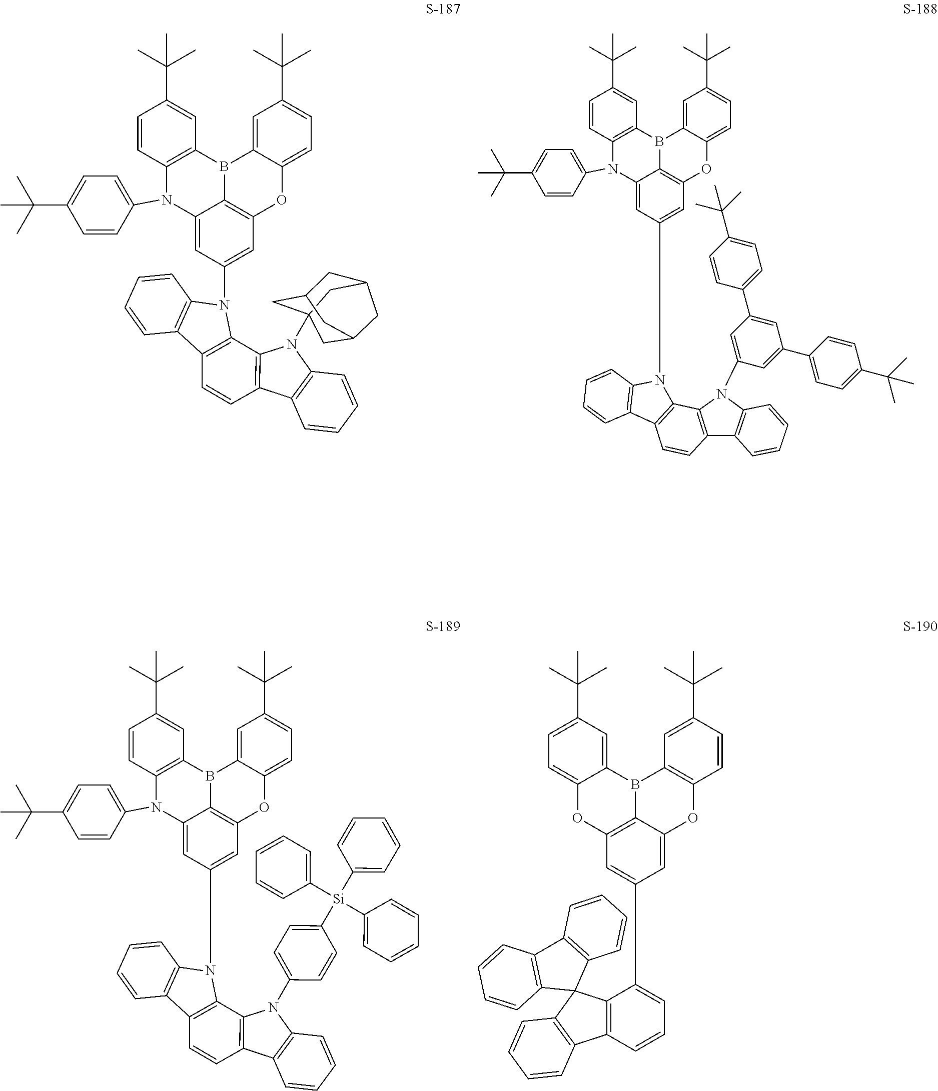

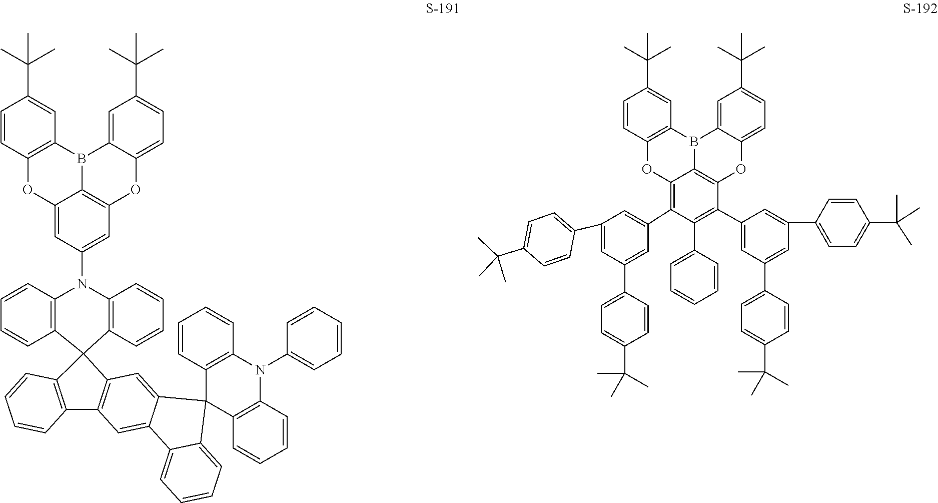 [SMILES: CC(C)(C)c1ccc2c(c1)B1c3cc(C(C)(C)C)ccc3Oc3cc(N4c5ccccc5C5(c6ccccc6-c6cc7c(cc65)C5(c6ccccc6-7)c6ccccc6N(c6ccccc6)c6ccccc65)c5ccccc54)cc(c31)O2 ; CC(C)(C)c1ccc(-c2cc(-c3ccc(C(C)(C)C)cc3)cc(-c3c4c5c(c(-c6cc(-c7ccc(C(C)(C)C)cc7)cc(-c7ccc(C(C)(C)C)cc7)c6)c3-c3ccccc3)Oc3ccc(C(C)(C)C)cc3B5c3cc(C(C)(C)C)ccc3O4)c2)cc1]

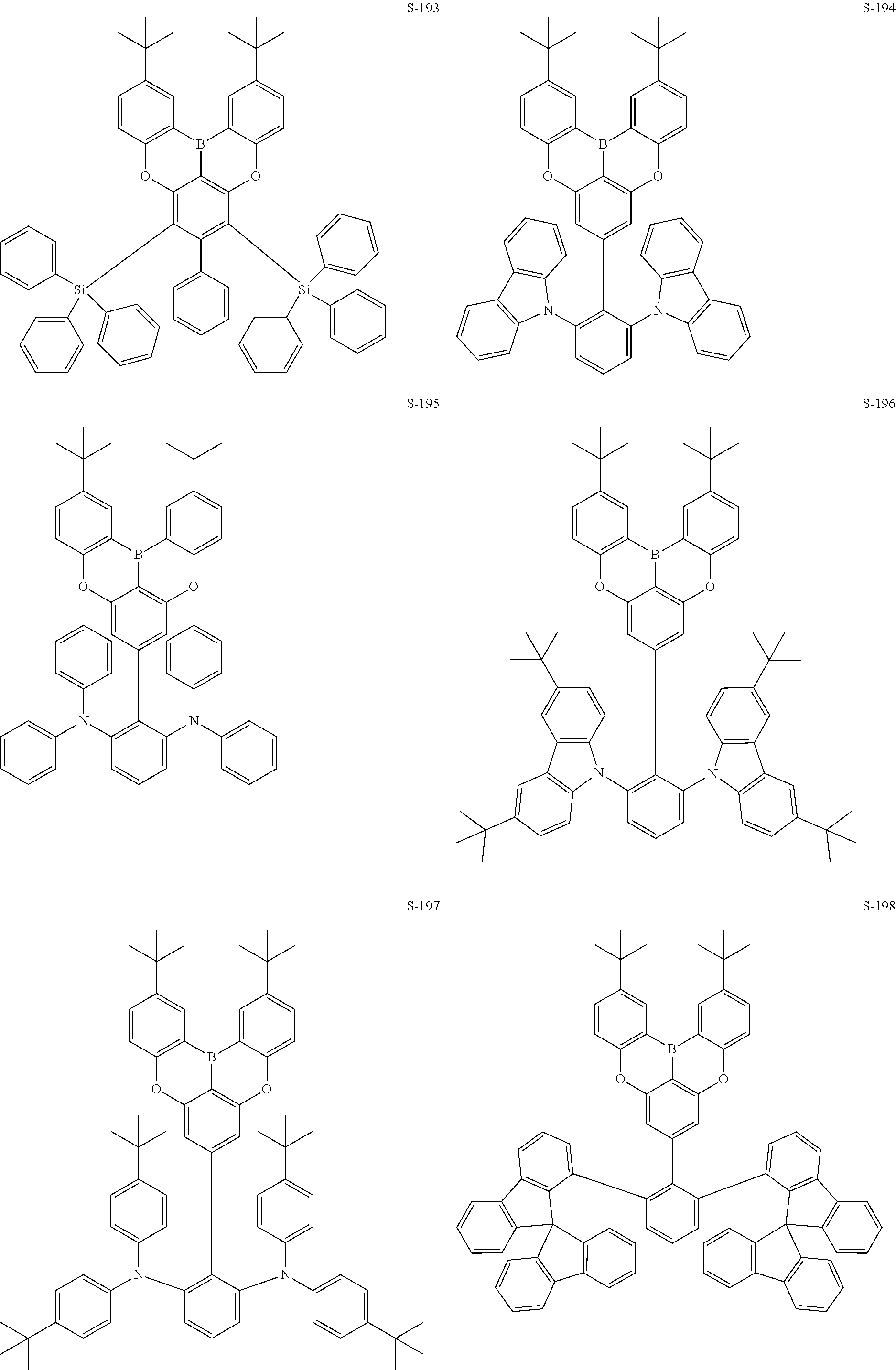

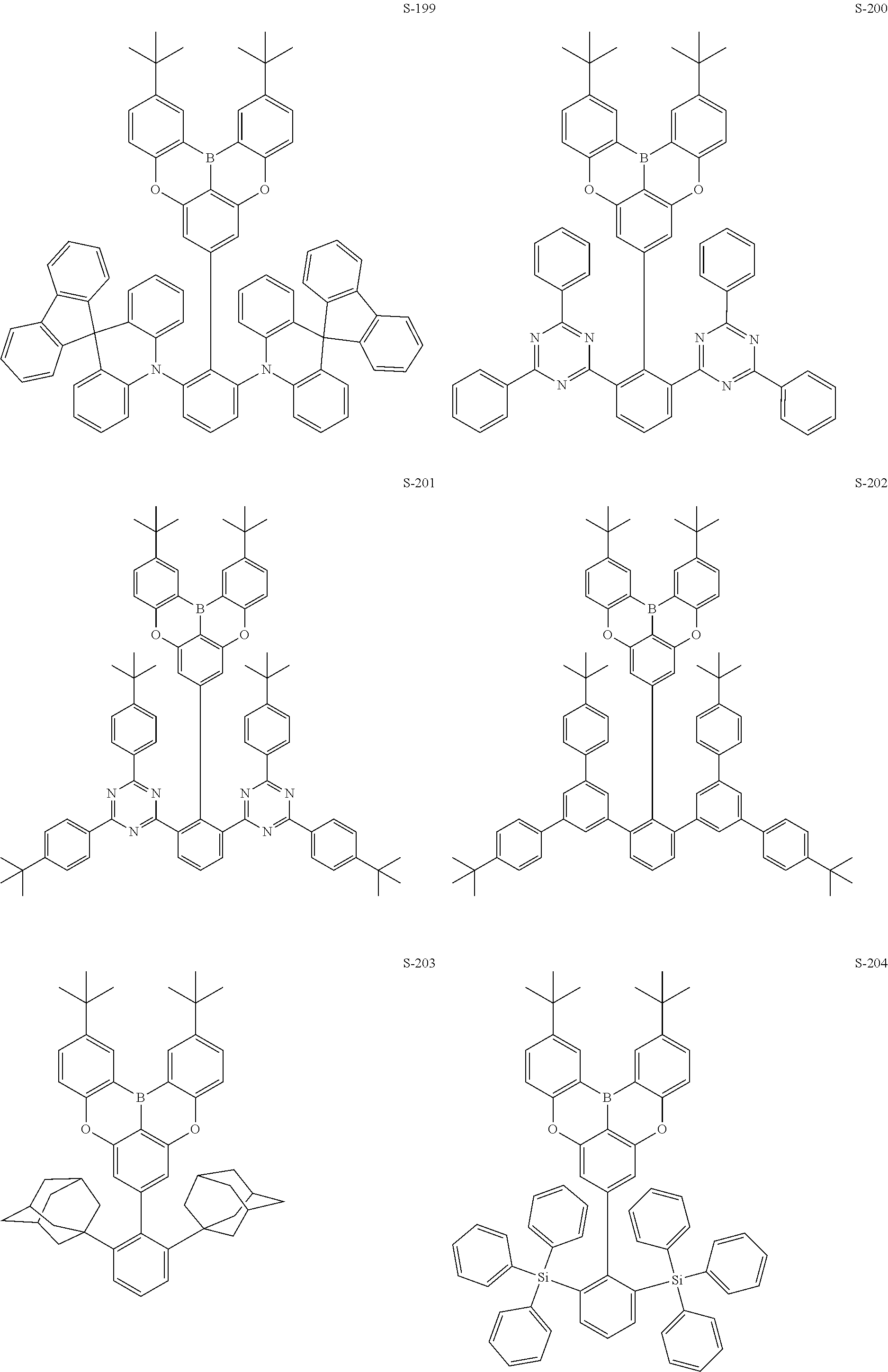

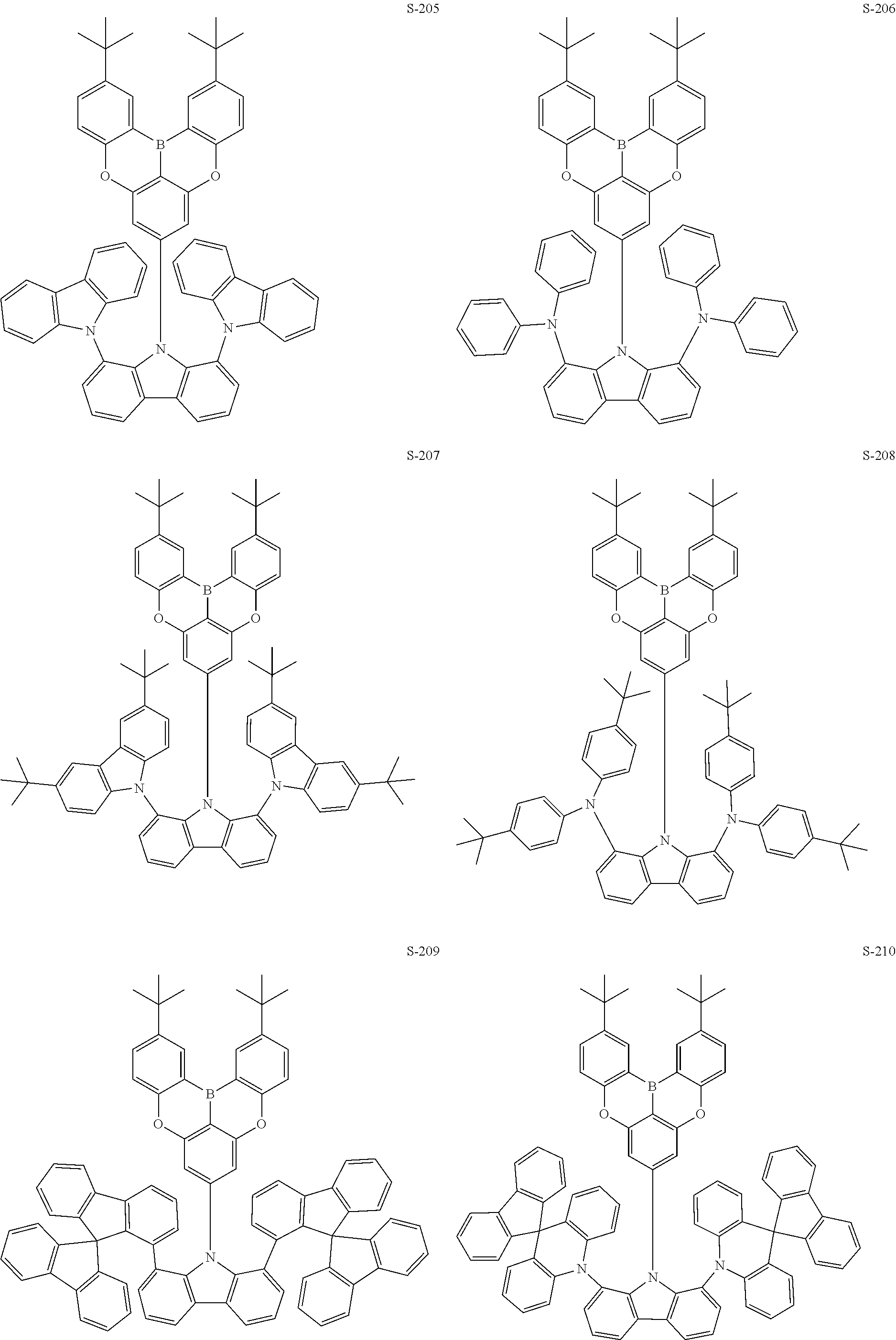

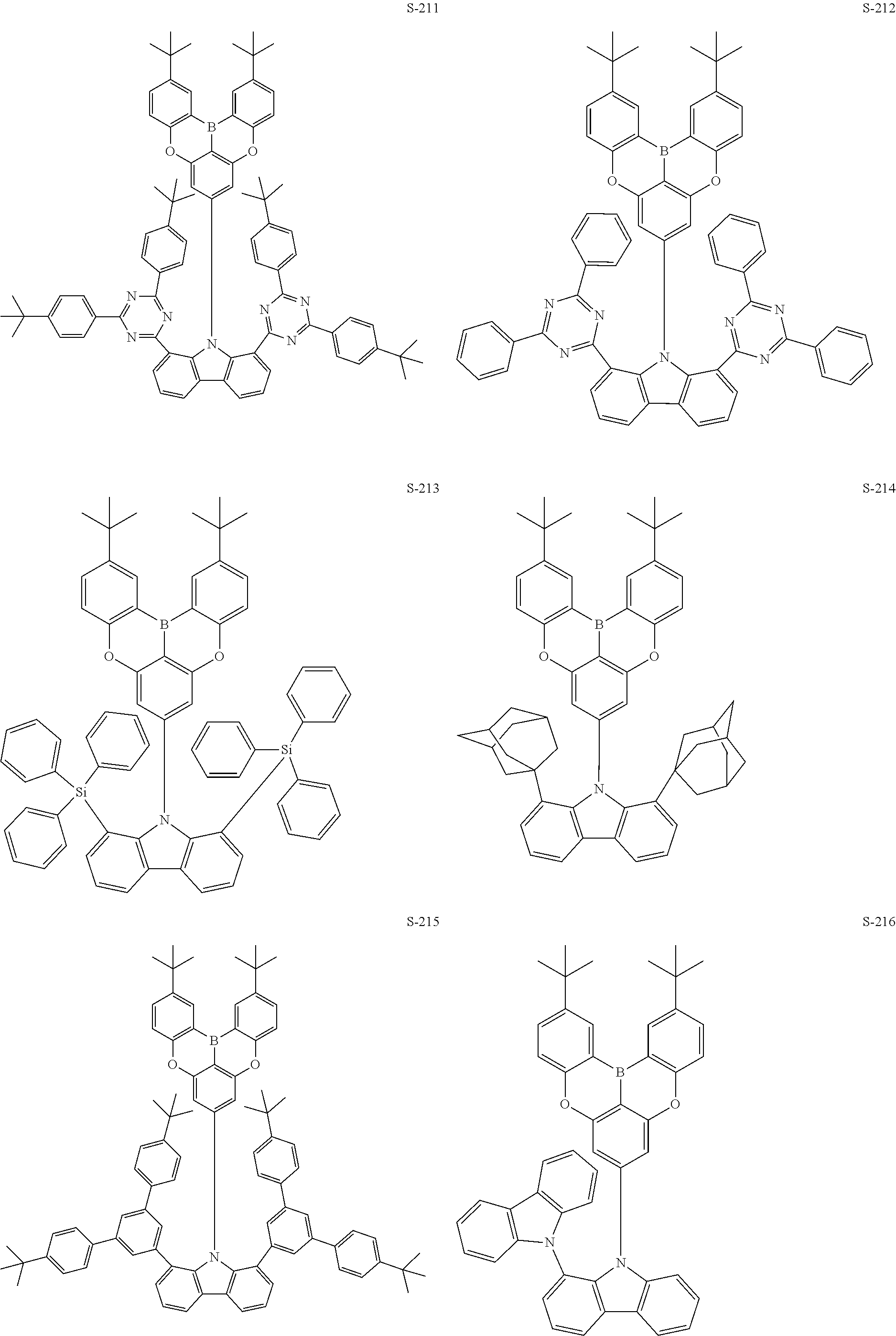

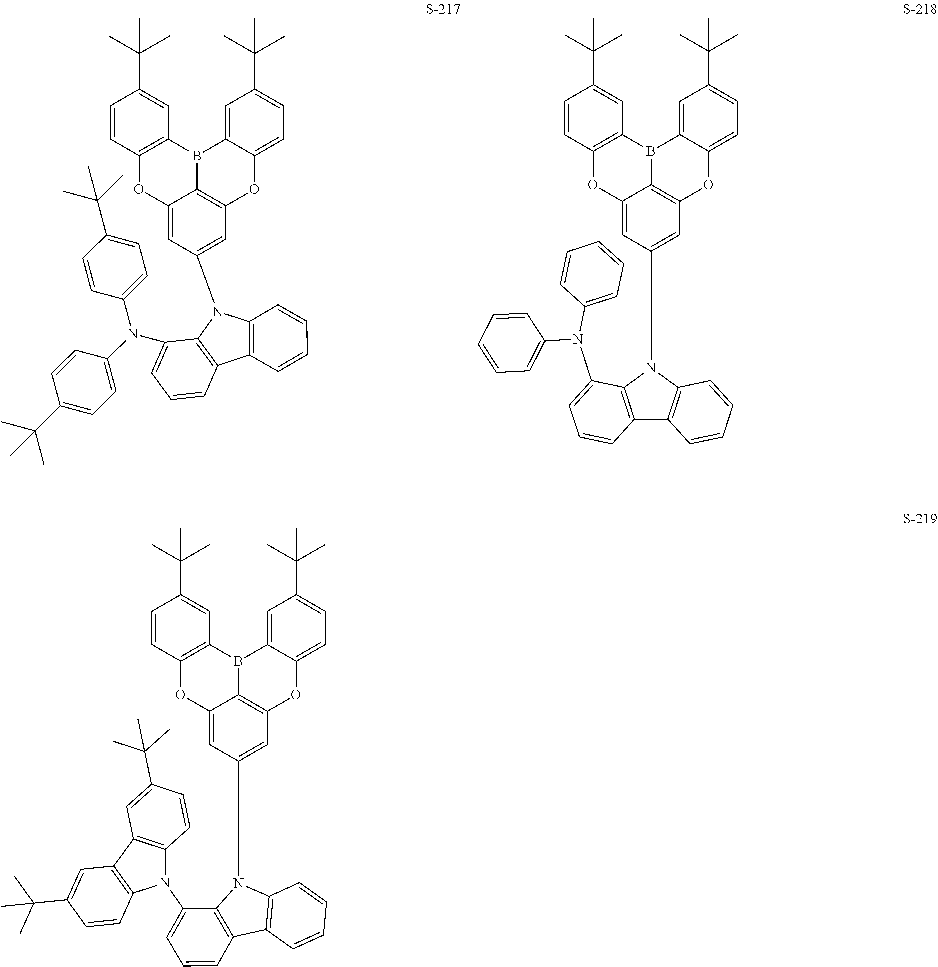 [SMILES: CC(C)(C)c1ccc(N(c2ccc(C(C)(C)C)cc2)c2cccc3c4ccccc4n(-c4cc5c6c(c4)Oc4ccc(C(C)(C)C)cc4B6c4cc(C(C)(C)C)ccc4O5)c23)cc1 ; CC(C)(C)c1ccc2c(c1)B1c3cc(C(C)(C)C)ccc3Oc3cc(-n4c5ccccc5c5cccc(N(c6ccccc6)c6ccccc6)c54)cc(c31)O2 ; CC(C)(C)c1ccc2c(c1)B1c3cc(C(C)(C)C)ccc3Oc3cc(-n4c5ccccc5c5cccc(-n6c7ccc(C(C)(C)C)cc7c7cc(C(C)(C)C)ccc76)c54)cc(c31)O2]

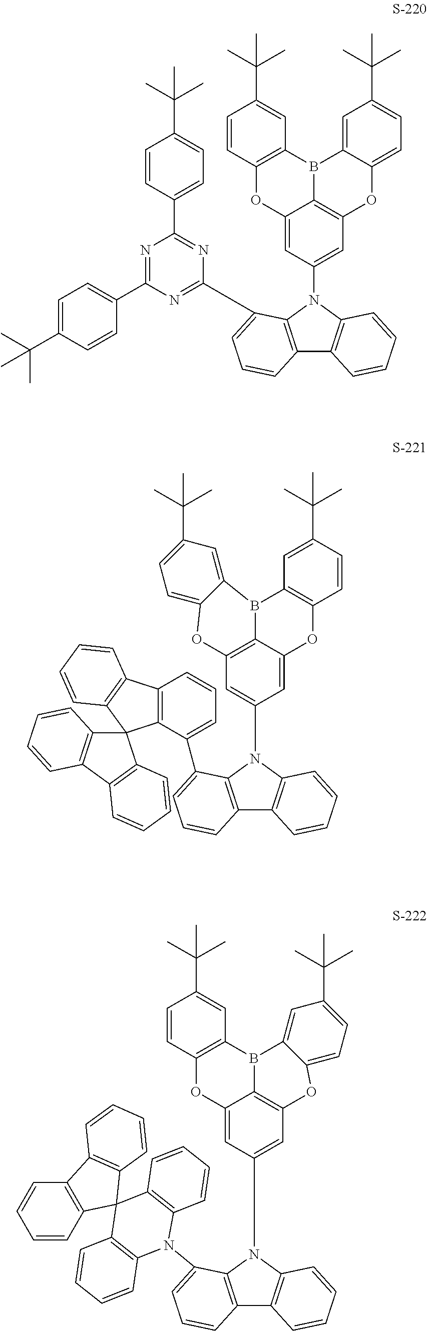 [SMILES: CC(C)(C)c1ccc(-c2nc(-c3ccc(C(C)(C)C)cc3)nc(-c3cccc4c5ccccc5n(-c5cc6c7c(c5)Oc5ccc(C(C)(C)C)cc5B7c5cc(C(C)(C)C)ccc5O6)c34)n2)cc1 ; CC(C)(C)c1ccc2c(c1)B1c3cc(C(C)(C)C)ccc3Oc3cc(-n4c5ccccc5c5cccc(-c6cccc7c6C6(c8ccccc8-c8ccccc86)c6ccccc6-7)c54)cc(c31)O2 ; CC(C)(C)c1ccc2c(c1)B1c3cc(C(C)(C)C)ccc3Oc3cc(-n4c5ccccc5c5cccc(N6c7ccccc7C7(c8ccccc8-c8ccccc87)c7ccccc76)c54)cc(c31)O2]

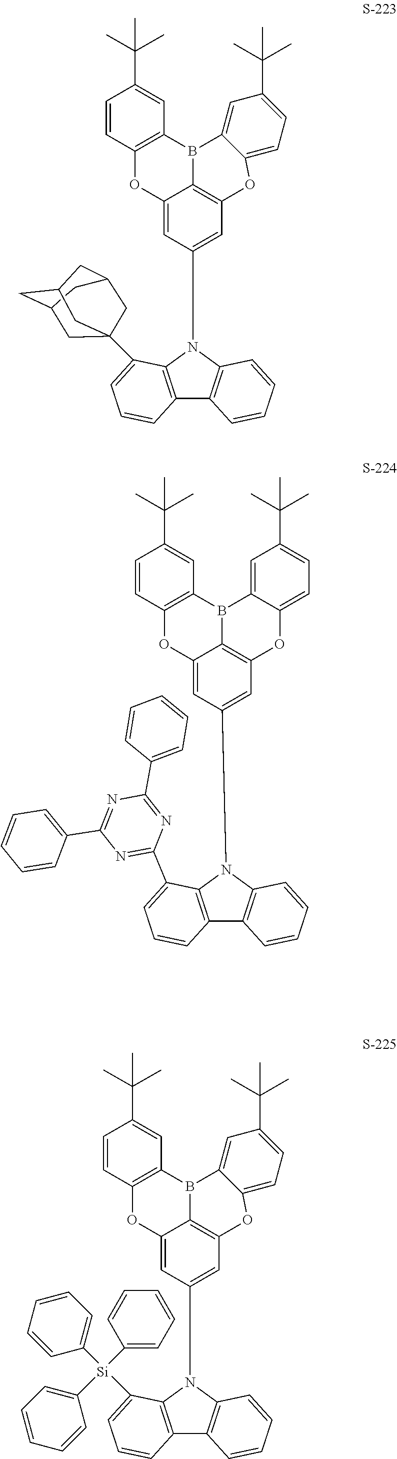 [SMILES: CC(C)(C)c1ccc2c(c1)B1c3cc(C(C)(C)C)ccc3Oc3cc(-n4c5ccccc5c5cccc(C67CC8CCC(CC(C8)C6)C7)c54)cc(c31)O2 ; CC(C)(C)c1ccc2c(c1)B1c3cc(C(C)(C)C)ccc3Oc3cc(-n4c5ccccc5c5cccc(-c6nc(-c7ccccc7)nc(-c7ccccc7)n6)c54)cc(c31)O2 ; CC(C)(C)c1ccc2c(c1)B1c3cc(C(C)(C)C)ccc3Oc3cc(-n4c5ccccc5c5cccc([Si](c6ccccc6)(c6ccccc6)c6ccccc6)c54)cc(c31)O2]

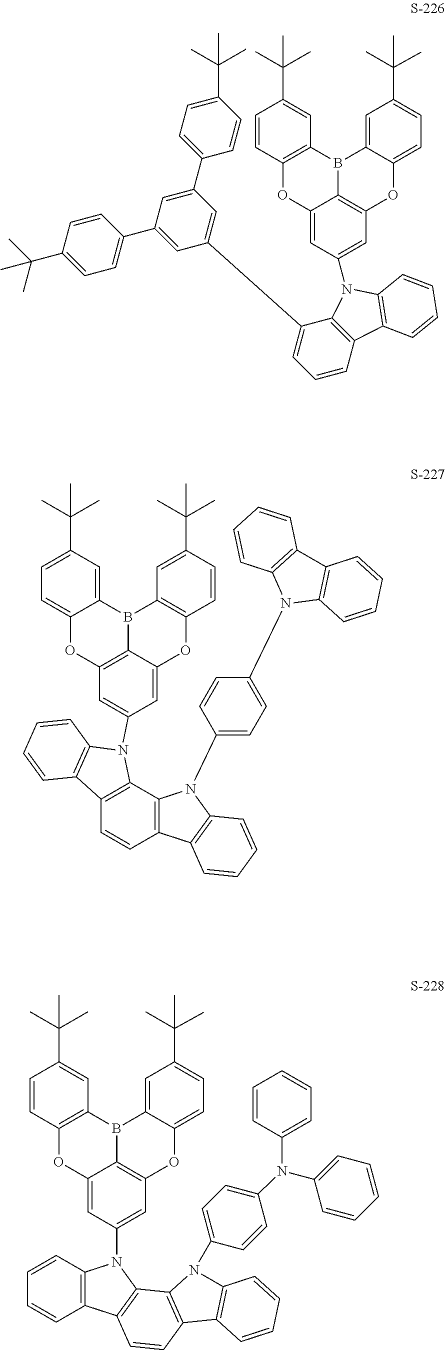 [SMILES: CC(C)(C)c1ccc(-c2cc(-c3ccc(C(C)(C)C)cc3)cc(-c3cccc4c5ccccc5n(-c5cc6c7c(c5)Oc5ccc(C(C)(C)C)cc5B7c5cc(C(C)(C)C)ccc5O6)c34)c2)cc1 ; CC(C)(C)c1ccc2c(c1)B1c3cc(C(C)(C)C)ccc3Oc3cc(-n4c5ccccc5c5ccc6c7ccccc7n(-c7ccc(-n8c9ccccc9c9ccccc98)cc7)c6c54)cc(c31)O2 ; CC(C)(C)c1ccc2c(c1)B1c3cc(C(C)(C)C)ccc3Oc3cc(-n4c5ccccc5c5ccc6c7ccccc7n(-c7ccc(N(c8ccccc8)c8ccccc8)cc7)c6c54)cc(c31)O2]

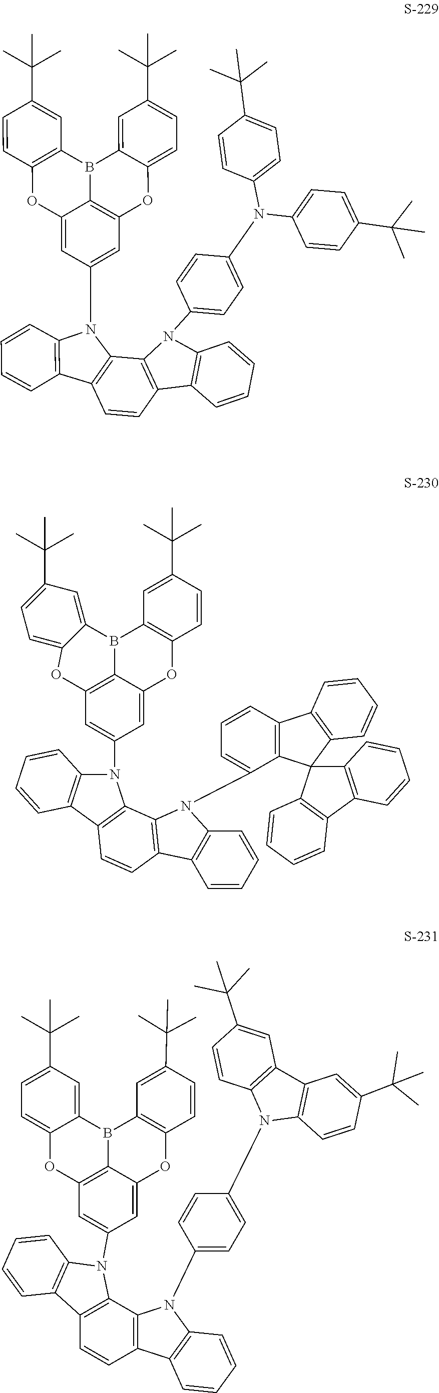

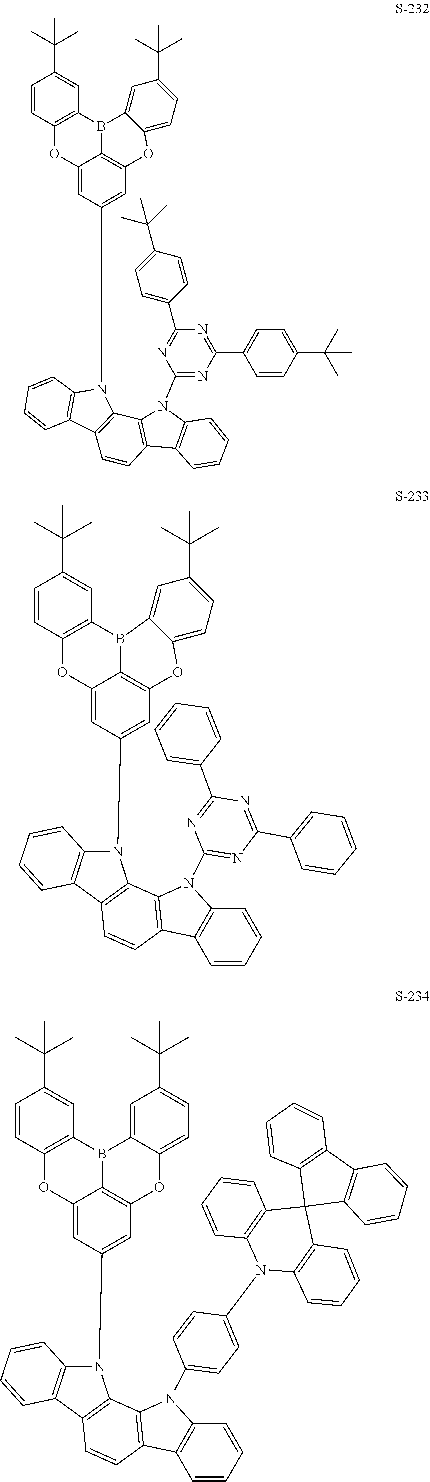

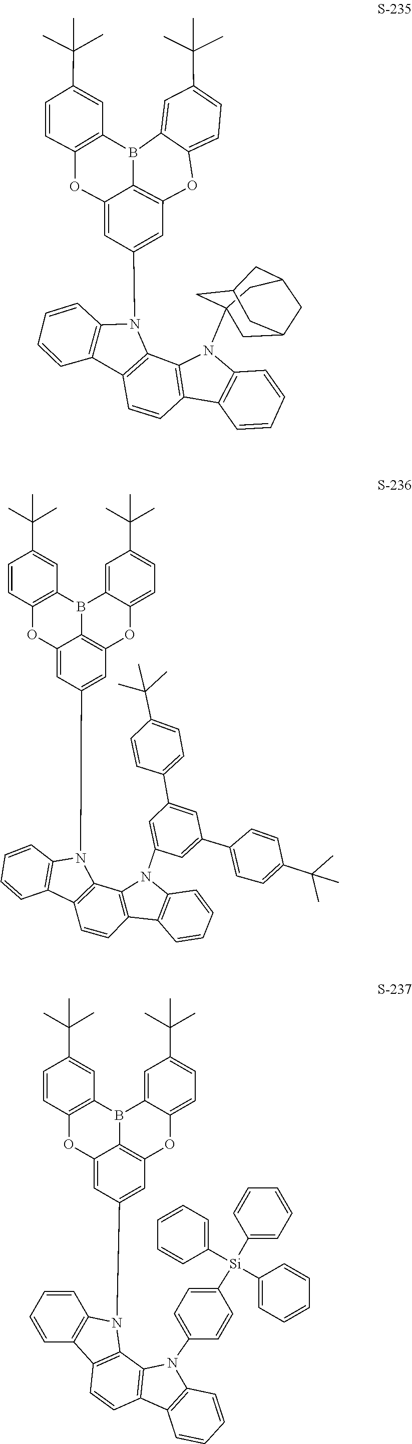 [SMILES: CC(C)(C)c1ccc2c(c1)B1c3cc(C(C)(C)C)ccc3Oc3cc(-n4c5ccccc5c5ccc6c7ccccc7n(C78CC9CC(CC(C9)C7)C8)c6c54)cc(c31)O2 ; CC(C)(C)c1ccc(-c2cc(-c3ccc(C(C)(C)C)cc3)cc(-n3c4ccccc4c4ccc5c6ccccc6n(-c6cc7c8c(c6)Oc6ccc(C(C)(C)C)cc6B8c6cc(C(C)(C)C)ccc6O7)c5c43)c2)cc1 ; CC(C)(C)c1ccc2c(c1)B1c3cc(C(C)(C)C)ccc3Oc3cc(-n4c5ccccc5c5ccc6c7ccccc7n(-c7ccc([Si](c8ccccc8)(c8ccccc8)c8ccccc8)cc7)c6c54)cc(c31)O2]

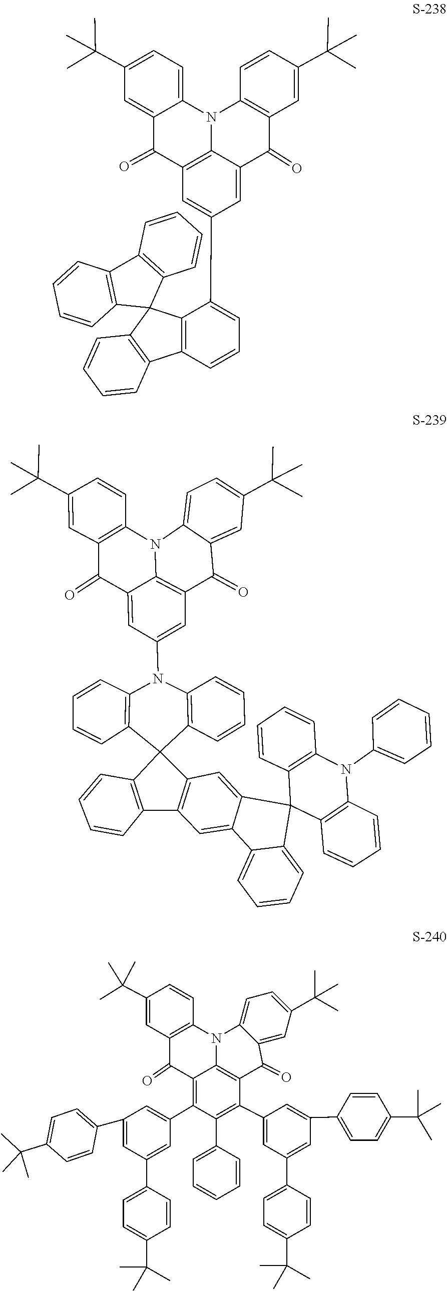

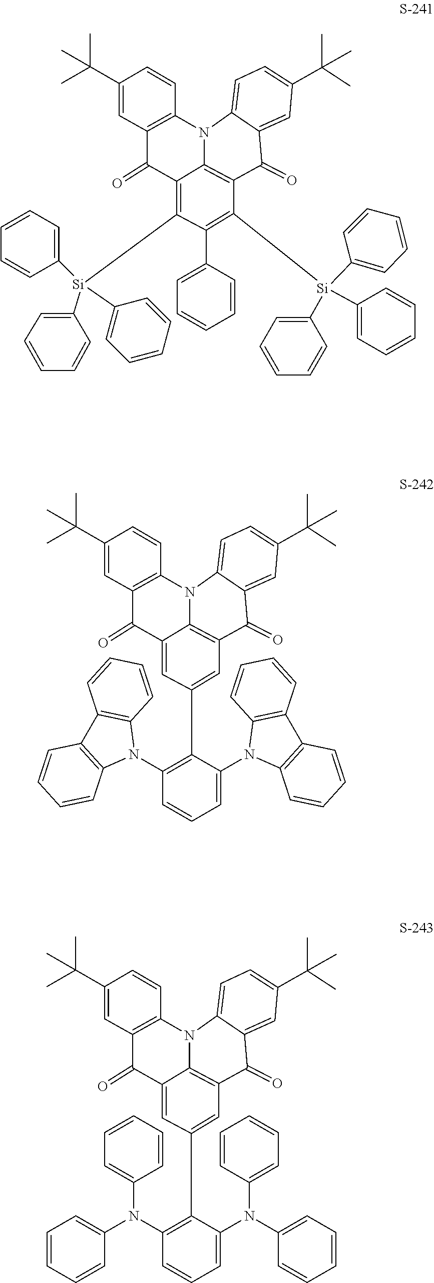 [SMILES: CC(C)(C)c1ccc2c(c1)c(=O)c1c([Si](c3ccccc3)(c3ccccc3)c3ccccc3)c(-c3ccccc3)c([Si](c3ccccc3)(c3ccccc3)c3ccccc3)c3c(=O)c4cc(C(C)(C)C)ccc4n2c13 ; CC(C)(C)c1ccc2c(c1)c(=O)c1cc(-c3c(-n4c5ccccc5c5ccccc54)cccc3-n3c4ccccc4c4ccccc43)cc3c(=O)c4cc(C(C)(C)C)ccc4n2c13 ; CC(C)(C)c1ccc2c(c1)c(=O)c1cc(-c3c(N(c4ccccc4)c4ccccc4)cccc3N(c3ccccc3)c3ccccc3)cc3c(=O)c4cc(C(C)(C)C)ccc4n2c13]

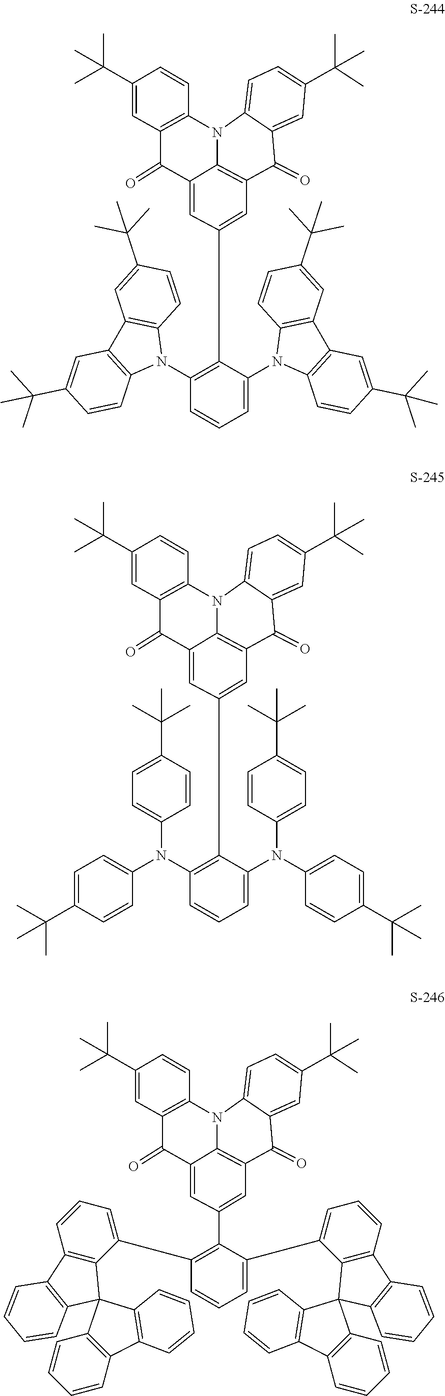

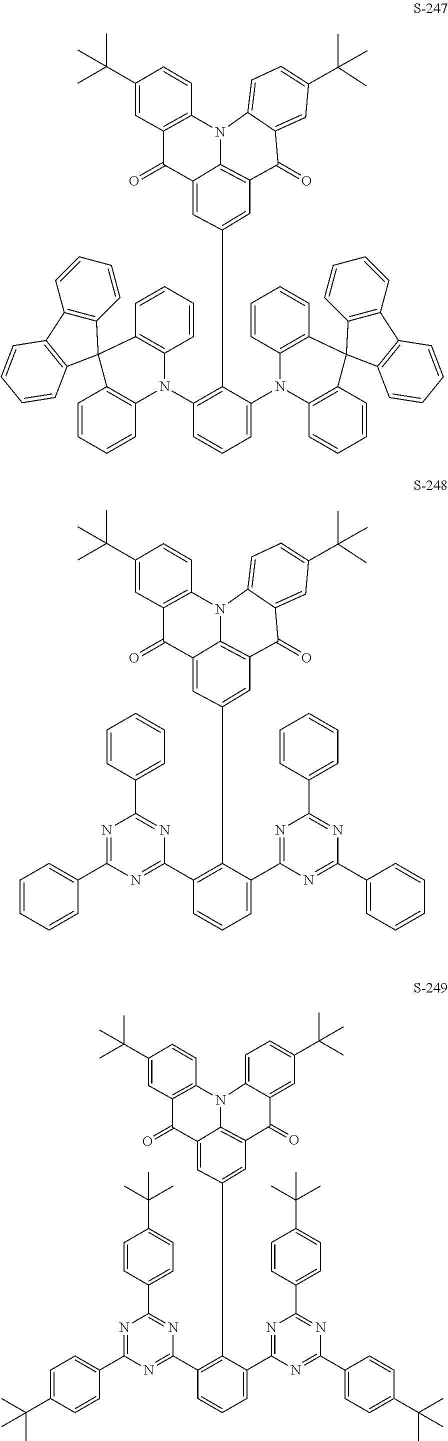 [SMILES: CC(C)(C)c1ccc2c(c1)c(=O)c1cc(-c3c(N4c5ccccc5C5(c6ccccc6-c6ccccc65)c5ccccc54)cccc3N3c4ccccc4C4(c5ccccc5-c5ccccc54)c4ccccc43)cc3c(=O)c4cc(C(C)(C)C)ccc4n2c13 ; CC(C)(C)c1ccc2c(c1)c(=O)c1cc(-c3c(-c4nc(-c5ccccc5)nc(-c5ccccc5)n4)cccc3-c3nc(-c4ccccc4)nc(-c4ccccc4)n3)cc3c(=O)c4cc(C(C)(C)C)ccc4n2c13 ; CC(C)(C)c1ccc(-c2nc(-c3ccc(C(C)(C)C)cc3)nc(-c3cccc(-c4nc(-c5ccc(C(C)(C)C)cc5)nc(-c5ccc(C(C)(C)C)cc5)n4)c3-c3cc4c(=O)c5cc(C(C)(C)C)ccc5n5c6ccc(C(C)(C)C)cc6c(=O)c(c3)c45)n2)cc1]

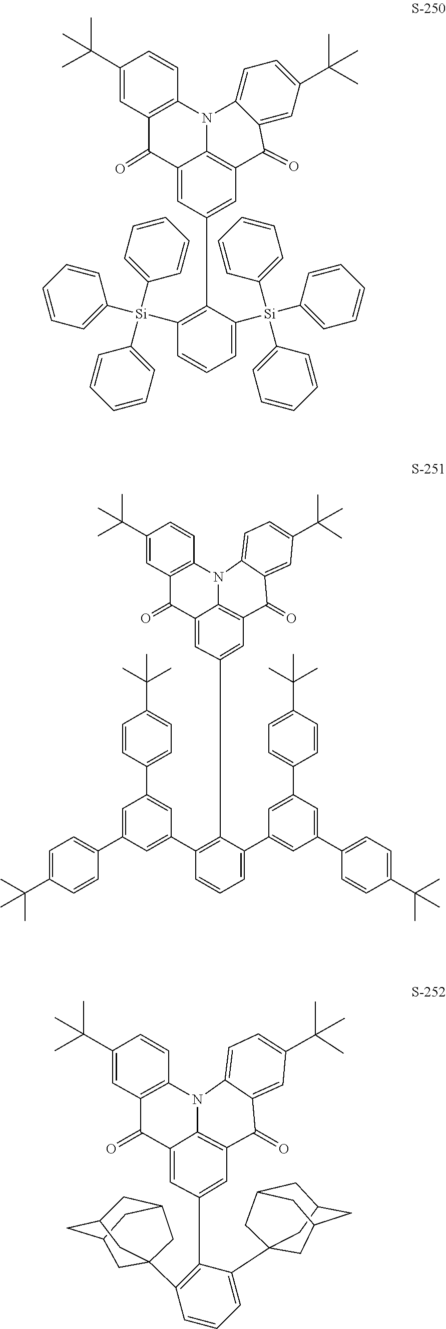

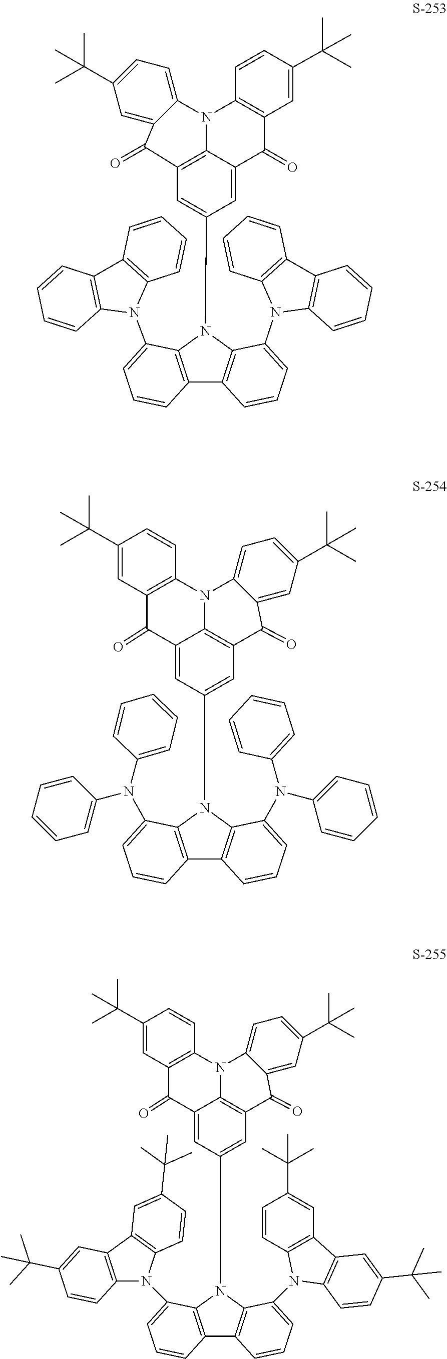 [SMILES: CC(C)(C)c1ccc2c(c1)c(=O)c1cc(-n3c4c(-n5c6ccccc6c6ccccc65)cccc4c4cccc(-n5c6ccccc6c6ccccc65)c43)cc3c(=O)c4cc(C(C)(C)C)ccc4n2c13 ; CC(C)(C)c1ccc2c(c1)c(=O)c1cc(-n3c4c(N(c5ccccc5)c5ccccc5)cccc4c4cccc(N(c5ccccc5)c5ccccc5)c43)cc3c(=O)c4cc(C(C)(C)C)ccc4n2c13 ; CC(C)(C)c1ccc2c(c1)c(=O)c1cc(-n3c4c(-n5c6ccc(C(C)(C)C)cc6c6cc(C(C)(C)C)ccc65)cccc4c4cccc(-n5c6ccc(C(C)(C)C)cc6c6cc(C(C)(C)C)ccc65)c43)cc3c(=O)c4cc(C(C)(C)C)ccc4n2c13]

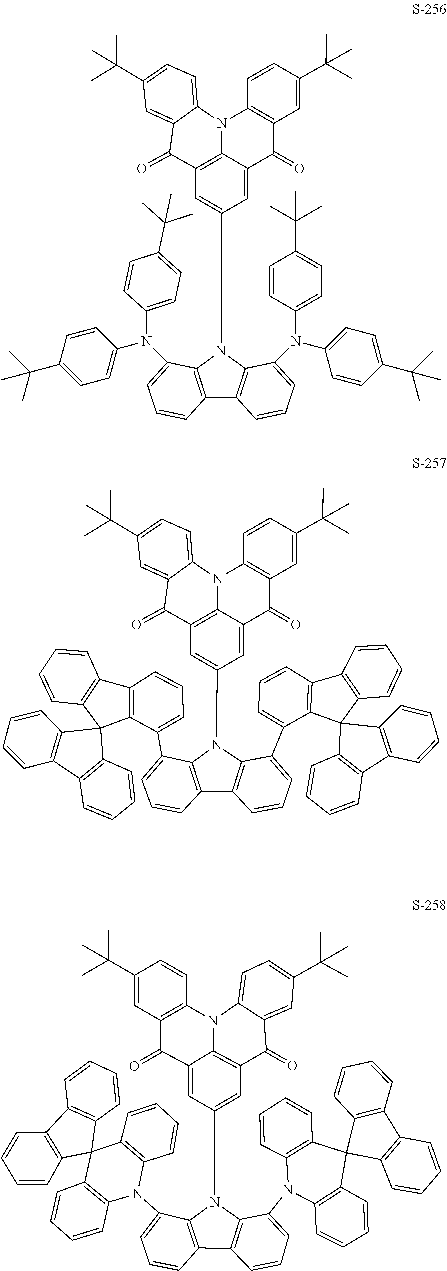

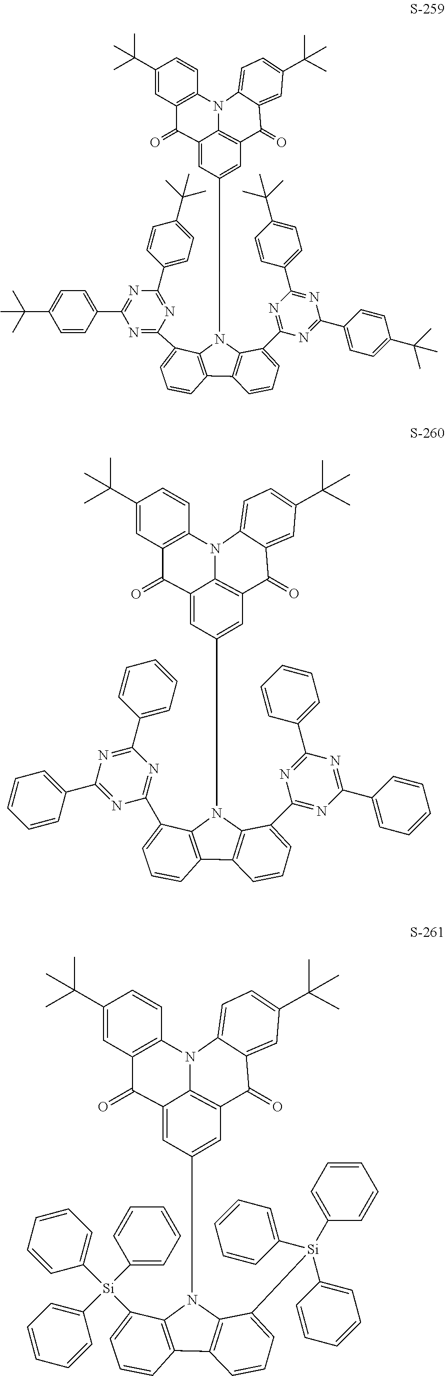 [SMILES: CC(C)(C)c1ccc(-c2nc(-c3ccc(C(C)(C)C)cc3)nc(-c3cccc4c5cccc(-c6nc(-c7ccc(C(C)(C)C)cc7)nc(-c7ccc(C(C)(C)C)cc7)n6)c5n(-c5cc6c(=O)c7cc(C(C)(C)C)ccc7n7c8ccc(C(C)(C)C)cc8c(=O)c(c5)c67)c34)n2)cc1 ; CC(C)(C)c1ccc2c(c1)c(=O)c1cc(-n3c4c(-c5nc(-c6ccccc6)nc(-c6ccccc6)n5)cccc4c4cccc(-c5nc(-c6ccccc6)nc(-c6ccccc6)n5)c43)cc3c(=O)c4cc(C(C)(C)C)ccc4n2c13 ; CC(C)(C)c1ccc2c(c1)c(=O)c1cc(-n3c4c([Si](c5ccccc5)(c5ccccc5)c5ccccc5)cccc4c4cccc([Si](c5ccccc5)(c5ccccc5)c5ccccc5)c43)cc3c(=O)c4cc(C(C)(C)C)ccc4n2c13]

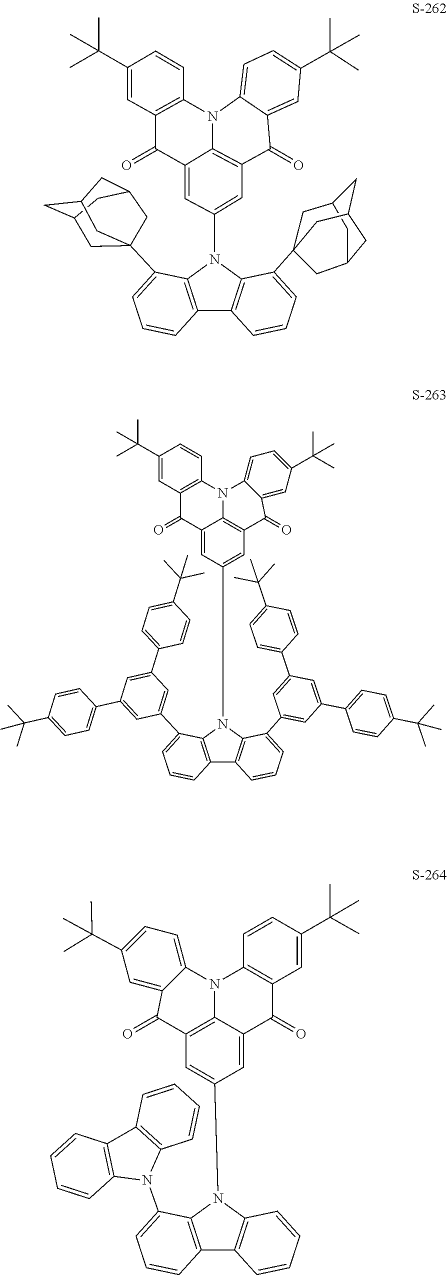 [SMILES: CC(C)(C)c1ccc2c(c1)c(=O)c1cc(-n3c4c(C56CC7CC(CC(C7)C5)C6)cccc4c4cccc(C56CC7CC(CC(C7)C5)C6)c43)cc3c(=O)c4cc(C(C)(C)C)ccc4n2c13 ; CC(C)(C)c1ccc(-c2cc(-c3ccc(C(C)(C)C)cc3)cc(-c3cccc4c5cccc(-c6cc(-c7ccc(C(C)(C)C)cc7)cc(-c7ccc(C(C)(C)C)cc7)c6)c5n(-c5cc6c(=O)c7cc(C(C)(C)C)ccc7n7c8ccc(C(C)(C)C)cc8c(=O)c(c5)c67)c34)c2)cc1 ; CC(C)(C)c1ccc2c(c1)c(=O)c1cc(-n3c4ccccc4c4cccc(-n5c6ccccc6c6ccccc65)c43)cc3c(=O)c4cc(C(C)(C)C)ccc4n2c13]

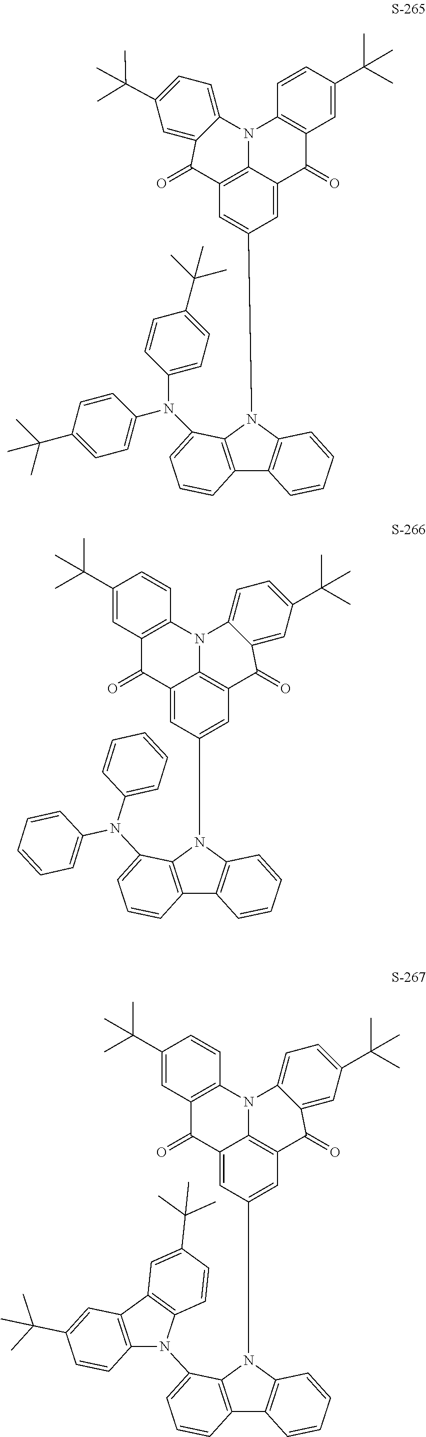

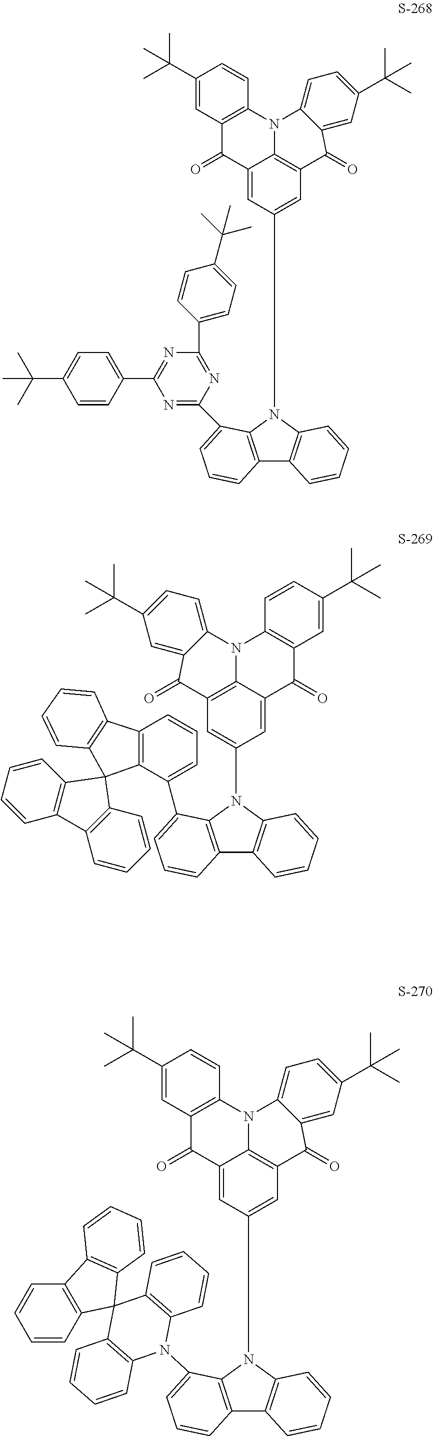

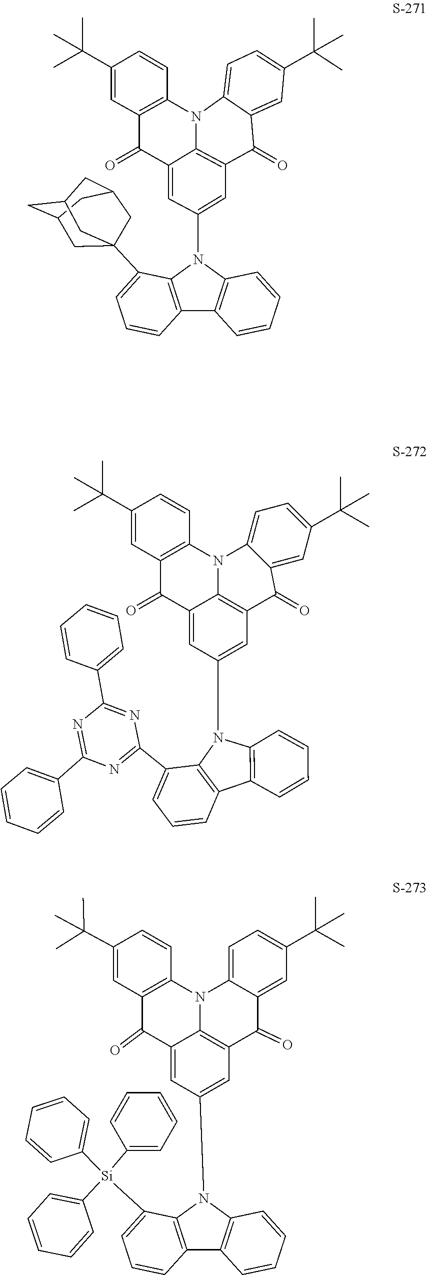 [SMILES: CC(C)(C)c1ccc2c(c1)c(=O)c1cc(-n3c4ccccc4c4cccc(C56CC7CC(CC(C7)C5)C6)c43)cc3c(=O)c4cc(C(C)(C)C)ccc4n2c13 ; CC(C)(C)c1ccc2c(c1)c(=O)c1cc(-n3c4ccccc4c4cccc(-c5nc(-c6ccccc6)nc(-c6ccccc6)n5)c43)cc3c(=O)c4cc(C(C)(C)C)ccc4n2c13 ; CC(C)(C)c1ccc2c(c1)c(=O)c1cc(-n3c4ccccc4c4cccc([Si](c5ccccc5)(c5ccccc5)c5ccccc5)c43)cc3c(=O)c4cc(C(C)(C)C)ccc4n2c13]

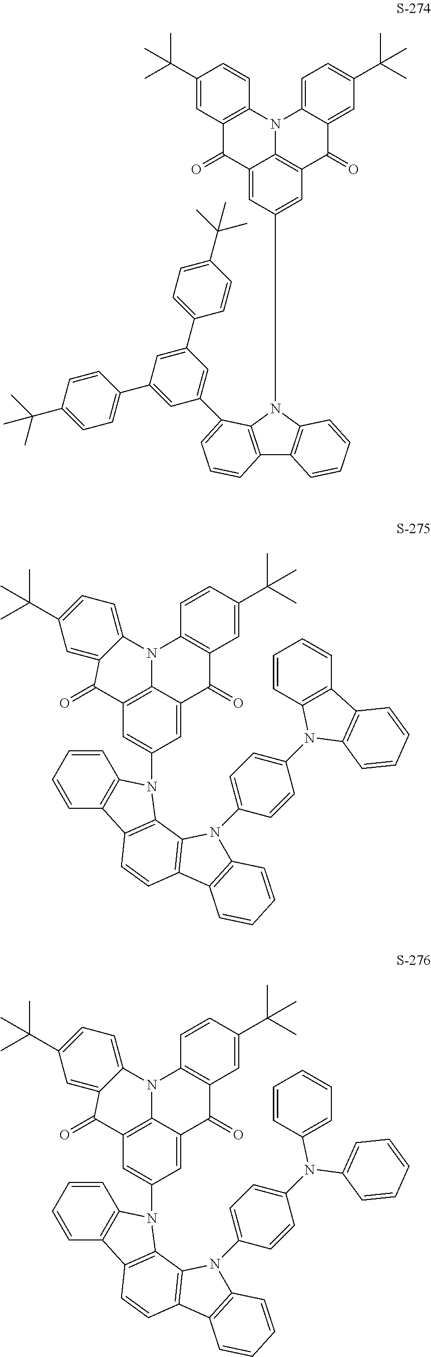

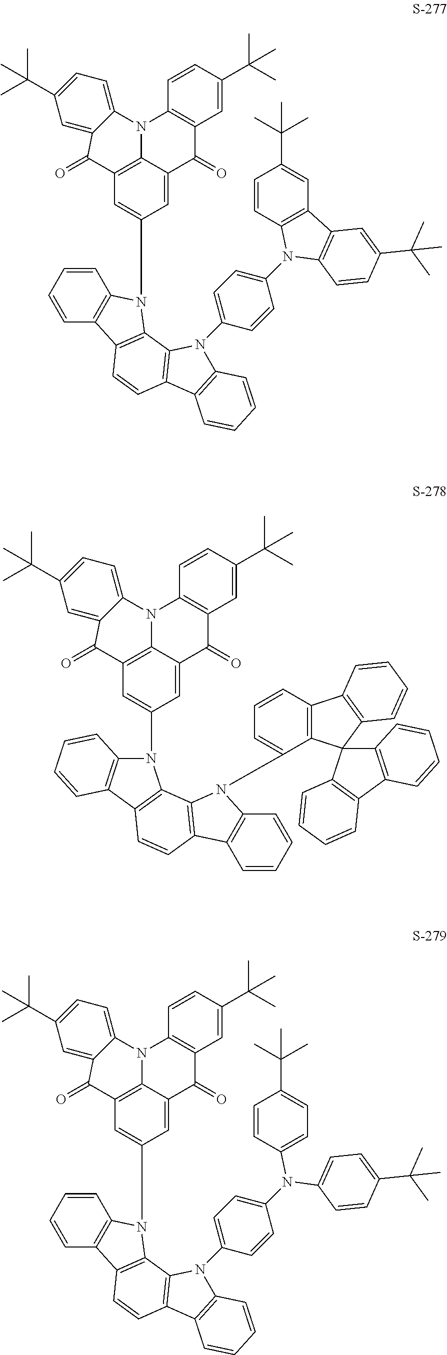

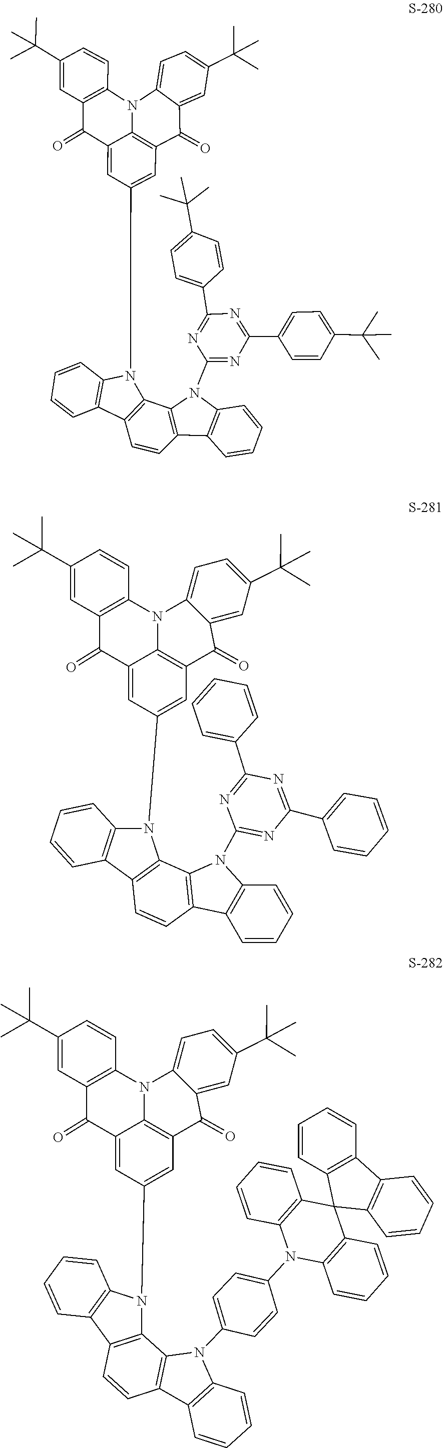

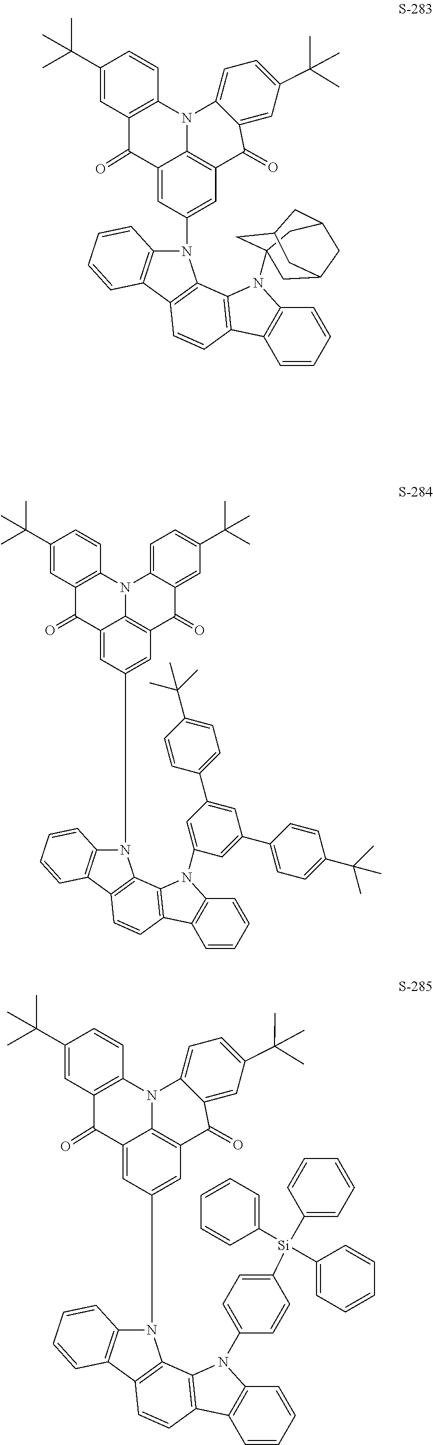

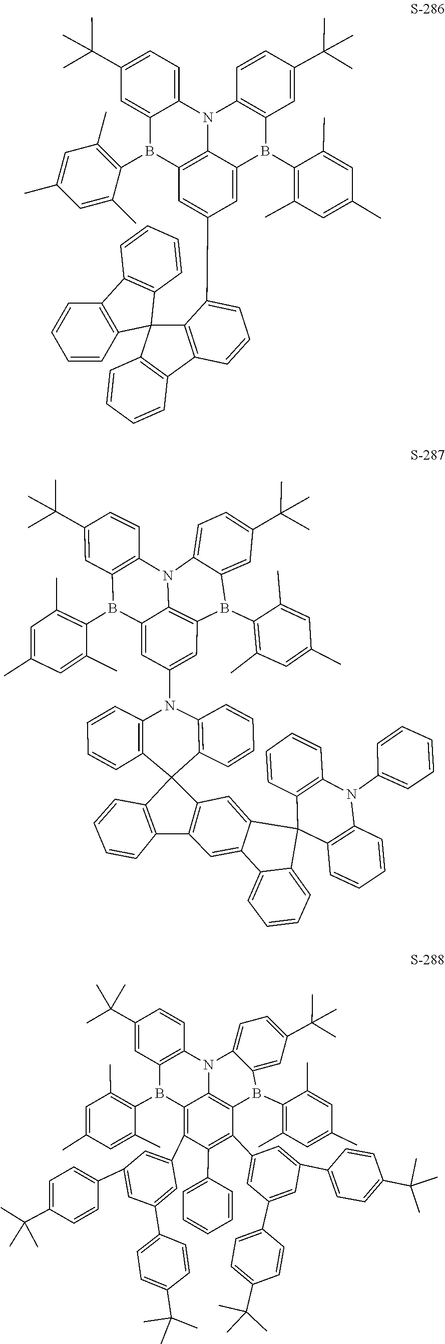

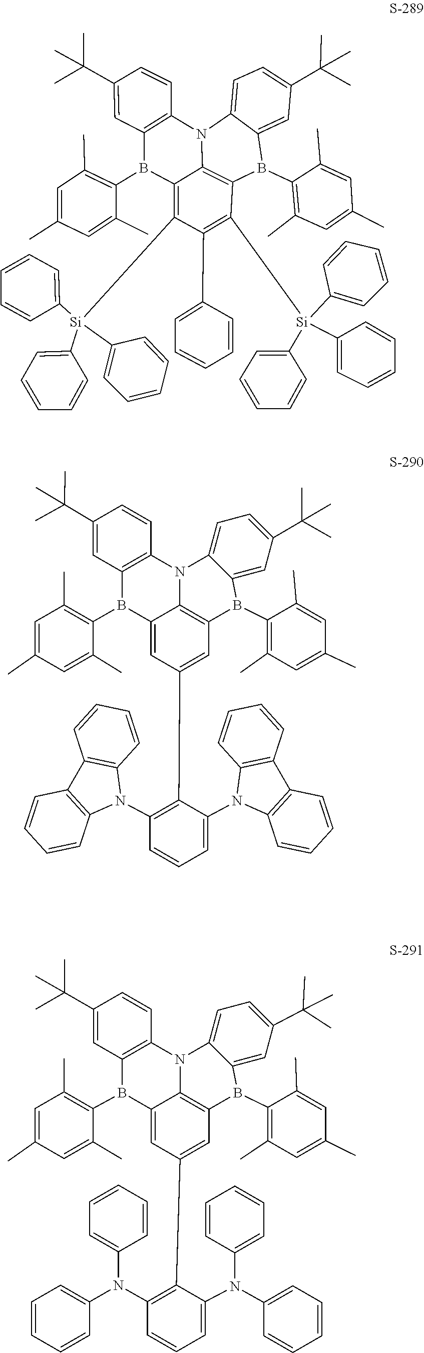

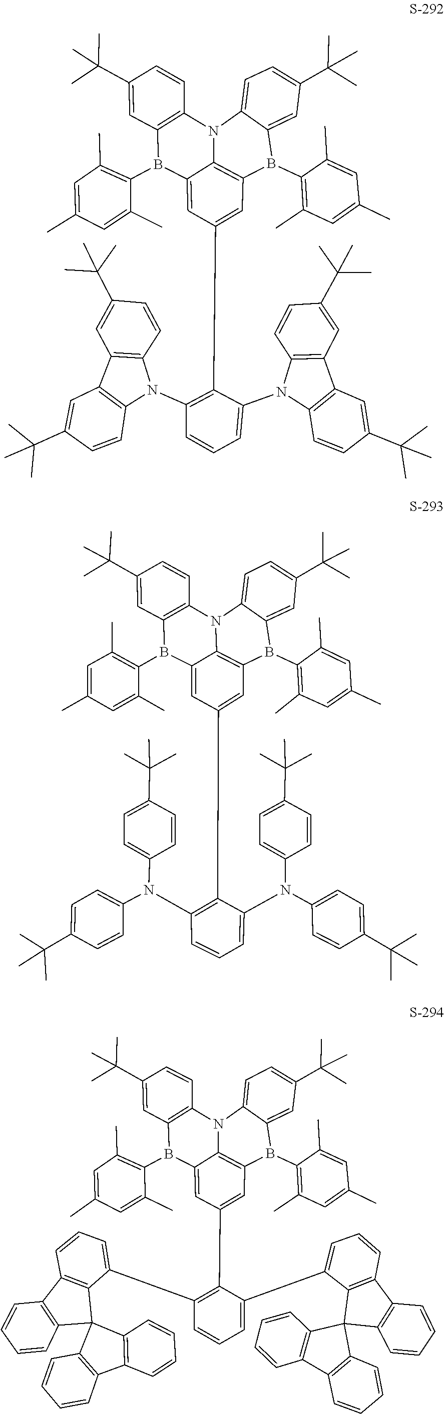

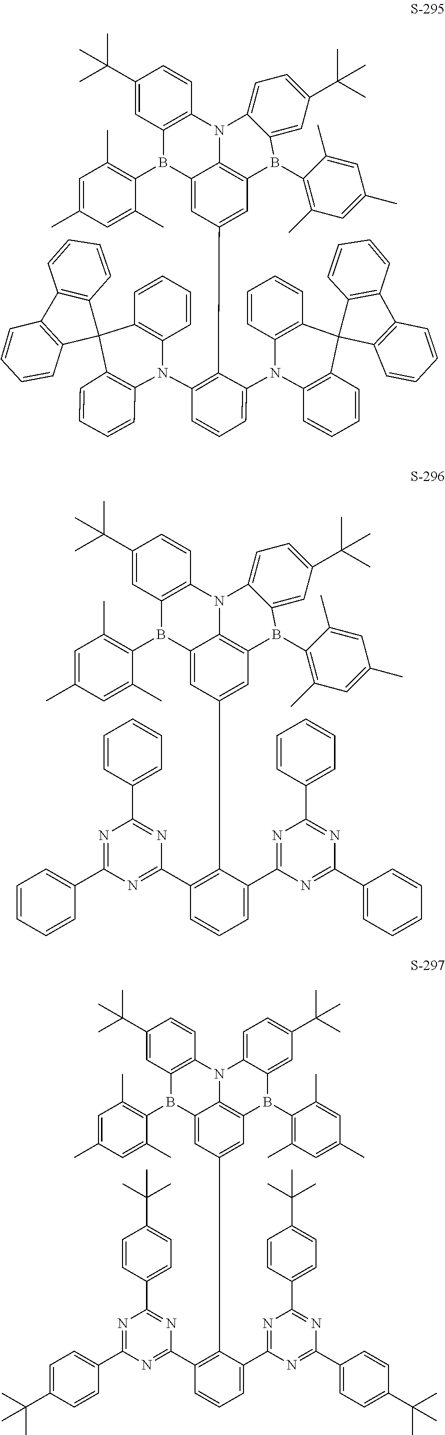

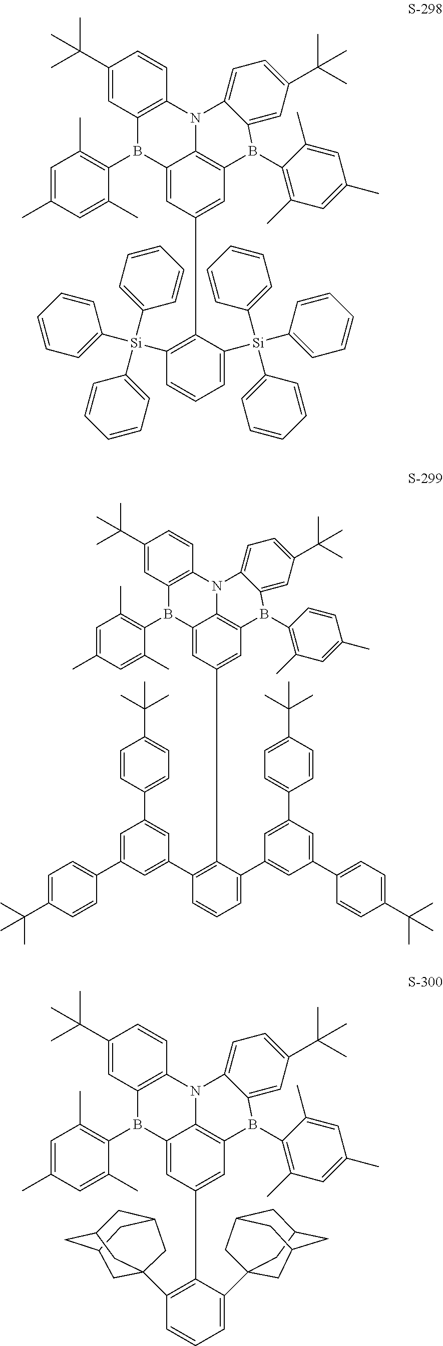

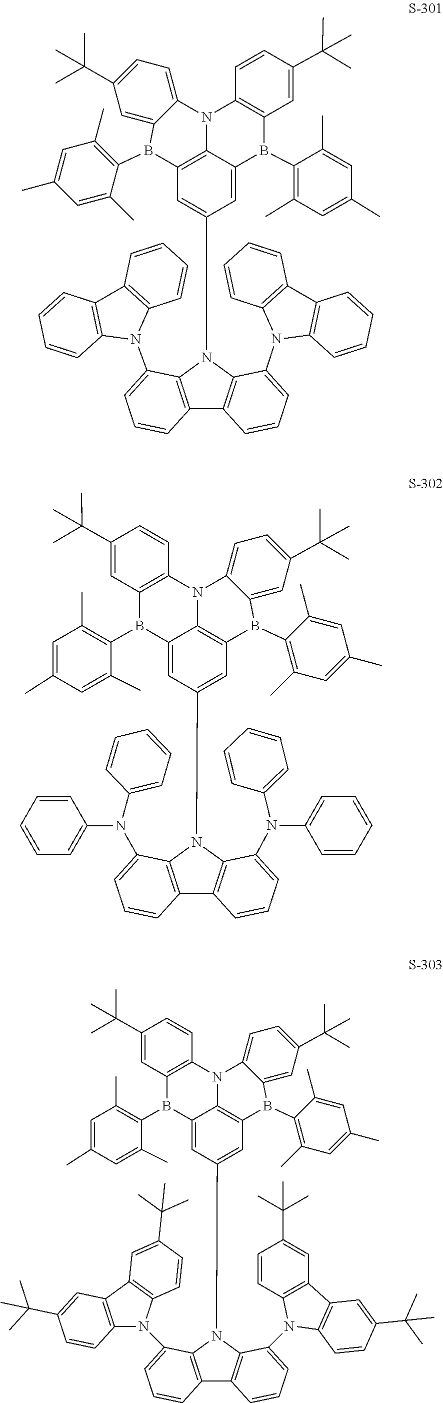

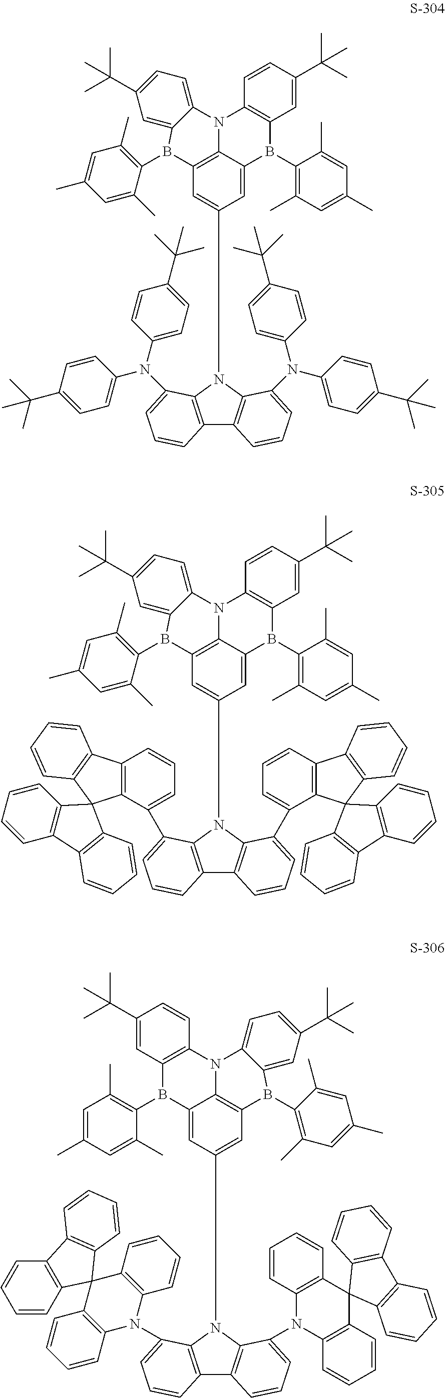

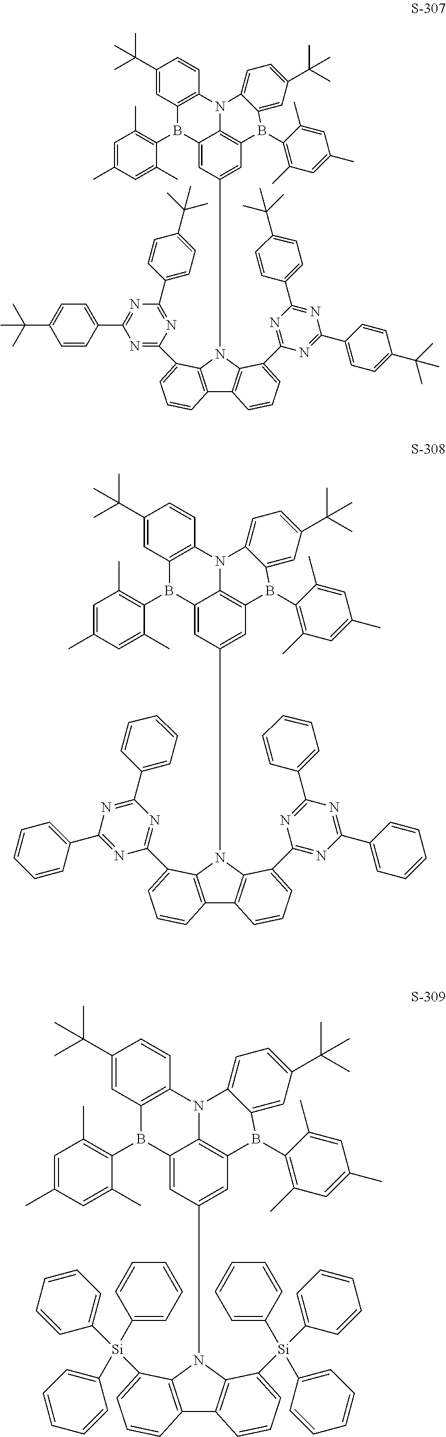

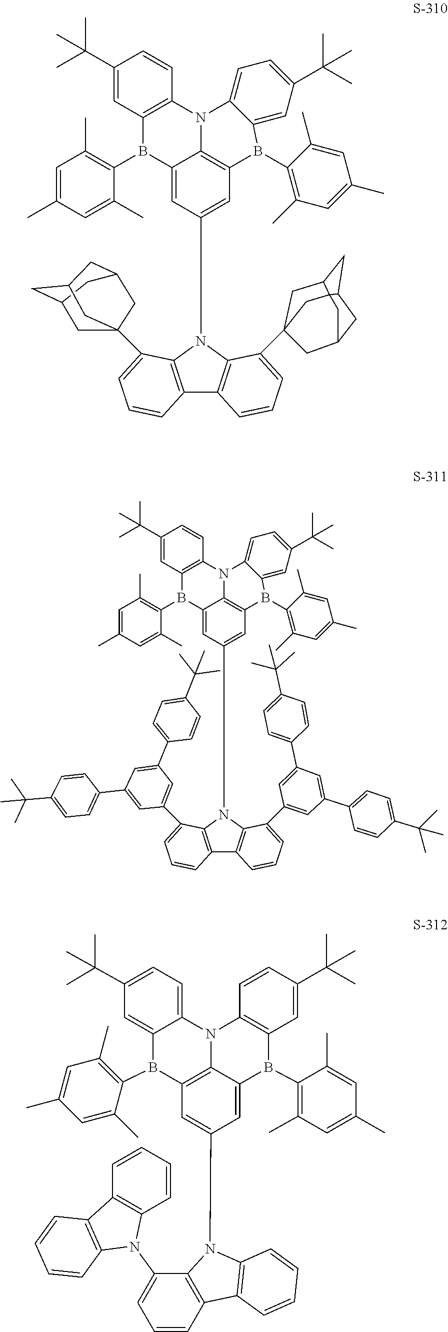

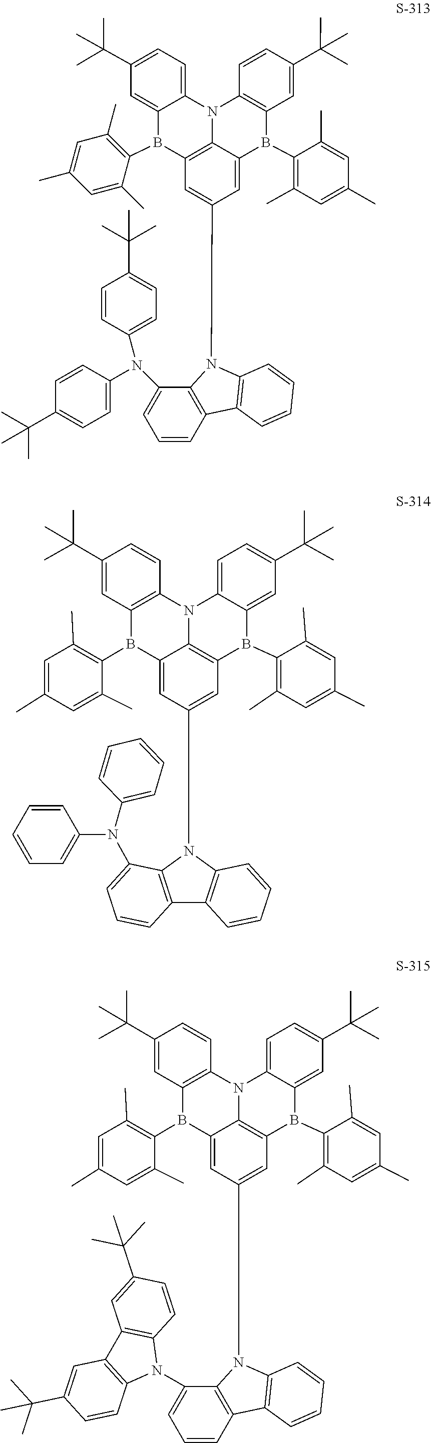

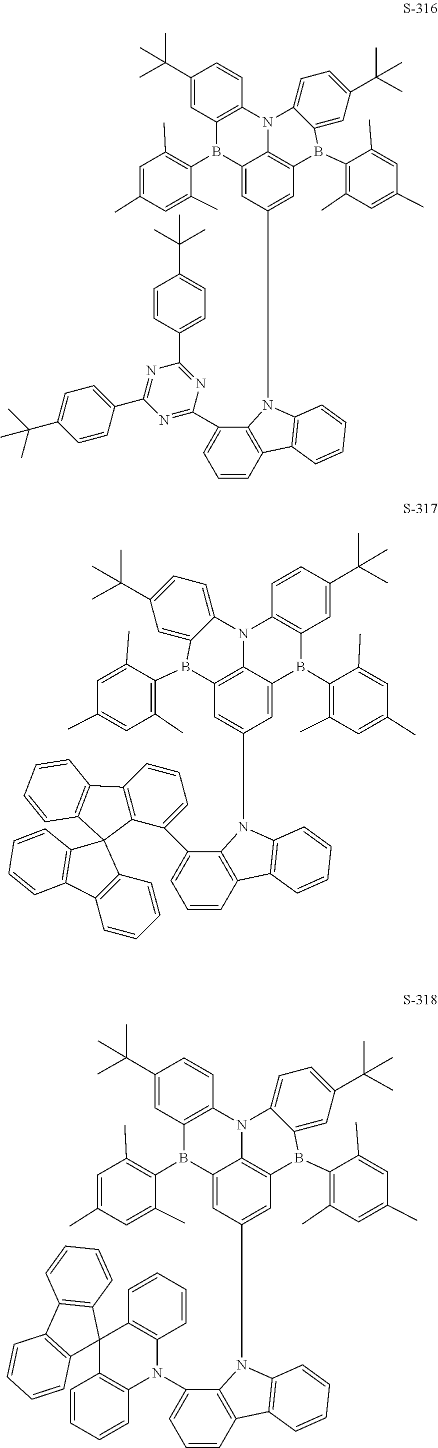 [SMILES: Cc1cc(C)c(B2c3cc(C(C)(C)C)ccc3N3c4ccc(C(C)(C)C)cc4B(c4c(C)cc(C)cc4C)c4cc(-n5c6ccccc6c6cccc(-c7nc(-c8ccc(C(C)(C)C)cc8)nc(-c8ccc(C(C)(C)C)cc8)n7)c65)cc2c43)c(C)c1 ; Cc1cc(C)c(B2c3cc(C(C)(C)C)ccc3N3c4ccc(C(C)(C)C)cc4B(c4c(C)cc(C)cc4C)c4cc(-n5c6ccccc6c6cccc(-c7cccc8c7C7(c9ccccc9-c9ccccc97)c7ccccc7-8)c65)cc2c43)c(C)c1 ; Cc1cc(C)c(B2c3cc(C(C)(C)C)ccc3N3c4ccc(C(C)(C)C)cc4B(c4c(C)cc(C)cc4C)c4cc(-n5c6ccccc6c6cccc(N7c8ccccc8C8(c9ccccc9-c9ccccc98)c8ccccc87)c65)cc2c43)c(C)c1]

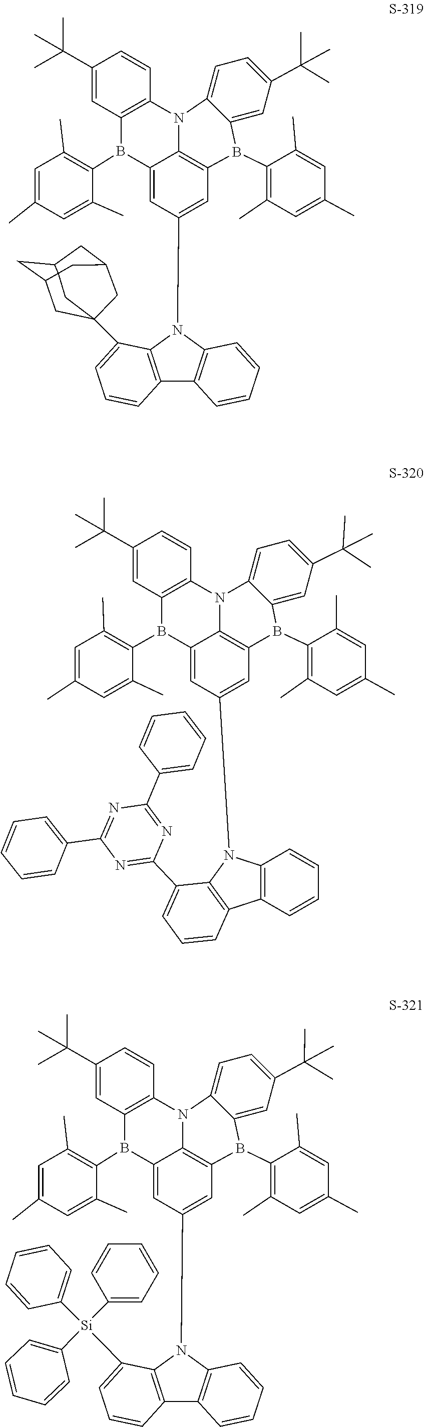

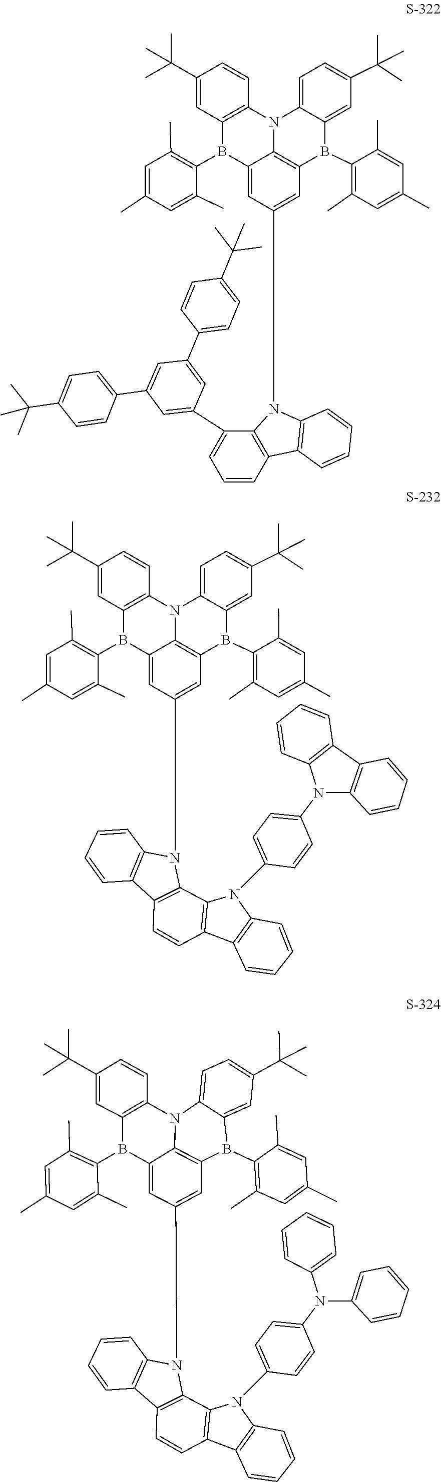

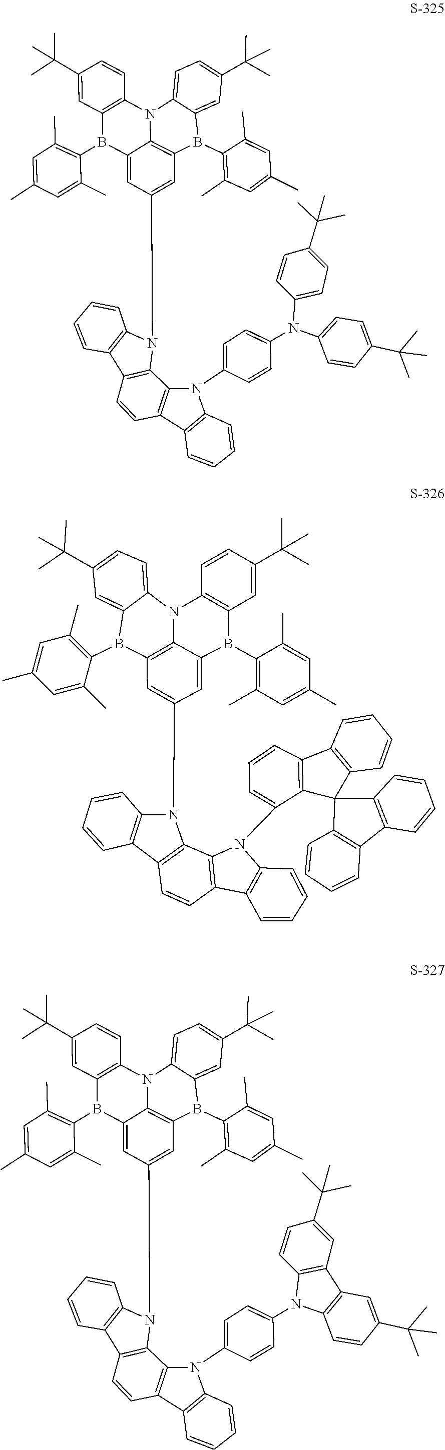

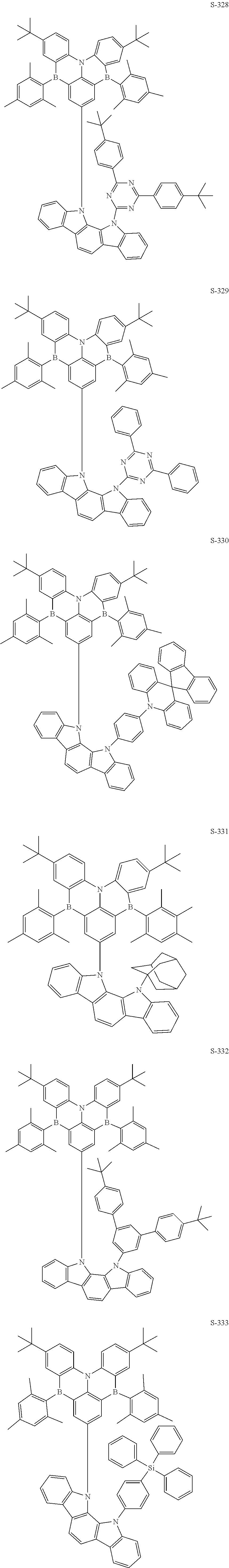

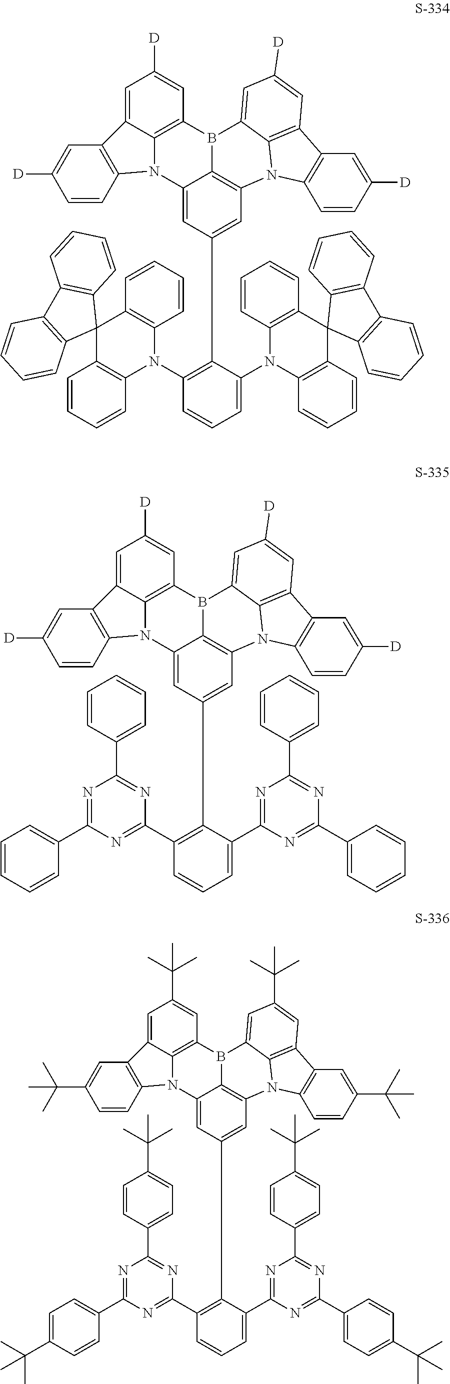

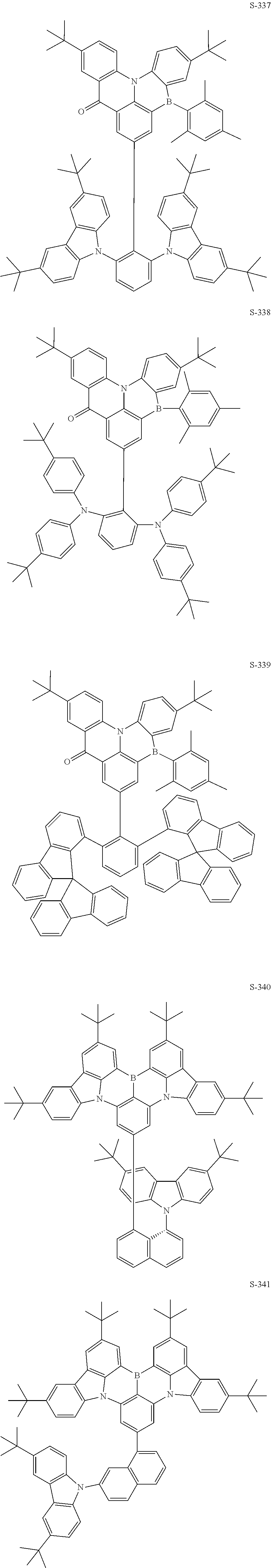

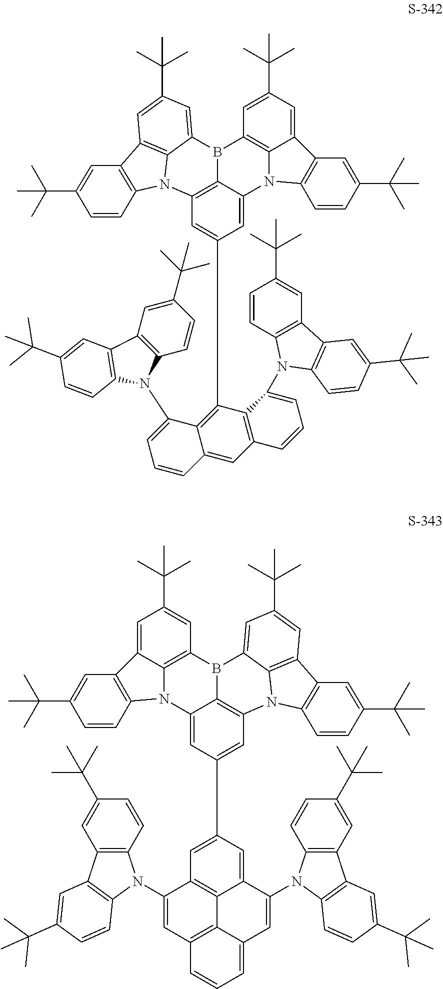

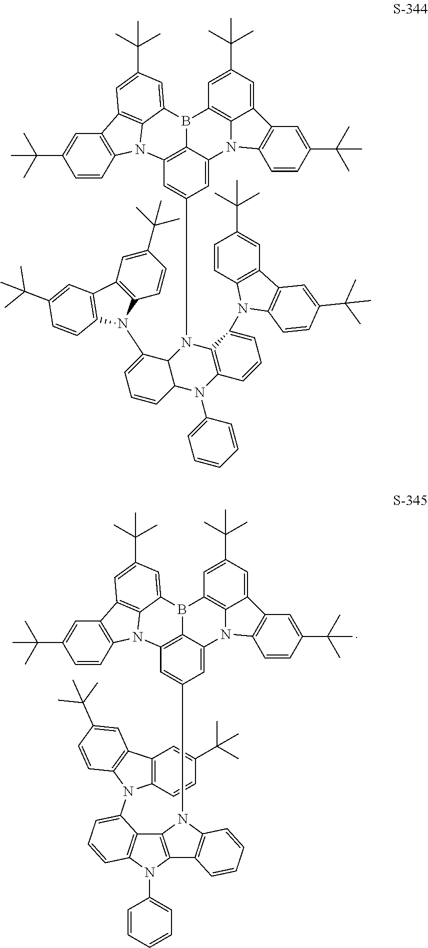

14. The organic electroluminescent device according to claim 1, wherein the triplet-triplet annihilation type host material is selected from a combination of at least one compound represented by BFH-1 to BFH-25:

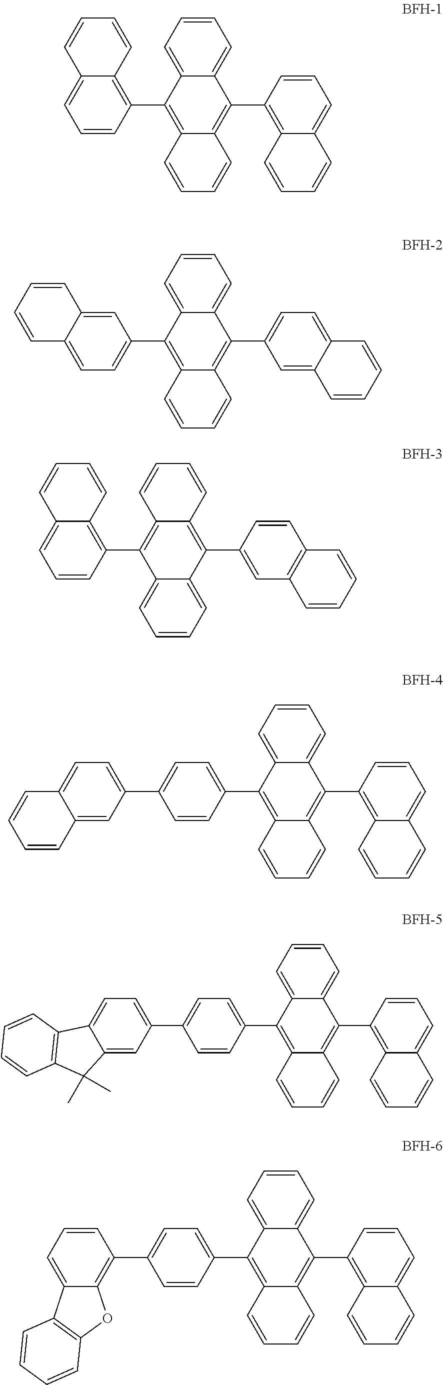

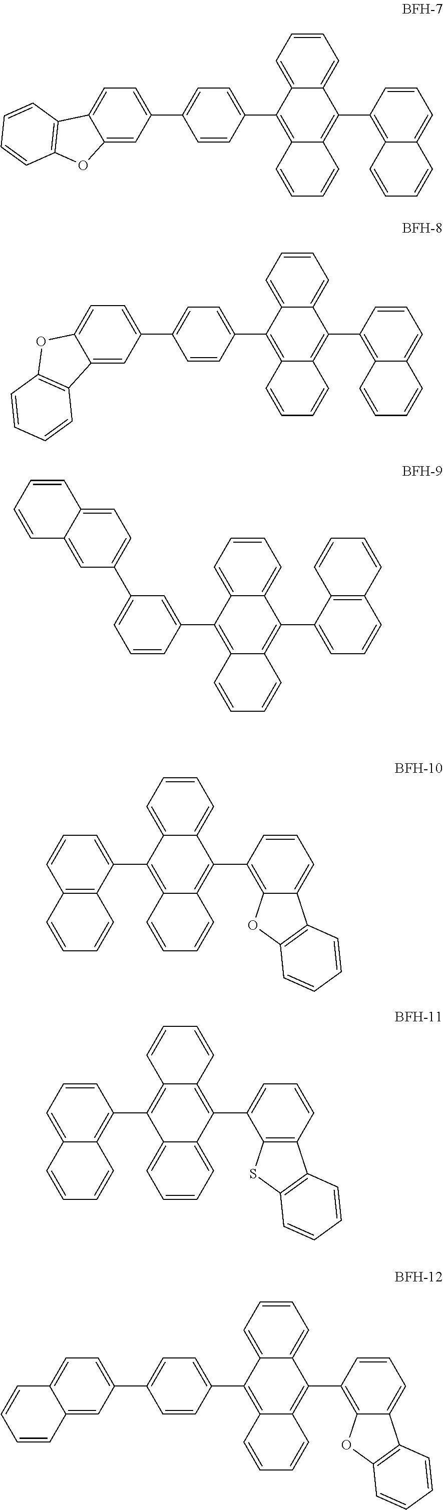

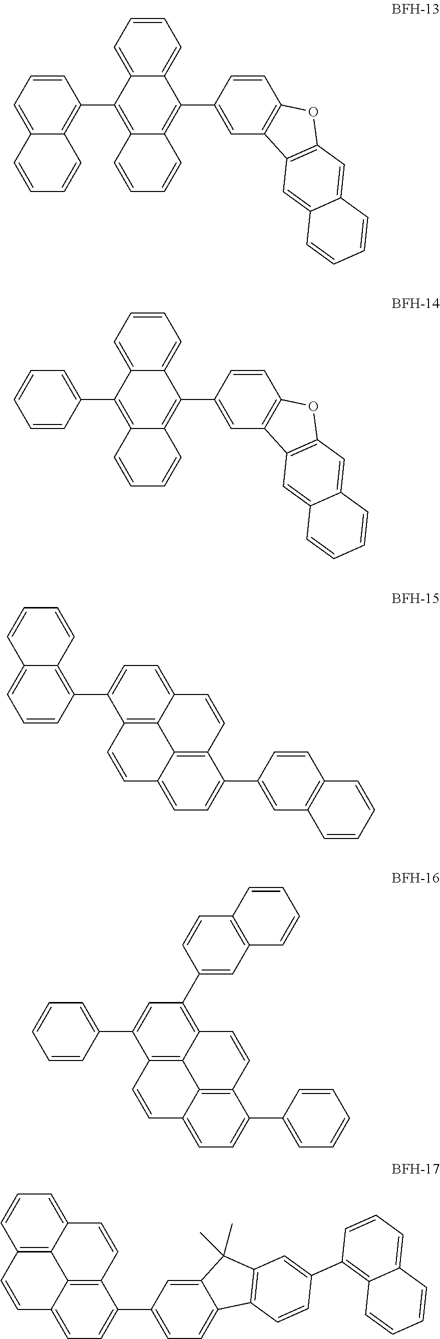

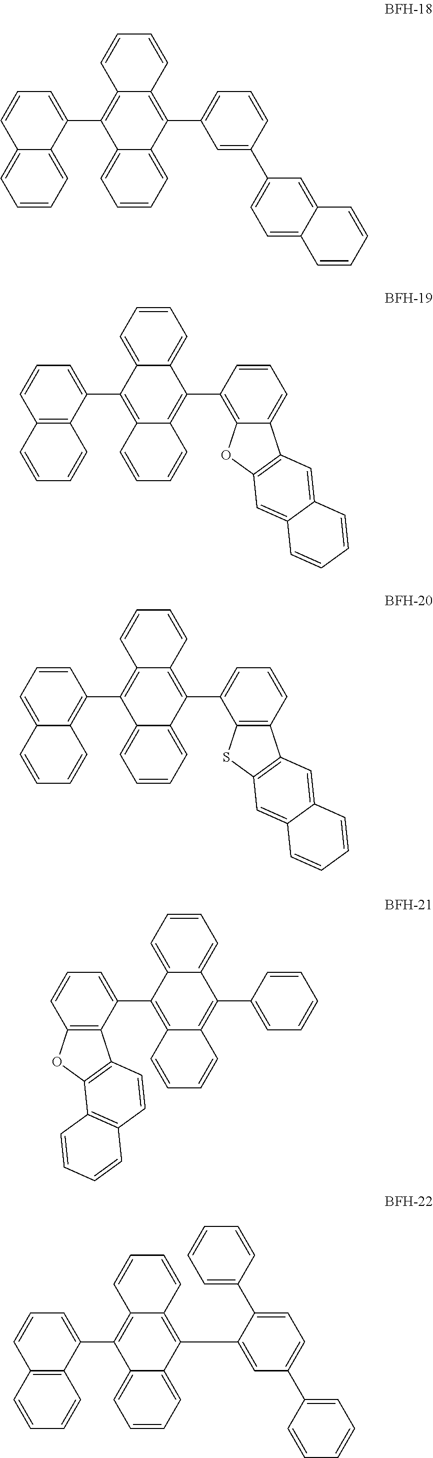

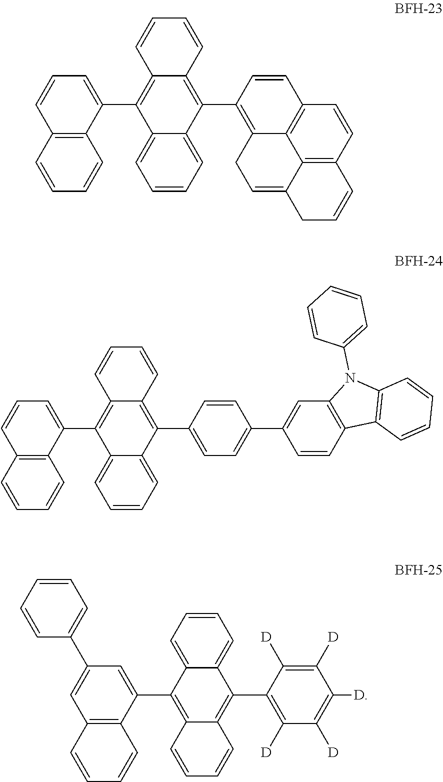

15. The organic electroluminescent device according to claim 1, wherein a mass ratio of the fluorescent dye to the light-emitting layer is 0.1%-50%.

16. The organic electroluminescent device according to claim 2, wherein a mass ratio of the fluorescent dye to the light-emitting layer is 0.1%-50%.

17. The organic electroluminescent device according to claim 3, wherein a mass ratio of the fluorescent dye to the light-emitting layer is 0.1%-50%.

18. The organic electroluminescent device according to claim 4, wherein a mass ratio of the fluorescent dye to the light-emitting layer is 0.1%-50%.

19. The organic electroluminescent device according to claim 15, wherein the mass ratio of the fluorescent dye to the light-emitting layer is 0.5%-20%.

20. A display apparatus, comprising the organic electroluminescent device according to claim 1.
